(19) United States
(12) Patent Application Publication
(10) Pub. No.: US 2025/0277230 A1
(43) Pub. Date: Sep. 4, 2025

(54) PARTHENOGENESIS METHODS AND COMPOSITIONS

(71) Applicant: PIONEER HI-BRED INTERNATONAL, INC., JOHNSTON, IA (US)

(72) Inventors: MAREN L. ARLING, DES MOINES, IA (US); MARISSA SIMON, GRIMES, IA (US)

(73) Assignee: PIONEER HI-BRED INTERNATONAL, INC., JOHNSTON, IA (US)

(21) Appl. No.: 18/857,700

(22) PCT Filed: Apr. 18, 2023
(86) PCT No.: PCT/US23/65923

§ 371 Date: Oct. 17, 2024

Related U.S. Application Data
(60) Provisional application No. 63/363,180, filed on Apr. 19, 2022.

(51) Int. Cl. C12N 15/82; C12N 9/22; C12N 15/11

(52) U.S. Cl. CPC C12N 15/8287; C12N 9/222; C12N 15/11; C12N 15/8205; C12N 2310/20

(57) ABSTRACT

Provided herein are methods and compositions for inducing parthenogenesis by expressing, in a female gametophyte, a Babyboom (BBM) polynucleotide operably linked to a promoter that expresses in an egg cell. Also included herein are promoters that express in a plant egg cell and methods of expressing a gene product in an egg cell of a plant using these promoters.

### CROSS REFERENCE

This application claims the benefit of U.S. Provisional Application No. 63/363,180 filed Apr. 19, 2022, which is incorporated herein by reference in its entirety.

### FIELD

The present disclosure relates to the field of plant molecular biology and plant breeding.

### REFERENCE TO SEQUENCE LISTING SUBMITTED ELECTRONICALLY

The official copy of the sequence listing is submitted electronically via EFS-Web as an XML formatted sequence listing with a file named 8893-WO PCT_SEQ_LIST_ST26.XML created on Apr. 11, 2023, and having a size of 287 kilobytes and is filed concurrently with the specification. The sequence listing comprised in this XML formatted document is part of the specification and is herein incorporated by reference in its entirety.

### BACKGROUND OF THE DISCLOSURE

In plants, germ line cells (germline) provide the transgenerational inheritance of genetic information in each subsequent generation by producing spore mother cells during sporogenesis. For example, sporogenesis provides the megaspore mother cell that develops the female gametes, the egg cell and central cell that give rise to the embryo and endosperm, respectively; or the microspore mother cell that develops the male gamete, giving rise to four haploid microspores, wherein each microspore further develops into a mature pollen grain. A key aspect for the unique role of germline cells is providing the genetic information a future offspring receives, wherein half of the genetic contribution is from the female gamete and half of the genetic contribution is from the male gamete. Fertilization of the egg cell with one sperm cell forms a diploid zygote, while a second sperm cells fuses with the two polar nuclei of the central cell to form a triploid endosperm. The endosperm is a terminally nourishing tissue for the embryo yet does not contribute to the germline. After fertilization, the zygote gives rise to an embryo, a process referred to as zygotic embryogenesis that is characteristic of sexual reproduction. A newly formed embryo undergoing such an embryogenesis developmental program comprising an underlying regulatory program affected by genetic determinants and epigenetic reprogramming leading from an embryogenic cell state to the acquisition of a differentiated cell fate, or cell fates, ultimately giving rise to a plant with all differentiated tissues thereof.

Parthenogenesis is a form of asexual reproduction wherein growth and development of female gametes (embryos) occur without fertilization by sperm. The female gamete produced parthenogenetically may be either haploid or diploid.

### SUMMARY

Described herein are methods of expressing a gene product in an egg cell of a plant, which includes introducing into the plant a polynucleotide comprising (a) a polynucleotide sequence of SEQ ID NO: 37, 38, or 39, (b) a polynucleotide sequence having at least 90% sequence identity to SEQ ID NO: 37, 38, or 39; (c) a fragment of the polynucleotide of (a) or (b), where the fragment retains its ability to drive expression of a heterologous polynucleotide sequence in an egg cell, where the polynucleotide sequence drives expression of an operably linked heterologous polynucleotide sequence. In some aspects, the operably linked heterologous polynucleotide sequence is a Babyboom (BBM) polynucleotide that encodes a BBM polypeptide that, when expressed in a female gametophyte, induces haploid induction. Also included herein are plants that include a promoter operably linked to a heterologous polynucleotide sequence, where the promoter is selected from (a) a polynucleotide sequence of SEQ ID NO: 37, 38, or 39, (b) a polynucleotide sequence having at least 90% sequence identity to SEQ ID NO: 37, 38, or 39, (c) a fragment of the polynucleotide of (a) or (b), where the fragment retains its ability to drive expression of the heterologous polynucleotide sequence in an egg cell of a plant.

Also described herein are methods of inducing parthenogenesis, where the method includes expressing, in a female gametophyte, a Babyboom (BBM) polynucleotide operably linked to a promoter that expresses in a plant egg cell, where the Babyboom (BBM) polynucleotide includes a nucleotide sequence encoding a Babyboom (BBM) polypeptide or a fragment thereof, where the female gametophyte is rendered parthenogenic and forms a haploid embryo without pollination, and where the Babyboom (BBM) polynucleotide is not naturally expressed in a female gametophyte. In some aspects, the Babyboom (BBM) polynucleotide or polypeptide or fragment thereof is a BBM1, BBM2, BMN2, BMN3, ODP2, and BBML polynucleotide or polypeptide or fragment thereof. In some aspects, the promoter that expresses in the plant egg cell is an egg cell-specific promoter or an egg cell-preferred promoter.

In some aspects, the female gametophyte has not been fertilized prior to or during expression of the parthenogenesis factor, e.g. the BBM polynucleotide. In some aspects, BBM is expressed in the egg cell prior to fertilization to induce parthenogenesis, the development of a zygote/embryo in the absence of fertilization. In some aspects, the methods include modifying a regulatory region of an endogenous Babyboom (BBM) polynucleotide so that the Babyboom (BBM) polynucleotide expresses in a female gametophyte. In some aspects, the methods include expressing the Babyboom (BBM) polynucleotide from a modified endogenous genomic BBM locus, where the modified endogenous genomic BBM locus includes a modified regulatory region of an endogenous polynucleotide encoding a BBM polypeptide, where one or more nucleotides in the regulatory region have been modified so that the BBM polypeptide expresses in a female gametophyte. In some aspects, the methods include expressing the Babyboom (BBM) polynucleotide from a modified endogenous genomic egg cell locus, where the egg cell's coding or genomic sequence in the endogenous genomic egg cell locus has been modified so that it encodes a BBM polypeptide, where the BBM polypeptide expresses in a female gametophyte. In some aspects, the female gametophyte is a monocot or dicot female gametophyte. In some aspects, the female gametophyte is wheat, maize, rice, oats, barley, triticale, sorghum, canola, Arabidopsis, cotton, sunflower, safflower, tobacco, cannabis, sugarcane, soy, turf grass, rye, millet, or a flax female gametophyte. In some aspects, the methods include (a) contacting the haploid embryo with a chromosome doubling agent for a period sufficient to generate a doubled haploid embryo, (b) isolating the doubled haploid embryo, and (c) regenerating a doubled haploid plant from the doubled haploid embryo of step (b). In some aspects, the methods include (a) regenerating a parthenogenic plant from a haploid embryo comprising the Babyboom (BBM) polynucleotide operably linked to the promoter that expresses in an egg cell; (b) pollinating the parthenogenic plant of (a) with pollen from a non-haploid inducer; and (c) rescuing a haploid embryo from the parthenogenic plant of (b).

Also included herein are methods for obtaining a wheat plant producing clonal, non-reduced, non-recombined gametes. In some aspects, the method includes (a) suppressing in a wheat plant cell the activity of: (1) all endogenous Spo11 or Prd1, Prd2, or Prd3 polynucleotides or polypeptides; (2) all endogenous Rec8 polynucleotides or polypeptides; (3) all endogenous Osd1 polynucleotides or polypeptides; (b) expressing, in a female gametophyte derived from the wheat plant cell, a Babyboom (BBM) polynucleotide operably linked to a promoter that expresses in an egg cell, where the Babyboom (BBM) polynucleotide comprises a nucleotide sequence encoding a Babyboom (BBM) polypeptide or a fragment thereof, where the female gametophyte is rendered parthenogenic and forms an embryo; and (c) obtaining a plant from the embryo, where the embryo comprises the egg cell expressed Babyboom (BBM) polynucleotide and suppressed Spo11, Rec8, Osd1, Prd1, Prd2, or Prd3 polynucleotides or polypeptides, thereby producing a wheat plant producing clonal, non-reduced, non-recombined gametes.

Also described herein are methods of obtaining a clonal apomictic plant from one or more gametophytic cells in a plant in the absence of egg cell fertilization. In some aspects, the methods include (a) expressing, in one or more gametophytic cell, a Babyboom (BBM) polynucleotide operably linked to a promoter that expresses in an egg cell, where the Babyboom (BBM) polynucleotide comprises a nucleotide sequence encoding a Babyboom (BBM) polypeptide or a fragment thereof that retains haploid induction activity, where the Babyboom (BBM) polynucleotide is not naturally expressed in a female gametophyte, (b) developing an embryo from the gametophytic cell in the absence of egg cell fertilization; and (c) obtaining a progeny plant from one or more gametophytic cells where the progeny plant contains the chromosomes from the gametophytic cell of (a), thereby achieving propagation of a flowering plant in the absence of egg cell fertilization. In some aspects, in the methods and compositions described herein, the plant is a monocot or dicot plant. In some aspects,, in the methods and compositions described herein, the plant is a wheat, cotton, sunflower, safflower, tobacco, Arabidopsis, soy, barley, oats, rice, maize, triticale, sorghum, canola, cannabis, sugarcane, rye, millet, turf grass, or a flax plant.

### DESCRIPTION OF THE FIGURES

FIG. 1 shows an alignment of BBM peptide sequences. (Consensus=SEQ ID NO: 112, TA-BBM6A=SEQ ID NO. 8, OS-BBM2=SEQ ID NO: 25, SV-BBM2=SEQ ID NO: 29; ZM-BBM2=SEQ ID NO: 21; TA-BBM3A=SEQ ID NO: 2; OS-ODP2=SEQ ID NO: 23; SV-BBM=SEQ ID NO: 29; ZM-ODP2=SEQ ID NO: 14). Some of the A motifs in the sequences in FIG. 1 are set forth in SEQ ID NOs: 107, 109, 116, 117, and 118. Some of the B motifs in the sequences in FIG. 1 are set forth in SEQ ID NOs: 108, 110, 119, 120, and 121. Some of the AP2 DNA binding domains in the sequences in FIG. 1 are set forth in SEQ ID NOs: 111 and 122.

FIG. 2 shows an alignment of wheat TA-BBM native and modified peptide sequences. (Consensus=SEQ ID NO: 113; TA-BBM3A=SEQ ID NO. 2; TA-BBM3A (ALT1)=SEQ ID NO: 42; TA-BBM3A (ALT2)=SEQ ID NO: 44; TA-BBM6A=SEQ ID NO: 8; TA-BBM6A (ALT1)=SEQ ID NO: 46; BBM6A (ALT2)=SEQ ID NO: 48). The A motifs in the sequences in FIG. 2 are set forth in SEQ ID NOs: 107 and 109. The B motifs in the sequences in FIG. 2 are set forth in SEQ ID NOs: 108 and 110. The conserved AP2 DNA binding domain in the sequences in FIG. 2 is set forth in SEQ ID NOs: 111.

### BRIEF DESCRIPTION OF THE SEQUENCE LISTING

The disclosure can be more fully understood from the following detailed description and the accompanying Sequence Listing which form a part of this application.

Table A presents SEQ ID NOs for various polynucleotide and polypeptide sequences. It is understood, as those skilled in the art will appreciate, that the disclosure encompasses more than these specific exemplary sequences.

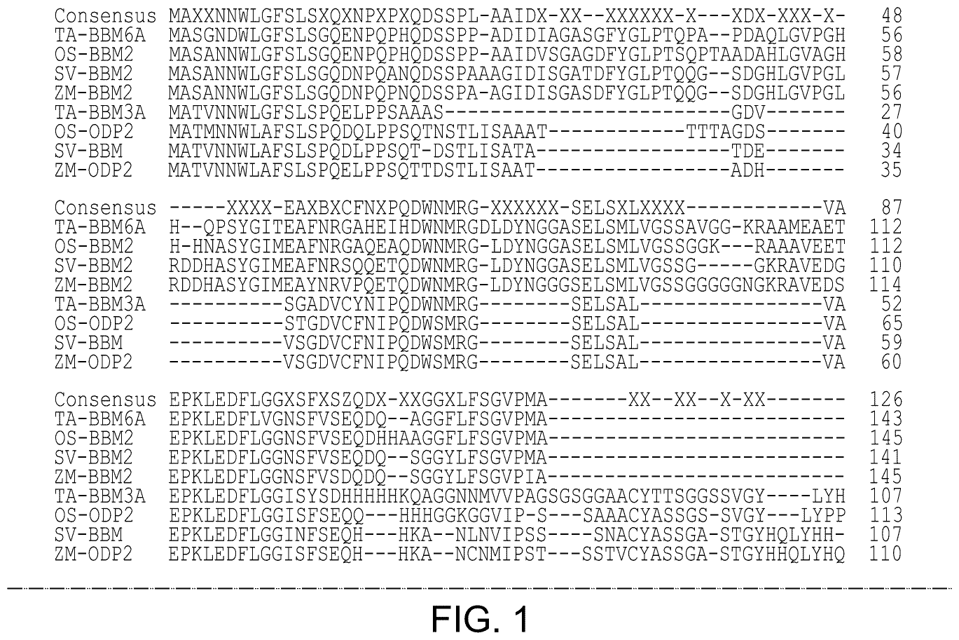

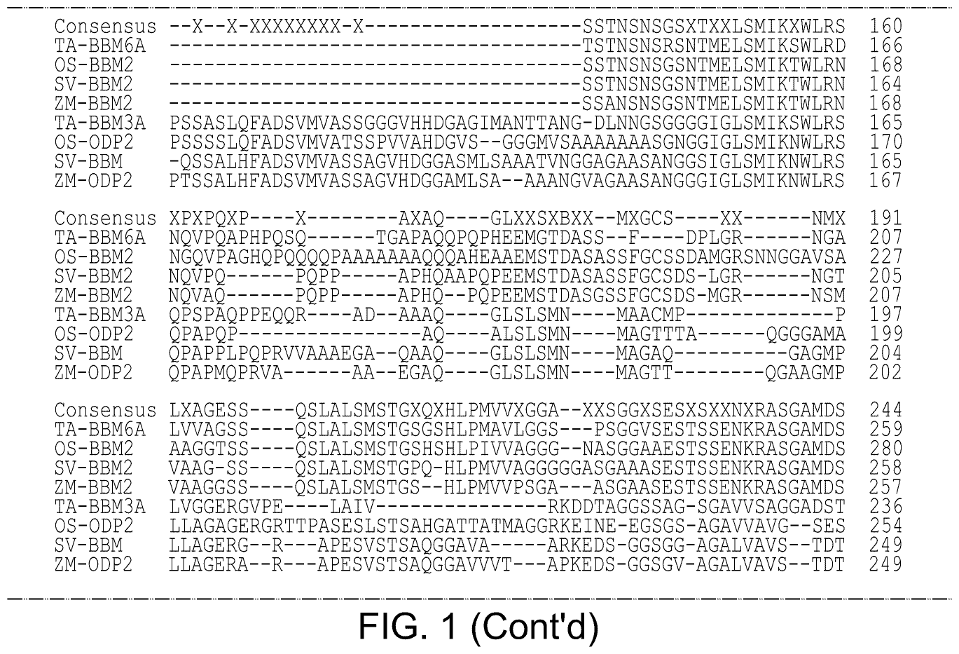

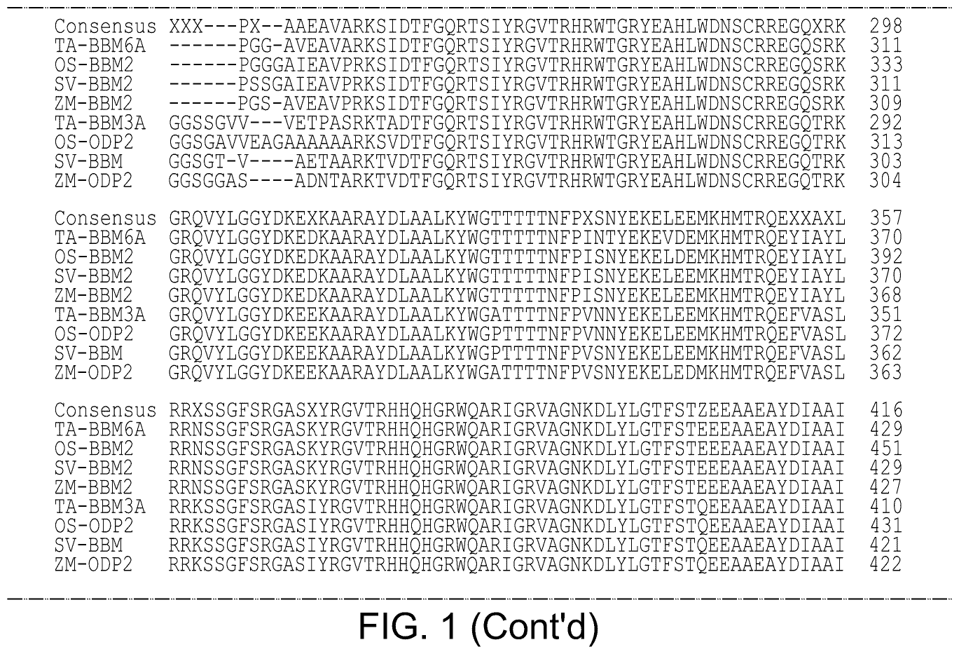

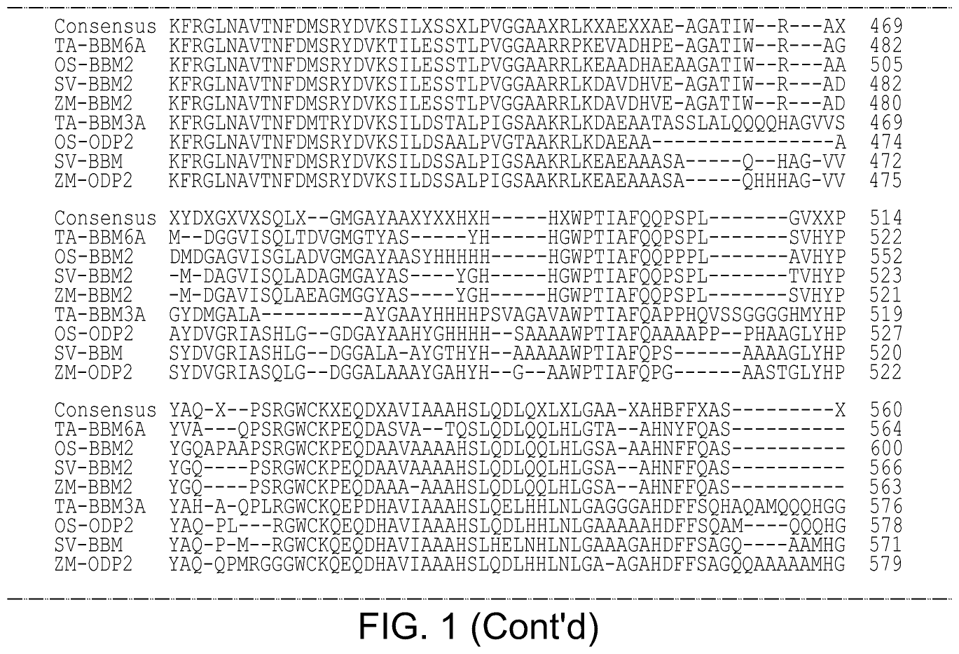

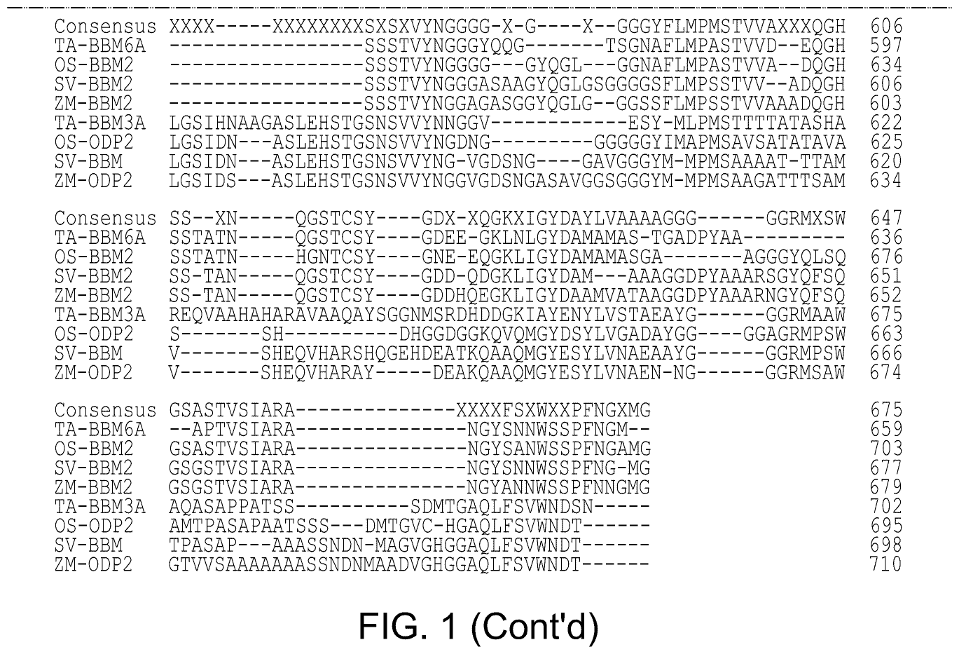

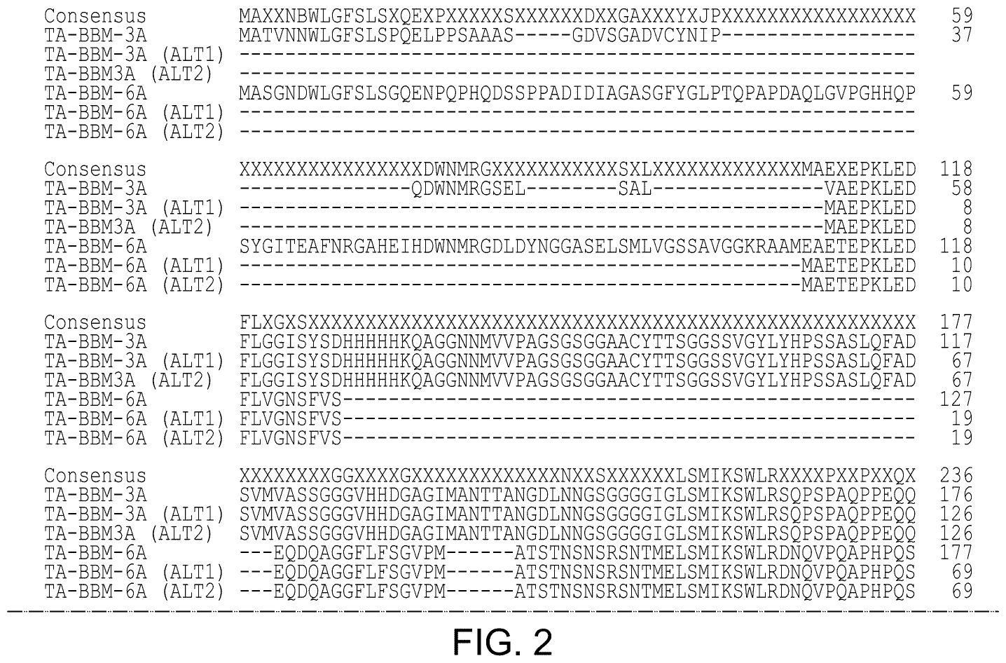

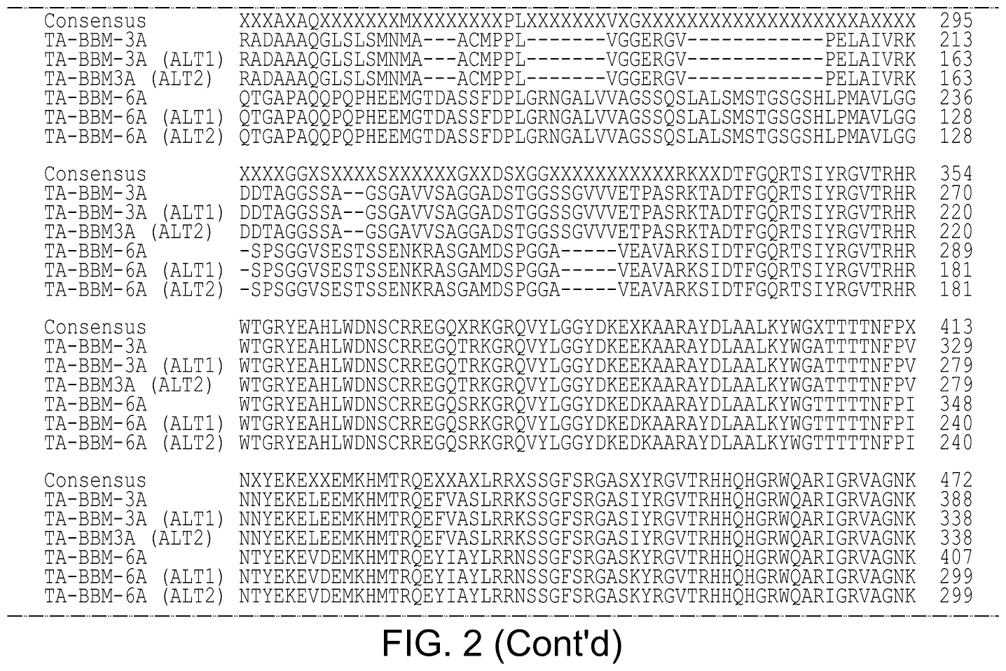

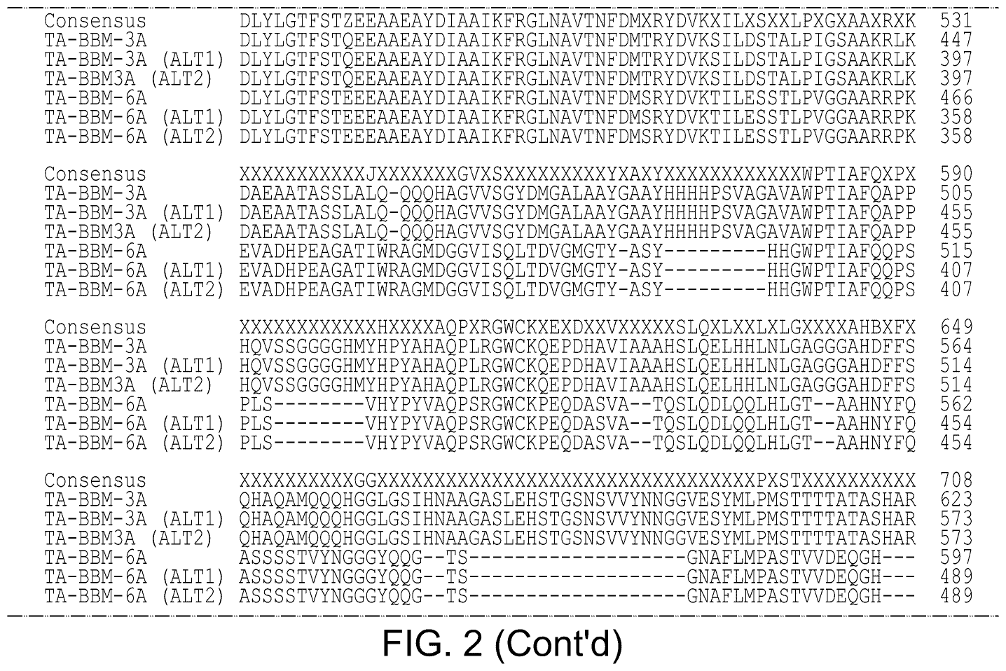

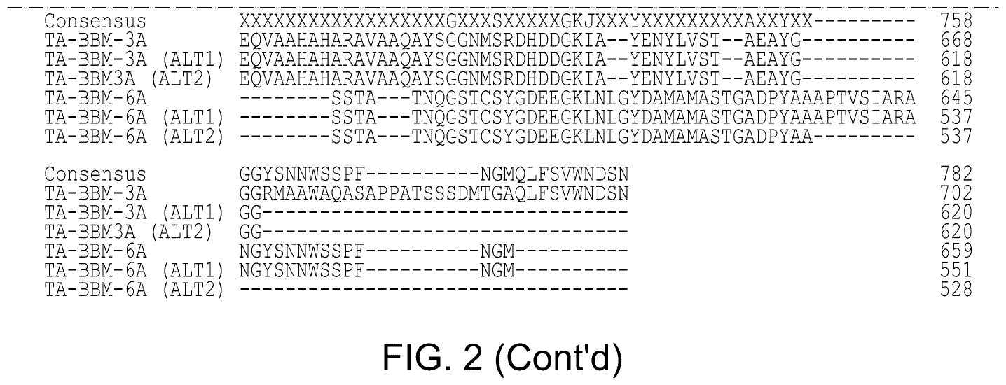

TABLE A

| | Polynucleotide | | |
|---|---|---|---|
| SEQ | (DNA) or | | |
| ID | Polypeptide | | |
| NO: | (PRT) | Name | Description |
| 1 | DNA | TA-BBM3 | Triticum aestivum BBM3A coding sequence |
| 2 | PRT | TA-BBM3A | Triticum aestivum BBM3A amino acid sequence |
| 3 | DNA | TA-BBM3B | Triticum aestivum BBM3B coding sequence |
| 4 | PRT | TA-BBM3B | Triticum aestivum BBM3B amino acid sequence |
| 5 | DNA | TA-BBM3D | Triticum aestivum BBM3D coding sequence |
| 6 | PRT | TA-BBM3D | Triticum aestivum BBM3D amino acid sequence |
| 7 | DNA | TA-BBM6A | Triticum aestivum BBM6A coding sequence |
| 8 | PRT | TA-BBM6A | Triticum aestivum BBM6A amino acid sequence |
| 9 | DNA | TA-BBM6B | Triticum aestivum BBM6B coding sequence |
| 10 | PRT | TA-BBM6B | Triticum aestivum BBM6B amino acid sequence |
| 11 | DNA | TA-BBM6D | Triticum aestivum BBM6D coding sequence |
| 12 | PRT | TA-BBM6D | Triticum aestivum BBM6D amino acid sequence |
| 13 | DNA | ZM-ODP2 | Zea mays ODP2 coding sequence |
| 14 | PRT | ZM-ODP2 | Zea mays ODP2 amino acid sequence |
| 15 | DNA | ZM-DD45 PRO | Zea mays DD45 promoter sequence |
| 16 | DNA | PV-EGG CELL | Panicum virgatum egg cell promoter sequence |
| | | PRO (TR1) | |
| 17 | DNA | EGG MIN PRO | Panicum virgatum egg cell promoter sequence |
| 18 | DNA | PV-PRO31696.1 | Panicum virgatum egg cell promoter sequence |
| | | 5UTR | |
| 19 | DNA | PvEC1 | Panicum virgatum egg cell sequence |
| | | PROMOTER | promoter sequence |
| 20 | DNA | ZM-BBM2 | Zea mays BBM2 coding sequence |
| 21 | PRT | ZM-BBM2 | Zea mays BBM2 amino acid sequence |
| 22 | DNA | OS-ODP2 | Oryza sativa ODP2 coding sequence |
| 23 | PRT | OS-ODP2 | Oryza sativa ODP2 amino acid sequence |
| 24 | DNA | OS-BBM2 | Oryza sativa BBM2 coding sequence |
| 25 | PRT | OS-BBM2 | Oryza sativa BBM2 amino acid sequence |
| 26 | DNA | SV-BBM1 | Setaria viridis BBM coding sequence |
| 27 | PRT | SV-BBM1 | Setaria viridis BBM amino acid sequence |
| 28 | DNA | SV-BBM2 | Setaria viridis BBM2 coding sequence |
| 29 | PRT | SV-BBM2 | Setaria viridis BBM2 amino acid sequence |
| 30 | DNA | ZM-AS2-EME | Zea mays AS2 EME nucleotide sequence |
| 31 | DNA | ZM-DD45 PRO:(1X | DD45 promoter sequence with 1X ZM-AS2-20 |
| | | ZM-AS2-20) | |
| 32 | DNA | ZM-DD45 PRO:(2X | DD45 promoter sequence with 2X ZM-AS2-20 |
| | | ZM-AS2-20) | |
| 33 | DNA | ZM-DD45 PRO:(3X | DD45 promoter sequence with 3X ZM-AS2-20 |
| | | ZM-AS2-20) | |
| 34 | DNA | ZM-DD45 PRO:(2X | DD45 promoter sequence with 2X ZM-AS2-20 |
| | | ZM-AS2-20) | |
| 35 | DNA | ZM-DD45 PRO:(1X | DD45 promoter sequence with 1X ZM-AS2-20 |
| | | ZM-AS2-20) | |
| 36 | DNA | ZM-DD45 PRO:(2X | DD45 promoter sequence with 2X ZM-AS2-100 |
| | | ZM-AS2-100) | |
| 37 | DNA | TA-EGG PRO | Triticum aestivum egg cell promoter sequence |
| 38 | DNA | TA-EGG4A PRO | Triticum aestivum egg cell promoter sequence |
| 39 | DNA | TA-EGG4B PRO | Triticum aestivum egg cell promoter sequence |
| 40 | DNA | TA-EC PRO | Triticum aestivum egg cell promoter sequence |
| 41 | DNA | TA-BBM3A (ALT1) | Triticum aestivum BBM3A coding sequence |
| 42 | PRT | TA-BBM3A (ALT1) | Triticum aestivum BBM3A amino acid sequence |
| 43 | DNA | TA-BBM3A (ALT2) | Triticum aestivum BBM3A coding sequence |
| 44 | PRT | TA-BBM3A (ALT2) | Triticum aestivum BBM3A amino acid sequence |
| 45 | DNA | TA-BBM6A (ALT1) | Triticum aestivum BBM6A coding sequence |
| 46 | PRT | TA-BBM6A (ALT1) | Triticum aestivum BBM6A amino acid sequence |
| 47 | DNA | TA-BBM6A (ALT2) | Triticum aestivum BBM6A coding sequence |
| 48 | PRT | TA-BBM6A (ALT2) | Triticum aestivum BBM6A amino acid sequence |
| 49 | DNA | TA-BBM3A | Nucleotide sequence of gRNA that |
| | | gRNA1 | targets wheat genomic BBM3A |
| 50 | DNA | TA-BBM3A | Nucleotide sequence of gRNA that |
| | | gRNA2 | targets wheat genomic BBM3A |
| 51 | DNA | TA-BBM3A | Nucleotide sequence of gRNA that |
| | | gRNA3 | targets wheat genomic BBM3A |
| 52 | DNA | TA-BBM3A | Nucleotide sequence of gRNA that |
| | | gRNA4 | targets wheat genomic BBM3A |
| 53 | DNA | TA-BBM3A | Nucleotide sequence of gRNA that |
| | | gRNA5 | targets wheat genomic BBM3A |
| 54 | DNA | TA-BBM3A | Nucleotide sequence of gRNA that |
| | | gRNA6 | targets wheat genomic BBM3A |
| 55 | DNA | TA-BBM3A | Nucleotide sequence of gRNA that |
| | | gRNA7 | targets wheat genomic BBM3A |
| 56 | DNA | TA-BBM3A | Nucleotide sequence of gRNA that |
| | | gRNA8 | targets wheat genomic BBM3A |
| 57 | DNA | TA-SPO11-1 A | Triticum aestivum Spo11-1 A genomic sequence |
| | | genomic | |
| 58 | DNA | TA-SPO11-1 B | Triticum aestivum Spo11-1 B genomic sequence |
| | | genomic | |
| 59 | DNA | TA-SPO11-1 D | Triticum aestivum Spo11-1 D genomic sequence |
| | | genomic | |
| 60 | DNA | TA-REC8 A | Triticum aestivum REC8 A genomic sequence |
| | | genomic | |
| 61 | DNA | TA-REC8 B | Triticum aestivum REC8 B genomic sequence |
| | | genomic | |
| 62 | DNA | TA-REC8 D | Triticum aestivum REC8 D genomic sequence |
| | | genomic | |
| 63 | DNA | TA-OSD A | Triticum aestivum OSD A genomic sequence |
| | | genomic | |
| 64 | DNA | TA-OSD B | Triticum aestivum OSD B genomic sequence |
| | | genomic | |
| 65 | DNA | TA-OSD D | Triticum aestivum OSD D genomic sequence |
| | | genomic | |
| 66 | DNA | TA-SPO11-1 | Nucleotide sequence of gRNA that |
| | | gRNA1 | targets wheat genomic SPO11-1 |
| 67 | DNA | TA-SPO11-1 | Nucleotide sequence of gRNA that |
| | | gRNA2 | targets wheat genomic SPO11-1 |
| 68 | DNA | TA-REC8 gRNA1 | Nucleotide sequence of gRNA that |
| | | | targets wheat genomic REC8 |
| 69 | DNA | TA-REC8 gRNA2 | Nucleotide sequence of gRNA that |
| | | | targets wheat genomic REC8 |
| 70 | DNA | TA-REC8 gRNA3 | Nucleotide sequence of gRNA that |
| | | | targets wheat genomic REC8 |
| 71 | DNA | TA-REC8 gRNA4 | Nucleotide sequence of gRNA that |
| | | | targets wheat genomic REC8 |
| 72 | DNA | TA-OSD gRNA1 | Nucleotide sequence of gRNA that |
| | | | targets wheat genomic OSD |
| 73 | DNA | TA-OSD gRNA2 | Nucleotide sequence of gRNA that |
| | | | targets wheat genomic OSD |
| 74 | DNA | TA-BBM3A | Triticum aestivum TA-BBM3A genomic sequence |
| | | genomic | |
| 75 | DNA | TA-BBM3A | Triticum aestivum BBM3A genomic |
| | | genomic coding | coding sequence |
| 76 | DNA | TA-BBM3B | Triticum aestivum BBM3B genomic sequence |
| | | genomic | |
| 77 | DNA | TA-BBM3B | Triticum aestivum TA-BBM3B genomic sequence |
| | | genomic coding | |
| 78 | DNA | TA-BBM3D | Triticum aestivum TA-BBM3D genomic sequence |
| | | genomic | |
| 79 | DNA | TA-BBM3D | Triticum aestivum TA-BBM3D genomic |
| | | genomic coding | coding sequence |
| 80 | DNA | TA-BBM6A | Triticum aestivum BBM6A genomic sequence |
| | | genomic | |
| 81 | DNA | TA-BBM6A | Triticum aestivum BBM6A genomic |
| | | genomic coding | coding sequence |
| 82 | DNA | TA-BBM6B | Triticum aestivum BBM6B genomic sequence |
| | | genomic | |
| 83 | DNA | TA-BBM6B | Triticum aestivum BBM6B genomic |
| | | genomic coding | coding sequence |
| 84 | DNA | TA-BBM6D | Triticum aestivum BBM6D genomic sequence |
| | | genomic | |
| 85 | DNA | TA-BBM6D | Triticum aestivum BBM6D genomic |
| | | genomic coding | coding sequence |
| 86 | DNA | ZM-EGG PRO | Zea mays EGG PRO sequence |
| 87 | DNA | ZM-ODP2 | Zea mays ODP2 genomic coding sequence |
| | | genomic coding | |
| 88 | DNA | ZM-BBM2 | Zea mays BBM2 genomic coding sequence |
| | | genomic coding | |
| 89 | DNA | ZM-ODP2 promoter | Zea mays ODP2 promoter sequence |
| 90 | DNA | ZM-BBM2 promoter | Zea mays BBM2 promoter sequence |
| 91 | DNA | ZM-ODP2 PRO | Nucleotide sequence of gRNA that |
| | | gRNA1 | targets Zea mays ODP2 promoter |
| 92 | DNA | ZM-ODP2 PRO | Nucleotide sequence of gRNA that |
| | | gRNA2 | targets Zea mays ODP2 promoter |
| 93 | DNA | ZM-ODP2 PRO | Nucleotide sequence of gRNA that |
| | | gRNA3 | targets Zea mays ODP2 promoter |
| 94 | DNA | ZM-ODP2 PRO | Nucleotide sequence of gRNA that |
| | | gRNA4 | targets Zea mays ODP2 promoter |
| 95 | DNA | ZM-BBM2 PRO | Nucleotide sequence of gRNA that |
| | | gRNA1 | targets Zea mays BBM2 promoter |
| 96 | DNA | ZM-BBM2 PRO | Nucleotide sequence of gRNA that |
| | | gRNA2 | targets Zea mays BBM2 promoter |
| 97 | DNA | ZM-BBM2 PRO | Nucleotide sequence of gRNA that |
| | | gRNA3 | targets Zea mays BBM2 promoter |
| 98 | DNA | ZM-BBM2 PRO | Nucleotide sequence of gRNA that |
| | | gRNA4 | targets Zea mays BBM2 promoter |
| 99 | DNA | ZM-EGG gRNA1 | Nucleotide sequence of gRNA that |
| | | | targets Zea mays Egg promoter |
| 100 | DNA | ZM-EGG gRNA2 | Nucleotide sequence of gRNA that |
| | | | targets Zea mays Egg promoter |
| 101 | DNA | ZM-EGG gRNA3 | Nucleotide sequence of gRNA that |
| | | | targets Zea mays Egg promoter |
| 102 | DNA | ZM-EGG gRNA4 | Nucleotide sequence of gRNA that |
| | | | targets Zea mays Egg promoter |
| 103 | DNA | ZM-DD45 gRNA1 | Nucleotide sequence of gRNA that |
| | | | targets Zea mays DD45 promoter |
| 104 | DNA | ZM-DD45 gRNA2 | Nucleotide sequence of gRNA that |
| | | | targets Zea mays DD45 promoter |
| 105 | DNA | ZM-DD45 gRNA3 | Nucleotide sequence of gRNA that |
| | | | targets Zea mays DD45 promoter |
| 106 | DNA | ZM-DD45 gRNA4 | Nucleotide sequence of gRNA that |
| | | | targets Zea mays DD45 promoter |
| 107 | PRT | TA-BBM3A | Triticum aestivum BBM3A MOTIF A sequence |
| | | MOTIF A | |
| 108 | PRT | TA-BBM3A | Triticum aestivum BBM3A MOTIF B sequence |
| | | MOTIF B | |
| 109 | PRT | TA-BBM6A | Triticum aestivum BBM6A MOTIF A sequence |
| | | MOTIF A | |
| 110 | PRT | TA-BBM6A | Triticum aestivum BBM6A MOTIF B sequence |
| | | MOTIF B | |
| 111 | PRT | AP2 conserved | Triticum aestivum AP2 DNA binding |
| | | domain | domain sequence |
| 112 | PRT | Consensus sequence | Consensus BBM peptide sequences |
| | | from FIG. 1 | |
| 113 | PRT | Consensus sequence | Consensus Triticum aestivum BBM |
| | | from FIG. 2 | native and modified peptide sequences |
| 114 | DNA | ZM-DD45 | Zea mays DD45 genomic coding sequence |
| 115 | DNA | ZM-EGG | Zea mays EGG genomic coding sequence |
| 116 | PRT | Motif A | Motif A consensus sequence |
| 117 | PRT | Motif A | Motif A consensus sequence BBM2 family |
| 118 | PRT | Motif A | Motif A consensus sequence ODP2 family |
| 119 | PRT | Motif B | Motif B consensus sequence |
| 120 | PRT | Motif B | Motif B consensus sequence BBM2 family |
| 121 | PRT | Motif B | Motif B consensus sequence ODP2 family |
| 122 | PRT | AP2 binding | AP2 DNA binding domain consensus sequence |
| | | domain | |

### DETAILED DESCRIPTION

The disclosures herein will be described more fully hereinafter with reference to the accompanying figures, in which some, but not all possible aspects are shown. Indeed, disclosures may be embodied in many different forms and should not be construed as limited to the aspects set forth herein; rather, these aspects are provided so that this disclosure will satisfy applicable legal requirements.

It is to be understood that the disclosures are not to be limited to the specific examples disclosed and that modifications and other aspects are intended to be included within the scope of the appended claims.

It is also to be understood that the terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed methods and compositions belong. In this specification and in the claims which follow, reference will be made to a number of terms which shall be defined herein.

As used herein the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

All patents, publications and patent applications mentioned in the specification are indicative of the level of those skilled in the art to which this disclosure pertains. All patents, publications and patent applications are herein incorporated by reference in the entirety to the same extent as if each individual patent, publication or patent application was specifically and individually indicated to be incorporated by reference in its entirety.

The present disclosure provides methods and compositions for inducing parthenogenesis that includes expressing, in a female gametophyte, a BBM polypeptide or a fragment thereof encoded by a Babyboom (BBM) polynucleotide operably linked to a promoter that expresses in an egg cell. As shown herein in Examples 1, expression of wheat, maize, rice and Setaria viridis BBM polypeptides in a female gametophyte of a wheat plant were useful for inducing maternal haploid induction, resulting in the production of haploid embryos without the use of pollination.

Any suitable polynucleotide that encodes BBM polypeptide or fragments thereof that are able to induce maternal haploid induction in female gametophytes when expressed by a promoter that expresses in an egg cell may be utilized in the methods and compositions of the present disclosure. In some examples, the BBM polynucleotide comprises a nucleotide sequence encoding a BBM1, BBM2, BBM3, BBM6, BMN2, BMN3, ODP2, or BBML polypeptide. In some embodiments, BBM polynucleotides and BBM polypeptides include any of those disclosed in Publication No. US20210180077, incorporated herein by reference in its entirety. In some aspects, the BBM polynucleotide encoding a BBM polypeptide comprises a nucleotide sequence as set forth in SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88. In other aspects, the BBM polynucleotide comprises a nucleotide sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88 when aligned across its full length.

Fragments of polynucleotide sequences encoding BBM polypeptides are also encompassed by the embodiments. “Fragment” as used herein in reference to BBM refers to a portion of the nucleotide sequence encoding a BBM polypeptide. The fragment may encode a biologically active portion of a BBM polypeptide. Nucleic acid molecules that are fragments of a nucleotide sequence encoding a BBM polypeptide comprise at least about 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2550, 2600, 2650, 2700, 2750, 2800, 2850, 2900, 2950, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, or 8200 contiguous nucleotides or up to the number of nucleotides present in a full-length nucleic acid sequence encoding a BBM polypeptide disclosed herein, for example, those in SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88. “Contiguous nucleotides” is used herein to refer to nucleotide residues that are immediately adjacent to one another. Fragments of the nucleic acid sequences of the embodiments that encode polypeptide fragments that retain the haploid induction activity of the BBM polypeptide and, hence, retain its ability to induce maternal haploid induction in a female gamctophyte when expressed by a promoter that expresses in an egg cell. In some examples, the BBM polypeptides or polypeptide fragments include one or more of following: the A motif, the B motif, and/or the AP2 DNA binding domain. Non-limiting examples of A motifs are set forth in FIG. 1 and FIG. 2, including in SEQ ID NOs: 107, 109, 116, 117, and 118. Non-limiting examples of B motifs are set forth in FIG. 1 and FIG. 2, including in SEQ ID NO: 108, 110, 119, 120, and 121. Non-limiting examples of the AP2 DNA binding domain is set forth in FIG. 1 and FIG. 2, including in SEQ ID NOs: 111 and 122.

In some embodiments, the BBM polynucleotide is altered to create a nucleotide sequence that encodes a variant BBM polypeptide. In some aspects, the BBM polynucleotide encoding a variant BBM polypeptide is derived from SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88 by alteration of one or more nucleotides by deletion, substitution, addition insertion, or combinations thereof. In some examples, nucleotide or amino acid sequences are maintained in one or more conserved regions of BBM polypeptides, such as the A motif, the B motif, or the AP2 DNA binding domain, and altered in non-conserved regions. See, for example, the alignment in FIGS. 1 and 2 and SEQ ID NOs: 107, 108, 109, 110, 111, 116, 117, 118, 119, 120, 121, and 122. In some examples, nucleotide or amino acid sequences are maintained in one or more consensus regions of BBM polypeptides, including in the A motif, the B motif, or the AP2 DNA binding domain, and altered in non-conserved regions. Variants of a BBM polypeptide useful in the methods and compositions described herein retain their ability to induce haploid induction in a female gametophyte when expressed by a promoter that expresses in an egg cell. Fragments and variants may be obtained via methods including but not limited to site-directed mutagenesis and synthetic construction.

In some aspects, the BBM polypeptide comprises an amino acid sequence as set forth in SEQ ID NO: 2, 4, 6, 8, 10, 12, 14, 21, 23, 25, 27, 29, 42, 44, 46, or 48. In other aspects, the BBM polypeptide comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid set forth in SEQ ID NO: 2, 4, 6, 8, 10, 12, 14, 21, 23, 25, 27, 29, 42, 44, 46, or 48 when aligned across the full length of the sequences.

Fragments of BBM polypeptides are also encompassed by the embodiments. “Fragment” as used herein in reference to BBM refers to a portion of the amino acid sequence for a BBM polypeptide. The fragment may encode a biologically active portion of a BBM polypeptide. Polypeptides that are fragments of the BBM polypeptides comprise at least about 150, 180, 200, 210, 225, 240, 250, 270, 290, 300, 315, 325, 330, 350, 360, 375, 400, 425, 450, 475, 500, 525, 550, 575, 600, 625, 650, 675, 700, 725, or 750 contiguous amino acids or up to the number of amino acids present in a full-length amino acid sequence of a BBM polypeptide disclosed herein, for example, those in SEQ ID NO: 2, 4, 6, 8, 10, 12, 14, 21, 23, 25, 27, 29, 42, 44, 46, or 48. “Contiguous amino acids” is used herein to refer to amino acid residues that are immediately adjacent to one another. Polypeptide fragments that retain the haploid induction activity of the BBM polypeptide and, hence, retain their ability to induce maternal haploid induction in a female gametophyte when expressed by a promoter that expresses in an egg cell. In some examples, the BBM polypeptide fragments include one or more of following: the A motif, the B motif, or the AP2 DNA binding domain. Non-limiting examples of BBM B motifs are set forth in FIG. 1 and FIG. 2, including in SEQ ID NO: 108 and 110. A non-limiting example of the BBM AP2 DNA binding domain is set forth in FIG. 1 and FIG. 2, including in SEQ ID NO:111.

In some embodiments, the BBM polynucleotide is altered to create a nucleotide sequence that encodes a variant BBM polypeptide. In some aspects, the variant BBM polypeptide is derived from SEQ ID NO: 2, 4, 6, 8, 10, 12, 14, 21, 23, 25, 27, 29, 42, 44, 46, or 48 by alteration of one or more amino acid residues, for example, by deletion, substitution, addition insertion of one or more nucleotides. In some examples, amino acid sequences are maintained in one or more conserved regions of BBM polypeptides, such as the A motif, the B motif, or the AP2 DNA binding domain, and altered in non-conserved regions. See, for example, FIGS. 1 and 2. Non-limiting examples of A motifs are set forth in FIG. 1 and FIG. 2, including in SEQ ID NO: 107 and 109. Non-limiting examples of B motifs are set forth in FIG. 1 and FIG. 2, including in SEQ ID NO: 108 and 110. A non-limiting example of the AP2 DNA binding domain is set forth in FIG. 1 and FIG. 2, including in SEQ ID NO:111. Variants of a BBM polypeptide useful in the methods and compositions described herein retain their ability to induce maternal haploid induction in a female gametophyte when expressed by a promoter that expresses in an egg cell. Fragments and variants may be obtained via methods including but not limited to site-directed mutagenesis and synthetic construction.

Any of the BBM polynucleotides described herein may be introduced into a female gametophyte, including the gametophyte's genome, using any suitable techniques, including but not limited to gene editing, or transformation. In some examples, the BBM polynucleotides are part of recombinant nucleic acids, such as heterologous polynucleotides and DNA constructs. As used herein, “heterologous” refers to a nucleic acid that originates from a foreign species, or, if from the same species, is substantially modified from its native form in composition and/or genomic locus by deliberate human intervention. For example, a promoter operably linked to a heterologous structural gene that is from a species different from that from which the structural gene was derived, or, if from the same species, one or both are substantially modified from their original form and/or genomic location.

Because BBM is not naturally expressed in a female gametophyte, its expression is modified by operatively linking the polynucleotide to a promoter that drives expression in an egg cell. As used herein, “egg cell” means the female gamete giving rise to the embryo.

As used herein “promoter” is an exemplary regulatory element and generally refers to a nucleotide sequence capable of controlling the expression of a coding sequence or functional RNA. In general, a coding sequence is located 3′ to a promoter sequence. The promoter sequence comprises proximal and more distal upstream elements, the latter elements are often referred to as enhancers.

An “enhancer” is a nucleotide sequence that may stimulate promoter activity and may be an innate element of the promoter or a heterologous element inserted to enhance the level or tissue-specificity of a promoter. An example of an enhancer is an EME described elsewhere herein.

A promoter generally includes a core promoter (also known as minimal promoter) sequence that includes a minimal regulatory region to initiate transcription, that is a transcription start site. Generally, a core promoter includes a TATA box. A core promoter is a minimal sequence required to direct transcription initiation and generally may not include enhancers or other UTRs. Promoters may be derived in their entirety from a native gene, or be composed of different elements derived from different promoters found in nature, or even comprise synthetic DNA segments.

A “plant promoter” is a promoter capable of initiating transcription in plant cells. Any suitable promoter that is capable of initiating transcription of a polynucleotide in a plant egg cell, i.e. expressing the polynucleotide in the egg cell, may be utilized in the methods and compositions of the present disclosure, including but not limited to egg cell promoters, egg cell-specific promoters, or egg cell-preferred promoters, and promoters active during female gamete development.

Exemplary promoters that express in a plant egg cell include but are not limited to SEQ ID NOs: 15, 16, 17, 18, 19, 31, 32, 33, 34, 35, 36, 37, 38, 39, 40, and 86. Also included in the embodiments are any of their fragments or variants.

Any egg cell promoters and/or egg cell-specific promoters known to one skilled in the art may be used, for example, the egg cell-specific EC1.1 and EC1.2 promoters disclosed in Sprunck et al., (2012) Science, 338, 1093-1097 and Steffen et al., (2007) Plant J., 51:281-92) and those disclosed in US2015/0152430 and US2018/0094273, each of which is incorporated herein by reference in its entirety.

In one embodiment, the present disclosure provides a method of inducing parthenogenesis that includes expressing, in a female gametophyte, a BBM polypeptide or a fragment thereof encoded by a Babyboom (BBM) polynucleotide operably linked to a promoter that expresses in an egg cell. As shown herein, expression of a BBM polypeptide in a female gametophyte of a plant is useful for inducing maternal haploid induction, resulting the production of a haploid embryo without the use of pollination.

The egg cell promoter nucleotide sequences and methods disclosed herein are useful in regulating expression of any heterologous nucleotide sequences (such as but not limited to BBM sequences) in egg cells of a plant. In some embodiments, the promoter that expresses in an egg cell is an egg cell-preferred promoter. In some embodiments, the promoter that expresses in an egg cell is an egg cell-specific promoter.

In one embodiment, this disclosure provides a method of expressing a gene product in a plant egg cell. In some aspects, the egg cell is in the plant. In other aspects, the egg cell is isolated from the plant. As used herein, gene product refers to a polynucleotide or a polypeptide. In some aspects, the method includes introducing into an egg cell a promoter comprising a polynucleotide sequence set forth in SEQ ID NO: 15, 16, 17, 18, 19, 31, 32, 33, 34, 35, 36, 37, 38, 39, 40, or 86.

In some aspects, the egg cell promoters comprise a nucleotide sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO: 15, 16, 17, 18, 19, 31, 32, 33, 34, 35, 36, 37, 38, 39, 40, or 86, when aligned across the full length of the sequences.

Fragments of the egg cell promoters are also encompassed by the embodiments. “Fragment” as used herein with reference to an egg cell promoter refers to a portion of the nucleotide sequence of the egg cell promoter. Nucleic acid molecules that are fragments of a nucleotide sequence of the egg cell promoters comprise at least about 50, 100, 150, 180, 210, 240, 270, 300, 330, 360, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or 2000 contiguous nucleotides or up to the number of nucleotides present in a full-length nucleic acid promoter sequence disclosed herein, for example, those in SEQ ID NO: 15, 16, 17, 18, 19, 31, 32, 33, 34, 35, 36, 37, 38, 39, 40, and 86. “Contiguous nucleotides” is used herein to refer to nucleotide residues that are immediately adjacent to one another. Fragments of the nucleic acid sequences of egg cell promoters that retain the ability to initiate transcription in an egg cell and, hence, retain their ability to expresses a heterologous polynucleotide in an egg cell.

In some embodiments, the polynucleotide of the egg cell promoter is altered to create a nucleotide sequence that encodes a variant. In some aspects, the egg cell promoter variant is derived from SEQ ID NO: 15, 16, 17, 18, 19, 31, 32, 33, 34, 35, 36, 37, 38, 39, 40, or 86 by alteration of one or more nucleotides by deletion, substitution, addition insertion, or combinations thereof.

A “variant” of an egg cell promoter may include changes in its nucleotide sequence in which one or more nucleotides of the original sequence is deleted, added, and/or substituted, while substantially maintaining promoter function in an egg cell. One or more base pairs can be inserted, deleted, or substituted internally to a promoter. In the case of a promoter fragment, variant promoters can include changes affecting the transcription of a minimal promoter to which it is operably linked. Variant promoters can be produced, for example, by standard DNA mutagenesis techniques or by chemically synthesizing the variant promoter or a portion thereof.

In some embodiments, the egg cell promoter may be operably linked to a heterologous polynucleotide sequence so that the polypeptide encoded by the heterologous polynucleotide sequence is expressed in a female reproductive tissue or cell of a plant, e.g. an egg cell.

Any of the egg cell promoter polynucleotides described herein may be introduced into a female gametophyte, including the gametophyte's genome, using any suitable techniques, including but not limited to, for example, gene editing, transformation. In some embodiments, the egg cell promoter polynucleotides may be part of chimeric polynucleotides, such as a heterologous polynucleotide and DNA constructs. Accordingly, provided herein is a recombinant DNA construct comprising a nucleotide sequence comprising any of the sequences set forth in SEQ ID NOs: 15, 16, 17, 18, 19, 31, 32, 33, 34, 35, 36, 37, 38, 39, 40, or 86; a nucleotide sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO: 15, 16, 17, 18, 19, 31, 32, 33, 34, 35, 36, 37, 38, 39, 40, or 86; or a functional fragment thereof, or variant thereof, operably linked to at least one heterologous sequence. Also provided herein are embryos, plants, and parts thereof comprising a nucleotide sequence comprising any of the sequences set forth in SEQ ID NOS: 15, 16, 17, 18, 19, 31, 32, 33, 34, 35, 36, 37, 38, 39, 40, or 86; a nucleotide sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO: 15, 16, 17, 18, 19, 31, 32, 33, 34, 35, 36, 37, 38, 39, 40, or 86; or a functional fragment thereof, or variant thereof, operably linked to at least one heterologous sequence. In some examples, the operably linked heterologous polynucleotide sequence encodes a BBM polypeptide, or functional BBM fragment thereof, or BBM variant. The egg cell promoter sequence and BBM polynucleotide sequence are heterologous with respect to one another as this combination is not naturally occurring. In another example, the egg cell promoter or BBM polynucleotide may be homologous, or native, or heterologous, or foreign, to the plant cell, for example, a maize egg cell promoter is heterologous to a wheat female gametophyte. Fragments and variants can be obtained via any suitable methods including but not limited to site-directed mutagenesis and synthetic construction.

The promoter sequences disclosed herein may be comprised in a recombinant DNA construct or in a chimeric polynucleotide, e.g. combined with a heterologous polynucleotide, for example, as part of an engineered genomic locus.

Also provided herein is a method of modulating expression of a polynucleotide encoding a polypeptide of interest in an egg cell. In some aspects, the method includes expressing the polynucleotide by operably linking the polynucleotide with a promoter nucleotide sequence that expresses in an egg cell, wherein the egg cell promoter includes one of more expression modulating/modulation elements (EMEs). “Expression modulating/modulation element” or “EME” as used herein refers to a nucleotide sequence that up or down-regulates the expression of one or more plant genes. EME may have one or more copies of the same sequence arranged head-to-head, tail-to-head, or head-to-tail or any combination of these configurations. EMEs may be derived from plant sequences, or from bacterial or viral enhancer elements. Exemplary EMEs for use in the methods and compositions include but are not limited to SEQ ID NO: 30 and those described in Publication No. WO 2018/183878, which is incorporated herein in its entirety. In some aspects, the EME is heterologous to the egg cell promoter.

In another aspect, provided herein is a recombinant DNA construct or chimeric polynucleotides comprising an egg cell promoter and an EME, wherein the EME is heterologous to the egg cell promoter. Also provided is a method of modulating the expression of a polynucleotide sequence of interest in a plant egg cell, the method comprising expressing the polynucleotide sequence, where expression of the polynucleotide sequence is regulated by a promoter that expresses in an egg cell, where the promoter comprises one of more EMEs, where the EME is heterologous to the polynucleotide sequence of interest and/or the egg cell promoter. In some aspects, the polynucleotide sequence of interest is a BBM polynucleotide, which is described elsewhere herein. The methods and compositions of the present disclosure may use different promoters with or without the EME sequences and/or enhancers.

In one embodiment, a method of modulating expression of an endogenous BBM polynucleotide in a plant cell is provided. The method may include altering one or more nucleotides in a regulatory region of the genomic locus of an endogenous BBM polynucleotide, e.g. a BBM gene, to create a modified regulatory region. The creation of the modified regulatory region results in expression of the BBM polypeptide in a female gametophyte. In some embodiments, a nucleotide sequence of a promoter that expresses in an egg cell is introduced into the BBM genomic locus so that the promoter is operably linked to the nucleotide sequence encoding the BBM polypeptide. In some embodiments, the egg cell promoter may be replaced for the endogenous BBM promoter sequence. In some embodiments, the endogenous BBM promoter nucleotide sequence may be altered to that of an egg cell promoter nucleotide sequence. Also provided herein is the resulting modified BBM genomic locus.

In one embodiment, a method of modulating expression of a coding or genomic sequence in an endogenous genomic egg cell locus in a plant cell is provided. The method may include altering one or more nucleotides in a genomic or coding region of the genomic locus of an egg cell polynucleotide, e.g. to create a modified genomic or coding nucleotide sequence that encodes a BBM polypeptide. The creation of the modification results in expression of the BBM polypeptide in a female gametophyte. In some embodiments, a heterologous polynucleotide encoding a BBM polypeptide is introduced into the egg cell genomic locus so that the heterologous polynucleotide encoding a BBM polypeptide is operably linked to the regulatory region or promoter of the egg cell locus and expressed. In some embodiments, the heterologous polynucleotide encoding a BBM polypeptide replaces the endogenous genomic or coding nucleotide sequence in the genomic egg cell locus. In some embodiments, the endogenous genomic or coding nucleotide sequence in the genomic egg cell locus is altered to that of the heterologous polynucleotide encoding a BBM polypeptide. Also provided herein is the resulting modified genomic egg cell locus.

One embodiment of the disclosure includes a method of obtaining a wheat plant that produces clonal, non-reduced, non-recombined male and/or female gametes. In certain embodiments, wheat plants that have suppressed expression and/or activity with respect to its endogenous Spo11 or Prd1, Prd2, or PRd3; Rec8; and OSD1 polynucleotides and polypeptides are either heterozygous or homozygous for the suppression.

The terms “suppress”, “suppressed”, “suppression”, “suppressing” and “silencing”, are used interchangeably herein and include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches and the like. “Silencing,” as used herein with respect to a target gene, such as Spo11 or Prd1, Prd2, or PRd3; Rec8; and OSD1, refers generally to the suppression of levels of mRNA or protein expressed by the target gene, and/or the level of protein functionality.

In certain embodiments, the methods include suppressing endogenous wheat Spo11 or Prd1, Prd2, or PRd3; Rec8; and OSD1 polynucleotides and polypeptides or combinations thereof in a plant or plant cell. In certain embodiments, the plant cell is a microspore mother cell (which gives rise to haploid microspores), megaspore mother cell (which gives rise to a haploid megaspore) or any other plant cell where the genes, e.g. Spo11 or Prd1, Prd2, or PRd3; Rec8; and OSD1, are expressed.

Methods are provided to suppress the activity of endogenous Spo11 or Prd1, Prd2, or PRd3; Rec8; and OSD1 polynucleotides or polypeptides in a wheat plant cell. In some aspects, suppressing activity also includes suppressing the expression level of the Spo11 or Prd1, Prd2, or PRd3; Rec8; and OSD1 polynucleotides or polypeptides. Any suitable method or technique may be used. In certain embodiments, Spo11 or Prd1, Prd2, or PRd3; Rec8; and OSD1 polynucleotides or polypeptides are suppressed using anti-sense, cosuppression, viral-suppression, hairpin suppression, stem-loop suppression, RNAi-based approaches, and small RNA-based approaches. In certain examples, genome editing approaches, including but not limited to, Cas endonuclease and guide RNA, are employed to introduce into a plant cell's genome polynucleotides that suppress Spo11 or Prd1, Prd2, or PRd3; Rec8; and OSD1 expression and/or activity. In certain examples, genome editing approaches, including but not limited to, Cas endonuclease and guide RNA, are employed to introduce, into a plant cell's genome, nucleotide deletions or modifications, e.g. additions or substitutions, that suppress Spo11 or Prd1, Prd2, or PRd3; Rec8; and OSD1 expression and/or activity. In one embodiment, Spo11 or Prd1, Prd2, or PRd3; Rec8; and OSD1 expression and/or activity may be suppressed using any combinations of RNA-based and gene editing approaches.

One of ordinary skill in the art would readily recognize a suitable control or reference to be utilized when assessing or measuring expression level or activity of Spo11 or Prd1, Prd2, or PRd3; Rec8; and OSD1 polynucleotides or polypeptides in any embodiment of the present disclosure. For example, by way of non-limiting illustrations, a plant or plant cell comprising a modified Spo11 or Prd1, Prd2, or PRd3; Rec8; and OSD1 polynucleotide or polypeptide; or Spo11 or Prd1, Prd2, or PRd3; Rec8; and OSD1 suppression polynucleotide would be typically measured relative to a plant or plant cell not comprising the modified Spo11 or Prd1, Prd2, or PRd3; Rec8; and OSD1 polynucleotide or polypeptide, or the Spo11 or Prd1, Prd2, or PRd3; Rec8; and OSD1 suppression polynucleotide as the control or reference plant or plant cell. In some examples, the control is a wild type plant or cell. One skilled in the art will be able to determine Spo11 or Prd1, Prd2, or PRd3; Rec8; and OSD1 expression level or activity using assays, such as PCR, Northern, and Western blot assays.

In certain embodiments, wheat plants that have suppressed activity with respect to endogenous Spo11 or Prd1, Prd2, or PRd3; Rec8; and OSD1 polynucleotides and polypeptides are either heterozygous or homozygous for the suppression. These plants may be crossed, intercrossed, or selfed until a wheat plant is obtained that comprises suppressed activity for each and all of these endogenous genes: (Spo11 or Prd1, Prd2, or PRd3); Rec8; and OSD1 activity. A female gametophyte derived from this wheat plant is modified so that a Babyboom (BBM) polynucleotide encoding a BBM polypeptide, or a fragment thereof, is expressed in the female gametophyte to produce an embryo.

The modification may be performed using any suitable approach. In one example, the method includes introducing a BBM polynucleotide encoding a BBM polypeptide into the coding or genomic nucleotide sequence of a genomic egg cell locus. In another example, the method includes introducing an egg cell promoter or regulatory region into a genomic BBM locus. In another example, a chimeric polynucleotide comprising a Babyboom (BBM) polynucleotide operably linked to a promoter that expresses in an egg cell, where the BBM polynucleotide comprises a nucleotide sequence encoding a Babyboom (BBM) polypeptide or a fragment thereof is introduced into the female gametophyte. In another example, a recombinant DNA construct comprising a Babyboom (BBM) polynucleotide operably linked to a promoter that expresses in an egg cell, where the BBM polynucleotide comprises a nucleotide sequence encoding a Babyboom (BBM) polypeptide or a fragment thereof is introduced into the female gametophyte.

Using methods described herein, a wheat plant derived from this embryo may be used to produce progeny that are non-reduced, non-recombined and clonal with respect to the parent wheat plant. Plants produced from this method allow for the clonal reproduction of the wheat plant through seed. The resulting seeds from the wheat plant may be grown into plants and the seed harvested from those plants, allowing for the clonal reproduction of the parent plant through seeds. In certain embodiments, the wheat plant is a hybrid. In certain embodiments, the wheat plant is an inbred or a variety.

As used herein, the term “wheat” refers to any species of the genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. mocha, T. compactum, T. sphaerococcum, T. vavilovii, Triticum boeoticum, or to the domesticated form, Triticum monococcum, and interspecies cross thereof. Tetraploid wheat includes T. durum (also referred to as durum wheat or Triticum turgidum ssp. durum), T. dicoccoides, T. dicoccum, T. polonicum, and interspecies cross thereof. In addition, the term “wheat” includes possible progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. A wheat cultivar for use in the present disclosure may belong to, but is not limited to, any of the above-listed species. Also encompassed are plants that are produced by conventional techniques using Triticum sp. as a parent in a sexual cross with a non-Triticum species, such as rye (Secale cereale), including but not limited to Triticale. In some embodiments, the wheat plant is suitable for commercial production of grain, such as commercial varieties of hexaploid wheat or durum wheat, having suitable agronomic characteristics which are known to those skilled in the art.

The present disclosure also includes plants obtained by any of the disclosed methods or compositions herein. The present disclosure also includes seeds from a plant obtained by any of the methods or compositions disclosed herein. As used herein, the term “plant” refers to whole plants, plant organs (e.g., leaves, stems, roots, etc.), plant tissues, plant cells, plant parts, seeds, propagules, embryos and progeny of the same. As used herein, the term “plant” refers to whole plants, plant organs (e.g., leaves, stems, roots, etc.), plant tissues, plant cells, plant parts, seeds, propagules, embryos and progeny of the same. Plant cells can be differentiated or undifferentiated (e.g. callus, undifferentiated callus, immature and mature embryos, immature zygotic embryo, immature cotyledon, embryonic axis, suspension culture cells, protoplasts, leaf, leaf cells, root cells, phloem cells and pollen).

Grain is intended to mean the mature seed produced by commercial growers for purposes other than growing or reproducing the species.

The compositions and methods of the present disclosure are applicable to a broad range of plant species, including dicotyledonous plants and monocotyledonous plants. Representative examples of plants that can be treated in accordance with the methods disclosed herein include, but are not limited to, wheat, cotton, sunflower, safflower, tobacco, Arabidopsis, barley, oats, rice, maize, triticale, sorghum, rye, millet, flax, sugarcane, banana, cassava, common bean, cowpea, tomato, potato, beet, grape, Eucalyptus, wheat grasses, turf grasses, alfalfa, clover, soybean, peanuts, citrus, papaya, Setaria sp, cacao, cucumber, apple, Capsicum, bamboo, melon, cannabis, ornamentals including commercial garden and flower bulb species, fruit trees, vegetable species, Brassica species, as well as interspecies hybrids. In one embodiment, the compositions and methods of the disclosure are applied to wheat plants.

The methods of the disclosure may include introducing a polypeptide, polynucleotide (i.e., DNA or RNA), or nucleotide construct (i.e., DNA or RNA) into a plant. As used herein, “introducing” means presenting to the plant the polynucleotide, polypeptide, or nucleotide construct in such a manner that the polynucleotide, polypeptide, or nucleotide construct gains access to the interior of a cell of the plant. The methods of the disclosure do not depend on a particular method for introducing the polynucleotide, polypeptide, or nucleotide construct into a plant, only that the polynucleotide, polypeptide, or nucleotide construct gains access to the interior of at least one cell of the plant. Methods for introducing polynucleotides, polypeptides, or nucleotide constructs into plants are known in the art including, but not limited to, gene editing, stable or transient transformation methods, and virus-mediated methods.

The compositions and methods of the present disclosure optionally include producing doubled haploid plants from haploid embryos.

The present disclosure also provides methods of contacting haploid cells with an amount of a chromosome doubling agent before, during, after, or overlapping with any portion of the isolation and embryogenesis induction process used for generating a paternal gamete (androgenic) or a maternal gamete (gynogenic) doubled haploid population.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

In some embodiments, parthenogenesis inducing methods may be used to improve maternal haploid embryo regeneration productivity and enable gene editing to provide regenerated gene-edited maternal haploids.

In an aspect, haploid cells may be contacted with an amount of a chromosome doubling agent to promote chromosome doubling followed by regenerating homozygous diploid plants from the treated haploid cells. The haploid microspore cells can be in contact with the doubling agent before, during, or after initiation of microspore embryogenesis or embryo maturation. After chromosome doubling, the doubled haploid embryo will contain 2 copies of paternally derived chromosomes. The efficiency of the process for obtaining doubled haploid plants from haploid embryos may be greater than 10%, 20%, 30%, 50%, 60%, 70%, 80%, or 90%. The duration of contact between the haploid cells and the chromosomal doubling agent may vary. Contact may be from less than 24 hours, for example 4-12 hours, to about a week. The duration of contact is generally from about 8 hours to 2 days.

Methods of chromosome doubling are disclosed in Antoine-Michard, S. et al., Plant cell, tissue organ cult., Cordrecht, the Netherlands, Kluwer Academic Publishers, 1997, 48 (3): 203-207; Kato, A., Maize Genetics Cooperation Newsletter 1997, 36-37; and Wan, Y. et al., TAG, 1989, 77:889-892. Wan, Y. et al., TAG, 1991, 81:205-211. The disclosures of which are incorporated herein by reference. Typical doubling methods involve contacting the cells with colchicine, anti-microtubule agents or anti-microtubule herbicides, pronamide, nitrous oxide, or any mitotic inhibitor to create homozygous doubled haploid cells. The amount of colchicine used in medium is generally 0.01%-0.2% or approximately 0.05% of amiprophos-methyl (APM) (5-225 μM) may be used. The amount of colchicine can range from approximately 400-600 mg/L or approximately 500 mg/L. The amount of pronamide in medium is approximately 0.5-20 μM. Examples of mitotic inhibitors are included in Table B. Other agents may be used with the mitotic inhibitors to improve doubling efficiency. Such agents include dimethyl sulfoxide (DMSO), adjuvants, surfactants, and the like.

TABLE B
Chemical chromosome doubling agents

| | | |
|---|---|---|
| Common Name/ | | |
| Trade name | CAS | IUPAC |

Colchicine and Colchicine Derivatives

| | |
|---|---|
| colchicine/acetyltri- | (S)-N-(5,6,7,9-tetrahydro-1,2,3,10- |
| methylcolchicinic acid | tetramethoxy-9-oxobenzo (a) |
| | heptalen-7-yl) acetamide |
| colchicine derivatives | |

Carbamates

| | | |
|---|---|---|
| Carbetamide | (R)-1-(ethylcarbamoyl)ethyl | (2R)-N-ethyl-2- |
| | carbanilate | [[(phenylamino)carbonyl]oxy]propanamide |
| chloropropham | | |
| Propham | | |

Benzamides

| | | |
|---|---|---|
| Pronamide/ | 3,5-dichloro-N-(1,1- | 3,5-dichloro-N-(1,1-dimethyl-2- |
| propyzamide | dimethylpropynyl)benzamide | propynyl)benzamide |
| Tebutam | | |

Benzoic Acids

| | | |
|---|---|---|
| Chlorthal dimethyl | | |
| (DCPA), | | |
| Dicamba/dianat/ | 3,6-dichloro-o-anisic acid | 3,6-dichloro-2-methoxybenzoic acid |
| disugran (dicamba- | | |
| methyl) (BANVEL, | | |
| CLARITY) | | |

Dinitroaniline chromosome doubling agents

| | | |
|---|---|---|
| benfluralin/benefin/ | N-butyl-N-ethyl-α,α,α-trifluoro- | N-butyl-N-ethyl-2,6-dinitro-4- |
| (BALAN) | 2,6-dinitro-p-toluidine | (trifluoromethyl)benzenamine |
| Butralin | (RS)-N-sec-butyl-4-tert-butyl- | 4-(1,1-dimethylethyl)-N-(1-methylpropyl)- |
| | 2,6-dinitroaniline | 2,6-dinitrobenzenamine |
| Chloralin | | |
| dinitramine | N1,N1-diethyl-2,6-dinitro-4- | N3,N3-diethyl-2,4-dinitro-6- |
| | trifluoromethyl-m- | (trifluoromethyl)-1,3-benzenediamine |
| | phenylenediamine | |
| ethalfluralin (Sonalan) | N-ethyl-α,α,α-trifluoro-N-(2- | N-ethyl-N-(2-methyl-2-propenyl)-2,6- |
| | methylallyl)-2,6-dinitro-p- | dinitro-4-(trifluoromethyl)benzenamine |
| | toluidine | |
| fluchloralin | N-(2-chloroethyl)-2,6-dinitro-N- | N-(2-chloroethyl)-2,6-dinitro-N-propyl-4- |
| | propyl-4-(trifluoromethyl)aniline | (trifluoromethyl)benzenamine |
| | or N-(2-chloroethyl)-α,α,α- | |
| | trifluoro-2,6-dinitro-N- | |
| | propyl-p-toluidine | |
| isopropalin | 4-isopropyl-2,6-dinitro-N,N- | 4-(1-methylethyl)-2,6-dinitro-N,N- |
| | dipropylaniline | dipropylbenzenamine |
| methalpropalin | α,α,α-trifluoro-N-(2- | N-(2-methyl-2-propenyl)-2,6-dinitro-N- |
| | methylallyl)-2,6-dinitro-N- | propyl-4-(trifluoromethyl)benzenamine |
| | propyl-p-toluidine | |
| nitralin | 4-methylsulfonyl-2,6-dinitro- | 4-(methylsulfonyl)-2,6-dinitro-N,N- |
| | N,N-dipropylaniline | dipropylbenzenamine |
| oryzalin (SURFLAN) | 3,5-dinitro-N4,N4- | 4-(dipropylamino)-3,5- |
| | dipropylsulfanilamide | dinitrobenzenesulfonamide |
| pendimethalin | N-(1-ethylpropyl)-2,6-dinitro- | N-(1-ethylpropyl)-3,4-dimethyl-2,6- |
| (PROWL) | 3,4-xylidine | dinitrobenzenamine |
| prodiamine | 5-dipropylamino-α,α,α-trifluoro- | 2,4-dinitro-N3,N3-dipropyl-6- |
| | 4,6-dinitro-o-toluidine or | (trifluoromethyl)-1,3-benzenediamine |
| | 2,6-dinitro-N1,N1-dipropyl- | |
| | 4-trifluoromethyl-m- | |
| | phenylenediamine | |
| profluralin | N-cyclopropylmethyl-α,α,α- | N-(cyclopropylmethyl)-2,6-dinitro-N- |
| | trifluoro-2,6-dinitro-N- | propyl-4-(trifluoromethyl)benzenamine |
| | propyl-p-toluidine or | |
| | N-cyclopropylmethyl-2,6- | |
| | dinitro-N-propyl-4- | |
| | trifluoromethylaniline | |
| trifluralin (TREFLAN, | α,α,α-trifluoro-2,6-dinitro- | 2,6-dinitro-N,N-dipropyl-4- |
| TRIFIC, TRILLIN) | N,N-dipropyl-p-toluidine | (trifluoromethyl)benzenamine |

Phosphoroamidates

| | | |
|---|---|---|
| APM | | |
| (Amiprofos methyl); | | |
| amiprophos-methyl | | |
| Butamifos | O-ethyl O-6-nitro-m-tolyl | O-ethyl O-(5-methyl-2-nitrophenyl) |
| | (RS)-sec- | (1-methylpropyl)phosphoramidothioate |
| | butylphosphoramidothioate | |

Pyridines

| | | |
|---|---|---|
| Dithiopyr | | |
| Thiazopyr | methyl 2-difluoromethyl-5-(4,5- | methyl 2-(difluoromethyl)-5-(4,5-dihydro- |
| | dihydro-1,3-thiazol-2-y1)-4- | 2-thiazolyl)-4-(2-methylpropyl)-6- |
| | isobutyl-6- | (trifluoromethyl)-3-pyridinecarboxylate |
| | trifluoromethylnicotinate | |

As an alternative to using chemical chromosome doubling agents, modulating expression of genes known to impact the plant cell cycle (genetic chromosome doubling protein), either through stimulation of the cell cycle (and cell division) or through stimulation of endoreduplication, can be used to double the chromosome complement in an embryo. Increasing ploidy level in plant cells can be achieved by modulating expression of genes that stimulate key control points in the cell cycle cell. It is expected that other plant genes known to simulate the cell cycle (or cell division) in plants may be used to produce a similar doubling of the chromosome number in the forming maternal haploid embryos. Examples of plant genes whose over-expression stimulates the cell cycle include cyclin-A in tobacco (Yu et al., 2003), cyclin-D in tobacco (Cockcroft et al., 2000, Nature 405:575-79; Schnittger et al., 2002, PNAS 99:6410-6415; Dewitte et al., 2003, Plant Cell 15:79-92)., E2FA in Arabidopsis (De Veylder et al., 2002, EMBO J 21:1360-1368), E2FB in Arabidopsis (Magyar et al., 2005, Plant Cell 17:2527-2541). Similarly, over-expression of viral genes known to modulate plant cell cycle machinery can be used, such as when over-expression of the Wheat Dwarf Virus RepA gene stimulates cell cycle progression (G1/S transition) and cell division in maize (Gordon-Kamm et al., 2002, PNAS 99:11975-11980). Conversely, plant genes whose encoded products are known to inhibit the cell cycle have been shown to result in increased cell division when the gene, such as Cyclin-Dependent Kinase Inhibitor (ICK1/KRP), is down-regulated in Arabidopsis (Cheng et al 2013, Plant J 75:642-655). Thus, down-regulation of the KRP gene using an egg cell specific promoter to drive expression may have a similar effect as over-expression of DZ470, resulting in chromosome doubling. Methods of down-regulation of a gene such as KRP are known in the art and include expression of an artificial micro-RNA targeted to the KRP mRNA, or expression of a dCas9-repressor fusion that is targeted to the KRP promoter by a gRNA to that sequence. Finally, there are plant genes that are known to specifically impact the process of endoreduplication. When using such genes, such as for example the ccs52gene or the Dell gene, over-expression of ccs52 may result in an increased ploidy level as observed in Medicago sativa (Cebolla et al., 1999, EMBO J 18:4476-4484), and down-regulation of Dell may result in an increased ploidy level as observed in Arabidopsis (Vlieghe et al., 2005, Current Biol 15:59-63). It is expected that other genes that are known to stimulate the cell cycle, the G1/S transition, or endoreduplication may be used in the methods disclosed herein to increase ploidy level.

Repressor motifs are known in the art, for example see Kagale and Rozwadowski (Epigenetics. 2011. 6:141-146). Ethylene-responsive element binding factor-associated Amphiphilic Repression (EAR) motif-mediated transcriptional repression is known in plants, including EAR motifs defined by the consensus sequence patterns of either LxLxL and DLNxxP (see Hiratsu et al., 2003. Plant J. 35:177-192). Of interest to the present disclosure are peptides including the amphiphilic repression motif disclosed in WO 2013/109754 A1 and all references cited therein and the Dr1/DRAP1 global repressor complex (see U.S. Pat. No. 7,288,695 B2 and all references cited therein), including the Dr1 motif that is similar to the motif found in Arabidopsis thaliana MYBL2 (see Matsui K, Umemura Y, Ohme-Takagi M. 2008. Plant J. 55:954-967).

Methods are known in the art for the targeted insertion of a polynucleotide at a specific location in the plant genome. The insertion of the polynucleotide at a desired genomic location may be achieved using a site-specific recombination system. See, for example, WO99/25821, WO99/25854, WO99/25840, WO99/25855 and WO99/25853, all of which are herein incorporated by reference in their entirety. Briefly, a polynucleotide of interest, flanked by two non-identical recombination sites, can be contained in a T-DNA transfer cassette. The T-DNA transfer cassette may be introduced into a plant having stably incorporated into its genome a target site which is flanked by two non-identical recombination sites that correspond to the sites of the transfer cassette. An appropriate recombinase is provided, and the transfer cassette is integrated at the target site. The polynucleotide of interest is thereby integrated at a specific chromosomal position in the plant genome.

Any suitable technique may be used to introduce, into any plant cell, polynucleotides that are useful to target a specific site for modification in the genome of a plant derived from the plant cell. Site specific modifications that can be introduced include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, other endonucleases, and the like. For example, the disclosed methods can be used to introduce a CRISPR-Cas system into a plant cell or plant, for the purpose of genome modification of a target sequence in the genome of a plant or plant cell, for selecting plants, for deleting a base or a sequence, for gene editing, and for inserting a polynucleotide of interest into the genome of a plant or plant cell. Thus, the disclosed methods can be used together with a CRISPR-Cas system to provide for an effective system for modifying or altering target sites and nucleotides of interest within the genome of a plant, plant cell or seed. Any suitable Cas endonuclease may be used in the methods and to create the compositions described herein, for example, any suitable Cas endonuclease that is capable of binding to and creating a double strand break in a genomic target sequence of the plant genome.

In some embodiments, polynucleotides, such as egg cell or BBM polynucleotides, may be introduced into the genome of a plant using genome editing technologies, or polynucleotides in the genome of a plant, such as egg cell or BBM polynucleotides, may be edited using genome editing technologies. For example, the polynucleotides may be introduced into a desired location in the genome of a plant through the use of a genome editing system such as TALENs, meganucleases, zinc finger nucleases, CRISPR-Cas, and the like. For example, the disclosed polynucleotides may be introduced into a desired location in a genome using a CRISPR-Cas system, for the purpose of site-specific insertion. The desired location in a plant genome can be any desired target site for insertion, such as a genomic region.

In some embodiments, where the polynucleotide has previously been introduced into a genome, genome editing or genome engineering technologies may be used to alter or modify the introduced polynucleotide sequence, including the flanking chromosomal genomic sequences. Site-specific modifications that can be introduced into the disclosed compositions include those produced using any method for introducing site-specific modification, including, but not limited to, through the use of gene repair oligonucleotides, or through the use of site-directed genome modification tools such as TALENs, meganucleases, zinc finger nucleases, CRISPR-Cas, and the like. Site-specific modifications to the disclosed polynucleotides (including genomic flanking and junction sequences) may include, but are not limited to, changes to codon usage, changes to regulatory elements such as promoters, introns, terminators, enhancers, 5′ or 3′ untranslated regions (UTRs), or other noncoding sequences, and other regions of the polynucleotide, where the modifications do not adversely affect the phenotypic characteristics of the resulting maize plant.

Cas polypeptides suitable for introducing site-specific modifications include, for example, Cas9, Cas 12f (Cas-alpha, Cas14), Cas121 (Cas-beta), Cas12a (Cpf1), Cas12b (a C2c1 protein), Cas13 (a C2c2 prot cin), Cas12c (a C2c3 protein), Cas12d, Cas12e, Cas12g, Cas12h, Cas12i, Cas12j, Cas12k, Cas3, Cas3-HD, Cas 5, Cas6, Cas7, Cas8, Cas10, or combinations or complexes of these. In some aspects, transposon-associated TnpB, a programmable RNA-guided DNA endonuclease can be used.

In some aspects, a genome editing system comprises a Cas-alpha (e.g., Cas12f) endonuclease and one or more guide polynucleotides that introduce one or more site-specific modifications in a target polynucleotide sequence, resulting in a modified target sequence. As used herein, “altered target site”, “altered target sequence”, “modified target site,” and “modified target sequence” are used interchangeably and refer to a target sequence as disclosed herein that comprises at least one alteration or modification when compared to a non-altered target sequence. Such alterations or modifications include, for example: (i) replacement or substitution of at least one nucleotide, (ii) deletion of at least one nucleotide, (iii) insertion of at least one nucleotide, or (iv) any combination of (i)-(iii).

In some aspects, a genome editing system comprises a Cas-alpha endonuclease, one or more guide polynucleotides, and optionally a donor DNA. Some exemplary Cas-alpha endonucleases are described, for example, in WO2020123887.

In some aspects, a genome editing system comprises a Cas polypeptide, one or more guide polynucleotides, and optionally donor DNA, and editing a target polynucleotide sequence comprises nonhomologous end-joining (NHEJ) or homologous recombination (HR) following a Cas polypeptide-mediated double-strand break. Once a double-strand break is induced in the DNA, the cell's DNA repair mechanism is activated to repair the break. The most common repair mechanism to bring the broken ends together is the nonhomologous end-joining pathway (Bleuyard et al., (2006) DNA Repair 5:1-12). The structural integrity of chromosomes is typically preserved by the repair, but deletions, insertions, or other rearrangements are possible (Siebert and Puchta, (2002) Plant Cell 14:1121-31; Pacher et al., (2007) Genetics 175:21-9). Alternatively, the double-strand break can be repaired by homologous recombination between homologous DNA sequences. Once the sequence around the double-strand break is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

In some aspects, the genome editing system comprises a Cas polypeptide, one or more guide polynucleotides, and a donor DNA. As used herein, “donor DNA” is a DNA construct that comprises a polynucleotide of interest to be inserted into the target site of a Cas polypeptide. Once a double-strand break is introduced in the target site by the endonuclease, the first and second regions of homology of the donor DNA can undergo homologous recombination with their corresponding genomic regions of homology resulting in exchange of DNA between the donor and the target genome. As such, the provided methods result in the integration of the polynucleotide of interest of the donor DNA into the double-strand break in the target site in the plant genome, thereby altering the original target site and producing an altered genomic target site.

In some aspects, a genome editing system comprises a base editing agent and a plurality of guide polynucleotides and editing a target polynucleotide sequence comprises introducing a plurality of nucleobase edits in the target polynucleotide sequence resulting in a variant nucleotide sequence.

One or more nucleobases of a target polynucleotide can be chemically altered, in some cases to change the base from one type to another, for example from a Cytosine to a Thymine, or an Adenine to a Guanine. In some aspects, a plurality of bases, for example 2 or more, 5 or more, 10 or more, 20 or more, 30 or more, 40 or more, 50 or more, 60 or more, 70 or more, 80 or more 90 or more, 100 or more, or even greater than 100, 200 or more, up to thousands of bases may be modified or altered, to produce a plant with a plurality of modified bases.

Any base editing complex, such as a base editing agent associated with an RNA-guided protein, may be used to target and bind to a desired locus in the genome of an organism and chemically modify one or more components of a target polynucleotide.

Site-specific base conversions can be achieved to engineer one or more nucleotide changes to create one or more edits into the genome. These include for example, a site-specific base edit mediated by an C·G to T·A or an A·T to G·C base editing deaminase enzymes (Gaudelli et al., Programmable base editing of A·T to G·C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016): 420-4. A catalytically “dead” or inactive Cas polypeptide, for example an inactive Cas9 (dCas9), Cas12f (dCas12f), or another Cas polypeptide disclosed herein, fused to a cytidine deaminase or an adenine deaminase protein becomes a specific base editor that can alter DNA bases without inducing a DNA break. Base editors convert C->T (or G->A on the opposite strand) or an adenine base editor that would convert adenine to inosine, resulting in an A->G change within an editing window specified by the gRNA. Any molecule that effects a change in a nucleobase is a “base editing agent”. The dCas forms a functional complex with a guide polynucleotide that shares homology with a polynucleotide sequence at the target site, and is further complexed with the deaminase molecule. The guided Cas polypeptide recognizes and binds to a double-stranded target sequence, opening the double-strand to expose individual bases. In the case of a cytidine deaminase, the deaminase deaminates the cytosine base and creates a uracil. Uracil glycosylase inhibitor (UGI) is provided to prevent the conversion of U back to C. DNA replication or repair mechanisms then convert the Uracil to a thymine (U to T), and subsequent repair of the opposing base (formerly G in the original G-C pair) to an Adenine, creating a T-A pair.

The Cas endonuclease is guided by the guide nucleotide to recognize and optionally introduce a double strand break at a specific target site into the genome of a cell. The CRISPR-Cas system provides for an effective system for modifying target sites within the genome of a plant, plant cell or seed. Further provided are methods employing a guide polynucleotide/Cas endonuclease system to provide an effective system for modifying target sites within the genome of a cell and for editing a nucleotide sequence in the genome of a cell. Once a genomic target site is identified, a variety of methods may be employed to further modify the target site. The disclosed methods can be used to introduce a CRISPR-Cas system for editing a nucleotide sequence in the genome of a cell. The nucleotide sequence to be edited (the nucleotide sequence of interest) can be located within or outside a target site that is recognized by a Cas endonuclease.

The term “Cas polypeptide” or “Cas endonuclease” refers to a polypeptide encoded by a Cas (CRISPR-associated) gene.

A Cas polypeptide includes but is not limited to: Cas9, Cas 12f (Cas-alpha, Cas14), Cas121 (Cas-beta), Cas12a (Cpf1), Cas12b (a C2c1 protein), Cas13 (a C2c2 protein), Cas12c (a C2c3 protein), Cas12d, Cas12e, Cas12g, Cas12h, Cas12i, Cas12j, Cas12k, Cas3, Cas3-HD, Cas 5, Cas6, Cas7, Cas8, Cas10, or combinations or complexes of these.

Endonucleases are enzymes that cleave the phosphodiester bond within a polynucleotide chain and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more (Patent application PCT/US 12/30061 filed on Mar. 22, 2012). Meganucleases have been classified into four families based on conserved sequence motifs. These motifs participate in the coordination of metal ions and hydrolysis of phosphodiester bonds. Meganucleases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. This cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families. TAL effector nucleases are a new class of sequence-specific nucleases that can be used to make double-strand breaks at specific target sequences in the genome of a plant or other organism. (Miller, et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a nonspecific endonuclease domain, for example nuclease domain from a Type Ms endonuclease such as Fokl.

A catalytically active and/or inactive Cas endonuclease can be fused to a heterologous sequence (US20140068797 published 6 Mar. 2014). A “Dead-CAS9” (dCAS9) as used herein, is used to supply a transcriptional repressor domain. The dCAS9 has been mutated so that can no longer cut DNA. The dCAS0 can still bind when guided to a sequence by the gRNA and can also be fused to repressor elements. The dCAS9 fused to the repressor element, as described herein, is abbreviated to dCAS9˜REP, where the repressor element (REP) can be any of the known repressor motifs that have been characterized in plants. An expressed guide RNA (gRNA) binds to the dCAS9˜REP protein and targets the binding of the dCAS9-REP fusion protein to a specific predetermined nucleotide sequence within a promoter (a promoter within the T-DNA). For example, if this is expressed beyond-the border using a ZM-UBI PRO::dCAS9˜REP::PINII TERM cassette along with a U6-POL PRO::gRNA::U6 TERM cassette and the gRNA is designed to guide the dCAS9-REP protein to bind the SB-UBI promoter in the expression cassette SB-UBI PRO::moPAT::PINII TERM within the T-DNA, any event that has integrated the beyond-the-border sequence would be bialaphos sensitive. Transgenic events that integrate only the T-DNA would express moPAT and be bialaphos resistant. The advantage of using a dCAS9 protein fused to a repressor (as opposed to a TETR or ESR) is the ability to target these repressors to any promoter within the T-DNA. TETR and ESR are restricted to cognate operator binding sequences. Alternatively, a synthetic Zinc-Finger Nuclease fused to a repressor domain can be used in place of the gRNA and dCAS9˜REP (Urritia et al., 2003, Genome Biol. 4:231) as described above.

The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target. As used herein, the term “guide nucleotide” relates to a synthetic fusion of two RNA molecules, a crRNA (CRISPR RNA) comprising a variable targeting domain, and a tracrRNA. In an aspect, the guide nucleotide comprises a variable targeting domain of 12 to 30 nucleotide sequences and a RNA fragment that can interact with a Cas endonuclease.

As used herein, the term “guide polynucleotide” relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (a RNA-DNA combination sequence).

In an aspect, the guide nucleotide and Cas endonuclease are capable of forming a complex that enables the Cas endonuclease to introduce a double strand break at a DNA target site. Cas endonucleases, either as single effector proteins or in an effector complex with other components, unwind the DNA duplex at the target sequence and optionally cleave at least one DNA strand, as mediated by recognition of the target sequence by a polynucleotide (such as, but not limited to, a crRNA or guide RNA) that is in complex with the Cas effector protein. Such recognition and cutting of a target sequence by a Cas endonuclease typically occurs if the correct protospacer-adjacent motif (PAM) is located at or adjacent to the 3′ end of the DNA target sequence. Alternatively, a Cas endonuclease herein may lack DNA cleavage or nicking activity, but can still specifically bind to a DNA target sequence when complexed with a suitable RNA component. (See also U.S. Patent Application US20150082478 published 19 Mar. 2015 and US20150059010 published 26 Feb. 2015).

Cas endonucleases may occur as individual effectors (Class 2 CRISPR systems) or as part of larger effector complexes (Class 1 CRISPR systems).

Cas endonucleases that have been described include, but are not limited to, for example: Cas3 (a feature of Class 1 type I systems), Cas9 (a feature of Class 2 type II systems) and Cas12 (Cpf1) (a feature of Class 2 type V systems).

Cas endonucleases and effector proteins can be used for targeted genome editing (via simplex and multiplex double-strand breaks and nicks) and targeted genome regulation (via tethering of epigenetic effector domains to either the Cas protein or sgRNA. A Cas endonuclease can also be engineered to function as an RNA-guided recombinase, and via RNA tethers could serve as a scaffold for the assembly of multiprotein and nucleic acid complexes (Mali et al., 2013, Nature Methods Vol. 10:957-963).

In some examples, Cas endonucleases, when complexed with a cognate guide RNA, recognize, bind to, and optionally nick or cleave a target polynucleotide.

In an aspect of the present disclosure the variable target domain is 12, 13, 14, 15, 16, 17, 18, 19, 20, 21, 22, 23, 24, 25, 26, 27, 28, 29 or 30 nucleotides in length.

The guide polynucleotide enables target recognition, binding, and optionally cleavage by the Cas endonuclease, and can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (US20150082478 published 19 Mar. 2015 and US20150059010 published 26 Feb. 2015). A guide polynucleotide may be engineered or synthetic.

The guide polynucleotide includes a chimeric non-naturally occurring guide RNA comprising regions that are not found together in nature (i.e., they are heterologous with each other). For example, a chimeric non-naturally occurring guide RNA comprising a first nucleotide sequence domain (referred to as Variable Targeting domain or VT domain) that can hybridize to a nucleotide sequence in a target DNA, linked to a second nucleotide sequence that can recognize the Cas endonuclease, such that the first and second nucleotide sequence are not found linked together in nature.

The guide polynucleotide can be a double molecule (also referred to as duplex guide polynucleotide) comprising a crNucleotide sequence (such as a crRNA) and a tracrNucleotide (such as a tracrRNA) sequence. In some cases, there is a linker polynucleotide that connects the crRNA and tracrRNA to form a single guide, for example an sgRNA.

In an aspect of the present disclosure, the guide nucleotide comprises a cRNA (or cRNA fragment) and a tracrRNA (or tracrRNA fragment) of the type II CRISPR/Cas system that can form a complex with a type II Cas endonuclease, wherein the guide nucleotide Cas endonuclease complex can direct the Cas endonuclease to a plant genomic target site, enabling the Cas endonuclease to introduce a double strand break into the genomic target site. The guide nucleotide can be introduced into a plant or plant cell directly using any method known in the art such as, but not limited to, particle bombardment or topical applications.

In an aspect, the guide nucleotide can be introduced indirectly by introducing a recombinant DNA molecule comprising the corresponding guide DNA sequence operably linked to a plant specific promoter that is capable of transcribing the guide nucleotide in the plant cell. The term “corresponding guide DNA” includes a DNA molecule that is identical to the RNA molecule but has a “T” substituted for each “U” of the RNA molecule.

In an aspect, the guide nucleotide is introduced via particle bombardment or using the disclosed methods for Agrobacterium transformation of a recombinant DNA construct comprising the corresponding guide DNA operably linked to a plant U6 polymerase III promoter.

In an aspect, the RNA that guides the RNA Cas9 endonuclease complex, is a duplexed RNA comprising a duplex crRNA-tracrRNA. One advantage of using a guide nucleotide versus a duplexed crRNA-tracrRNA is that only one expression cassette needs to be made to express the fused guide nucleotide.

The terms “target site,” “target sequence,” “target DNA,” “target locus,” “genomic target site,” “genomic target sequence,” and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature.

As used herein, terms “endogenous target sequence” and “native target sequence” are used interchangeably herein to refer to a target sequence that is endogenous or native to the genome of a plant and is at the endogenous or native position of that target sequence in the genome of the plant.

An “artificial target site” or “artificial target sequence” are used interchangeably herein and refer to a target sequence that has been introduced into the genome of a plant. Such an artificial target sequence can be identical in sequence to an endogenous or native target sequence in the genome of a plant but be located in a different position (i.e., a non-endogenous or non-native position) in the genome of a plant.

In an aspect, the disclosed methods can be used to introduce into plants polynucleotides useful for gene suppression of a target gene in a plant. Reduction of the activity of specific genes (also known as gene silencing, or gene suppression) is desirable for several aspects of genetic engineering in plants. Many techniques for gene silencing are well known to one of skill in the art, including but not limited to antisense technology, siRNA, microRNA, and the like. Other approaches may also be used, for example, using a dCas fused to a transcriptional repressor and/or chromatin modifying domain to induce a targeted reduction in transcript. See, for example, WO2022087616, incorporated herein by reference in its entirety.

The crNucleotide includes a first nucleotide sequence domain (referred to as Variable Targeting domain or VT domain) that can hybridize to a nucleotide sequence in a target DNA and a second nucleotide sequence (also referred to as a tracr mate sequence) that is part of a Cas endonuclease recognition (CER) domain. The tracr mate sequence can hybridized to a tracrNucleotide along a region of complementarity and together form the Cas endonuclease recognition domain or CER domain. The CER domain is capable of interacting with a Cas endonuclease polypeptide. The crNucleotide and the tracrNucleotide of the duplex guide polynucleotide can be RNA, DNA, and/or RNA-DNA-combination sequences. In some embodiments, the crNucleotide molecule of the duplex guide polynucleotide is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the crRNA naturally occurring in Bacteria and Archaca. The size of the fragment of the crRNA naturally occurring in Bacteria and Archaea that can be present in a crNucleotide disclosed herein can range from, but is not limited to, 2, 3, 4, 5, 6, 7, 8, 9, 10, 11, 12, 13, 14, 15, 16, 17, 18, 19, 20 or more nucleotides.

In some embodiments the tracrNucleotide is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In one embodiment, the RNA that guides the RNA/Cas9 endonuclease complex is a duplexed RNA comprising a duplex crRNA-tracrRNA. The tracrRNA (trans-activating CRISPR RNA) comprises, in the 5′-to-3′ direction, (i) a sequence that anneals with the repeat region of CRISPR type II crRNA and (ii) a stem loop-comprising portion (Deltcheva et al., Nature 471:602-607). The duplex guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double-strand break) into the target site. (US20150082478 published 19 Mar. 2015 and US20150059010 published 26 Feb. 2015).

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide.

A “protospacer adjacent motif” (PAM) herein refers to a short nucleotide sequence adjacent to a target sequence (protospacer) that can be recognized (targeted) by a guide polynucleotide/Cas endonuclease system. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

A “randomized PAM” and “randomized protospacer adjacent motif” are used interchangeably herein, and refer to a random DNA sequence adjacent to a target sequence (protospacer) that is recognized (targeted) by a guide polynucleotide/Cas endonuclease system. The randomized PAM sequence can be of any length but is typically 1, 2, 3, 4, 5, 6, 7, 8, 9, 10, 11, 12, 13, 14, 15, 16, 17, 18, 19 or 20 nucleotides long. A randomized nucleotide includes anyone of the nucleotides A, C, G or T.

A guide polynucleotide/Cas endonuclease complex described herein is capable of recognizing, binding to, and optionally nicking, unwinding, or cleaving all or part of a target sequence.

Proteins may be altered in various ways including amino acid substitutions, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not anticipated to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates comprising target sites.

The nucleotide to be edited can be located within or outside a target site recognized and cleaved by a Cas endonuclease. In one embodiment, the at least one nucleotide modification is not a modification at a target site recognized and cleaved by a Cas endonuclease. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

A knock-out may be produced by an indel (insertion or deletion of nucleotide bases in a target DNA sequence through NHEJ), or by specific removal of sequence that reduces or completely destroys the function of sequence at or near the targeting site.

A guide polynucleotide/Cas endonuclease induced targeted mutation can occur in a nucleotide sequence that is located within or outside a genomic target site that is recognized and cleaved by the Cas endonuclease.

The method for editing a nucleotide sequence in the genome of a cell can be a method without the use of an exogenous selectable marker by restoring function to a non-functional gene product.

In one embodiment, the invention describes a method for modifying a target site in the genome of a cell, the method comprising introducing into a cell at least one PGEN described herein and at least one donor DNA, wherein said donor DNA comprises a polynucleotide of interest, and optionally, further comprising identifying at least one cell that said polynucleotide of interest integrated in or near said target site.

In one aspect, the methods disclosed herein may employ homologous recombination (HR) to provide integration of the polynucleotide of interest at the target site.

Various methods and compositions can be employed to produce a cell or organism having a polynucleotide of interest inserted in a target site via activity of a CRISPR-Cas system component described herein. In one method described herein, a polynucleotide of interest is introduced into the organism cell via a donor DNA construct. The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10:957-963).

In one embodiment, the disclosure comprises a method for editing a nucleotide sequence in the genome of a cell, the method comprising introducing into at least one PGEN described herein, and a polynucleotide modification template, wherein said polynucleotide modification template comprises at least one nucleotide modification of said nucleotide sequence, and optionally further comprising selecting at least one cell that comprises the edited nucleotide sequence.

The guide polynucleotide/Cas endonuclease system can be used in combination with at least one polynucleotide modification template to allow for editing (modification) of a genomic nucleotide sequence of interest. (See also US20150082478, published 19 Mar. 2015 and WO2015026886 published 26 Feb. 2015).

Further uses for guide RNA/Cas endonuclease systems have been described (See for example: US20150082478 published 19 Mar. 2015, WO2015026886 published 26 Feb. 2015, US20150059010 published 26 Feb. 2015, WO2016007347 published 14 Jan. 2016, and PCT application WO2016025131 published 18 Feb. 2016) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

The methods and compositions described herein do not depend on a particular method for introducing a sequence into an organism or cell, only that the polynucleotide or polypeptide gains access to the interior of at least one cell of the organism. Introducing includes reference to the incorporation of a nucleic acid into a eukaryotic or prokaryotic cell where the nucleic acid may be incorporated into the genome of the cell, and includes reference to the transient (direct) provision of a nucleic acid, protein or polynucleotide-protein complex (PGEN, RGEN) to the cell.

Methods for introducing polynucleotides or polypeptides or a polynucleotide-protein complex into cells or organisms are known in the art including, but not limited to, microinjection, electroporation, stable transformation methods, transient transformation methods, ballistic particle acceleration (particle bombardment), whiskers mediated transformation, Agrobacterium-mediated transformation, direct gene transfer, viral-mediated introduction, transfection, transduction, cell-penetrating peptides, mesoporous silica nanoparticle (MSN)-mediated direct protein delivery, topical applications, sexual crossing, sexual breeding, and any combination thereof.

The polynucleotide or recombinant DNA construct can be provided to or introduced into a prokaryotic and eukaryotic cell or organism using a variety of transient transformation methods. Such transient transformation methods include, but are not limited to, the introduction of the polynucleotide construct directly into the plant. Nucleic acids and proteins can be provided to a cell by any method including methods using molecules to facilitate the uptake of anyone or all components of a guided Cas system (protein and/or nucleic acids), such as cell-penetrating peptides and nanocarriers. See also US20110035836 published 10 Feb. 2011, and EP2821486A1 published 7 Jan. 2015.

Other methods of introducing polynucleotides into a prokaryotic and eukaryotic cell or organism or plant part can be used, including plastid transformation methods, and the methods for introducing polynucleotides into tissues from seedlings or mature seeds.

Stable transformation is intended to mean that the nucleotide construct introduced into an organism integrates into a genome of the organism and is capable of being inherited by the progeny thereof. Transient transformation is intended to mean that a polynucleotide is introduced into the organism and does not integrate into a genome of the organism or a polypeptide is introduced into an organism. Transient transformation indicates that the introduced composition is only temporarily expressed or present in the organism.

A variety of methods are available to identify those cells having an altered genome at or near a target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

In an aspect, the disclosed methods can be used to introduce into plants polynucleotides useful for the targeted integration of nucleotide sequences into a plant. For example, the disclosed methods can be used to introduce T-DNA expression cassettes comprising nucleotide sequences of interest flanked by non-identical recombination sites to transform a plant comprising a target site. See, for example, WO2022087616, incorporated herein by reference in its entirety.

In an aspect, the genomic target site contains at least a set of non-identical recombination sites corresponding to those on the T-DNA expression cassette. A recombinase mediates the exchange of the nucleotide sequences flanked by the recombination sites. Thus, the disclosed methods can be used for the introduction of T-DNA expression cassettes for targeted integration of nucleotide sequences, wherein the T-DNA expression cassettes flanked by non-identical recombination sites are recognized by a recombinase which implements recombination at the nonidentical recombination sites. Accordingly, the disclosed methods and composition can be used to improve efficiency and speed of development of plants containing non-identical recombination sites.

Thus, the disclosed methods can further comprise methods for the directional, targeted integration of exogenous nucleotides into a plant cell. In an aspect, the disclosed methods use recombination sites in a gene targeting system which facilitates directional targeting of desired genes and nucleotide sequences into corresponding recombination sites previously introduced into the target plant genome.

By suitable non-identical recombination site implies that in the presence of active recombinase, excision of sequences between two non-identical recombination sites occurs, if at all, with an efficiency considerably lower than the recombinationally-mediated exchange targeting arrangement of nucleotide sequences into the plant genome. Thus, suitable non-identical sites for use in the present disclosure include those sites where the efficiency of recombination between the sites is low; for example, where the efficiency is less than about 30 to about 50%, preferably less than about 10 to about 30%, more preferably less than about 5 to about 10%.

As noted above, in one embodiment, the recombination sites in the targeting cassette correspond to those in the target site of the transformed plant. That is, if the target site of the transformed plant contains flanking non-identical recombination sites of FRT and a mutant FRT, the targeting cassette will contain the same FRT and mutant FRT non-identical recombination sites. If FRT sites are utilized, the FLP recombinase will be needed. In the same manner, where lox sites are utilized, the Cre recombinase is required. If the non-identical recombination sites comprise both a FRT and a lox site, both the FLP and Cre recombinase will be required in the plant cell.

Where appropriate, the nucleotide sequences to be inserted in the plant genome may be optimized for increased expression. Where mammalian, yeast, or bacterial genes are used in the present disclosure, they can be synthesized using plant preferred codons for improved expression. It is recognized that for expression in monocots, dicot genes can also be synthesized using monocot preferred codons. It is further recognized that all or any part of the gene sequence may be optimized or synthetic. That is, fully optimized or partially optimized sequences may also be used.

Additional sequence modifications are known to enhance gene expression in a cellular host and can be used in the present disclosure. These include elimination of sequences encoding spurious polyadenylation signals, exon-intron splice site signals, transposon-like repeats, and other such well-characterized sequences, which may be deleterious to gene expression. The G-C content of the sequence may be adjusted to levels average for a given cellular host, as calculated by reference to known genes expressed in the host cell. When possible, the sequence is modified to avoid predicted hairpin secondary RNA structures.

The present disclosure also encompasses FLP recombination target sites (FRT). The FRT has been identified as a minimal sequence comprising two 13 base pair repeats, separated by an eight (8) base spacer. The nucleotides in the spacer region can be replaced with a combination of nucleotides, so long as the two 13-base repeats are separated by eight nucleotides.

FRT mutant sites can be used in the practice of the disclosed methods. Although mutant FRT sites are known (see SEQ ID NO: 2, 3, 4 and 5 of WO1999/025821), it is recognized that other mutant FRT sites may be used in the practice of the present disclosure. The present disclosure is not restricted to the use of a particular FRT or recombination site, but rather that non-identical recombination sites or FRT sites can be utilized for targeted insertion and expression of nucleotide sequences in a plant genome. Thus, other mutant FRT sites can be constructed and utilized based upon the present disclosure.

As discussed above, bringing genomic DNA containing a target site with non-identical recombination sites together with a vector containing a T-DNA expression cassette with corresponding non-identical recombination sites, in the presence of the recombinase, results in recombination. The nucleotide sequence of the T-DNA expression cassette located between the flanking recombination sites is exchanged with the nucleotide sequence of the target site located between the flanking recombination sites. In this manner, nucleotide sequences of interest may be precisely incorporated into the genome of the host.

It is recognized that many variations of the present disclosure can be practiced. For example, target sites can be constructed having multiple non-identical recombination sites. Thus, multiple genes or nucleotide sequences can be stacked or ordered at precise locations in the plant genome. Likewise, once a target site has been established within the genome, additional recombination sites may be introduced by incorporating such sites within the nucleotide sequence of the T-DNA expression cassette and the transfer of the sites to the target sequence. Thus, once a target site has been established, it is possible to subsequently add sites, or alter sites through recombination.

Another variation includes providing a promoter or transcription initiation region operably linked with the target site in an organism. Preferably, the promoter will be 5′ to the first recombination site. By transforming the organism with a T-DNA expression cassette comprising a coding region, expression of the coding region will occur upon integration of the T-DNA expression cassette into the target site. This aspect provides for a method to select transformed cells, particularly plant cells, by providing a selectable marker sequence as the coding sequence.

The present disclosure is further illustrated in the following embodiments. It should be understood that these embodiments are given by way of illustration only and not by way of limitation.

Embodiment 1. A method of inducing parthenogenesis, the method comprising:
expressing, in a female gametophyte, a Babyboom (BBM) polynucleotide operably linked to a promoter that expresses in a plant egg cell, wherein the Babyboom (BBM) polynucleotide comprises a nucleotide sequence encoding a Babyboom (BBM) polypeptide or a fragment thereof, wherein the female gametophyte is rendered parthenogenic and forms a haploid embryo without pollination, and wherein the Babyboom (BBM) polynucleotide is not naturally expressed in a female gametophyte.

Embodiment 2. The method of embodiment 1, wherein the nucleotide sequence encoding the Babyboom (BBM) polypeptide or fragment thereof is selected from the group consisting of: BBM1, BBM2, BMN2, BMN3, ODP2, and BBML.

Embodiment 3. The method of embodiment 1, wherein the Babyboom (BBM) polynucleotide is selected from the group consisting of:
(a) a nucleotide sequence as set forth in SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(b) a nucleotide sequence that has at least 95% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(c) a nucleotide sequence that has at least 85% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(d) a fragment of any of the nucleotide sequences of (a), (b), or (c), wherein the nucleotide sequence encodes an amino acid fragment that has haploid induction activity;
(e) a fragment of any of the nucleotide sequences of (a), (b), (c), or (d), wherein the nucleotide sequence encodes an amino acid fragment that has haploid induction activity, wherein the amino acid fragment comprises an A motif, a B motif, or an AP2 DNA binding domain, or combinations thereof.

Embodiment 4. The method of embodiment 1, wherein the Babyboom (BBM) polypeptide is selected from the group consisting of:
(a) an amino acid sequence as set forth in SEQ ID NO: 2, 4, 6, 8, 10, 12, 14, 21, 23, 25, 27, 29, 42, 44, 46, or 48;
(b) an amino acid sequence that has at least 95% amino acid identity to any one of SEQ ID NO: 2, 4, 6, 8, 10, 12, 14, 21, 23, 25, 27, 29, 42, 44, 46, or 48;
(c) an amino acid sequence that has at least 85% amino acid identity to any one of SEQ ID NO: 2, 4, 6, 8, 10, 12, 14, 21, 23, 25, 27, 29, 42, 44, 46, or 48;
(d) a fragment of any of the amino acid sequences of (a), (b), or (c), wherein the fragment has haploid induction activity; and
(e) a fragment of any of the amino acid sequences of (a), (b), or (c) or (d), wherein the fragment comprises an A motif, a B motif, or an AP2 DNA binding domain, or combinations thereof.

Embodiment 5. The method of embodiment 1, wherein the promoter that expresses in the egg cell is an egg cell-specific promoter or an egg cell-preferred promoter.

Embodiment 6. The method of embodiment 5, wherein the promoter that expresses in the egg cell comprises:
(a) a polynucleotide sequence of SEQ ID NO: 15, 16, 17, 18, 19, 31, 32, 33, 34, 35, 36, 37, 38, 39, 40, or 86, or variants thereof;
(b) a polynucleotide sequence having at least 90% sequence identity to SEQ ID NO: 15, 16, 17, 18, 19, 31, 32, 33, 34, 35, 36, 37, 38, 39, 40, or 86, or variants thereof;
(c) a fragment of the polynucleotide of (a) or (b), e.g., a fragment of at least 100, 200, 300, 400, 500, 600, 700, 800, 900, 1000, 1100, 1200, 1300, 1400, 1500, 1600, 1700, 1800, 1900, or 2000 contiguous nucleotides of (a) or (b), wherein the fragment retains its ability to drive expression of the operably linked Babyboom (BBM) polynucleotide encoding the Babyboom (BBM) polypeptide or the fragment thereof.

Embodiment 7. The method of embodiment 1, wherein the promoter that expresses in the egg cell further comprises an expression modulation element.

Embodiment 8. The method of embodiment 1, wherein the expression of the Babyboom (BBM) polynucleotide by an egg cell promoter comprising an expression modulation element increases haploid induction frequency of the female gametophyte, as compared to haploid induction frequency resulting from expression of a Babyboom (BBM) polynucleotide driven by an egg cell promoter without an expression modulation element.

Embodiment 9. The method of embodiment 1, wherein the female gametophyte has not been fertilized prior to or during expression of the parthenogenesis factor, e.g. the BBM polynucleotide, in the egg cell.

Embodiment 10. The method of embodiment 1, further comprising: modifying a regulatory region of an endogenous Babyboom (BBM) polynucleotide so that the Babyboom (BBM) polynucleotide expresses in a female gametophyte.

Embodiment 11. The method of embodiment 1, the method comprising:
expressing the Babyboom (BBM) polynucleotide from a modified endogenous genomic BBM locus, wherein the genomic BBM locus comprises a modified regulatory region of an endogenous polynucleotide encoding a BBM polypeptide, wherein one or more nucleotides in the regulatory region have been modified so that the BBM polypeptide expresses in a female gametophyte.

Embodiment 12. The method of embodiment 1, the method comprising:
expressing the Babyboom (BBM) polynucleotide from a modified endogenous genomic egg cell locus, wherein the egg cell's coding or genomic sequence has been modified so that it encodes a BBM polypeptide, wherein the BBM polypeptide expresses in a female gametophyte.

Embodiment 13. The method of embodiment 1, wherein the Babyboom (BBM) polynucleotide has been modified from its native form using a gene editing technology.

Embodiment 14. The method of embodiment 13, wherein the gene editing technology uses a DNA modification enzyme that is a site-directed nuclease selected from the group comprising meganucleases (MNs), zinc-finger nucleases (ZFN), transcription-activator like effector nucleases (TALENs), Cas polypeptides, such as, dCas9-FokI, dCpf1-FokI, chimeric Cas9-cytidine deaminase, chimeric Cas9 adenine deaminase, chimeric FEN1-Fok1, Mega-TALs, a nickase Cas9 (nCas9), chimeric dCas9 non-FokI nuclease, dCpf1-non-FokI nuclease, Cas9, Cas12f (Cas-alpha, Cas14), Cas121 (Cas-beta), Cas12a (Cpf1), Cas12b (a C2c1 protein), Cas13 (a C2c2 protein), Cas12c (a C2c3 protein), Cas12d, Cas12e, Cas12g, Cas12h, Cas12i, Cas12j, Cas12k, Cas3, Cas3-HD, Cas 5, Cas6, Cas7, Cas8, Cas10, or combinations or complexes of these.

Embodiment 15. The method of embodiment 1, wherein the female gametophyte is a monocot or dicot female gametophyte.

Embodiment 16. The method of embodiment 1, wherein the female gametophyte is a wheat, cotton, sunflower, safflower, tobacco, Arabidopsis, cannabis, sugarcane, soy, barley, oats, rice, maize, triticale, sorghum, rye, millet, or a flax female gametophyte.

Embodiment 17. The method of embodiment 1, further comprising:
(a) contacting the haploid embryo with a chromosome doubling agent for a period sufficient to generate a doubled haploid embryo;
(b) isolating the doubled haploid embryo; and
(c) regenerating a doubled haploid plant from the doubled haploid embryo of step (b).

Embodiment 18. The method of embodiment 1, further comprising:
(a) regenerating a parthenogenic plant from a haploid embryo comprising the Babyboom (BBM) polynucleotide operably linked to the promoter that expresses in an egg cell;
(b) pollinating the parthenogenic plant of (a) with pollen from a non-haploid inducer; and
(c) rescuing a haploid embryo from the parthenogenic plant of (b).

Embodiment 19. A method for obtaining a wheat plant producing clonal, non-reduced, non-recombined gametes, the method comprising:

(a) suppressing in a wheat plant cell the activity of:

(1) all endogenous Spo11 or Prd1, Prd2, or Prd3 polynucleotides or polypeptides;
(2) all endogenous Rec8 polynucleotides or polypeptides;
(3) all endogenous Osd1 polynucleotides or polypeptides;
(b) expressing, in a female gametophyte derived from the wheat plant cell, a Babyboom (BBM) polynucleotide operably linked to a promoter that expresses in an egg cell, wherein the Babyboom (BBM) polynucleotide comprises a nucleotide sequence encoding a Babyboom (BBM) polypeptide or a fragment thereof, wherein the female gametophyte is rendered parthenogenic and forms an embryo;
(c) obtaining a plant from the embryo, wherein the embryo comprises the egg cell expressed Babyboom (BBM) polynucleotide and suppressed Spo11, Rec8, Osd1, Prd1, Prd2, or Prd3 polynucleotides or polypeptides, thereby producing a wheat plant producing clonal, non-reduced, non-recombined gametes.

Embodiment 20. The method of embodiment 19, wherein the plant is a first filial generation hybrid plant.

Embodiment 21. The method of embodiment 19, wherein the activity of the endogenous Spo11 polynucleotides or polypeptides, Rec8 polynucleotides or polypeptides, Osd1 polynucleotides or polypeptides, Prd1, Prd2, or Prd3 polynucleotides or polypeptides, or combinations thereof is suppressed by introducing a nucleotide modification into its polynucleotide sequence or an amino acid modification into its polypeptide sequence.

Embodiment 22. The method of embodiment 21, wherein the nucleotide modification is a deletion, addition, or substitution of one or more nucleotides, and wherein the amino acid modification is a deletion, addition, or substitution of one or more amino acids

Embodiment 23. The method of embodiment 21, wherein said the nucleotide modification is introduced by a nuclease selected from the group consisting of: a TALEN, a meganuclease, a zinc finger nuclease, and a CRISPR-associated nuclease.

Embodiment 24. The method of embodiment 21, wherein the nucleotide modification is introduced by a Cas endonuclease, e.g., Cas9, Cas12f (Cas-alpha, Cas14), Cas121 (Cas-beta), Cas12a (Cpf1), Cas12b (a C2c1 protein), Cas13 (a C2c2 protein), Cas12c (a C2c3 protein), Cas12d, Cas12e, Cas12g, Cas12h, Cas12i, Cas12j, Cas12k, Cas3, Cas3-HD, Cas 5, Cas6, Cas7, Cas8, Cas10, or combinations or complexes of these, guided by at least one guide RNA.

Embodiment 25. The method of embodiment 21, wherein the promoter that expresses in the egg cell comprises:
(a) a polynucleotide sequence of SEQ ID NO: 15, 16, 17, 18, 19, 31, 32, 33, 34, 35, 36, 37, 38, 39, 40, or 86, or variants thereof;
(b) a polynucleotide sequence having at least 90% sequence identity to SEQ ID NO: 15, 16, 17, 18, 19, 31, 32, 33, 34, 35, 36, 37, 38, 39, 40, or 86, or variants thereof;
(c) a fragment of the polynucleotide of (a) or (b), e.g., a fragment of at least 100, 200, 300, 400, 500, 600, 700, 800, 900, 1000, 1100, 1200, 1300, 1400, 1500, 1600, 1700, 1800, 1900, or 2000 contiguous nucleotides of (a) or (b), wherein the fragment retains its ability to drive expression of the operably linked Babyboom (BBM) polynucleotide encoding the Babyboom (BBM) polypeptide or the fragment thereof.

Embodiment 26. The method of embodiment 19, wherein the nucleotide sequence encoding the Babyboom (BBM) polypeptide or fragment thereof is selected from the group consisting of BBM1, BBM2, BMN2, BMN3, ODP2, and BBML.

Embodiment 27. The method of embodiment 19, wherein the Babyboom (BBM) polynucleotide is selected from the group consisting of:
(a) a nucleotide sequence as set forth in SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(b) a nucleotide sequence that has at least 95% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(c) a nucleotide sequence that has at least 85% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(d) a fragment of any of the nucleotide sequences of (a), (b), or (c), wherein the nucleotide sequence encodes an amino acid fragment that has haploid induction activity;
(e) a fragment of any of the nucleotide sequences of (a), (b), (c), or (d), wherein the nucleotide sequence encodes an amino acid fragment that has haploid induction activity, wherein the amino acid fragment comprises an A motif, a B motif, or an AP2 DNA binding domain, or combinations thereof.

Embodiment 28. The method of embodiment 19, wherein the Babyboom (BBM) is selected from the group consisting of:

(a) an amino acid sequence as set forth in SEQ ID NO: 2, 4, 6, 8, 10, 12, 14, 21, 23, 25, 27, 29, 42, 44, 46, or 48;

(b) an amino acid sequence that has at least 95% amino acid identity to any one of SEQ ID NO: 2, 4, 6, 8, 10, 12, 14, 21, 23, 25, 27, 29, 42, 44, 46, or 48;

(c) an amino acid sequence that has at least 85% amino acid identity to any one of SEQ ID NO: 2, 4, 6, 8, 10, 12, 14, 21, 23, 25, 27, 29, 42, 44, 46, or 48;

(d) a fragment of any of the amino acid sequences of (a), (b), or (c), wherein the fragment has haploid induction activity; and

(e) a fragment of any of the amino acid sequences of (a), (b), or (c) or (d), wherein the fragment comprises an A motif, a B motif, or an AP2 DNA binding domain, or combinations thereof.

Embodiment 29. A method of obtaining a clonal apomictic plant from one or more gametophytic cells in a plant in the absence of egg cell fertilization comprising: (a) expressing, in one or more gametophytic cell, a Babyboom (BBM) polynucleotide operably linked to a promoter that expresses in an egg cell, wherein the Babyboom (BBM) polynucleotide comprises a nucleotide sequence encoding a Babyboom (BBM) polypeptide or a fragment thereof that retains haploid induction activity, wherein the Babyboom (BBM) polynucleotide is not naturally expressed in a female gametophyte;
(b) developing an embryo from the gametophytic cell in the absence of egg cell fertilization; and
(c) obtaining progeny plant from one or more gametophytic cells wherein the progeny plant contains the chromosomes from the gametophytic cell of (a), thereby achieving propagation of a flowering plant in the absence of egg cell fertilization.

Embodiment 30. The method of embodiment 29, wherein the embryo is formed from an unreduced plant cell

Embodiment 31. The method of embodiment 30, wherein the unreduced plant cell is an egg cell or is formed from a somatic cell.

Embodiment 32. The method of embodiment 29, wherein the promoter that expresses in the egg cell comprises:
(a) a polynucleotide sequence of SEQ ID NO: 15, 16, 17, 18, 19, 31, 32, 33, 34, 35, 36, 37, 38, 39, 40, or 86, or variants thereof;
(b) a polynucleotide sequence having at least 90% sequence identity to SEQ ID NO: 15, 16, 17, 18, 19, 31, 32, 33, 34, 35, 36, 37, 38, 39, 40, or 86, or variants thereof;
(c) a fragment of the polynucleotide of (a) or (b), e.g., a fragment of at least 100, 200, 300, 400, 500, 600, 700, 800, 900, 1000, 1100, 1200, 1300, 1400, 1500, 1600, 1700, 1800, 1900, or 2000 contiguous nucleotides of (a) or (b), wherein the fragment retains its ability to drive expression of the operably linked Babyboom (BBM) polynucleotide encoding the Babyboom (BBM) polypeptide or the fragment thereof.

Embodiment 33. The method of embodiment 29, wherein the nucleotide sequence encoding the Babyboom (BBM) polypeptide or fragment thereof is selected from the group consisting of BBM1, BBM2, BMN2, BMN3, ODP2, and BBML.

Embodiment 34. The method of embodiment 29, wherein the Babyboom (BBM) polynucleotide is selected from the group consisting of:
(a) a nucleotide sequence as set forth in SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(b) a nucleotide sequence that has at least 95% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(c) a nucleotide sequence that has at least 85% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(d) a fragment of any of the nucleotide sequences of (a), (b), or (c), wherein the nucleotide sequence encodes an amino acid fragment that has haploid induction activity;
(e) a fragment of any of the nucleotide sequences of (a), (b), (c), or (d), wherein the nucleotide sequence encodes an amino acid fragment that has haploid induction activity, wherein the amino acid fragment comprises an A motif, a B motif, or an AP2 DNA binding domain, or combinations thereof.

Embodiment 35. The method of embodiment 29, wherein the Babyboom (BBM) is selected from the group consisting of:

(a) an amino acid sequence as set forth in SEQ ID NO: 2, 4, 6, 8, 10, 12, 14, 21, 23, 25, 27, 29, 42, 44, 46, or 48;

(b) an amino acid sequence that has at least 95% amino acid identity to any one of SEQ ID NO: 2, 4, 6, 8, 10, 12, 14, 21, 23, 25, 27, 29, 42, 44, 46, or 48;

(c) an amino acid sequence that has at least 85% amino acid identity to any one of SEQ ID NO: 2, 4, 6, 8, 10, 12, 14, 21, 23, 25, 27, 29, 42, 44, 46, or 48;

(d) a fragment of any of the amino acid sequences of (a), (b), or (c), wherein the fragment has haploid induction activity; and

(e) a fragment of any of the amino acid sequences of (a), (b), or (c) or (d), wherein the fragment comprises an A motif, a B motif, or an AP2 DNA binding domain, or combinations thereof.

Embodiment 36. A method of modulating expression of an endogenous polynucleotide in a genomic locus of a plant cell, the method comprising:
modifying one or more nucleotides in a regulatory region of the genomic locus comprising an endogenous polynucleotide encoding a BBM polypeptide to create a modified regulatory region, wherein creating the modified regulatory region results in expression of the BBM polypeptide in a female gametophyte.

Embodiment 37. The method of embodiment 36, wherein the modification of one or more nucleotides is by introducing one or more nucleotides, altering one or more nucleotides, or deleting one or more nucleotides, or combinations thereof.

Embodiment 38. The method of embodiment 36, wherein the BBM polypeptide is selected from the group consisting of:
(a) an amino acid sequence as set forth in SEQ ID NO: 2, 4, 6, 8, 10, 12, 14, 21, 23, 25, 27, 29, 42, 44, 46, or 48;
(b) an amino acid sequence that has at least 95% amino acid identity to any one of SEQ ID NO: 2, 4, 6, 8, 10, 12, 14, 21, 23, 25, 27, 29, 42, 44, 46, or 48;
(c) an amino acid sequence that has at least 85% amino acid identity to any one of SEQ ID NO: 2, 4, 6, 8, 10, 12, 14, 21, 23, 25, 27, 29, 42, 44, 46, or 48;
(d) a fragment of any of the amino acid sequences of (a), (b), or (c), wherein the fragment has haploid induction activity; and
(e) a fragment of any of the amino acid sequences of (a), (b), or (c) or (d), wherein the fragment comprises an A motif, a B motif, or an AP2 DNA binding domain, or combinations thereof.

Embodiment 39. The method of embodiment 36, wherein the regulatory region has been modified to comprise egg cell-specific promoters or egg cell-specific sequence motifs.

Embodiment 40. The method of embodiment 36, wherein the regulatory region has been modified to comprise a promoter that expresses in the egg cell, wherein the promoter comprises:
(a) a polynucleotide sequence of SEQ ID NO: 15, 16, 17, 18, 19, 31, 32, 33, 34, 35, 36, 37, 38, 39, 40, or 86, or variants thereof;
(b) a polynucleotide sequence having at least 90% sequence identity to SEQ ID NO: 15, 16, 17, 18, 19, 31, 32, 33, 34, 35, 36, 37, 38, 39, 40, or 86, or variants thereof;
(c) a fragment of the polynucleotide of (a) or (b), e.g., a fragment of at least 100, 200, 300, 400, 500, 600, 700, 800, 900, 1000, 1100, 1200, 1300, 1400, 1500, 1600, 1700, 1800, 1900, or 2000 contiguous nucleotides of (a) or (b), wherein the fragment retains its ability to drive expression of the operably linked Babyboom (BBM) polynucleotide encoding the Babyboom (BBM) polypeptide or the fragment thereof.

Embodiment 41. The method of embodiment 36, wherein the regulatory region has been modified to comprise an expression modulating element.

Embodiment 42. A modified genomic locus of a plant cell, wherein the genomic locus comprises a modified regulatory region of an endogenous polynucleotide encoding a BBM polypeptide, wherein one or more nucleotides in the regulatory region have been modified so that the BBM polypeptide expresses in a female gametophyte.

Embodiment 43. The modified genomic locus of embodiment 42, wherein the BBM polypeptide is selected from the group consisting of:
(a) an amino acid sequence as set forth in SEQ ID NO: 2, 4, 6, 8, 10, 12, 14, 21, 23, 25, 27, 29, 42, 44, 46, or 48;
(b) an amino acid sequence that has at least 95% amino acid identity to any one of SEQ ID NO: 2, 4, 6, 8, 10, 12, 14, 21, 23, 25, 27, 29, 42, 44, 46, or 48;
(c) an amino acid sequence that has at least 85% amino acid identity to any one of SEQ ID NO: 2, 4, 6, 8, 10, 12, 14, 21, 23, 25, 27, 29, 42, 44, 46, or 48;
(d) a fragment of any of the amino acid sequences of (a), (b), or (c), wherein the fragment has haploid induction activity; and
(e) a fragment of any of the amino acid sequences of (a), (b), or (c) or (d), wherein the fragment comprises an A motif, a B motif, or an AP2 DNA binding domain, or combinations thereof.

Embodiment 44. The modified genomic locus of embodiment 42, wherein the regulatory region has been modified to comprise egg cell-specific promoters or egg cell-specific sequence motifs.

Embodiment 45. The modified genomic locus of embodiment 42, wherein the regulatory region has been modified to comprise a promoter that expresses in the egg cell, wherein the promoter comprises:
(a) a polynucleotide sequence of SEQ ID NO: 15, 16, 17, 18, 19, 31, 32, 33, 34, 35, 36, 37, 38, 39, 40, or 86, or variants thereof;
(b) a polynucleotide sequence having at least 90% sequence identity to SEQ ID NO: 15, 16, 17, 18, 19, 31, 32, 33, 34, 35, 36, 37, 38, 39, 40, or 86, or variants thereof;
(c) a fragment of the polynucleotide of (a) or (b), e.g., a fragment of at least 100, 200, 300, 400, 500, 600, 700, 800, 900, 1000, 1100, 1200, 1300, 1400, 1500, 1600, 1700, 1800, 1900, or 2000 contiguous nucleotides of (a) or (b), wherein the fragment retains its ability to drive expression of the operably linked Babyboom (BBM) polynucleotide encoding the Babyboom (BBM) polypeptide or the fragment thereof.

Embodiment 46. The modified genomic locus of embodiment 42, wherein the regulatory region has been modified to comprise an enhancer expressing element.

Embodiment 47. A method of modulating a genomic or coding sequence of an endogenous polynucleotide in a genomic locus of a plant cell, the method comprising: modifying a genomic or coding sequence of an endogenous genomic egg cell locus to create a modified genomic or coding sequence comprising a heterologous polynucleotide encoding a BBM polypeptide, wherein the modification results in expression of the BBM polypeptide in a female gametophyte.

Embodiment 48. The method of embodiment 47, wherein the modification of the endogenous genomic or coding sequence is by
(a) replacing one or more nucleotides of the endogenous coding sequence;
(b) introducing one or more nucleotides into the endogenous coding sequence; (c) altering one or more nucleotides of the endogenous coding sequence; or
(d) deleting one or more nucleotides of the endogenous coding sequence;
or combinations thereof.

Embodiment 49. The method of embodiment 47, wherein the heterologous polynucleotide is selected from the group consisting of:
(a) a nucleotide sequence as set forth in SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(b) a nucleotide sequence that has at least 95% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(c) a nucleotide sequence that has at least 85% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(d) a fragment of any of the nucleotide sequences of (a), (b), or (c), wherein the nucleotide sequence encodes an amino acid fragment that has haploid induction activity;
(e) a fragment of any of the nucleotide sequences of (a), (b), (c), or (d), wherein the nucleotide sequence encodes an amino acid fragment that has haploid induction activity, wherein the amino acid fragment comprises an A motif, a B motif, or an AP2 DNA binding domain, or combinations thereof.

Embodiment 50. The method of embodiment 47, wherein the BBM polypeptide is selected from the group consisting of:
(a) an amino acid sequence as set forth in SEQ ID NO: 2, 4, 6, 8, 10, 12, 14, 21, 23, 25, 27, 29, 42, 44, 46, or 48;
(b) an amino acid sequence that has at least 95% amino acid identity to any one of SEQ ID NO: 2, 4, 6, 8, 10, 12, 14, 21, 23, 25, 27, 29, 42, 44, 46, or 48;
(c) an amino acid sequence that has at least 85% amino acid identity to any one of SEQ ID NO: 2, 4, 6, 8, 10, 12, 14, 21, 23, 25, 27, 29, 42, 44, 46, or 48;
(d) a fragment of any of the amino acid sequences of (a), (b), or (c), wherein the fragment has haploid induction activity; and
(e) a fragment of any of the amino acid sequences of (a), (b), or (c) or (d), wherein the fragment comprises an A motif, a B motif, or an AP2 DNA binding domain, or combinations thereof.

Embodiment 51. The method of embodiment 47, wherein the genomic egg cell locus has been modified to comprise an enhancer expressing element so that the heterologous polynucleotide expresses in the female gametophyte and/or the genomic egg cell locus comprises an endogenous promoter that expresses in the egg cell, wherein the endogenous promoter comprises:
(a) a polynucleotide sequence of SEQ ID NO: 15, 16, 17, 18, 19, 31, 32, 33, 34, 35, 36, 37, 38, 39, 40, or 86, or variants thereof;
(b) a polynucleotide sequence having at least 90% sequence identity to SEQ ID NO: 15, 16, 17, 18, 19, 31, 32, 33, 34, 35, 36, 37, 38, 39, 40, or 86, or variants thereof;
(c) a fragment of the polynucleotide of (a) or (b), e.g., a fragment of at least 100, 200, 300, 400, 500, 600, 700, 800, 900, 1000, 1100, 1200, 1300, 1400, 1500, 1600, 1700, 1800, 1900, or 2000 contiguous nucleotides of (a) or (b), wherein the fragment retains its ability to drive expression of the operably linked Babyboom (BBM) polynucleotide encoding the Babyboom (BBM) polypeptide or the fragment thereof.

Embodiment 52. A modified genomic locus of a plant cell, wherein the genomic locus comprises a modified coding sequence of an endogenous genomic egg cell locus, wherein one or more nucleotides in the endogenous egg cell's coding sequence have been modified to express a BBM polypeptide, wherein the BBM polypeptide expresses in a female gametophyte. See, for example, SEQ ID NOs: 114 and 115.

Embodiment 53. The modified genomic locus of embodiment 52, wherein the heterologous polynucleotide is selected from the group consisting of:
(a) a nucleotide sequence as set forth in SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(b) a nucleotide sequence that has at least 95% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(c) a nucleotide sequence that has at least 85% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(d) a fragment of any of the nucleotide sequences of (a), (b), or (c), wherein the nucleotide sequence encodes an amino acid fragment that has haploid induction activity;
(e) a fragment of any of the nucleotide sequences of (a), (b), (c), or (d), wherein the nucleotide sequence encodes an amino acid fragment that has haploid induction activity, wherein the amino acid fragment comprises an A motif, a B motif, or an AP2 DNA binding domain, or combinations thereof.

Embodiment 54. The modified genomic locus of embodiment 52, wherein the BBM polypeptide is selected from the group consisting of:
(a) an amino acid sequence as set forth in SEQ ID NO: 2, 4, 6, 8, 10, 12, 14, 21, 23, 25, 27, 29, 42, 44, 46, or 48;
(b) an amino acid sequence that has at least 95% amino acid identity to any one of SEQ ID NO: 2, 4, 6, 8, 10, 12, 14, 21, 23, 25, 27, 29, 42, 44, 46, or 48;
(c) an amino acid sequence that has at least 85% amino acid identity to any one of SEQ ID NO: 2, 4, 6, 8, 10, 12, 14, 21, 23, 25, 27, 29, 42, 44, 46, or 48;
(d) a fragment of any of the amino acid sequences of (a), (b), or (c), wherein the fragment has haploid induction activity; and
(e) a fragment of any of the amino acid sequences of (a), (b), or (c) or (d), wherein the fragment comprises an A motif, a B motif, or an AP2 DNA binding domain, or combinations thereof.

Embodiment 55. The modified genomic locus of embodiment 52, wherein the endogenous genomic egg cell locus has been modified to comprise an enhancer expressing element.

Embodiment 56. A plant comprising a promoter operably linked to a heterologous polynucleotide sequence, wherein the promoter is selected from the group consisting of:
(a) a polynucleotide sequence of SEQ ID NO: 37, 38, or 39;
(b) a polynucleotide sequence having at least 90% sequence identity to SEQ ID NO: 37, 38, or 39;
(c) a fragment of the polynucleotide of (a) or (b), e.g., a fragment of at least 100, 200, 300, 400, 500, 600, 700, 800, 900, 1000, 1100, 1200, 1300, 1400, 1500, 1600, 1700, 1800, 1900, or 2000 contiguous nucleotides of (a) or (b), wherein the fragments retains its ability to drive expression of the heterologous polynucleotide sequence in an egg cell of a plant.

Embodiment 57. A recombinant DNA construct comprising a promoter operably linked to a heterologous polynucleotide sequence, wherein the promoter is selected from the group consisting of:
(a) a polynucleotide sequence of SEQ ID NO: 37, 38, or 39;
(b) a polynucleotide sequence having at least 90% sequence identity to SEQ ID NO: 37, 38, or 39;
(c) a fragment of the polynucleotide of (a) or (b)), e.g., a fragment of at least 100, 200, 300, 400, 500, 600, 700, 800, 900, 1000, 1100, 1200, 1300, 1400, 1500, 1600, 1700, 1800, 1900, or 2000 contiguous nucleotides of (a) or (b), wherein the fragment retains its ability to drive expression of the heterologous polynucleotide sequence in an egg cell of a plant.

Embodiment 58. A method of expressing a gene product in an egg cell of a plant, comprising:

introducing into the plant or plant cell or egg cell of the plant a polynucleotide comprising a polynucleotide sequence selected from the group consisting of:

(a) a polynucleotide sequence of SEQ ID NO: 37, 38, or 39;
(b) a polynucleotide sequence having at least 90% sequence identity to SEQ ID NO: 37, 38, or 39;
(c) a fragment of the polynucleotide of (a) or (b)), e.g., a fragment of at least 100, 200, 300, 400, 500, 600, 700, 800, 900, 1000, 1100, 1200, 1300, 1400, 1500, 1600, 1700, 1800, 1900, or 2000 contiguous nucleotides of (a) or (b), wherein the fragment retains its ability to drive expression of a heterologous polynucleotide sequence in an egg cell;
wherein said polynucleotide sequence drives expression of an operably linked heterologous polynucleotide sequence.

Embodiment 59. The method of embodiment 56, 57, or 58, wherein the operably linked sequence is a Babyboom (BBM) polynucleotide that encodes a polypeptide that, when expressed in a female gametophyte, induces haploid induction.

Embodiment 60. The method of embodiment 60, wherein the operably linked sequence encodes a BBM polypeptide or functional fragment thereof.

Embodiment 61. The method of embodiment 56, 57, or 58, wherein the plant is a monocot or dicot plant.

Embodiment 62. The method of embodiment 56, 57, or 58, wherein the plant is a wheat, cotton, sunflower, safflower, tobacco, Arabidopsis, soy, barley, oats, rice, maize, triticale, sorghum, cannabis, sugarcane, rye, millet, or a flax plant.

The following examples are offered by way of illustration and not by way of limitation.

### EXAMPLES

The aspects of the disclosure are further defined in the following Examples, in which parts and percentages are by weight and degrees are Celsius, unless otherwise stated. These Examples, while indicating aspects of the disclosure, are given by way of illustration only. From the above discussion and these Examples, one skilled in the art can ascertain the essential characteristics of the aspects of the disclosure, and without departing from the spirit and scope thereof, can make various changes and modifications of them to adapt to various usages and conditions. Thus, various modifications in addition to those shown and described herein will be apparent to those skilled in the art from the foregoing description. Such modifications are also intended to fall within the scope of the appended claims.

### Example 1: Methods of Inducing Parthenogenesis in Wheat

Polynucleotides encoding BBM peptides, linked to regulatory elements active in maternal tissues or haploid cells, were evaluated for their ability to induce parthenogenesis in the egg cells of wheat.

Maternal Haploid Induction In Response to Wheat BBM Peptides Polynucleotides encoding BBM type transcription factor peptides were identified in the wheat genome on homocologous chromosome 3 (chromosome 3A (SEQ ID NO: 1, 2, 74, 75), 3B (SEQ ID NO: 3, 4, 76, 77) and 3D (SEQ ID NO: 5, 6, 78, 79)) and homocologous chromosome 6 (chromosome 6A (SEQ ID NO: 7, 8, 80, 81), 6B (SEQ ID NO: 9, 10, 82, 83) and 6D (SEQ ID NO: 11, 12, 84, 85)). An alignment of wheat BBM peptides with BBM peptides from additional monocot species is shown in FIG. 1. DNA polynucleotide sequences encoding TA-BBM peptides were operably linked to various different promoters active during female gamete development.

Expression cassettes were prepared to test parthenogenesis using the wheat TA-BBM3A sequence, an ortholog of the ZM-ODP2 (SEQ ID NO: 13, 14). In one example, a ZM-DD45 PRO: TA-BBM3A expression cassette contained a polynucleotide encoding a TA-BBM3A peptide (SEQ ID NO: 1) operably linked to regulatory elements ZM-DD45 PRO (SEQ ID NO: 15). In another example, a PvEC1 PRO: TA-BBM3A expression cassette contained a polynucleotide encoding a TA-BBM3A peptide (SEQ ID NO: 1) operably linked to regulatory elements comprising PV-EGG CELL PRO (TR1) (SEQ ID NO: 16), EGG MIN PRO (SEQ ID NO: 17), and PV-PRO31696.1 5UTR (SEQ ID NO: 18), this combination of regulatory elements SEQ ID NO: 16, SEQ ID NO: 17, and SEQ ID NO: 18, is called the “PvEC1 promoter” (SEQ ID NO: 19).

Similar expression cassettes were prepared to evaluate parthenogenesis using the wheat TA-BBM6A sequence, an ortholog of the ZM-BBM2 (SEQ ID NO: 20, 21) sequence in wheat. In one example, the ZM-DD45 PRO PRO: TA-BBM6A expression cassette contained a polynucleotide encoding a TA-BBM6A peptide (SEQ ID NO: 7) operably linked to regulatory elements ZM-DD45 PRO (SEQ ID NO: 15). In another example, a PvEC1 PRO: TA-BBM6A expression cassette contained a polynucleotide encoding a TA-BBM6A peptide (SEQ ID NO:7) was operably linked to regulatory elements comprising PV-EGG CELL PRO (TR1) (SEQ ID NO: 16), EGG MIN PRO (SEQ ID NO: 17), and PV-PRO31696.1 5UTR (SEQ ID NO: 18), this combination of regulatory elements SEQ ID NO: 16, SEQ ID NO: 17, and SEQ ID NO: 18, is called the “PvEC1 promoter” (SEQ ID NO: 19).

These expression cassettes were designed and used as a parthenogenesis factor to induce egg cells to differentiate into embryo cells and to initiate embryogenesis. These expression cassettes were included in constructs for Agrobacterium-mediated transformation of the wheat line SBC0456D, resulting in random integration of T-DNA within the wheat genome. Leaf samples were collected from TO seedlings to determine copy number and transgene integrity of multiple different elements within the left border and right border of the T-DNA integrated into the plant genome. Only plants determined as single copy for the multiple different elements were characterized further. After transformation, each regenerated hemizygous TO plant that contained a single, stably inserted T-DNA was considered a unique event, was grown to maturity, allowed to self-pollinate, and mature T1 seed was harvested. In plants containing a hemizygous BBM expression cassette, the transgene is expected to segregate as a 1:1 ratio (transgene positive: transgene negative) in the female gametes, and thus 50% is the expected maximum frequency of haploid progeny if fully penetrant and expressive for the parthenogenetic phenotype. If parthenogenesis occurred, pollination may only entail the fertilization of the central cell, and initiate the normal development of endosperm, but not fertilization of the egg cell which has already initiated embryogenesis. During female gametogenesis, expression of the wheat BBM polynucleotides in or near the embryo sac cell, particularly the egg cell, driven by the egg-cell specific promoter stimulates embryogenesis and parthenogenesis of the egg cell. In bread wheat, progeny derived from parthenogenetic egg cells will be haploid (In=3x, ABD) instead of diploid (2n=6x, AABBDD), and contain half the number of chromosomes and relative DNA content, as compared to the parental plant. The ploidy of individual plants in the T1 progeny was determined using flow cytometry analysis of nuclei isolated from leaf tissue.

Evaluation of haploid progeny demonstrated the ability of wheat BBM polynucleotide sequences to induce parthenogenesis. The percent of haploid progeny (%) were scored for each unique event using the number of haploid progeny divided by the total number of progeny examined to determine the haploid induction (HI) frequency. Progeny from transgenic events containing expression cassettes ZM-DD45 PRO::TA-BBM3A, Pv-EC1 PRO: TA-BBM3A, were evaluated by flow cytometry to determine the HI frequency. For the ZM-DD45 PRO::TA-BBM3A expression cassette, individual events showed a range of 0% to 10% HI frequency, and showed an average rate of 1.94% HI frequency with a standard deviation of 4%, as shown in Table 1. For the Pv-EC1 PRO: TA-BBM3A expression cassette, individual events showed a range of 31.03% to 48.57% HI frequency, and showed an average rate of 41.56% HI frequency with a standard deviation of 6.97%, as shown in Table 1. Similarly, progeny from transgenic events containing expression cassettes ZM-DD45 PRO::TA-BBM6A, Pv-EC1 PRO: TA-BBM6A, were evaluated by flow cytometry to determine the frequency of haploid progeny. For the ZM-DD45 PRO::TA-BBM6A expression cassette, individual events showed a range of 0% to 6.5% HI frequency, and showed an average rate of 0.89% HI frequency with a standard deviation of 2.22%, as shown in Table 1. For the Pv-EC1 PRO: TA-BBM6A expression cassette, individual events showed a range of 16.7% to 56.3% HI frequency, and showed an average rate of 38.1% HI frequency with a standard deviation of 12.91%, as shown in Table 1.

TABLE 1
Haploid Induction (HI) in T1 progeny of
transgenic events with wheat BBM peptides

| | Number of | Number | | |
|---|---|---|---|---|
| | independent | of T1 | | |
| | transgenic | seeds | Mean | SD |
| Expression cassette | events | screened | (% HI) | (% HI) |
| ZM-DD45 | 11 | 328 | 0.89 | 2.22 |
| PRO::TA-BBM6A | | | | |
| PvEC1 | 8 | 493 | 38.1 | 12.91 |
| PRO::TA-BBM6A | | | | |
| ZM-DD45 | 6 | 352 | 1.94 | 4 |
| PRO::TA-BBM3A | | | | |
| PvEC1 | 6 | 225 | 41.56 | 6.97 |
| PRO::TA-BBM3A | | | | |

As shown above, a regulatory element expressed in the egg cell of the plant is useful for regulating TA-BBM peptide activity to induce maternal haploid induction, resulting in a percentage of the progeny produced being haploid (having half the number of chromosomes compared to the parent). In addition, alternative regulatory elements can be used to further optimize parthenogenetic maternal haploid induction levels. For example, regulatory elements such as those disclosed in US2015/0152430 (promoters including, but not limited to the AT-DD5 promoter, the AT-DD31 promoter, and the AT-DD65 promoter) and those disclosed in US2018/0094273 (Zea mays egg cell promoters) can be used in the methods of the present disclosure (See, for example, US2015/0152430 and US2018/0094273 incorporated herein by reference in their entireties).

In summary, wheat BBM polynucleotides were demonstrated to induce parthenogenesis and produce viable haploid progeny when expressed in the egg cell of the wheat female gametophyte. In addition, it was unexpected that the egg cell-specific regulatory element PvEC1 promoter improved the parthenogenetic haploid frequency in comparison to the egg cell-specific regulatory elements ZM-DD45 PRO (Table 1).

A. Maternal Haploid Induction In Response to Non-Wheat BBM Peptides

BBM type transcription factors such as those disclosed in Publication No. US20210180077, incorporated herein by reference in its entirety, and polynucleotides encoding BBM type transcription factors identified in additional species were used in this example. Haploid progeny assays were performed to evaluate whether BBM genes from various species would initiate parthenogenesis in wheat. Expression cassettes of DNA polynucleotide sequences encoding additional BBM peptides (SEQ ID NO: 13-14, 20-29) were operably linked to the PvEC1 promoter (SEQ ID NO: 19) active during female gamete development described in Table 2.

TABLE 2
Summary of BBM polynucleotides and peptides.

| DNA | DNA | | Protein |
|---|---|---|---|
| Polynucleotide | Polynucleotide | Peptide | SEQ |
| Name | SEQ ID NO: | Name | ID NO: |
| TA-BBM3A | 1 | TA-BBM3A | 2 |
| TA-BBM6A | 7 | TA-BBM6A | 8 |
| ZM-ODP2 | 13 | ZM-ODP2 | 14 |
| ZM-BBM2 | 20 | ZM-BBM2 | 21 |
| OS-ODP2 | 22 | OS-ODP2 | 23 |
| OS-BBM2 | 24 | OS-BBM2 | 25 |
| SV-BBM1 | 26 | SV-BBM1 | 27 |
| SV-BBM2 | 28 | SV-BBM2 | 29 |

Progeny from transgenic events containing Zea mays BBM type sequences, with expression cassettes PvEC1 PRO: ZM-ODP2 and Pv-EC1 PRO: ZM-BBM2, were evaluated by flow cytometry to determine the frequency of haploid progeny in wheat. The PvEC1 PRO::ZM-ODP2 expression cassette showed an average rate of 43.1% HI frequency with a standard deviation of 12.2%, and the Pv-EC1 PRO: ZM-BBM2 expression cassette showed an average rate of 42.8% HI frequency with a standard deviation of 10.4%, as shown in Table 3. Additionally, progeny from transgenic events containing Oryza sativa and Setaria viridis BBM type sequences, with expression cassettes PvEC1 PRO::OS-ODP2, Pv-EC1 PRO: OS-BBM2, PvEC1 PRO::SV-BBM1 and Pv-EC1 PRO: SV-BBM2, were evaluated by flow cytometry to determine the frequency of haploid progeny in wheat. The PvEC1 PRO::OS-ODP2 expression cassette showed an average rate of 46.8% HI frequency with a standard deviation of 8.5%, and the Pv-EC1 PRO: OS-BBM2 expression cassette showed an average rate of 49.7% HI frequency with a standard deviation of 18.6%, as shown in Table 3. The PvEC1 PRO::SV-BBM1 expression cassette showed an average rate of 36.5% HI frequency with a standard deviation of 11.0%, and the Pv-EC1 PRO: SV-BBM2 expression cassette showed an average rate of 44.1% HI frequency with a standard deviation of 10.3%, as shown in Table 3.

TABLE 3
Haploid Induction (HI) in T1 progeny of transgenic
events with BBM polynucleotides

| | Number of | Number | | |
|---|---|---|---|---|
| | independent | of T1 | | |
| | transgenic | seeds | Mean | SD |
| Expression cassette | events | screened | (% HI) | (% HI) |
| PvEC1 PRO::ZM-ODP2 | 8 | 336 | 43.1 | 12.2 |
| PvEC1 PRO::ZM-BBM2 | 10 | 824 | 42.8 | 10.4 |
| PvEC1 PRO::OS-ODP2 | 10 | 505 | 46.8 | 8.5 |
| PvEC1 PRO::OS-BBM2 | 10 | 326 | 49.7 | 18.6 |
| PvEC1 PRO::SV-BBM1 | 10 | 234 | 36.5 | 11.0 |
| PvEC1 PRO::SV-BBM2 | 10 | 658 | 44.1 | 10.3 |

In summary, BBM polynucleotides from Zea mays, Oryza sativa and Setaria viridis were demonstrated to induce parthenogenesis and produce viable haploid progeny when expressed in the egg cell of the wheat female gametophyte.

### Example 2: Methods of Inducing Parthenogenesis in Wheat With Modified Enhanced Promoters

As shown in Example 1, the wheat TA-BBM3A polynucleotide sequence was operably linked to the ZM-DD45 PRO regulatory element, and demonstrated a low frequency of parthenogenesis as measured by frequency of haploid progeny. In addition, alternative regulatory elements are used to further optimize parthenogenic maternal haploid induction levels. For example, regulatory elements such as those disclosed in US2015/0152430 (promoters including, but not limited to the AT-DD5 promoter, the AT-DD31 promoter, and the AT-DD65 promoter) and those disclosed in US2018/0094273 (Zea mays egg cell promoters) are used in the methods of the present disclosure (US2015/0152430 and US2018/0094273 incorporated herein by reference in their entireties). In addition, the further modification of regulatory elements to include an expression modulating elements (EME) are used to further optimize parthenogenetic maternal haploid induction levels. For example, expression modulating elements such as those disclosed in Publication No. WO2018/183878, incorporated herein by reference in its entirety, may be used in the methods of the present disclosure.

Expression cassettes encoding a TA-BBM3A operably linked to a regulatory element containing at least one EME are useful in the methods of the present disclosure. A modified ZM-DD45 PRO including at least one EME is used to alter mRNA transcription levels during female gametogenesis, thereby further improving and/or optimizing parthenogenic maternal haploid induction in comparison to the ZM-DD45 PRO: TA-BBM3A expression cassette as shown in Example 1.

For example, exemplary promoters are shown in Table 4. As shown in Table 4, the ZM-DD45 PRO (SEQ ID NO: 15) is modified using the ZM-AS2 EME (SEQ ID NO: 30) having one, two or three copies, shown as 1X ZM-AS2, 2X ZM-AS2, or 3X ZM-AS2, respectively, at varying positions. The EME location indicates the number of DNA base pairs upstream of the TATA box where each respective EME sequence is inserted. The TATA sequence used for each promoter is shown in the TATA box column.

TABLE 4
Exemplary promoters that include EME modification

| SEQ | | | | | |
|---|---|---|---|---|---|
| ID | Regulatory | Promoter | | EME | TATA |
| NO: | Element | Source | EME | location | box |
| 15 | ZM-DD45 PRO | ZM-DD45 | not | not | TATTTATA |
| | | PRO | applicable | applicable | |
| 31 | ZM-DD45 PRO: | ZM-DD45 | 1X ZM- | −20 | TATTTATA |
| | Z(1XM-AS2-20) | PRO | AS2 | | |
| 32 | ZM-DD45 PRO: | ZM-DD45 | 2X ZM- | −20 | TATTTATA |
| | (2XZM-AS2-20) | PRO | AS2 | | |
| 33 | ZM-DD45 PRO: | ZM-DD45 | 3X ZM- | −20 | TATTTATA |
| | (3XZM-AS2-20) | PRO | AS2 | | |
| 34 | ZM-DD45 PRO: | ZM-DD45 | 2X ZM- | −20 | TATATATA |
| | (2XZM-AS2-20): | PRO | AS2 | | |
| | TATATATA | | | | |
| 35 | ZM-DD45 PRO: | ZM-DD45 | 1X ZM- | −20 | TATAGCGC |
| | (1XZM-AS2-20): | PRO | AS2 | | |
| | TATAGCGC | | | | |
| 36 | ZM-DD45 PRO: | ZM-DD45 | 2X ZM- | −100 | TATTTATA |
| | (2XZM-AS2-100) | PRO | AS2 | | |

The modified regulatory elements shown in Table 4 were used to alter mRNA transcription levels during female gametogenesis, thereby further improving and/or optimizing parthenogenetic maternal haploid induction in comparison to TA-BBM3A polynucleotide (SEQ ID NO:1) operably linked to a regulatory element comprising DNA fragments named ZM-DD45 PRO (SEQ ID NO: 15) as described in Example 1. Expression cassettes were prepared to evaluate parthenogenesis using the wheat TA-BBM3A polynucleotide sequence with the modified ZM-DD45 PRO regulatory sequences. In one example, a ZM-DD45 PRO: (1X ZM-AS2-20)::TA-BBM3A expression cassette contained a polynucleotide encoding a TA-BBM3A peptide (SEQ ID NO: 1) operably linked to regulatory element ZM-DD45 PRO: (1X ZM-AS2-20) (SEQ ID NO: 31). In another example, a ZM-DD45 PRO: (2X ZM-AS2-100)::TA-BBM3A expression cassette contained a polynucleotide encoding a TA-BBM3A peptide (SEQ ID NO: 1) operably linked to regulatory element ZM-DD45 PRO: 2X ZM-AS2-100) (SEQ ID NO: 36).

Evaluation of haploid progeny demonstrate the ability of modified regulatory elements operably linked to the wheat TA-BBM3A polynucleotide sequence to induce parthenogenesis. The percent of haploid progeny (%) were scored for each unique event using the number of haploid progeny divided by the total number of progeny examined to determine the haploid induction (HI) frequency. Progeny from transgenic events containing expression cassettes ZM-DD45 PRO::TA-BBM3A, ZM-DD45 PRO: (1X ZM-AS2-20)::TA-BBM3A, and ZM-DD45 PRO: (2X ZM-AS2-100)::TA-BBM3A were evaluated by flow cytometry to determine the frequency of haploid progeny. As shown in Table 5, transgenic events showed an average rate of 28.5% HI frequency for the ZM-DD45 PRO: (1X ZM-AS2-20)::TA-BBM3A expression cassette, and an average rate of 4.2% HI frequency for ZM-DD45 PRO (2X ZM-AS2-100) PRO::TA-BBM3A expression cassette, relative to the average rate of 1.9% HI frequency for the unmodified ZM-DD45 PRO::TA-BBM3A (as shown in Example 1). Thus, modification to the regulatory elements operably linked to the BBM polynucleotide sequence can improve rate of parthenogenesis and haploid progeny.

TABLE 5
Haploid Induction (HI) in T1 progeny of transgenic
events with modified regulatory element ZM-DD45 PRO

| | Number of | | |
|---|---|---|---|
| | independent | | |
| | transgenic | Mean | SD |
| Expression cassette | events | (% HI) | (% HI) |
| ZM-DD45 PRO::TA-BBM3A | 6 | 1.94 | 4 |
| ZM-DD45 PRO:(1X | 9 | 28.5 | 15.5 |
| ZM-AS2-20):: TA-BBM3A | | | |
| ZM-DD45 PRO:(2X | 9 | 4.2 | 6.7 |
| ZM-AS2-100):: TA-BBM3A | | | |

The methods of the present disclosure may also use different promoters with or without the EME sequences, to improve haploid parthenogenesis as described herein.

### Example 3: Identification of a Wheat Egg Cell Preferred Promoter

An egg cell-specific or -preferred expressed gene was identified in the wheat Triticum aestivum genomic DNA reference. Polynucleotides encoding regulatory element sequence were identified in the wheat genome on homoeologous chromosome 4 (SEQ ID NO: 37, 38, 39). The spatiotemporal pattern of gene expression can be visualized by operably linking DNA polynucleotide sequences encoding a TA-EGG regulatory sequences with a fluorescent protein, and observing fluorescent signal during female gamete development.

### Example 4: Methods of Inducing Parthenogenesis with Wheat Egg Cell Promoters

As shown in Example 1, the wheat TA-BBM3A polynucleotide sequence was operably linked to the PvEC1 PRO regulatory element and demonstrated a high frequency of parthenogenesis as measured by frequency of haploid progeny. In addition, alternative regulatory elements are used to further optimize parthenogenic maternal haploid induction levels. DNA polynucleotide sequences encoding a TA-BBM3A and TA-BBM6A peptides were operably linked to native wheat promoters active during female gamete development, TA-EGG PRO and TA-EC PRO.

Expression cassettes were prepared to evaluate parthenogenesis using the wheat TA-EGG PRO (SEQ ID NO: 37) and TA-EC PRO (SEQ ID NO: 40) regulatory element sequences. In one example, the TA-EGG PRO: TA-BBM3A and TA-EGG PRO: TA-BBM6A expression cassettes contained a polynucleotide encoding either a TA-BBM3A or TA-BBM6A peptide (SEQ ID NO: 1, 7) operably linked to regulatory elements TA-EGG PRO (SEQ ID NO: 37). In another example, the TA-EC PRO: TA-BBM3A and TA-EC PRO: TA-BBM6A expression cassettes contained a polynucleotide encoding either a TA-BBM3A or TA-BBM6A peptide (SEQ ID NO: 1, 7) operably linked to regulatory elements TA-EC PRO (SEQ ID NO: 40).

These expression cassettes were designed and used as a parthenogenesis factor to induce egg cells to differentiate into embryo cells and to initiate embryogenesis. These expression cassettes were included in constructs for Agrobacterium-mediated transformation of the wheat line SBC0456D, resulting in random integration of T-DNA within the wheat genome. Leaf samples were collected from TO seedlings to determine copy number and transgene integrity of multiple different elements within the left border and right border of the T-DNA integrated into the plant genome.

Only plants determined as single copy (hemizygous) for the multiple different elements were characterized further. After transformation, each regenerated plant that was hemizygous TO, containing a single stably inserted T-DNA, was considered a unique event, grown to maturity, allowed to self-pollinate, and mature T1 seed was harvested. If parthenogenesis occurred, pollination may only entail the fertilization of the central cell, and initiate the normal development of endosperm, but not fertilization of the egg cell which has already initiated embryogenesis. During female gametogenesis, expression of the wheat BBM polynucleotides in or near the embryo sac cell, particularly the egg cell, driven by the egg-cell specific promoter stimulates embryogenesis and parthenogenesis of the egg cell. In bread wheat, progeny derived from parthenogenetic egg cells will be haploid (In=3x, ABD) instead of diploid (2n=6x, AABBDD), and contain half the number of chromosomes and relative DNA content, as compared to the parental plant. The ploidy of individual plants in the T1 progeny was determined using flow cytometry analysis of nuclei isolated from leaf tissue.

Evaluation of haploid progeny demonstrate the ability of native wheat egg cell promoters combined with native wheat BBM polynucleotide sequences to induce parthenogenesis. HI frequency was determined. Evaluation of transgenic of events containing expression cassette TA-EGG PRO: TA-BBM6A had an average of 45.3% HI with a standard deviation of 12.4%, and transgenic events containing expression cassette TA-EGG PRO::TA-BBM3A had an average of 35.1% HI with a standard deviation of 9.5% (Table 6). Evaluation of transgenic of events containing expression cassette TA-EC PRO::TA-BBM6A had an average of 46.6% HI with a standard deviation of 10.0%, and transgenic events containing expression cassette TA-EC PRO::TA-BBM3A had an average of 40.6% HI with a standard deviation of 18.7% (Table 6). Furthermore, multiple hemizygous T1 plants from four individual events containing expression cassette TA-EGG PRO::TA-BBM6A were grown to produce the T2 generation of progeny seed, and evaluated by flow cytometry. Stability of the parthenogenesis phenotype was observed as each individual event maintained a comparable HI frequency in the T1 and T2 generations.

TABLE 6
Haploid Induction (HI) in T1 progeny of transgenic
events with wheat egg cell promoters

| | Number of | Number | | |
|---|---|---|---|---|
| | independent | of T1 | | |
| | transgenic | seeds | Mean | SD |
| Expression cassette | events | screened | (% HI) | (% HI) |
| TA-EGG | 10 | 578 | 35.1% | 9.5% |
| PRO::TA-BBM3A | | | | |
| TA-EGG | 10 | 568 | 45.3% | 12.4% |
| PRO::TA-BBM6A | | | | |
| TA-EC | 8 | 539 | 40.6% | 18.7% |
| PRO::TA-BBM3A | | | | |
| TA-EC | 7 | 614 | 46.6% | 10.0% |
| PRO::TA-BBM6A | | | | |

Thus, expression cassettes containing native wheat promoter and BBM related polynucleotide sequences are sufficient to drive a high HI rate in a wheat system. Furthermore, the penetrance of the parthenogenesis phenotype shows that the wheat TA-EGG and TA-EC promoter sequences contain regulatory elements that are capable of driving expression in the egg cell. Though TA-BBM3A and TA-BBM6A only share 34.9% sequence homology, they were both able to drive a high HI rate in a wheat system.

### Example 5: Methods of Inducing Parthenogenesis in Wheat With Modified BBM Peptides

Wheat polynucleotides encoding BBM peptides are linked to regulatory elements active in a haploid cell or tissue, for example a promoter active during female gamete development. To increase activity of BBM, the polynucleotide and peptide sequences of TA-BBM3A (SEQ ID NO: 1, 2) and TA-BBM6A (SEQ ID NO: 7, 8) are modified. An alignment of native and modified wheat BBM peptides is shown in FIG. 2. The PvEC1 promoter was operably linked to these modified wheat TA-BBM3A and TA-BBM6A sequences and screened for induction of parthenogenesis.

TA-BBM3A (ALT1) (SEQ ID NO: 41, 42) is created by truncating the N- and C-terminal domains of TA-BBM3A (SEQ ID NO: 1, 2) to only contain a start codon, base pairs 154 through 2010, and a stop codon. It retains a start codon, and conserved motifs B, A, and AP2 DNA binding domains. In another variation, TA-BBM3A (ALT2) (SEQ ID NO: 43, 44) is created by truncating TA-BBM3A (SEQ ID NO: 1, 2) to leave a start codon, and base pairs 154-231, 442-504, and 684-2010, and a stop codon. TA-BBM6A (ALT1) (SEQ ID NO: 45, 46) is created by truncating the N- and C-domains of TA-BBM6A (SEQ ID NO: 7, 8) to only contain a start codon, and base pairs 328 through 1980, and a stop codon. In another variation, TA-BBM6A (ALT2) (SEQ ID NO: 47, 48) is created by truncating TA-BBM6A (SEQ ID NO: 7, 8) to leave a start codon, base pairs 328-1908, and a stop codon.

TABLE 7
Summary of Wheat TA-BBM3A and TA-BBM6A
polynucleotide and peptide truncations

| | DNA | DNA | | PROTEIN | Peptide |
|---|---|---|---|---|---|
| DNA | SEQ | length | Peptide | SEQ ID | length |
| Polynucleotide | ID NO: | (bp) | Name | NO: | (aa) |
| TA-BBM3A | 1 | 2109 | TA-BBM3A | 2 | 702 |
| TA-BBM3A | 41 | 1863 | TA-BBM3A | 42 | 620 |
| (154-2010) | | | (ALT1) | | |
| TA-BBM3A | 43 | 1473 | TA-BBM3A | 44 | 490 |
| (154-231)- | | | (ALT2) | | |
| (442-504)- | | | | | |
| (684-2010) | | | | | |
| TA-BBM6A | 7 | 1980 | TA-BBM6A | 8 | 659 |
| TA-BBM6A | 45 | 1656 | TA-BBM6A | 46 | 551 |
| (328-1980) | | | (ALT1) | | |
| TA-BBM6A | 47 | 1587 | TA-BBM6A | 48 | 528 |
| (328-1908) | | | (ALT2) | | |

Expression cassettes were prepared to evaluate parthenogenesis using the modified wheat TA-BBM3A sequence. In one example, a PvEC1 PRO: TA-BBM3A (ALT1) expression cassette contained a polynucleotide encoding a TA-BBM3A (ALT1) peptide (SEQ ID NO: 41) operably linked to regulatory elements PvEC1 PRO (SEQ ID NO: 19). In another example, a PvEC1 PRO: TA-BBM3A (ALT2) expression cassette contained a polynucleotide encoding a TA-BBM3A (ALT2) peptide (SEQ ID NO: 43) operably linked to regulatory elements PvEC1 PRO (SEQ ID NO: 19). Similarly, expression cassettes were prepared to evaluate parthenogenesis using the modified wheat TA-BBM6A sequence. In one example, a PvEC1 PRO: TA-BBM6A (ALT1) expression cassette contained a polynucleotide encoding a TA-BBM6A (ALT1) peptide (SEQ ID NO: 45) operably linked to regulatory elements PvEC1 PRO (SEQ ID NO: 19). In another example, a PvEC1 PRO: TA-BBM6A (ALT2) expression cassette contained a polynucleotide encoding a TA-BBM6A (ALT2) peptide (SEQ ID NO: 47) operably linked to regulatory elements PvEC1 PRO (SEQ ID NO: 19).

Evaluation of haploid progeny demonstrate the ability of modified BBM peptides, when operably linked to PvEC1 PRO regulatory element, to induce parthenogenesis. HI frequency was determined as previously described. Evaluation of transgenic events containing expression cassette PvEC1 PRO::TA-BBM3A (ALT1) had an average of 27.2% HI with a standard deviation of 9.8%, and transgenic events containing PvEC1 PRO::TA-BBM3A (ALT2) had an average of 0.0% HI with a standard deviation of 0.0% (Table 8). In addition, evaluation of transgenic events containing expression cassette PvEC1 PRO::TA-BBM6A (ALT1) had an average of 2.6% HI with a standard deviation of 3.1%, and transgenic events containing PvEC1 PRO::TA-BBM6A (ALT2) had an average of 0.0% HI with a standard deviation of 0.0% (Table 8).

TABLE 8
Haploid Induction (HI) in T1 progeny of transgenic events
with wheat BBM polynucleotide and peptide truncations

| | Number of | Number | | |
|---|---|---|---|---|
| | independent | of T1 | | |
| | transgenic | seeds | Mean | SD |
| Expression cassette | events | screened | (% HI) | (% HI) |
| PvEC1 PRO::TA-BBM3A | 7 | 439 | 27.2% | 9.8% |
| (ALT1) | | | | |
| PvEC1 PRO::TA-BBM3A | 7 | 474 | 0.0% | 0.0% |
| (ALT2) | | | | |
| PvEC1 PRO::TA-BBM6A | 9 | 409 | 2.6% | 3.1% |
| (ALT1) | | | | |
| PvEC1 PRO::TA-BBM6A | 9 | 491 | 0.0% | 0.0% |
| (ALT1) | | | | |

In summary, minimal N- and C-terminal truncations of the wheat BBM polypeptides (ALT1 sequences) were sufficient to induce parthenogenesis and produce viable haploid progeny, but were less efficacious than the full length BBM polynucleotide sequence, when expressed in the egg cell of the wheat female gametophyte. However, further truncations of the N- or C-termini of the wheat BBM polypeptide between motifs B and A were not sufficient to induce parthenogenesis or haploid progeny.

### Example 6: Method for Engineering a Native Parthenogenesis Locus in Wheat

To achieve optimal parthenogenesis phenotypes, promoters have been evaluated for driving TA-BBM egg cell-specific or -preferred expression operably linked to TA-BBM polynucleotides in transgenic wheat plants, as shown in Example 4.

A. Modifying an Endogenous Wheat BBM locus

Here we present a gene editing method to modify the promoter region of the endogenous BBM locus to attain the desired expression pattern for parthenogenesis phenotypes. In wheat, it is expected that modification of the promoter of a single homoeologous TA-BBM locus to include alternative egg cell-specific/preferred promoters or egg cell-specific/preferred sequence motifs, in addition to the native promoter sequence or in place of the native promoter sequence, would modify the expression pattern. This modified egg cell expression pattern of a single TA-BBM homeolog is expected to induce parthenogenesis in wheat.

The DNA reagents used during the modification of the native locus, including but not limited to the guideRNA, the Cas polypeptide, such as, Cas9, Cas12f (Cas-alpha, Cas14), Cas121 (Cas-beta), Cas12a (Cpf1), Cas12b (a C2c1 protein), Cas13 (a C2c2 protein), Cas12c (a C2c3 protein), Cas12d, Cas12e, Cas12g, Cas12h, Cas12i, Cas12j, Cas12k, Cas3, Cas3-HD, Cas 5, Cas6, Cas7, Cas8, Cas10, or combinations or complexes of these, and transformation selectable marker are not required for the function of the newly generated BBM allele and can be eliminated from the genome by segregation through breeding methods known to one skilled in the art. Because the promoter TA-EGG (SEQ ID NO: 37), having the same sequence as the endogenous TA-EGG locus, is inserted into the TA-BBM locus, for example the TraesCS3A02G395500 or the TraesCS6A02G229500 as described in the IWGSCv1.0 Chinese Spring wheat reference sequence assembly and annotation, through homologous recombination, this TA-BBM allele is indistinguishable from natural mutant alleles.

To substitute the native promoter of TA-BBM with the TA-EGG promoter into the 5′UTR of the wheat TA-BBM gene, two guide RNAs (gRNA) can be prepared using the maize U6 promoter and terminator. One of the gRNAs can include the 5′-end of the gRNA containing a 20-bp variable targeting domain targeting the genomic sequence in the 5′ upstream region of the TA-BBM promoter, and one of the gRNAs can include the 5′-end of the gRNA containing a 20-bp variable targeting domain targeting the genomic sequence in the 3′ upstream region of the TA-BBM promoter, preferably near the ATG start codon. For example, potential gRNA targeting domain sequences for the TA-BBM3A promoter and 5′UTR sequence are described but not limited to sequences in Table 7 (SEQ ID NO: 49-56). A polynucleotide modification template containing the TA-EGG PRO will be flanked by two genomic DNA fragments (HR1 and HR2) derived and homologous to the upstream and downstream region of the genomic target sequence. Transformation can be initiated with constructs that include the gRNA, polynucleotide modification template, Cas cassette, such as as a Cas9 cassette, and a transformation selectable marker. Transgenic plants that are produced by this method may be evaluated by PCR analysis to confirm the promoter swap by identifying PCR amplification products that include both the TA-EGG promoter sequence and the TA-BBM genomic coding sequence, but do not include amplification products for the of the TA-BBM promoter and the TA-BBM genomic coding sequence. Successful promoter swap events can be further characterized by sequencing the amplification products described above. It is expected that successful TA-EGG PRO: TA-BBM promoter swap events will induce parthenogenesis and result in haploid progeny as described in Example 4.

TABLE 9
Example of gRNA targeting domain sequences
for the TA-BBM3A promoter region

| | SEQ | |
|---|---|---|
| | ID | |
| DNA Polynucleotide | NO: | Sequence |
| TA-BBM3A gRNA 1 | 49 | GTAATCCTAAAA |
| (Nucleotide sequence of | | TAATCCGTGGG |
| gRNA that targets wheat | | |
| genomic BBM3A) | | |
| TA-BBM3A gRNA 2 | 50 | GAGTAAGTGGAC |
| (Nucleotide sequence of | | TCTCTGCTTGG |
| gRNA that targets wheat | | |
| genomic BBM3A) | | |
| TA-BBM3A gRNA 3 | 51 | GCACGACATGTC |
| (Nucleotide sequence of | | CAAAGCGTAGG |
| gRNA that targets wheat | | |
| genomic BBM3A) | | |
| TA-BBM3A gRNA 4 | 52 | GTAGTGTCGACA |
| (Nucleotide sequence of | | CCAGTGTATGG |
| gRNA that targets wheat | | |
| genomic BBM3A) | | |
| TA-BBM3A gRNA 5 | 53 | GGGCTAGTACAT |
| (Nucleotide sequence of | | CCAAATGTAGG |
| gRNA that targets wheat | | |
| genomic BBM3A) | | |
| TA-BBM3A gRNA 6 | 54 | GGCTAGTACATC |
| (Nucleotide sequence of | | CAAATGTAGGG |
| gRNA that targets wheat | | |
| genomic BBM3A) | | |
| TA-BBM3A gRNA 7 | 55 | GTTAAACCTTGT |
| (Nucleotide sequence of | | CAGGGATTCGG |
| gRNA that targets wheat | | |
| genomic BBM3A) | | |
| TA-BBM3A gRNA 8 | 56 | GTCCGACATAGG |
| (Nucleotide sequence of | | AGTGTTAATGG |
| gRNA that targets wheat | | |
| genomic BBM3A) | | |

### B. Modifying an Endogenous Wheat EGG CELL Locus

Here we present a gene editing method to modify the coding region of the endogenous EGG CELL locus to attain the desired expression pattern for parthenogenesis phenotypes. In wheat, it is expected that modification of the coding sequence of a single homoeologous TA-EGG locus to include alternative BBM coding sequence in place of the native coding sequence, would modify the expression pattern of a BBM polynucleotide. The introduction of a BBM coding sequence downstream of an egg cell-specific/preferred promoter is expected to induce parthenogenesis in wheat.

To substitute the native coding sequence of TA-EGG with the TA-BBM coding sequence into the 5′UTR of the wheat TA-BBM gene, two guide RNAs (gRNA) can be prepared using the maize U6 promoter and terminator. One of the gRNAs can include the 5′-end of the gRNA containing a 20-bp variable targeting domain targeting the genomic sequence in the 3′ upstream region of the TA-EGG promoter, preferably near the ATG start codon, and one of the gRNAs can include the 5′-end of the gRNA containing a 20-bp variable targeting domain targeting the genomic sequence in the 3′ region of the TA-EGG locus, preferably near the stop codon. A polynucleotide modification template containing the TA-BBM coding sequence will be flanked by two genomic DNA fragments (HR1 and HR2) derived and homologous to the upstream and downstream region of the genomic target sequence. Transformation can be initiated with constructs that include the gRNA, polynucleotide modification template, Cas cassette, such as a Cas 9 cassette, and a transformation selectable marker. Transgenic plants that are produced by this method can be evaluated by PCR analysis to confirm the coding sequence swap by identifying PCR amplification products that include both the TA-EGG promoter sequence and the TA-BBM genomic coding sequence, but do not include amplification products of the TA-EGG promoter and the TA-EGG genomic coding sequence. Successful promoter swap events can be further characterized by sequencing the amplification products described above. It is expected that successful TA-EGG PRO: TA-BBM coding sequence swap events will induce parthenogenesis and result in haploid progeny as described in Example 4.

### Example 7: Method for Producing Apomictic Plants

Apomixis is asexual reproduction resulting in progeny that are genetically identical to the parent. Methods disclosed herein are used to obtain an apomictic plant having an apomciosis component with inhibited or mutated gene products that induce mitosis instead of meiosis, the MiMe phenotype, and containing a BBM parthenogenesis component as described in Examples 1, 2, 4-6.

In hexaploid wheat it is expected that MiMe phenotypes may require inhibition or mutation in each of the three homocologous gene loci. Genomic sequence for the coding region of the SPO11-1 homocologous gene loci (SEQ ID NO: 57, 58, 59) was identified from the IWGSCv1.0 Chinese Spring reference sequence and assembly. Genomic sequence for the coding region of the REC8 homocologous gene loci (SEQ ID NO: 60, 61, 62) was identified from the IWGSCv1.0 Chinese Spring reference sequence and assembly. Genomic sequence for the coding region of the OSD homocologous gene loci (SEQ ID NO: 63, 64, 65) was identified from the IWGSCv1.0 Chinese Spring reference sequence and assembly.

Natural mutations, or mutations induced by chemicals or radiation, in the individual homocologous loci of the MiMe genes may be identified, and then crossed together to create individual MiMe gene knockout lines. The individual MiMe gene knockout lines for Spo11, Rec8, Prd1, Prd2, Prd3 are expected to be both male and female sterile, and individual MiMe gene knockout lines for Osd1 are expected to increase the ploidy of the gametes. However, the combination of mutations to create a MiMe phenotype may be created by crossing plants that are heterozygous for alleles of the MiMe loci. In this example, mutations with knockout or knockdown alleles of MiMe loci may be combined with BBM parthenogenesis components to achieve an apomictic plant, e.g. a flowering plant, in the absence of egg cell fertilization comprising with clonal gametes and progeny.

Another example to improve the process to create knockout or knockdown alleles in the MiMe loci is to utilize gene editing components. Methods of the present disclosure use a gene editing trait comprising a first expression cassette encoding a CRISPR-Cas9 gene editing polynucleotide (or other Cas polypeptide) and a second expression cassette encoding gRNA molecules having sequence homology to the MiMe genes. For example, gRNA targeting domain sequences (SEQ ID NO: 66-73) for the TA-SPO11-1, TA-REC8 and TA-OSD genomic coding sequences are described but not limited to sequences shown in Table 8. It is expected that mutations at the MiMe gene target sites provide methods of obtaining a non-recombination and non-reduction. Alternatively, the methods of the present disclosure use a gene editing trait comprising a first expression cassette encoding a Cas alpha gene editing polynucleotide and a second expression cassette encoding gRNA molecules having sequence homology to the MiMe genes. It is expected that mutations at the MiMe gene target sites provide methods of obtaining a non-recombined and non-reduced gamete.

TABLE 10
Example of gRNA targeting domain sequences
for the TA-SPO11-1, TA-REC8 and TA-OSD
genomic coding region

| | SEQ | |
|---|---|---|
| | ID | |
| DNA Polynucleotide | NO: | Sequence |
| TA-SPO11-1 gRNA 1 | 66 | GCCCCAAGCCATCGTATCTC |
| (Nucleotide sequence of | | |
| gRNA that targets wheat | | |
| genomic SPO11-1) | | |
| TA-SPO11-1 gRNA 2 | 67 | GTGTTCCTTTACCTCCATTC |
| (Nucleotide sequence of | | |
| gRNA that targets wheat | | |
| genomic SPO11-1) | | |
| TA-REC8 gRNA 1 | 68 | GAGACTCAAAATTATCGACC |
| (Nucleotide sequence of | | |
| gRNA that targets wheat | | |
| genomic REC8) | | |
| TA-REC8 gRNA 2 | 69 | GTCGAGAGTAATATGGACTG |
| (Nucleotide sequence of | | |
| gRNA that targets wheat | | |
| genomic REC8) | | |
| TA-REC8 gRNA 3 | 70 | GATGACTGATATACATTTCC |
| (Nucleotide sequence of | | |
| gRNA that targets wheat | | |
| genomic REC8) | | |
| TA-REC8 gRNA 4 | 71 | GCTCTAAAAGGCGTCAAGTC |
| (Nucleotide sequence of | | |
| gRNA that targets wheat | | |
| genomic REC8) | | |
| TA-OSD gRNA 1 | 72 | TGCTGGATCCTCAGGCTCCG |
| (Nucleotide sequence of | | |
| gRNA that targets wheat | | |
| genomic OSD) | | |
| TA-OSD gRNA 2 | 73 | GAATCCACCGCCAGAGCGAT |
| (Nucleotide sequence of | | |
| gRNA that targets wheat | | |
| genomic OSD) | | |

Constructs used for gene editing processes may include an additional cassette containing a BBM parthenogenesis component to achieve an apomictic plant with clonal gametes and progeny. Constructs used for gene editing or parthenogenesis processes may also be transformed into lines that are previously transformed or gene edited to achieve an apomictic plant with clonal gametes and progeny. Alternatively, gene edits with knockout or knockdown alleles of the MiMe loci may be combined with BBM parthenogenesis components by crossing to achieve an apomictic plant with clonal gametes and progeny. In this example, gene edits with knockout or knockdown alleles of MiMe loci may be combined with BBM parthenogenesis components to achieve an apomictic plant with clonal gametes and progeny.

### Example 8: Method for Engineering a Native Parthenogenesis Locus in Maize

To achieve optimal parthenogenesis phenotypes, promoters are evaluated for driving ZM-BBM or ZM-ODP2 egg cell-specific or -preferred expression operably linked to ZM-BBM2 or ZM-ODP2 polynucleotides in transgenic maize plants.

### A. Modifying an Endogenous Maize BBM Locus

Here we present a gene editing method to modify the promoter region of the endogenous maize BBM2 locus or ODP2 locus to obtain the desired expression pattern for parthenogenesis phenotypes. In maize, it is expected that modification of the promoter of a single homocologous ZM-BBM2 or ZM-ODP2 locus to include alternative egg cell-specific/preferred promoters or egg cell-specific/preferred sequence motifs, in addition to the native promoter sequence or in place of the native promoter sequence, would modify the expression pattern. This modified egg cell expression pattern of a single ZM-BBM2 or ZM-ODP2 homeolog is expected to induce parthenogenesis in maize.

The DNA reagents used during the modification of the native locus, including but not limited to the guideRNAs, a Cas endonuclease (such as Cas9, Cas12f (Cas-alpha, Cas14), Cas121 (Cas-beta), Cas12a (Cpf1), Cas12b (a C2c1 protein), Cas13 (a C2c2 protein), Cas12c (a C2c3 protein), Cas12d, Cas12c, Cas12g, Cas12h, Cas12i, Cas12j, Cas12k, Cas3, Cas3-HD, Cas 5, Cas6, Cas7, Cas8, Cas10, or combinations or complexes of these), and transformation selectable marker are not required for the function of the newly generated BBM allele and can be eliminated from the genome by segregation through standard breeding methods. For example, the promoter ZM-EGG PRO (SEQ ID NO: 86) is inserted upstream of the start codon of the endogenous ZM-BBM2 genomic locus or endogenous ZM-ODP2 genomic locus. In another example, the promoter ZM-DD45 (SEQ ID NO: 15) is inserted upstream of the start codon of the endogenous Zm-BBM2 or ZM-ODP2 genomic locus. In either of these cases, one gRNA could be used near the start codon or in the 5′UTR to insert the promoter sequence, or two gRNAs could be used to first excise out the native ZM-BBM2 or ZM-ODP2 promoter before insertion of the egg cell promoter. Because the promoter ZM-EGG PRO, having the same sequence as the endogenous ZM-EGG locus, is inserted into the ZM-BBM2 or ZM-ODP2 locus for example, through homologous recombination, this allele is indistinguishable from natural mutant alleles.

To substitute the native promoter of ZM-BBM2 with the ZM-EGG promoter (SEQ ID NO: 86) into the 5′UTR of the maize ZM-BBM2 gene, two guide RNAs (gRNA) can be prepared using the maize U6 promoter and terminator. One of the gRNAs can include the 5′-end of the gRNA containing a 20-bp variable targeting domain targeting the genomic sequence in the 5′ upstream region of the ZM-BBM2 promoter, and one of the gRNAs can include the 5′-end of the gRNA containing a 20-bp variable targeting domain targeting the genomic sequence in the 3′ upstream region of the ZM-BBM2 promoter, preferably near the ATG start codon. For example, potential gRNA targeting domain sequences for the ZM-BBM2 promoter and 5′UTR sequence are described but not limited to sequences in Table 9 (SEQ ID NO:91, 92, 93, 94, 95, 96, 97, and 98).

TABLE 11
Example of gRNA targeting domain sequences
for the ZM-ODP2 and ZM-BBM2 promoter regions.

| | SEQ | |
|---|---|---|
| | ID | |
| DNA Polynucleotide | NO: | Sequence |
| ZM-ODP2 PRO gRNA 1 | 91 | TGTTCATACT |
| (Nucleotide sequence of | | TACGCGGTGA |
| gRNA that targets Zea mays | | |
| ODP2 promoter) | | |
| ZM-ODP2 PRO gRNA 2 | 92 | GTTACCTGTT |
| (Nucleotide sequence of | | CATACTTACG |
| gRNA that targets Zea mays | | |
| ODP2 promoter) | | |
| ZM-ODP2 PRO gRNA 3 | 93 | CCAGTTGTTC |
| (Nucleotide sequence of | | ACAGTGGCCA |
| gRNA that targets Zea mays | | |
| ODP2 promoter) | | |
| ZM-ODP2 PRO gRNA 4 | 94 | AGCGAGCCAG |
| (Nucleotide sequence of | | TTGTTCACAG |
| gRNA that targets Zea mays | | |
| ODP2 promoter) | | |
| ZM-BBM2 PRO gRNA 1 | 95 | TATGCACTTG |
| (Nucleotide sequence of | | TCGGCGCACA |
| gRNA that targets Zea mays | | |
| BBM2 promoter) | | |
| ZM-BBM2 PRO gRNA 2 | 96 | CACTTGTCGG |
| (Nucleotide sequence of | | CGCACACGGC |
| gRNA that targets Zea mays | | |
| BBM2 promoter) | | |
| ZM-BBM2 PRO gRNA 3 | 97 | TGCCTTGCTT |
| (Nucleotide sequence of | | GTGATCAGAG |
| gRNA that targets Zea mays | | |
| BBM2 promoter) | | |
| ZM-BBM2 PRO gRNA 4 | 98 | GGGCGTTGAT |
| (Nucleotide sequence of | | AGAACAGTGA |
| gRNA that targets Zea mays | | |
| BBM2 promoter) | | |

A polynucleotide modification template containing the ZM-EGG PRO will be flanked by two genomic DNA fragments (HR1 and HR2) derived and homologous to the upstream and downstream region of the genomic target sequence. Transformation can be initiated with constructs that include the gRNA, polynucleotide modification template, Cas cassette and a transformation selectable marker. Transgenic plants that are produced by this method may be evaluated by PCR analysis to confirm the promoter swap by identifying PCR amplification products that include both the ZM-EGG promoter sequence and the ZM-BBM2 genomic coding sequence. Successful promoter swap events can be further characterized by sequencing the amplification products described above. It is expected that successful ZM-EGG PRO: ZM-BBM2 promoter swap events will induce parthenogenesis and result in haploid progeny.

In another example, a similar strategy can be applied for inserting ZM-DD45 PRO or ZM-EGG PRO into the ZM-ODP2 genomic locus.

TABLE 2
Example of gRNA targeting domain sequences
for the ZM-EGG and ZM-DD45 regions.

| | SEQ | |
|---|---|---|
| | ID | |
| DNA Polynucleotide | NO: | Sequence |
| ZM-EGG gRNA 1 | 99 | GGAGCCCTTC |
| (Nucleotide sequence of | | CTTCCCGCCA |
| gRNA that targets Zea mays | | |
| Egg promoter) | | |
| ZM-EGG gRNA 2 | 100 | GCGTCATCAG |
| (Nucleotide sequence of | | CACCGCGAGG |
| gRNA that targets Zea mays | | |
| Egg promoter) | | |
| ZM-EGG gRNA 3 | 101 | ATCTAAGACC |
| (Nucleotide sequence of | | AGTGAAGACT |
| gRNA that targets Zea mays | | |
| Egg promoter) | | |
| ZM-EGG gRNA 4 | 102 | TGGGATGATG |
| (Nucleotide sequence of | | AATGGAGCTC |
| gRNA that targets Zea mays | | |
| Egg promoter) | | |
| ZM-DD45 gRNA 1 | 103 | CGTACGTACA |
| (Nucleotide sequence of | | GGAGCACACC |
| gRNA that targets Zea mays | | |
| DD45 promoter) | | |
| ZM-DD45 gRNA 2 | 104 | AGCACACCAG |
| (Nucleotide sequence of | | GCAACGGCAA |
| gRNA that targets Zea mays | | |
| DD45 promoter) | | |
| ZM-DD45 gRNA 3 | 105 | GGTACCAGCT |
| (Nucleotide sequence of | | CACCCCCTCA |
| gRNA that targets Zea mays | | |
| DD45 promoter) | | |
| ZM-DD45 gRNA 4 | 106 | AGCTGGCTGC |
| (Nucleotide sequence of | | CGTAGCCGTG |
| gRNA that targets Zea mays | | |
| DD45 promoter) | | |

### B. Modifying an Endogenous Maize EGG CELL Locus

Here we present a gene editing method to modify the coding region of the endogenous EGG CELL locus to attain the desired expression pattern for parthenogenesis phenotypes in maize. In maize, it is expected that modification of the coding sequence of a single homoeologous ZM-EGG locus to include alternative BBM2 or ODP2 coding sequence in place of the native coding sequence, would modify the expression pattern of a BBM polynucleotide. The introduction of a BBM2 or ODP2 coding sequence downstream of an egg cell-specific/preferred promoter is expected to induce parthenogenesis in maize.

To substitute the native coding sequence of ZM-EGG with the ZM-BBM coding sequence into the 5′UTR of the maize ZM-BBM2 gene, two guide RNAs (gRNA) can be prepared using the maize U6 promoter and terminator. One of the gRNAs can include the 5′-end of the gRNA containing a 20-bp variable targeting domain targeting the genomic sequence in the 3′ upstream region of the ZM-EGG promoter, preferably near the ATG start codon, and one of the gRNAs can include the 5′-end of the gRNA containing a 20-bp variable targeting domain targeting the genomic sequence in the 3′ region of the ZM-EGG locus, preferably near the stop codon. A polynucleotide modification template containing the ZM-BBM coding sequence will be flanked by two genomic DNA fragments (HR1 and HR2) derived and homologous to the upstream and downstream region of the genomic target sequence. Transformation can be initiated with constructs that include the gRNAs, polynucleotide modification template, Cas endonuclease cassette and a transformation selectable marker. Transgenic plants that are produced by this method can be evaluated by PCR analysis to confirm the coding sequence swap by identifying PCR amplification products that include both the ZM-EGG promoter sequence and the ZM-BBM2 coding sequence. Successful coding sequence swap events can be further characterized by sequencing the amplification products described above. It is expected that successful ZM-EGG PRO: ZM-BBM2 coding sequence swap events will induce parthenogenesis and result in haploid progeny.

In another example, similar to above, but a single gRNA is used to create a DNA break between the promoter of an egg promoter such as ZM-EGG or ZM-DD45 and the coding sequence of ZM-EGG or DD45. A coding sequence, either genomic or CDS of ZM-ODP2 or ZM-BBM2 with their terminators are introduced and integrates into the cut site. In these examples, the coding sequence of the ZM-EGG or ZM-DD45 is not eliminated from the genome but is separated from its functional promoter. Instead, the functional egg promoter is driving expression of either ZM-ODP2 or ZM-BBM2. Transgenic plants that are produced by this method can be evaluated by PCR analysis to confirm the coding sequence swap by identifying PCR amplification products that include both the ZM-EGG promoter sequence and the ZM-BBM2/ZM-ODP2 coding sequence. Successful coding sequence swap events can be further characterized by sequencing the amplification products described above. It is expected that successful ZM-EGG PRO: ZM-BBM2, ZM-DD45: ZM-BBM2, ZM-EGG: ZM-ODP2, and ZM-EGG: ZM-ODP2 coding sequence swap events will induce parthenogenesis and result in haploid progeny.

1. A method of expressing a gene product in an egg cell of a plant, comprising:
introducing into the plant a polynucleotide comprising a polynucleotide sequence selected from the group consisting of:
(a) a polynucleotide sequence of SEQ ID NO: 37, 38, or 39;
(b) a polynucleotide sequence having at least 90% sequence identity to SEQ ID NO: 37, 38, or 39;
(c) a fragment of the polynucleotide of (a) or (b), wherein the fragment retains its ability to drive expression of a heterologous polynucleotide sequence in an egg cell;
wherein said polynucleotide sequence drives expression of an operably linked heterologous polynucleotide sequence.

2. The method of claim 1, wherein the operably linked heterologous polynucleotide sequence is a Babyboom (BBM) polynucleotide that encodes a polypeptide that, when expressed in a female gametophyte, induces haploid induction.

3. The method of claim 2, wherein the operably linked heterologous polynucleotide sequence encodes a BBM polypeptide or functional fragment thereof.

4. The method of claim 1, wherein the plant is a monocot or dicot plant.

5. The method of claim 1, wherein the plant is a wheat, cotton, sunflower, safflower, tobacco, Arabidopsis, soy, barley, oats, rice, maize, triticale, sorghum, canola, cannabis, sugarcane, rye, millet, turf grass, or a flax plant.

6. A plant comprising a promoter operably linked to a heterologous polynucleotide sequence, wherein the promoter is selected from the group consisting of:
(a) a polynucleotide sequence of SEQ ID NO: 37, 38, or 39;
(b) a polynucleotide sequence having at least 90% sequence identity to SEQ ID NO: 37, 38, or 39;
(c) a fragment of the polynucleotide of (a) or (b), wherein the fragment retains its ability to drive expression of the heterologous polynucleotide sequence in an egg cell of a plant.

7. A method of inducing parthenogenesis, the method comprising:
expressing, in a female gametophyte, a Babyboom (BBM) polynucleotide operably linked to a promoter that expresses in a plant egg cell, wherein the Babyboom (BBM) polynucleotide comprises a nucleotide sequence encoding a Babyboom (BBM) polypeptide or a fragment thereof, wherein the female gametophyte is rendered parthenogenic and forms a haploid embryo without pollination, and wherein the Babyboom (BBM) polynucleotide is not naturally expressed in a female gametophyte.

8. The method of claim 7, wherein the nucleotide sequence encoding the Babyboom (BBM) polypeptide or fragment thereof is selected from the group consisting of: BBM1, BBM2, BMN2, BMN3, ODP2, and BBML.

9. The method of claim 7, wherein the Babyboom (BBM) polynucleotide is selected from the group consisting of:
(a) a nucleotide sequence as set forth in SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(b) a nucleotide sequence that has at least 95% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(c) a nucleotide sequence that has at least 85% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(d) a fragment of any of the nucleotide sequences of (a), (b), or (c), wherein the nucleotide sequence encodes an amino acid fragment that has haploid induction activity;
(e) a fragment of any of the nucleotide sequences of (a), (b), (c), or (d), wherein the nucleotide sequence encodes an amino acid fragment that has haploid induction activity, wherein the amino acid fragment comprises an A motif, a B motif, or an AP2 DNA binding domain, or combinations thereof.

10. The method of claim 7, wherein the Babyboom (BBM) polypeptide is selected from the group consisting of:
(a) an amino acid sequence as set forth in SEQ ID NO: 2, 4, 6, 8, 10, 12, 14, 21, 23, 25, 27, 29, 42, 44, 46, or 48;
(b) an amino acid sequence that has at least 95% amino acid identity to any one of SEQ ID NO: 2, 4, 6, 8, 10, 12, 14, 21, 23, 25, 27, 29, 42, 44, 46, or 48;
(c) an amino acid sequence that has at least 85% amino acid identity to any one of SEQ ID NO: 2, 4, 6, 8, 10, 12, 14, 21, 23, 25, 27, 29, 42, 44, 46, or 48;
(d) a fragment of any of the amino acid sequences of (a), (b), or (c), wherein the fragment has haploid induction activity; and
(e) a fragment of any of the amino acid sequences of (a), (b), or (c) or (d), wherein the fragment comprises an A motif, a B motif, or an AP2 DNA binding domain, or combinations thereof.

11. The method of claim 7, wherein the promoter that expresses in the plant egg cell is an egg cell-specific promoter or an egg cell-preferred promoter.

12. The method of claim 7, wherein the promoter that expresses in the egg cell comprises a polynucleotide comprising:
(a) a polynucleotide sequence of SEQ ID NO: 15, 16, 17, 18, 19, 31, 32, 33, 34, 35, 36, 37, 38, 39, 40, or 86;
(b) a polynucleotide sequence having at least 90% sequence identity to SEQ ID NO: 15, 16, 17, 18, 19, 31, 32, 33, 34, 35, 36, 37, 38, 39, 40, or 86;
(c) a fragment of the polynucleotide of (a) or (b), wherein the fragment retains its ability to drive expression of the operably linked polynucleotide sequence in an egg cell;
wherein said promoter drives expression of the operably linked Babyboom (BBM) polynucleotide encoding the Babyboom (BBM) polypeptide or the fragment thereof.

13. The method of claim 7, wherein the female gametophyte has not been fertilized prior to or during expression of the Babyboom (BBM) polynucleotide in an egg cell.

14. The method of claim 7, further comprising: modifying a regulatory region of an endogenous Babyboom (BBM) polynucleotide so that the Babyboom (BBM) polynucleotide expresses in a female gametophyte.

15. The method of claim 7, the method comprising:
expressing the Babyboom (BBM) polynucleotide from a modified endogenous genomic BBM locus, wherein the modified endogenous genomic BBM locus comprises a modified regulatory region of an endogenous polynucleotide encoding a BBM polypeptide, wherein one or more nucleotides in the regulatory region have been modified so that the BBM polypeptide expresses in a female gametophyte.

16. The method of claim 7, the method comprising:
expressing the Babyboom (BBM) polynucleotide from a modified endogenous genomic egg cell locus, wherein the egg cell's coding or genomic sequence in the endogenous genomic egg cell locus has been modified so that it encodes a BBM polypeptide, wherein the BBM polypeptide expresses in a female gametophyte.

17. The method of claim 7, wherein the Babyboom (BBM) polynucleotide has been modified from its native form using a gene editing technique.

18. (canceled)

19. The method of claim 7, wherein the female gametophyte is a monocot or dicot female gametophyte.

20. The method of claim 7, wherein the female gametophyte is a wheat, cotton, sunflower, safflower, tobacco, Arabidopsis, cannabis, canola, sugarcane, soy, barley, oats, rice, maize, triticale, sorghum, turf grass, rye, millet, or a flax female gametophyte.

21. The method of claim 7, further comprising:
(a) contacting the haploid embryo with a chromosome doubling agent for a period sufficient to generate a doubled haploid embryo;
(b) isolating the doubled haploid embryo; and
(c) regenerating a doubled haploid plant from the doubled haploid embryo of step (b).

22. The method of claim 7, further comprising:
(a) regenerating a parthenogenic plant from a haploid embryo comprising the Babyboom (BBM) polynucleotide operably linked to the promoter that expresses in an egg cell;
(b) pollinating the parthenogenic plant of (a) with pollen from a non-haploid inducer; and
(c) rescuing a haploid embryo from the parthenogenic plant of (b).

23. A method for obtaining a wheat plant producing clonal, non-reduced, non-recombined gametes, the method comprising:
(a) suppressing in a wheat plant cell the activity of:
(1) all endogenous Spo11 or Prd1, Prd2, or Prd3 polynucleotides or polypeptides;
(2) all endogenous Rec8 polynucleotides or polypeptides;
(3) all endogenous Osd1 polynucleotides or polypeptides;
(b) expressing, in a female gametophyte derived from the wheat plant cell, a Babyboom (BBM) polynucleotide operably linked to a promoter that expresses in an egg cell, wherein the Babyboom (BBM) polynucleotide comprises a nucleotide sequence encoding a Babyboom (BBM) polypeptide or a fragment thereof, wherein the female gametophyte is rendered parthenogenic and forms an embryo; and
(c) obtaining a plant from the embryo, wherein the embryo comprises the egg cell expressed Babyboom (BBM) polynucleotide and suppressed Spo11, Rec8, Osd1, Prd1, Prd2, or Prd3 polynucleotides or polypeptides, thereby producing a wheat plant producing clonal, non-reduced, non-recombined gametes.

24. The method of claim 23, wherein the plant is a first filial generation hybrid plant.

25. The method of claim 23, wherein the activity of the endogenous Spo11 polynucleotides or polypeptides, Rec8 polynucleotides or polypeptides, Osd1 polynucleotides or polypeptides, Prd1, Prd2, or Prd3 polynucleotides or polypeptides, or combinations thereof is suppressed by introducing a nucleotide modification into its polynucleotide sequence or an amino acid modification into its polypeptide sequence.

26.-29. (canceled)

30. The method of claim 23, wherein the nucleotide sequence encoding the Babyboom (BBM) polypeptide or fragment thereof is selected from the group consisting of BBM1, BBM2, BMN2, BMN3, ODP2, and BBML.

31. The method of claim 23, wherein the Babyboom (BBM) polynucleotide is selected from the group consisting of:
(a) a nucleotide sequence as set forth in SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(b) a nucleotide sequence that has at least 95% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(c) a nucleotide sequence that has at least 85% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(d) a fragment of any of the nucleotide sequences of (a), (b), or (c), wherein the nucleotide sequence encodes an amino acid fragment that has haploid induction activity;
(e) a fragment of any of the nucleotide sequences of (a), (b), (c), or (d), wherein the nucleotide sequence encodes an amino acid fragment that has haploid induction activity, wherein the amino acid fragment comprises an A motif, a B motif, or an AP2 DNA binding domain, or combinations thereof.

32. The method of claim 23, wherein the Babyboom (BBM) polypeptide is selected from the group consisting of:
(a) an amino acid sequence as set forth in SEQ ID NO: 2, 4, 6, 8, 10, 12, 14, 21, 23, 25, 27, 29, 42, 44, 46, or 48;
(b) an amino acid sequence that has at least 95% amino acid identity to any one of SEQ ID NO: 2, 4, 6, 8, 10, 12, 14, 21, 23, 25, 27, 29, 42, 44, 46, or 48;
(c) an amino acid sequence that has at least 85% amino acid identity to any one of SEQ ID NO: 2, 4, 6, 8, 10, 12, 14, 21, 23, 25, 27, 29, 42, 44, 46, or 48;
(d) a fragment of any of the amino acid sequences of (a), (b), or (c), wherein the fragment has haploid induction activity; and
(e) a fragment of any of the amino acid sequences of (a), (b), or (c) or (d), wherein the fragment comprises an A motif, a B motif, or an AP2 DNA binding domain, or combinations thereof.

33. A method of obtaining a clonal apomictic plant from one or more gametophytic cells in a plant in the absence of egg cell fertilization comprising:
(a) expressing, in one or more gametophytic cell, a Babyboom (BBM) polynucleotide operably linked to a promoter that expresses in an egg cell, wherein the Babyboom (BBM) polynucleotide comprises a nucleotide sequence encoding a Babyboom (BBM) polypeptide or a fragment thereof that retains haploid induction activity, wherein the Babyboom (BBM) polynucleotide is not naturally expressed in a female gametophyte;
(b) developing an embryo from the gametophytic cell in the absence of egg cell fertilization; and
(c) obtaining a progeny plant from one or more gametophytic cells wherein the progeny plant contains the chromosomes from the gametophytic cell of (a), thereby achieving propagation of a flowering plant in the absence of egg cell fertilization.

34. The method of claim 33, wherein the embryo is formed from an unreduced plant cell, wherein the unreduced plant cell is an egg cell or formed from a somatic cell.

35.-37. (canceled)

38. The method of claim 33, wherein the Babyboom (BBM) polynucleotide is selected from the group consisting of:
(a) a nucleotide sequence as set forth in SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(b) a nucleotide sequence that has at least 95% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(c) a nucleotide sequence that has at least 85% sequence identity to any one of SEQ ID NO: 1, 3, 5, 7, 9, 11, 13, 20, 22, 24, 26, 28, 41, 43, 45, 47, 74, 75, 76, 77, 78, 79, 80, 81, 82, 83, 84, 85, 87, or 88;
(d) a fragment of any of the nucleotide sequences of (a), (b), or (c), wherein the nucleotide sequence encodes an amino acid fragment that has haploid induction activity;
(e) a fragment of any of the nucleotide sequences of (a), (b), (c), or (d), wherein the nucleotide sequence encodes an amino acid fragment that has haploid induction activity, wherein the amino acid fragment comprises an A motif, a B motif, or an AP2 DNA binding domain, or combinations thereof.

39. The method of claim 33, wherein the Babyboom (BBM) polypeptide is selected from the group consisting of:
(a) an amino acid sequence as set forth in SEQ ID NO: 2, 4, 6, 8, 10, 12, 14, 21, 23, 25, 27, 29, 42, 44, 46, or 48;
(b) an amino acid sequence that has at least 95% amino acid identity to any one of SEQ ID NO: 2, 4, 6, 8, 10, 12, 14, 21, 23, 25, 27, 29, 42, 44, 46, or 48;
(c) an amino acid sequence that has at least 85% amino acid identity to any one of SEQ ID NO: 2, 4, 6, 8, 10, 12, 14, 21, 23, 25, 27, 29, 42, 44, 46, or 48;
(d) a fragment of any of the amino acid sequences of (a), (b), or (c), wherein the fragment has haploid induction activity; and
(e) a fragment of any of the amino acid sequences of (a), (b), or (c) or (d), wherein the fragment comprises an A motif, a B motif, or an AP2 DNA binding domain, or combinations thereof.


SEQUENCE LISTING (122 sequences)

SEQ ID NO: 1 - DNA, 2109 bp, Triticum aestivum
  source 1..2109: mol_type = other DNA
atggccaccgtgaacaactggttgggcttctccctctccccgcaggagctccctccctccgccgccgcctcaggtgacgtctccggcgccgacgtctgctacaacatcccacaagattggaacatgaggggatcagagctatctgccctggtggcggagcctaagctggaggacttcctcggcggcatatcctactccgaccaccaccaccaccacaagcaggccggcggcaacaacatggtcgtccctgcaggtagcggcagcggcggcgccgcatgctacacgacctcgggcggcagtagcgtgggctacctgtaccaccccagctcggcctccctccagtttgccgactccgtcatggtggcttcctccggcggtggcgtccaccacgacggcgctggcatcatggcgaacaccacagccaacggtgaccttaataatggcagcggcgggggtggcatcgggctgtccatgatcaagagctggctgcggagccagccatccccggcacaaccaccggagcagcagagggcggatgcggcggcacagggtctctcgctgtccatgaacatggcggcgtgcatgccgccgctcgttggcggggagcgtggtgtgccggagttggcgattgtccggaaggatgacacggccggtggcagcagcgctggttctggcgccgtggtgtcggccggtggtgcggatagcactggcggaagcagtggcgtggtggtggagacgccggcgtccaggaagacggctgacacgtttgggcagcggacctcgatttaccgtggtgtgaccaggcatagatggactgggagatatgaggctcacttatgggacaacagttgcagaagagaagggcaaacacgcaagggtcgtcaagtctatctaggtggctatgacaaagaggagaaagctgcaagagcttatgatttggctgctctcaaatattggggtgcgacgacgacgacaaatttcccagtaaacaactacgaaaaagagctagaggagatgaagcacatgacaaggcaggagtttgtggcgtctttgcgaaggaagagcagtggtttctccagaggcgcatccatttatcgtggagtaactaggcaccaccaacatgggagatggcaagcgaggatagggagagttgcagggaacaaggatctctacttaggcaccttcagcacgcaggaggaggctgcggaggcgtacgacatcgcggcgatcaagttccgggggctcaatgctgtcacaaacttcgacatgacgcgctacgatgtcaagagcatcctcgacagcaccgcgctccccatcggtagcgctgccaagcgcctcaaggacgccgaggcggctacggcgtcgtccctcgcgctgcagcagcagcagcacgcaggcgtggtgagcggctatgacatgggcgccctggccgcctacggtgccgcataccaccaccatcacccctctgtcgccggcgctgttgcgtggccgaccatcgcgttccaggcgccgcctcatcaggtttccagcggcggtggtggacacatgtaccacccctacgcccatgcgcagccgctgcgcgggtggtgtaagcaggagccggaccacgcggtgatcgcggctgcgcacagcctgcaggagctccaccacctcaacctcggcgccggtggcggcgcacatgacttcttctcgcagcatgcccaggcgatgcagcagcagcacggtggcctcggcagcatccacaacgcggccggcgcctcgctcgagcacagcaccggctccaactccgtcgtgtacaacaacggcggcgtcgagagctacatgcttcccatgagcaccaccaccaccgcgacggcgagccacgcccgcgagcaggtggcagcgcatgcccacgcacgcgcggtggcggcacaggcatacagcggcggcaacatgagcagggatcacgacgacgggaagatcgcctacgagaactacctcgtgagcaccgcagaagcctacggcggggggaggatggctgcctgggcacaggcctcagctccaccggcgacaagcagcagcgacatgaccggcgcgcagctcttcagcgtctggaacgactctaattag

SEQ ID NO: 2 - protein, 702 aa, Triticum aestivum
MATVNNWLGFSLSPQELPPSAAASGDVSGADVCYNIPQDWNMRGSELSALVAEPKLEDFLGGISYSDHHHHHKQAGGNNMVVPAGSGSGGAACYTTSGGSSVGYLYHPSSASLQFADSVMVASSGGGVHHDGAGIMANTTANGDLNNGSGGGGIGLSMIKSWLRSQPSPAQPPEQQRADAAAQGLSLSMNMAACMPPLVGGERGVPELAIVRKDDTAGGSSAGSGAVVSAGGADSTGGSSGVVVETPASRKTADTFGQRTSIYRGVTRHRWTGRYEAHLWDNSCRREGQTRKGRQVYLGGYDKEEKAARAYDLAALKYWGATTTTNFPVNNYEKELEEMKHMTRQEFVASLRRKSSGFSRGASIYRGVTRHHQHGRWQARIGRVAGNKDLYLGTFSTQEEAAEAYDIAAIKFRGLNAVTNFDMTRYDVKSILDSTALPIGSAAKRLKDAEAATASSLALQQQQHAGVVSGYDMGALAAYGAAYHHHHPSVAGAVAWPTIAFQAPPHQVSSGGGGHMYHPYAHAQPLRGWCKQEPDHAVIAAAHSLQELHHLNLGAGGGAHDFFSQHAQAMQQQHGGLGSIHNAAGASLEHSTGSNSVVYNNGGVESYMLPMSTTTTATASHAREQVAAHAHARAVAAQAYSGGNMSRDHDDGKIAYENYLVSTAEAYGGGRMAAWAQASAPPATSSSDMTGAQLFSVWNDSN

SEQ ID NO: 3 - DNA, 2250 bp, Triticum aestivum
  source 1..2250: mol_type = other DNA
atgggggatcctatgcgaggtttagggttccgctcgcgcgtttctctatgcgtttctggagaggatcgacgccaaggtttttctatggccggaagacgggggtccgtgtcctcttctagggtgcaggtagcttcgagcatggccaccgtgaacaactggttgggcttctccctctccccgcaggagctccctccctccgccgccgcctcaggtgacgtctccggcgccgacgtctgctacaacatcccgcaagattggaacatgaggggatcggagctatcagcgctggtggcggagccgaagctggaggacttccttggcggcatatcttattccgaccatcaccaccaccagcacaagcagcctggcggcaacaacatggtcgtccctgcaggcagcgccagtggcggtgccgcttgctacgcgaactctggcggcagtagcgtgggctacctgtaccaccccagctcggcctccctccagtttgccgactccgtcatggtggcttcctccggcggtggcgtccaccacgacggcgctggcatcatggcgaacaccacagccaacggtgaccttaatggcggcggcgggggtggcatcgggctgtccatgatcaagagctggctgcggagccagccatccccggcacaagcacaaccaccggagcaacagagggcagaggcggcggcgcaggggctctcgctgtccatgaacatggcggcgtgcataccgccgcttgttggcggggagcgtggcgtgccggagttggcgattgtacggaaggatgacacggctggtggcagcagtgctggttctggcgccgtagtgtcggccggtgcggacagcactggcggaagcagtggcgcggtggtcgagactccagcgtcgaggaagacagccgacacgtttgggcagcggacttcgatttatcgtggcgtgaccaggcatagatggaccgggagatatgaggctcacttgtgggacaacagttgcagaagagaagggcaaactcgcaagggtcgtcaagtctatctaggtggctatgacaaagaggagaaagctgcaagagcttatgatttggctgctctcaagtattggggtgccacgacgacgacaaatttcccagtaaacaactatgaaaaagagctagacgagatgaagcacatgacaaggcaggagtttgtggcatctctgcgaagaaagagcagtggtttctccagaggtgcatccatttatcgtggagtaactaggcaccaccaacatgggagatggcaagcgaggatagggagagttgcagggaacaaggacctctacttgggcaccttcagcacgcaggaggaggctgccgaggcgtacgacatcgcggcgatcaagttcaggggccttaacgctgtcaccaacttcgatatgacgcgctacgacgtcaagagcatcctcgacagcaccgcgctccccatcggcagcgccgccaagcgcctcaaggacaaagaggcagccacggcgtcgtccctcgcgctgcagcagcagcagcacgcaggcgtggtgagcggctacgacatgggcgccctggccgcctacggtgcggcgtaccaccaccatcacccctccgtcgccggcgctgttgcgtggccgaccatcgcgttccagacgccgcctcagcaggtttccggcggcggtggtggacacatgtaccacccctacgcccatgcgcagccgctgcgcgggtggtgcaagcaggagccggaccacgcggtgatcgcggccgcgcacagcctgcaggagctccaccacctcaacctcggcgccggtggcggcgcacacgacttcttctcgcagcatgcccaggcgatgcagcagcagcatggcggccttggcagcatcgacaacggggccggcgcctcgctggagcacagcactggctccaactccgtagtgtacaacaacggcgcggtcgagagctatatgctgccaatgagcaccaccaccactgcggcggtgagccacgcccacgagcaggcggcagcgcatgcccacgcacgggcggtggcggcacaggcgtatagcggcggcaacatgagcagggatcacgacgacgggaagatcgcctacgagaactacctcgtcagcaccgcagaagcctacggtggggggaggatggctgcgtgggcgccggcctcagctccgccggcgacaagcagcagcgacatgaccggcacgcagctcttcagcgtctggaacgacactaattag

SEQ ID NO: 4 - protein, 749 aa, Triticum aestivum
MGDPMRGLGFRSRVSLCVSGEDRRQGFSMAGRRGSVSSSRVQVASSMATVNNWLGFSLSPQELPPSAAASGDVSGADVCYNIPQDWNMRGSELSALVAEPKLEDFLGGISYSDHHHHQHKQPGGNNMVVPAGSASGGAACYANSGGSSVGYLYHPSSASLQFADSVMVASSGGGVHHDGAGIMANTTANGDLNGGGGGGIGLSMIKSWLRSQPSPAQAQPPEQQRAEAAAQGLSLSMNMAACIPPLVGGERGVPELAIVRKDDTAGGSSAGSGAVVSAGADSTGGSSGAVVETPASRKTADTFGQRTSIYRGVTRHRWTGRYEAHLWDNSCRREGQTRKGRQVYLGGYDKEEKAARAYDLAALKYWGATTTTNFPVNNYEKELDEMKHMTRQEFVASLRRKSSGFSRGASIYRGVTRHHQHGRWQARIGRVAGNKDLYLGTFSTQEEAAEAYDIAAIKFRGLNAVTNFDMTRYDVKSILDSTALPIGSAAKRLKDKEAATASSLALQQQQHAGVVSGYDMGALAAYGAAYHHHHPSVAGAVAWPTIAFQTPPQQVSGGGGGHMYHPYAHAQPLRGWCKQEPDHAVIAAAHSLQELHHLNLGAGGGAHDFFSQHAQAMQQQHGGLGSIDNGAGASLEHSTGSNSVVYNNGAVESYMLPMSTTTTAAVSHAHEQAAAHAHARAVAAQAYSGGNMSRDHDDGKIAYENYLVSTAEAYGGGRMAAWAPASAPPATSSSDMTGTQLFSVWNDTN

SEQ ID NO: 5 - DNA, 2247 bp, Triticum aestivum
  source 1..2247: mol_type = other DNA
atgggggatcctatgcgaggtttagggttccgctcgcgcgtttctctatgcgtttctggagaggatcgacgccaaggtttttctatggccggaagacgagggtccgtgtcctcttctagggtgcaggtagcttcgaccatggccaccgtgaacaactggttgggcttctccctctccccgcaggagctccctccctccgccgccgcctcaggtgacgtctccggtgccgacgtctgctacaacatcccgcaagattggaacatgaggggatcggagctatcagctctggtggcggagccgaagctggaggacttcctcggcggcatatcctattcggaccatcaccaccaccagcacaagcagcccggcggcaacaatatggtcgtccctgcaggcagcgccagcggcggtgccgcttgctacgcgagctctggcggcagtagcgtgggctacctgtaccaccccagctcggcctccctccagtttgccgactccgtcatggtggcttcctccggcggtggcgtccaccacgacggcgctggcatcatggcaaacaccacagccaacggtgaccttaataatggcggcggcgggggtggcatcgggctgtccatgatcaagagctggctgcggagccagccatccccggcacaaccaccggagcagcagagggccgacgcggcggcgcagggtctctcgctgtccatgaacatggcggcgtgcatgccgccgctcgcttgcggggagcgtggcgtgccggagttagcgattgtcaggaaggatgacacggccggtggcagcagcgctggttctggggctgtggtgtcggccggtgcggatagcaccggcggaagcagtggcgcggtggtggagaccccagcatcgaggaagacggccgatacgtttgggcagcgaacttcgatttaccgtggcgtgaccaggcatagatggactgggagatatgaggctcacttatgggacaacagttgcagaagagaagggcaaactcgcaagggtcgtcaagtctatctaggtgggtatgacaaagaggagaaagctgcaagagcttatgatttggctgctctcaagtattggggtgccacgacgacgacaaatttcccagtaaacaactatgaaaaagagctagaggagatgaaacacatgacaaggcaggagtttgtggcgtctctgcgaaggaagagcagtggtttctccagaggtgcatccatttatcgtggagtaactaggcaccaccaacatgggaggtggcaagcgaggatagggagagttgcagggaacaaggatctctacttgggcaccttcagcacgcaggaggaggctgccgaggcgtacgacatcgcggcgatcaagttccgggggctcaacgctgtcaccaacttcgatatgacgcgctacgacgtcaagagcatcctcgacagcaccgcgctccccataggcagcgccgccaagcgcctcaaggacgcagaggcggccacggcgtcgtccctcgcgctgcagcagcagcagcacgcaggcgttgtgagcggctacgacatgggcgccctggccgcctacggtgcggcgtaccaccaccatcacccctccgtcgccggcgctgttgcgtggccgaccatcgcgttccaggcgccgcctcagcaggtttctggcggcggtggtggacacatgtaccacccctacgcccatgcgcagccgctgcgcgggtggtgcaagcaggagccggaccacgcggtgatcgcggccgcgcacagcctgcaggagctccaccacctcaacctcggtgctggtggcggcgcgcacgacttcttctcgcagcatgcccaggcgatgcagcagcagcacggcggccttggcagcatcgacaacggggccggcgcctcgctggagcacagcactggctccaactccgtagtgtacaacaacggcgccgtcgagagctatatgctgccaatgagcaccaccagcaccgcggcggtgagccacgcccacgagcaggcggcagcgcatgcccacgcacgcgcggtggctgcacaggcgtacagcggcggcaacatgagcagggatcacgacgacgggaagatcgcctacgagaactacctcgtcagcaccgcagaagcctacggtgggggaaggatggctgcgtgggcgccggcctcagctccgcccgcgacaagcagcagcgacatgaccggcacgcagctcttcagcgtctggaacgacactaattag

SEQ ID NO: 6 - protein, 748 aa, Triticum aestivum
MGDPMRGLGFRSRVSLCVSGEDRRQGFSMAGRRGSVSSSRVQVASTMATVNNWLGFSLSPQELPPSAAASGDVSGADVCYNIPQDWNMRGSELSALVAEPKLEDFLGGISYSDHHHHQHKQPGGNNMVVPAGSASGGAACYASSGGSSVGYLYHPSSASLQFADSVMVASSGGGVHHDGAGIMANTTANGDLNNGGGGGGIGLSMIKSWLRSQPSPAQPPEQQRADAAAQGLSLSMNMAACMPPLACGERGVPELAIVRKDDTAGGSSAGSGAVVSAGADSTGGSSGAVVETPASRKTADTFGQRTSIYRGVTRHRWTGRYEAHLWDNSCRREGQTRKGRQVYLGGYDKEEKAARAYDLAALKYWGATTTTNFPVNNYEKELEEMKHMTRQEFVASLRRKSSGFSRGASIYRGVTRHHQHGRWQARIGRVAGNKDLYLGTFSTQEEAAEAYDIAAIKFRGLNAVTNFDMTRYDVKSILDSTALPIGSAAKRLKDAEAATASSLALQQQQHAGVVSGYDMGALAAYGAAYHHHHPSVAGAVAWPTIAFQAPPQQVSGGGGGHMYHPYAHAQPLRGWCKQEPDHAVIAAAHSLQELHHLNLGAGGGAHDFFSQHAQAMQQQHGGLGSIDNGAGASLEHSTGSNSVVYNNGAVESYMLPMSTTSTAAVSHAHEQAAAHAHARAVAAQAYSGGNMSRDHDDGKIAYENYLVSTAEAYGGGRMAAWAPASAPPATSSSDMTGTQLFSVWNDTN

SEQ ID NO: 7 - DNA, 1980 bp, Triticum aestivum
  source 1..1980: mol_type = other DNA
atggcttctgggaacgactggttggggttctcgctctccggccaagagaacccacagccgcatcaggatagctcgccgcccgccgacatcgacatcgctggcgccagtggcttctatggcctacccacgcagccagctcccgacgctcaactcggtgtgccaggtcatcatcagccttcatacgggatcacggaggccttcaataggggagctcatgaaatccacgattggaacatgaggggtgatctggactacaacggcggtgcgtccgagctctcgatgctcgtcgggtcgagcgcagttggcggcaagagggcggccatggaggccgagaccgagcccaagcttgaggacttcctcgttggcaactcgttcgtctccgagcaagatcaggccggtggcttcttgttctccggtgtcccgatggcgaccagcaccaacagcaacagcaggagcaacaccatggagctctctatgatcaagagctggctccgtgacaaccaggtaccccaagcgccgcacccgcagtcgcagaccggggcgcccgcgcagcagccgcaaccccacgaggagatgggcaccgacgcgagcagcttcgatccgctggggagaaacggcgcgttggtggtggcggggagctcgcagagcctggcgctctcgatgagcacgggctcaggatcgcacttgccgatggctgtgctcggcggcagtcccagtgggggagtctcagagagcacctcctcggagaacaagcgggccagcggcgccatggattcgccgggcggtgcggtagaagctgtcgcgaggaagtccatcgacacattcgggcaaaggacgtcgatatatcgaggtgtaacaagacatagatggacagggcgttatgaggctcatctctgggacaatagctgcagaagagaagggcagagtcgcaagggtaggcaagtttatctgggtggctatgacaaggaggacaaggcagcaagggcttatgatttggcagcactcaagtattggggtacaacaacaacaacaaatttcccaattaatacctacgaaaaagaggtggatgaaatgaaacatatgactaggcaggaatacatcgcatacctaagaaggaatagcagcggattttcccgtggtgcgtcaaaatatcgtggtgtaactaggcaccatcagcatgggagatggcaagcaagaattgggagggttgcaggaaacaaggatctctacttaggcaccttcagcaccgaggaggaggcggcggaggcctacgacatcgcggcgatcaagttccgtgggctgaacgccgtcaccaacttcgacatgagtcgctacgacgtcaagaccatcctcgagagcagcaccctgccggtgggtggcgcggcgaggcgccccaaggaggtcgctgatcatccagaggctggcgccacgatctggcgggccggcatggacggcggcgtcatctcccagctgaccgacgtcgggatgggcacgtacgcgtcgtaccaccacggctggccaaccatcgcattccagcagccctcgccgctgtcggtgcactacccatacgtcgcccagccgtctcgcgggtggtgcaagcccgagcaggacgccagcgtcgccacgcaaagcctgcaggacctccagcagctgcacctcgggaccgccgctcacaactatttccaggcatcgtcgagctccacggtctacaacggcggcgggtaccagcaaggcaccagtggcaacgccttcttgatgccggctagcaccgtggtggatgaacaggggcacagcagcactgccaccaaccagggaagcacatgcagctacggggacgaagaggggaagctcaatctcgggtacgatgccatggcgatggcgagcaccggcgccgacccatacgcggcggcgccaactgtgagcattgcgagggcgaacgggtactccaacaactggagctcgccgttcaatggcatgtga

SEQ ID NO: 8 - protein, 659 aa, Triticum aestivum
MASGNDWLGFSLSGQENPQPHQDSSPPADIDIAGASGFYGLPTQPAPDAQLGVPGHHQPSYGITEAFNRGAHEIHDWNMRGDLDYNGGASELSMLVGSSAVGGKRAAMEAETEPKLEDFLVGNSFVSEQDQAGGFLFSGVPMATSTNSNSRSNTMELSMIKSWLRDNQVPQAPHPQSQTGAPAQQPQPHEEMGTDASSFDPLGRNGALVVAGSSQSLALSMSTGSGSHLPMAVLGGSPSGGVSESTSSENKRASGAMDSPGGAVEAVARKSIDTFGQRTSIYRGVTRHRWTGRYEAHLWDNSCRREGQSRKGRQVYLGGYDKEDKAARAYDLAALKYWGTTTTTNFPINTYEKEVDEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKTILESSTLPVGGAARRPKEVADHPEAGATIWRAGMDGGVISQLTDVGMGTYASYHHGWPTIAFQQPSPLSVHYPYVAQPSRGWCKPEQDASVATQSLQDLQQLHLGTAAHNYFQASSSSTVYNGGGYQQGTSGNAFLMPASTVVDEQGHSSTATNQGSTCSYGDEEGKLNLGYDAMAMASTGADPYAAAPTVSIARANGYSNNWSSPFNGM

SEQ ID NO: 9 - DNA, 1980 bp, Triticum aestivum
  source 1..1980: mol_type = other DNA
atggcttctggaaacgactggttggggttctcgctctccggccaagaaaacccacagccgcatcaggatagctcgccgcccgccggcatcgacatctccggcgccagtgacttctatggcctacccacgcagccagcccccgacgctcaacttggcgtgccaggccatcagccttcgtacgggatcacggaggccttcactcatgaaacccacgattggaacatgaggggtgatctggactataacggcggtgcgtccgagctctcgatgctcgtcgggtcgagcgcagtcggcggcaagagggcggccatggaggccgagaccgagcccaagcttgaggacttcctcggtggcaactcgttcgtctccgagcaagatcaggccggcggcttcttgttctccggagtcccgatggcgaccagcgccaacagcaacagcgggagcaacaccatggagctctctatgatcaagagctggctccgtaacaaccaggtaccccaagcgccgcacccgcagtcgcataccggggcgcccgtgcagcagccgcagccccacgaggagatgagcaccgacgcaagcgcgagcagcttcgatgcgctggggagaaacggcgcgttggtggtggcggggagctcgcagagcctggcgctctcgatgagcacgggctcaggatcgcacttgccgatggctgcggtcggcggcagtgccactgggggagtctcagagagcacctcctcggagaacaagcgggccagcggcaccatggattcgccgggcggtgccgtagaagctgtcgcgaggaagtccatcgacacattcgggcaaaggacgtcgatatatcgaggtgtaacaagacatagatggacagggcgttatgaggctcatctctgggacaatagctgcagaagagaagggcagagtcgcaagggtaggcaaggtggctatgacaaggaggacaaggcagcaagggcttatgatttggcagcactcaagtattggggtacaacaacaacaacaaatttcccaattaatacctacgaaaaagaggtggatgaaatgaaacatatgactaggcaggaatacatcgcatacctaagaaggaatagcagcggattttctcgtggtgcgtcaaaatatcgtggtgtgactaggcaccatcagcatgggagatggcaagcaagaattgggcgggttgcaggaaacaaggatctctacttaggcaccttcagcaccgaggaggaggcggcggaggcctacgacatcgcggcgatcaagttccgtgggctcaatgccgtcaccaacttcgacatgagtcgctacgacgtcaagaccatcctccagagcagcaccctaccggtggcggtgagtggcgcggcgaggcgcctcaaggaggccgctgatcatccagaggctggcgccacgatctggcgggccggcatggatggcggcgtcatctcccagctgaccgacgtcgggatgggcgcttacgcgtcgtaccaccacggctggccgacaatcgcattccagcagccgtcgccgctctcggtgcactacccatacgtcgcccagccgtctcgcgggtggtgcaagcccgagcaggacgccaccgtcgccacgcaaaacctgcaggacctccaggagctgcacctcgggatcgccgctcacaactatttccaggcatcgtcgagctccacggtctacaacggcggcggcggcggcgggtaccagcagggcaccggtggcaacgccttcctgatgccggctagcgccgtcgtggacgaacaggggcacagcagcactgccaccaaccagggaagcacctgcagctacggggacgaagaagggaagctcaatattgggtacgacgccatggcgatggcgagcaccggcgccgacccatacgcggcggcgtcaacggtgagcatcgcgagggcgaacgggtactccaacaactggagctcgccgttcaatggcatgtga

SEQ ID NO: 10 - protein, 659 aa, Triticum aestivum
MASGNDWLGFSLSGQENPQPHQDSSPPAGIDISGASDFYGLPTQPAPDAQLGVPGHQPSYGITEAFTHETHDWNMRGDLDYNGGASELSMLVGSSAVGGKRAAMEAETEPKLEDFLGGNSFVSEQDQAGGFLFSGVPMATSANSNSGSNTMELSMIKSWLRNNQVPQAPHPQSHTGAPVQQPQPHEEMSTDASASSFDALGRNGALVVAGSSQSLALSMSTGSGSHLPMAAVGGSATGGVSESTSSENKRASGTMDSPGGAVEAVARKSIDTFGQRTSIYRGVTRHRWTGRYEAHLWDNSCRREGQSRKGRQGGYDKEDKAARAYDLAALKYWGTTTTTNFPINTYEKEVDEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKTILQSSTLPVAVSGAARRLKEAADHPEAGATIWRAGMDGGVISQLTDVGMGAYASYHHGWPTIAFQQPSPLSVHYPYVAQPSRGWCKPEQDATVATQNLQDLQELHLGIAAHNYFQASSSSTVYNGGGGGGYQQGTGGNAFLMPASAVVDEQGHSSTATNQGSTCSYGDEEGKLNIGYDAMAMASTGADPYAAASTVSIARANGYSNNWSSPFNGM

SEQ ID NO: 11 - DNA, 1986 bp, Triticum aestivum
  source 1..1986: mol_type = other DNA
atggcttctggaaacgactggttggggttctcgctctccggccaagaaaacccacagccgcatcaggatagcccgccgcccgccggcatcgacatctccggcgccagtgacttctatggcgtacccacgcagccagctcccgacgctcaactcggcgtgccaggtcatcagccttcatacgggatcacggaggacttcaataggggagctcatgaaacccacgattggaacatgaggggtgatctggactataacggcggtgcgtccgagctctcgatgctcgtcgggtcgagcgcagtcggcggcaagagggcggccatggaggccgagaccgagcccaagcttgaggacttcctcggttgcaactcgttcgtctccgagcaagatcaggccggcggcttcttgttctccggagtcccgatgaccaccagcagcaacagcaacaccgggagcaacaccatggagctctctatgatcaagagctggctccgtgacaaccagctaccccaagcgccgcacccgcagtcgcagaccggggcgcccgcgcagcagccgcaaccccacgaggagatgggcaccgacgcgagcagcttcgatccgcgggggagaaacggcgcgttggtggtggcggggagctcgcagagcctggcgctctcgatgagcacgggttcaggatcgcacttgccaatggctgtggtcagcggcagtgccagtgggggagtctcagagagcacctcctcggagaacaagcgagccagcggcgccatggattcgccgggcggtgcggtagaagctgtcgcgaggaagtccatcgacacattcgggcaaaggacgtcgatatatcgaggtgtaacaagacatagatggacaggccgttatgaggctcatctctgggacaatagctgcagaagagaagggcagagtcgcaagggccgacaagtttatcttggtggctatgacaaggaggacaaggcagcaagggcttatgatttggcagcactcaagtattggggtacaacaacaactacaaatttcccaattaatacctacgaaaaagaggtggatgaaatgaaacatatgactaggcaggaatatatcgcatacttaagaaggaatagcagcggattttctcgtggtgcgtcaaaatatcgtggtgtaactaggcaccatcagcatgggagatggcaagcaagaattgggcgggttgcaggaaacaaagatctctacttaggcaccttcagcaccgaggaggaggcggcggaggcctacgacatcgcggcgatcaagttccgcgggctcaacgccgtcaccaacttcgacatgagtcgctacgacgtcaagaccatcctcgagagcagcaccctgccggtgggtggcgcggctaggcgcctcaaggaggccgctgatcatccggaggccggcgccacgatctggcgagccggcatggacggcgccatcatctcccagctgaccgacgtcgggatgggcgcgtacgcgtcgtaccaccacggctggccgaccatcgcattccagcagccgtcgccgctctcggtgcactacccatacgtcgcccagccgccccgcgggtggtgcaagcccgagcaggacgccaccgtcgccacgcaaagcctgcaggacctccagcagctgcacctcgggaccgccgctcacaactatttccaggcatcgtcgagctcaacggtctacaacggcggcggcgtcggcgggtaccagcagggcaccggtggcaacgccttcttgatgccggctagcaccgttgtggatgaacaggggcacagcagcactgccgccaaccagggaagcacctgcagctacggggacgaagaggggaagctcaatcttgggtacgatgccatggcgatggcgagcaccggcgccgacccatacgcggcggcgtcaactgtgagcatcgcgagggcgaacgggtactccaacaactggagctcgccgttcagtggcatgtga

SEQ ID NO: 12 - protein, 661 aa, Triticum aestivum
MASGNDWLGFSLSGQENPQPHQDSPPPAGIDISGASDFYGVPTQPAPDAQLGVPGHQPSYGITEDFNRGAHETHDWNMRGDLDYNGGASELSMLVGSSAVGGKRAAMEAETEPKLEDFLGCNSFVSEQDQAGGFLFSGVPMTTSSNSNTGSNTMELSMIKSWLRDNQLPQAPHPQSQTGAPAQQPQPHEEMGTDASSFDPRGRNGALVVAGSSQSLALSMSTGSGSHLPMAVVSGSASGGVSESTSSENKRASGAMDSPGGAVEAVARKSIDTFGQRTSIYRGVTRHRWTGRYEAHLWDNSCRREGQSRKGRQVYLGGYDKEDKAARAYDLAALKYWGTTTTTNFPINTYEKEVDEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKTILESSTLPVGGAARRLKEAADHPEAGATIWRAGMDGAIISQLTDVGMGAYASYHHGWPTIAFQQPSPLSVHYPYVAQPPRGWCKPEQDATVATQSLQDLQQLHLGTAAHNYFQASSSSTVYNGGGVGGYQQGTGGNAFLMPASTVVDEQGHSSTAANQGSTCSYGDEEGKLNLGYDAMAMASTGADPYAAASTVSIARANGYSNNWSSPFSGM

SEQ ID NO: 13 - DNA, 2133 bp, Zea mays
  source 1..2133: mol_type = other DNA
atggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaa

SEQ ID NO: 14 - protein, 710 aa, Zea mays
MATVNNWLAFSLSPQELPPSQTTDSTLISAATADHVSGDVCFNIPQDWSMRGSELSALVAEPKLEDFLGGISFSEQHHKANCNMIPSTSSTVCYASSGASTGYHHQLYHQPTSSALHFADSVMVASSAGVHDGGAMLSAAAANGVAGAASANGGGIGLSMIKNWLRSQPAPMQPRVAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAASTGLYHPYAQQPMRGGGWCKQEQDHAVIAAAHSLQDLHHLNLGAAGAHDFFSAGQQAAAAAMHGLGSIDSASLEHSTGSNSVVYNGGVGDSNGASAVGGSGGGYMMPMSAAGATTTSAMVSHEQVHARAYDEAKQAAQMGYESYLVNAENNGGGRMSAWGTVVSAAAAAAASSNDNMAADVGHGGAQLFSVWNDT

SEQ ID NO: 15 - DNA, 1358 bp, Zea mays
  source 1..1358: mol_type = genomic DNA
acacaggaccaagaacttgaagatgcatttgaaggcctttatcttgttgactcccaagggccctagactttgtaatcttgcatttgtgctctgctgatctggtctgatactgatgtaactgatcaatgaactaattgtattagaactggattgtactctttttttcctttatatggttttctcataaggcgagtttttacctagaaaggtttttaataagacagccattgcacaaacagctataatattttatttaaagtctatgagactgactccgtgtgtgctactgcctactggctactactatctgtgaaattgtgacctgtgaactttgaaatgtgaaatttgtgacttgagaactatgattttatgacatatgaagttgtgaactgtgtatttgatacctgtgtgaatttatgacctatttaggccttgttcgtttacaccaatccagctctggattgacatggattggaattaaatacatgtcacaatctatgtcccaaaataatccaagcctactcatttttttatttggttaaacccatcatagattataacccaaggatttaggaaatttttaaactatggaagacatgaattctattcatagcttattaggtatggaataaatccatgaatatattgcacaagtttatattagaattcatgaatcaaaagaataactagttttgagagatacatggattaaatggtagatttaatctcactatgggattgagtgtgatatatggatttattcaatccaaatccggattaaatccatggtggatctatatatattggtgtgctcttagctcggttgtgtaggtgggccatgtttgacgtgccgagctggcacgatcggaccttttacccgtgccgtgctcgtgcaaggggtgttgcccgtcaggaggcaccgtgagttaatcggactcaattggaccggactcctcggatcgcgccgtgccgccgtttggatttctatacctgcacctgtggcctgtggggagtggggactgcgaatgacattcttgcatccctcctcaccaatcaaggcggcaacataccggccctttggccttccatgaacatgaacgcggcggaacgccacgccggcgtgcactactcacctgcatgaattcgccgcccactcacagcgccaacccaacttgattgcacgcactaccatcaattcgccgccgcggccatcccttctgccagctgctatttatacgcctcgccccgctccagtctcagcagaaccaccagtcctccactccatcttctactccgaccacaaccacagcgaccacgaccgtgcacgtacgtacatcagcacaccaggcaacggcacc

SEQ ID NO: 16 - DNA, 1299 bp, Panicum virgatum
  source 1..1299: mol_type = genomic DNA
cctttatgtattgtgaattgcctcatgactctgttttacatcaggaccttgtcagactagttggcaacagtgtataaatgaaagcagcagtgtgcaggtgacttctaatattgaagtgctcaatcaaaatcatgaggaaggcaaccaaaatagtgaaggccaaattactcctattcgaagcattgtgtctgaagatgcagatactgaaagatcagaaagggaaataggtctgacccctcctgcaggaccgtcagcattgccggatgagcagcttaacacgaaggtggagtgccacgaagctgaacgcaactgttgctatgatgaggacacttcgagcttatttgatactgaatcgctgtacagcaaagcatccagtttgcacgaagacactcgtaaggatacttctccaagtgatctatctgctccgagatctcccgaggaatctacagtttttctgaactcttctgttccgggatcagtaggtatcattgctgattctgattttgttcacgttgctctgacataatgtcttgttcaatcttttattgtagtatctgtgagagcctcctgactacatctttcattttgctttttcattaggaatctgtcgaagcagcaggagaagaggtatagacgaactccgcgccaagctgcagagtttcaaagtctccagcactgtaaaaggaagctacattgctatgagtgcccctcgcccgaagcaaggtgacaatctgagccaatctgcaatcgcgttgctccggaacagcgagaacgcacctgctgttaaagtaggccatcctggcaagccggaccctgatcttccggtcgacaagcgctgcagcccatcagtggaagatcaagggatcactgataggcagtagggcgtacgccgtagtttattattgtggacagttagcgtttgtgctcaacattagttggtggtggtggtgttcaacctttggctgaaggttattaggtgtgcatctgaccggaaactatccgtgtgtttgcgcagcgtgtagtgacgaagctgctctgtgcatttgttattagcagttaacatattgtgggtgtggtggccgtggtggaaatgttaagcaactggcagccgtacagttggttaattggttatgcaggcgtttggttttggcttttttgtaatcctgttatcgtgaacagatccgttgtgccccgtgctgttattcgtacacgtgctcacttgtttaatctcttgcaatggcttacccatgtactgggtccagaagacaggcgaggagcatggctattcggaacagaaga

SEQ ID NO: 17 - DNA, 100 bp, Panicum virgatum
  source 1..100: mol_type = genomic DNA
gccgcaggtcgtttggcgttccatgagcagacactgcagtgagtggatagcaatcaatagcgatgctctttggccttccatgaaacgcacgcactcatgt

SEQ ID NO: 18 - DNA, 163 bp, Panicum virgatum
  source 1..163: mol_type = genomic DNA
cgccgcccacaatccaagtcggtgtacccccgaatcgtcgatcatattaattccggccgcctagcgctgctatatatagagagacctcgcggcactccactcggatcctgcaaggctgcaacgcacacacttccggccgactcgatccgtccaagtcgcagca

SEQ ID NO: 19 - DNA, 1398 bp, Panicum virgatum
  source 1..1398: mol_type = genomic DNA
cctttatgtattgtgaattgcctcatgactctgttttacatcaggaccttgtcagactagttggcaacagtgtataaatgaaagcagcagtgtgcaggtgacttctaatattgaagtgctcaatcaaaatcatgaggaaggcaaccaaaatagtgaaggccaaattactcctattcgaagcattgtgtctgaagatgcagatactgaaagatcagaaagggaaataggtctgacccctcctgcaggaccgtcagcattgccggatgagcagcttaacacgaaggtggagtgccacgaagctgaacgcaactgttgctatgatgaggacacttcgagcttatttgatactgaatcgctgtacagcaaagcatccagtttgcacgaagacactcgtaaggatacttctccaagtgatctatctgctccgagatctcccgaggaatctacagtttttctgaactcttctgttccgggatcagtaggtatcattgctgattctgattttgttcacgttgctctgacataatgtcttgttcaatcttttattgtagtatctgtgagagcctcctgactacatctttcattttgctttttcattaggaatctgtcgaagcagcaggagaagaggtatagacgaactccgcgccaagctgcagagtttcaaagtctccagcactgtaaaaggaagctacattgctatgagtgcccctcgcccgaagcaaggtgacaatctgagccaatctgcaatcgcgttgctccggaacagcgagaacgcacctgctgttaaagtaggccatcctggcaagccggaccctgatcttccggtcgacaagcgctgcagcccatcagtggaagatcaagggatcctgataggcagtagggcgtacgccgtagtttattattgtggacagttagcgtttgtgctcaacattagttggtggtggtggtgttcaacctttggctgaaggttattaggtgtgcatctgaccggaaactatccgtgtgtttgcgcagcgtgtagtgacgaagctgctctgtgcatttgttattagcagttaacatattgtgggtgtggtggccgtggtggaaatgttaagcaactggcagccgtacagttggttaattggttatgcaggcgtttggttttggcttttttgtaatcctgttatcgtgaacagatccgttgtgccccgtgctgttattcgtacacgtgctcacttgtttaatctcttgcaatggcttacccatgtactgggtccagaagacaggcgaggagcatggctattcggaacagaagagccgcaggtcgtttggcgttccatgagcagacactgcagtgagtggatagcaatcaatagcgatgctctttggccttccatgaaacgcacgcactcatgt

SEQ ID NO: 20 - DNA, 2040 bp, Zea mays
  source 1..2040: mol_type = other DNA
atggcttcagcgaacaactggctgggcttctcgctctcgggccaggataacccgcagcctaaccaggacagctcgcctgccgccggtatcgacatctccggcgccagcgacttctatggcctgcccacgcagcagggctccgacgggcatctcggcgtgccgggcctgcgggacgatcacgcttcttatggtatcatggaggcctacaacagggttcctcaagaaacccaagattggaacatgaggggcttggactacaacggcggtggctcggagctctcgatgcttgtggggtccagcggcggcggcgggggcaacggcaagagggccgtggaagacagcgagcccaagctcgaagatttcctcggcggcaactcgttcgtctccgatcaagatcagtccggcggttacctgttctctggagtcccgatagccagcagcgccaatagcaacagcgggagcaacaccatggagctctccatgatcaagacctggctacggaacaaccaggtggcccagccccagccgccagctccacatcagccgcagcctgaggaaatgagcaccgacgccagcggcagcagctttggatgctcggattcgatgggaaggaacagcatggtggcggctggtgggagctcgcagagcctggcgctctcgatgagcacgggctcgcacctgcccatggttgtgcccagcggcgccgccagcggagcggcctcggagagcacatcgtcggagaacaagcgagcgagcggtgccatggattcgcccggcagcgcggtagaagccgtaccgaggaagtccatcgacacgttcgggcaaaggacctctatatatcgaggtgtaacaaggcatagatggacagggcggtatgaggctcatctatgggataatagttgtagaagggaagggcagagtcgcaagggtaggcaagtatacttaggtggctatgacaaggaggacaaggcagcaagggcttacgatttggcagctctcaagtattggggcactacgacaacaacaaatttccctataagcaactacgaaaaagagctagaagaaatgaaacatatgactagacaggagtacattgcatacctaagaagaaatagcagtggattttctcgtggggcatcaaagtatcgtggagtaactagacatcatcagcatgggagatggcaagcaaggatagggagagttgcaggaaacaaggatctttacttgggcacattcagcaccgaggaggaggcggcggaggcctacgacatcgccgcgatcaagttccgcggtctcaacgccgtcaccaacttcgacatgagccgctacgacgtgaagagcatcctcgagagcagcacactgcctgtcggcggtgcggccaggcgcctcaaggacgccgtggaccacgtggaggccggcgccaccatctggcgcgccgacatggacggcgccgtgatctcccagctggccgaagccgggatgggcggctacgcctcgtacggccaccacggctggccgaccatcgcgttccagcagccgtcgccgctctccgtccactacccgtacggccagccgtcccgcgggtggtgcaaacccgagcaggacgcggccgccgccgcggcgcacagcctgcaggacctccagcagctgcacctcggcagcgcggcccacaacttcttccaggcgtcgtcgagctccacagtctacaacggcggcgccggcgccagtggtggctaccagggcctcggtggtggcagctctttcctcatgccgtcgagcactgtcgtggcggcggccgaccaggggcacagcagcacggccaaccaggggagcacgtgcagctacggggacgaccaccaggaggggaagctcatcggttacgacgccgccatggtggcgaccgcagctggtggagacccgtacgctgcggcgaggaacggctaccagttctcgcagggctcgggttccacggtgagcatcgcgagggcgaacgggtacgctaacaactggagctctcctttcaacaacggcatggggtga

SEQ ID NO: 21 - protein, 679 aa, Zea mays
MASANNWLGFSLSGQDNPQPNQDSSPAAGIDISGASDFYGLPTQQGSDGHLGVPGLRDDHASYGIMEAYNRVPQETQDWNMRGLDYNGGGSELSMLVGSSGGGGGNGKRAVEDSEPKLEDFLGGNSFVSDQDQSGGYLFSGVPIASSANSNSGSNTMELSMIKTWLRNNQVAQPQPPAPHQPQPEEMSTDASGSSFGCSDSMGRNSMVAAGGSSQSLALSMSTGSHLPMVVPSGAASGAASESTSSENKRASGAMDSPGSAVEAVPRKSIDTFGQRTSIYRGVTRHRWTGRYEAHLWDNSCRREGQSRKGRQVYLGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDGAVISQLAEAGMGGYASYGHHGWPTIAFQQPSPLSVHYPYGQPSRGWCKPEQDAAAAAAHSLQDLQQLHLGSAAHNFFQASSSSTVYNGGAGASGGYQGLGGGSSFLMPSSTVVAAADQGHSSTANQGSTCSYGDDHQEGKLIGYDAAMVATAAGGDPYAAARNGYQFSQGSGSTVSIARANGYANNWSSPFNNGMG

SEQ ID NO: 22 - DNA, 2088 bp, Oryza sativa
  source 1..2088: mol_type = other DNA
atggccactatgaacaactggctcgccttctcgctctcgccgcaggaccaactcccaccgtcgcagaccaatagcactctcatctccgctgctgcaaccaccacaaccgcaggcgattcgtcaacgggcgacgtctgcttcaacatccctcaagactggtccatgcgcggaagcgagcttagcgctctcgtcgcggagcccaagttggaggatttcttgggaggcatctccttctcggagcaacagcatcatcacggcggaaagggcggtgttatcccaagctctgctgccgcatgctatgcaagctccggctccagcgtgggctacctctaccctccgccttcatcctcgtcacttcagtttgcagacagcgtgatggtcgcaacctcatctccagtggttgcgcacgatggcgtgagcggtggcggtatggtctcagcagcagcggctgcagcagcttcgggtaatggcgggattggcctctccatgatcaagaactggctcaggagccaaccggctccgcaacctgcgcaagcactcagcctgtcgatgaacatggctggtactactaccgctcaaggtggaggcgcaatggcacttctcgcaggcgctggcgaaagaggaaggaccacaccagcatccgagagcctctctacttccgcgcacggagccaccacggctacaatggctggcgggaggaaagagatcaacgaggaaggatctggttccgctggtgccgtggttgcagttggctcagaatcaggtggttccggcgctgttgttgaagctggtgccgctgcggcagcggctcggaagagcgttgatactttcggccaaagaacgagcatctacagaggcgttactcggcaccgctggaccggcaggtacgaggcacacttgtgggacaacagctgtcgccgcgagggccaaactaggaagggaagacaggtctatctaggaggatatgacaaagaggagaaggctgccagagcgtacgacctggccgcgttgaagtactggggtccaacaacgacgaccaacttcccggtgaacaactacgagaaggagctggaagagatgaagcacatgacgcggcaggagttcgtcgcttctctcaggcgcaagtcatctggtttctccagaggtgcgtcgatctatagaggagttacccgccaccaccagcacggaaggtggcaggcaagaatcgggagagtcgccggtaacaaggacctgtacttgggaaccttctcgactcaggaggaggcagcggaagcgtatgacattgcggcgatcaagttccgcggtctcaatgccgtgaccaacttcgacatgtcacgctatgatgtcaagtcgattctggatagcgctgcgttgcctgtgggaaccgctgccaaacgcctcaaggacgcggaagcagctgccgcgtacgatgttggcaggattgcctcacatctcggtggagatggagcttacgctgcccactacgggcatcatcaccactctgcagccgcagcttggcctacaatagcattccaagcggcagcggctcctcctccacacgctgctggtctttaccatccgtacgcgcaacctctccgcggttggtgtaagcaggaacaagatcatgcggtgattgcggctgcacacagcttgcaagatctgcatcacctcaatctgggagccgcagcagctgcccatgacttcttctcacaagccatgcagcagcagcatggcctgggcagcatagacaatgcgtctctggagcactccaccggatcgaactcggtggtgtacaatggagacaacggcggaggaggtggaggttacatcatggcacctatgtcagcggtctctgctaccgctacggcggtggcctcatcccacgaccacggtggagacggcggcaagcaggtccaaatgggctacgactcctaccttgtgggagctgacgcttacggcggaggaggagctggtcgcatgcctagctgggccatgacgcctgcttctgctcctgcggctacgagctcgtcggatatgacaggagtgtgtcatggcgcccaactgttctcggtgtggaatgatacatag

SEQ ID NO: 23 - protein, 695 aa, Oryza sativa
MATMNNWLAFSLSPQDQLPPSQTNSTLISAAATTTTAGDSSTGDVCFNIPQDWSMRGSELSALVAEPKLEDFLGGISFSEQQHHHGGKGGVIPSSAAACYASSGSSVGYLYPPPSSSSLQFADSVMVATSSPVVAHDGVSGGGMVSAAAAAAASGNGGIGLSMIKNWLRSQPAPQPAQALSLSMNMAGTTTAQGGGAMALLAGAGERGRTTPASESLSTSAHGATTATMAGGRKEINEEGSGSAGAVVAVGSESGGSGAVVEAGAAAAAARKSVDTFGQRTSIYRGVTRHRWTGRYEAHLWDNSCRREGQTRKGRQVYLGGYDKEEKAARAYDLAALKYWGPTTTTNFPVNNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSAALPVGTAAKRLKDAEAAAAYDVGRIASHLGGDGAYAAHYGHHHHSAAAAWPTIAFQAAAAPPPHAAGLYHPYAQPLRGWCKQEQDHAVIAAAHSLQDLHHLNLGAAAAAHDFFSQAMQQQHGLGSIDNASLEHSTGSNSVVYNGDNGGGGGGYIMAPMSAVSATATAVASSHDHGGDGGKQVQMGYDSYLVGADAYGGGGAGRMPSWAMTPASAPAATSSSDMTGVCHGAQLFSVWNDT

SEQ ID NO: 24 - DNA, 2112 bp, Oryza sativa
  source 1..2112: mol_type = other DNA
atggcttctgcaaacaactggctgggcttctcgctctccggccaagagaatccgcagcctcaccaggatagctcgcctccggcagccatcgacgtctccggcgccggcgacttctatggcctgccgacgtcgcagccgacggcggccgacgcgcacctcggcgtggcggggcatcatcacaacgcctcgtatggcatcatggaggccttcaataggggagctcaagaggcacaagattggaacatgagggggctggactacaacggcggcgcctcggagctgtcgatgctcgtcggctccagcggcggcaagagggcggcggcggtggaggagaccgagccgaagctggaggacttcctcggcggcaactcgttcgtctccgagcaagatcatcacgcggcggggggcttcctcttctccggcgtcccgatggccagcagcaccaacagcaacagcgggagcaacactatggagctctccatgatcaagacctggctccggaacaacggccaggtgcccgccggccaccagccgcagcagcagcagccggcggccgcggccgccgccgcgcagcagcaggcgcacgaggcggcggagatgagcaccgacgcgagcgcgagcagcttcgggtgctcctccgacgcgatggggaggagtaacaacggcggcgcggtctcggcggcggccggcgggacgagctcgcagagcctggcgctctcgatgagcacgggctcgcactcgcacctgcctatcgtcgtcgccggcggcgggaacgccagcggcggagcggccgagagcacatcgtcggagaacaagcgggccagcggcgccatggattcgccgggcggtggcgcgatagaggccgtgccgaggaagtccatcgacacgttcgggcaaaggacctcgatatatcgaggtgtaacaaggcatagatggacagggcgatatgaggctcatctctgggataatagctgtagaagagaagggcagagtcgcaagggtaggcaagtttatcttggtggctatgacaaggaggataaagcagcgagagcttatgatttggcagctctgaagtattggggcacaacaacaacaacaaatttcccaataagtaactatgaaaaagagctagatgaaatgaaacatatgaccaggcaggagtatattgcatacctaagaaggaatagcagtggattttctcgtggtgcatcgaaatatcgtggtgtaaccaggcaccatcagcatgggagatggcaagcaaggatagggagggttgcaggaaacaaggacctctacttaggcaccttcagcaccgaggaggaggcggcggaggcgtacgacatcgcggcgatcaagttccgggggctcaacgccgtcaccaactttgacatgagccgctacgacgtcaagagcatcctggagagcagcacgctgccggtgggcggcgcggcgaggcggctgaaggaggcggcggaccacgcggaggcggccggcgccaccatctggcgcgccgccgacatggacggcgccggcgtcatctccggcctggccgacgtcgggatgggcgcctacgccgcctcgtaccaccaccaccaccaccacggctggccgaccatcgcgttccagcagccgccgccgctcgccgtgcactacccgtacggccaggcgccggcggcgccgtcgcgcgggtggtgcaagcccgagcaggacgccgccgtcgctgccgccgcgcacagcctccaggacctccagcagctgcacctcggcagcgccgccgcccacaacttcttccaggcgtcgtcgagctcgacggtctacaacggcggcggcggcgggtatcagggcctcggtggcaacgccttcttgatgccggcgagcaccgtcgtggccgaccaggggcacagcagcacggccaccaaccatggaaacacctgcagctacggcaacgaggagcaggggaagctcatcgggtacgacgccatggcgatggcgagcggcgccgccggcggcgggtatcagctgtcgcagggctcggcgtcgacggtgagcatcgcgagggcgaacggctactcggccaactggagctcgcctttcaatggcgccatgggatga

SEQ ID NO: 25 - protein, 703 aa, Oryza sativa
MASANNWLGFSLSGQENPQPHQDSSPPAAIDVSGAGDFYGLPTSQPTAADAHLGVAGHHHNASYGIMEAFNRGAQEAQDWNMRGLDYNGGASELSMLVGSSGGKRAAAVEETEPKLEDFLGGNSFVSEQDHHAAGGFLFSGVPMASSTNSNSGSNTMELSMIKTWLRNNGQVPAGHQPQQQQPAAAAAAAQQQAHEAAEMSTDASASSFGCSSDAMGRSNNGGAVSAAAGGTSSQSLALSMSTGSHSHLPIVVAGGGNASGGAAESTSSENKRASGAMDSPGGGAIEAVPRKSIDTFGQRTSIYRGVTRHRWTGRYEAHLWDNSCRREGQSRKGRQVYLGGYDKEDKAARAYDLAALKYWGTTTTTNFPISNYEKELDEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKEAADHAEAAGATIWRAADMDGAGVISGLADVGMGAYAASYHHHHHHGWPTIAFQQPPPLAVHYPYGQAPAAPSRGWCKPEQDAAVAAAAHSLQDLQQLHLGSAAAHNFFQASSSSTVYNGGGGGYQGLGGNAFLMPASTVVADQGHSSTATNHGNTCSYGNEEQGKLIGYDAMAMASGAAGGGYQLSQGSASTVSIARANGYSANWSSPFNGAMG

SEQ ID NO: 26 - DNA, 2097 bp, Setaria viridis
  source 1..2097: mol_type = other DNA
atggctactgtgaacaactggctggctttctccctctccccgcaggatctgccgccttcccagacggattccaccctcatctccgccaccgccaccgacgaggtctccggcgacgtctgcttcaacatcccccaagattggagcatgaggggatcggaactctcagcgctagtcgccgagccgaagctggaggacttcctcggcggcatcaacttctcggagcagcaccacaaggccaacctcaacgtgatccccagcagtagtaacgcttgctacgcgagctcaggcgctagcaccggctaccaccagctgtaccaccaccaaagctccgcgctccatttcgctgactccgtcatggtggcctcctccgccggcgtccatgacggcggtgccagcatgctcagtgcagcggccaccgtcaatggcggcgctggcgcagccagtgccaacggcggcagcatcgggctgtccatgatcaagaactggctgcggagtcaaccagctccgccgctgccgcagccgagagtagtggcggccgcagagggcgcgcaggcggcgcaggggctttctttgtccatgaacatggcaggggcgcaaggcgctggcatgccgcttctcgccggagagcgaggccgggcgcccgagagcgtgtccacatcagcacagggtggagcagtcgccgctcggaaggaggatagcggtggcagcggcggtgccggcgccctagtagccgtgagcacagacacgggtggcagcggcacggttgctgagacggcggcgaggaagacggtagacacgttcggccagcgcacgtcgatttaccgcggcgtgaccaggcatagatggacagggaggtacgaggctcatctgtgggacaacagttgcagaagggaaggacaaactcgcaagggtcgtcaagtctatctaggtggttatgacaaagaggagaaagctgctagggcttatgatttggctgctcttaagtattggggtcccacgacgaccacaaattttccagtgagtaactacgaaaaggagctggaggaaatgaagcacatgacaaggcaggagtttgtagcgtctctgagaaggaagagcagtggcttctccagaggtgcatccatttacagaggagtaactaggcatcaccaacacgggagatggcaagcacggattggacgagttgcagggaacaaggatctctacttgggcaccttcagcacgcaggaggaggcagcagaagcgtacgacatcgcggcgatcaagttccgcgggctcaacgccgtcaccaacttcgacatgagccgctacgacgtgaagagcatcctggacagcagcgcgctccccatcggcagcgccgccaagcgcctcaaggaggccgaggccgccgcgtccgcgcagcacgccggcgtggtgagctacgacgtaggccgcatcgcctcgcacctcggcgacggcggcgccctggcagcctacggcacgcactaccacgcagccgcagccgcctggccgaccatcgcgttccagccgagcgccgccgccgccggcctgtaccacccgtacgcgcagccgatgcgtgggtggtgcaagcaggagcaggatcacgcggtgatcgccgccgcgcacagcctgcacgagctcaaccacctgaacctgggcgccgccgccggtgcgcacgacttcttctcggcggggcaggccgcgatgcacggcctcggcagcatcgacaacgcctcactcgagcatagcaccggctccaactccgtcgtgtacaatggggttggagacagcaacggtggcgccgtcggcggcggctacatgatgccgatgagcgctgccgcggcgaccacgacggcaatggttagtcacgagcaggtgcatgcgcggtcacatcaaggggagcacgacgaagccaccaagcaggcggcgcagatggggtacgagagctacctggtgaacgcggaggcggcctacggtggcgggaggatgccttcgtggacgccggcctcggccccggcagcggcaagcagcaacgacaacatggccggcgttggccatggcggcgcgcagctcttcagtgtctggaatgacacataa

SEQ ID NO: 27 - protein, 698 aa, Setaria viridis
MATVNNWLAFSLSPQDLPPSQTDSTLISATATDEVSGDVCFNIPQDWSMRGSELSALVAEPKLEDFLGGINFSEQHHKANLNVIPSSSNACYASSGASTGYHQLYHHQSSALHFADSVMVASSAGVHDGGASMLSAAATVNGGAGAASANGGSIGLSMIKNWLRSQPAPPLPQPRVVAAAEGAQAAQGLSLSMNMAGAQGAGMPLLAGERGRAPESVSTSAQGGAVAARKEDSGGSGGAGALVAVSTDTGGSGTVAETAARKTVDTFGQRTSIYRGVTRHRWTGRYEAHLWDNSCRREGQTRKGRQVYLGGYDKEEKAARAYDLAALKYWGPTTTTNFPVSNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHAGVVSYDVGRIASHLGDGGALAAYGTHYHAAAAAWPTIAFQPSAAAAGLYHPYAQPMRGWCKQEQDHAVIAAAHSLHELNHLNLGAAAGAHDFFSAGQAAMHGLGSIDNASLEHSTGSNSVVYNGVGDSNGGAVGGGYMMPMSAAAATTTAMVSHEQVHARSHQGEHDEATKQAAQMGYESYLVNAEAAYGGGRMPSWTPASAPAAASSNDNMAGVGHGGAQLFSVWNDT

SEQ ID NO: 28 - DNA, 2034 bp, Setaria viridis
  source 1..2034: mol_type = other DNA
atggcttccgccaacaactggttgggcttctcgctctcgggccaggacaacccgcaggctaatcaggatagctcgcctgctgccgccggcatcgacatctccggcgccaccgacttctacggcctgccgacgcagcagggctccgacgggcatctcggcgtgccgggcctgcgggacgatcacgcttcctatggcatcatggaggccttcaacaggagccagcaagaaacacaagattggaacatgagaggtctggactacaacggcggcgcctcggagctctcgatgctcgtcgggtcgagcggcggcaagagggccgtggaggacggcgagcccaagctcgaggacttcctcggcggcaactcgttcgtctccgagcaagaccaatccggcggctacctcttctccggggtcccgatggccagcagcaccaacagcaacagcgggagcaacaccatggagctctccatgatcaagacctggctccggaacaaccaggtgccccagccgcagccgccggctccgcaccaggcggcgccgcagcccgaggagatgagcaccgacgccagcgcgagcagcttcggctgctcggattcgctggggaggaacggcacggtggcggcggggagctcgcagagcctggcgctgtcgatgagcacggggccccagcaccttcccatggtcgtggccggcggcggaggcggcgccagtggagcggcggcctcggagagcacgtcgtcggagaacaagcgggcgagtggcgccatggattcgcccagcagcggcgcgatagaagccgtcccgaggaagtccatcgacacgtttgggcaaaggacctctatataccgaggtgtaacgagacatagatggacagggcgttatgaggctcatctctgggataatagttgtagaagagaagggcagagtcgcaagggtaggcaagtttatcttggtggctatgacaaggaggacaaggcagcaagggcttatgatttggcagctctcaagtattggggcactactacaacaactaatttcccgataagtaactacgagaaagagctagaggaaatgaaacatatgactagacaggaatacattgcgtacctaagaaggaatagcagcggattttctcgtggggcatcaaagtatcgaggagtaactagacaccatcagcatgggagatggcaagcaaggatagggagagttgcaggaaacaaggatctctacttgggcacattcagcaccgaggaggaggcggcggaggcctacgacatcgccgccatcaagttccgcgggctcaacgccgtcaccaacttcgacatgagccgctacgacgtgaagagcatcctcgagagcagcacgctgcctgtcggcggcgcggccaggcgcctcaaggacgccgtggaccacgtggaggccggcgccaccatctggcgcgccgacatggacgccggcgtgatctcgcagctggccgacgccgggatgggcgcctacgcgtcgtacggccaccacggctggccgaccatcgcgtttcagcagccttcgccgctcaccgtgcactacccgtacggccagccgtcccgcgggtggtgcaagcccgagcaggacgccgccgtggccgccgccgcgcacagcctgcaggacctgcagcagctgcacctcggcagcgccgcccacaacttcttccaggcgtcgtcgagctcgacggtctacaacggcggcggcgccagcgctgccgggtatcagggcctcggtagcggcggcggcggctccttcctgatgccgtcgagcaccgtcgtggcagaccagggccacagcagcacggccaaccaggggagcacctgcagctacggggacgaccaggatgggaagctcatcgggtacgacgccatggcggccgccggcggggacccgtacgccgccgcgaggagcgggtatcagttctcgcagggctcgggatccacggtgagcatcgcgagggcgaacgggtactccaacaactggagctctcctttcaacggcatggggtga

SEQ ID NO: 29 - protein, 677 aa, Setaria viridis
MASANNWLGFSLSGQDNPQANQDSSPAAAGIDISGATDFYGLPTQQGSDGHLGVPGLRDDHASYGIMEAFNRSQQETQDWNMRGLDYNGGASELSMLVGSSGGKRAVEDGEPKLEDFLGGNSFVSEQDQSGGYLFSGVPMASSTNSNSGSNTMELSMIKTWLRNNQVPQPQPPAPHQAAPQPEEMSTDASASSFGCSDSLGRNGTVAAGSSQSLALSMSTGPQHLPMVVAGGGGGASGAAASESTSSENKRASGAMDSPSSGAIEAVPRKSIDTFGQRTSIYRGVTRHRWTGRYEAHLWDNSCRREGQSRKGRQVYLGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDAGVISQLADAGMGAYASYGHHGWPTIAFQQPSPLTVHYPYGQPSRGWCKPEQDAAVAAAAHSLQDLQQLHLGSAAHNFFQASSSSTVYNGGGASAAGYQGLGSGGGGSFLMPSSTVVADQGHSSTANQGSTCSYGDDQDGKLIGYDAMAAAGGDPYAAARSGYQFSQGSGSTVSIARANGYSNNWSSPFNGMG

SEQ ID NO: 30 - DNA, 16 bp, Zea mays
  source 1..16: mol_type = genomic DNA
acgtaagcgcttacgc

SEQ ID NO: 31 - DNA, 1371 bp, synthetic construct
  source 1..1371: mol_type = other DNA
acacaggaccaagaacttgaagatgcatttgaaggcctttatcttgttgactcccaagggccctagactttgtaatcttgcatttgtgctctgctgatctggtctgatactgatgtaactgatcaatgaactaattgtattagaactggattgtactctttttttcctttatatggttttctcataaggcgagtttttacctagaaaggtttttaataagacagccattgcacaaacagctataatattttatttaaagtctatgagactgactccgtgtgtgctactgcctactggctactactatctgtgaaattgtgacctgtgaactttgaaatgtgaaatttgtgacttgagaactatgattttatgacatatgaagttgtgaactgtgtatttgatacctgtgtgaatttatgacctatttaggccttgttcgtttacaccaatccagctctggattgacatggattggaattaaatacatgtcacaatctatgtcccaaaataatccaagcctactcatttttttatttggttaaacccatcatagattataacccaaggatttaggaaatttttaaactatggaagacatgaattctattcatagcttattaggtatggaataaatccatgaatatattgcacaagtttatattagaattcatgaatcaaaagaataactagttttgagagatacatggattaaatggtagatttaatctcactatgggattgagtgtgatatatggatttattcaatccaaatccggattaaatccttggtggatctatatatattggtgtgctcttagctcggttgtgtaggtgggccatgtttgacgtgccgagctggcacgatcggaccttttacccgtgccgtgctcgtgcaaggggtgttgcccgtcaggaggcaccgtgagttaatcggactcaattggaccggtctcctcggatcgcgccgtgccgccgtttggatttctatacctgcacctgtggcctgtggggagtggggactgcgaatgacattcttgcatccctcctcaccaatcaaggcggcaacataccggccctttggccttccatgaacatgaacgcggcggaacgccacgccggcgtgcactactcacctgcatgaattcgccgcccactcacagcgccaacccaacttgattgcacgcactaccatcaattcgccgccgcggacgtaagcgcttacgcccatcccttctgccagctgctatttatacgcctcgccccgctccagtctcagcagaaccaccagtcctccactccatcttctactccgaccacaaccacagcgaccacgaccgtgcacgtacgtacatcagcacaccaggcaacggc

SEQ ID NO: 32 - DNA, 1397 bp, synthetic construct
  source 1..1397: mol_type = other DNA
acacaggaccaagaacttgaagatgcatttgaaggcctttatcttgttgactcccaagggccctagactttgtaatcttgcatttgtgctctgctgatctggtctgatactgatgtaactgatcaatgaactaattgtattagaactggattgtactctttttttcctttatatggttttctcataaggcgagtttttacctagaaaggtttttaataagacagccattgcacaaacagctataatattttatttaaagtctatgagactgactccgtgtgtgctactgcctactggctactactatctgtgaaattgtgacctgtgaactttgaaatgtgaaatttgtgacttgagaactatgattttatgacatatgaagttgtgaactgtgtatttgatacctgtgtgaatttatgacctatttaggccttgttcgtttacaccaatccagctctggattgacatggattggaattaaatacatgtcacaatctatgtcccaaaataatccaagcctactcatttttttatttggttaaacccatcatagattataacccaaggatttaggaaatttttaaactatggaagacatgaattctattcatagcttattaggtatggaataaatccatgaatatattgcacaagtttatattagaattcatgaatcaaaagaataactagttttgagagatacatggattaaatggtagatttaatctcactatgggattgagtgtgatatatggatttattcaatccaaatccggattaaatccttggtggatctatatatattggtgtgctcttagctcggttgtgtaggtgggccatgtttgacgtgccgagctggcacgatcggaccttttacccgtgccgtgctcgtgcaaggggtgttgcccgtcaggaggcaccgtgagttaatcggactcaattggaccggtctcctcggatcgcgccgtgccgccgtttggatttctatacctgcacctgtggcctgtggggagtggggactgcgaatgacattcttgcatccctcctcaccaatcaaggcggcaacataccggccctttggccttccatgaacatgaacgcggcggaacgccacgccggcgtgcactactcacctgcatgaattcgccgcccactcacagcgccaacccaacttgattgcacgcactaccatcaattcgccgccgcggacgtaagcgcttacgcaccgctccttacgtaagcgcttacgcccatcccttctgccagctgctatttatacgcctcgccccgctccagtctcagcagaaccaccagtcctccactccatcttctactccgaccacaaccacagcgaccacgaccgtgcacgtacgtacatcagcacaccaggcaacggc

SEQ ID NO: 33 - DNA, 1423 bp, synthetic construct
  source 1..1423: mol_type = other DNA
acacaggaccaagaacttgaagatgcatttgaaggcctttatcttgttgactcccaagggccctagactttgtaatcttgcatttgtgctctgctgatctggtctgatactgatgtaactgatcaatgaactaattgtattagaactggattgtactctttttttcctttatatggttttctcataaggcgagtttttacctagaaaggtttttaataagacagccattgcacaaacagctataatattttatttaaagtctatgagactgactccgtgtgtgctactgcctactggctactactatctgtgaaattgtgacctgtgaactttgaaatgtgaaatttgtgacttgagaactatgattttatgacatatgaagttgtgaactgtgtatttgatacctgtgtgaatttatgacctatttaggccttgttcgtttacaccaatccagctctggattgacatggattggaattaaatacatgtcacaatctatgtcccaaaataatccaagcctactcatttttttatttggttaaacccatcatagattataacccaaggatttaggaaatttttaaactatggaagacatgaattctattcatagcttattaggtatggaataaatccatgaatatattgcacaagtttatattagaattcatgaatcaaaagaataactagttttgagagatacatggattaaatggtagatttaatctcactatgggattgagtgtgatatatggatttattcaatccaaatccggattaaatccttggtggatctatatatattggtgtgctcttagctcggttgtgtaggtgggccatgtttgacgtgccgagctggcacgatcggaccttttacccgtgccgtgctcgtgcaaggggtgttgcccgtcaggaggcaccgtgagttaatcggactcaattggaccggtctcctcggatcgcgccgtgccgccgtttggatttctatacctgcacctgtggcctgtggggagtggggactgcgaatgacattcttgcatccctcctcaccaatcaaggcggcaacataccggccctttggccttccatgaacatgaacgcggcggaacgccacgccggcgtgcactactcacctgcatgaattcgccgcccactcacagcgccaacccaacttgattgcacgcactaccatcaattcgccgccgcggacgtaagcgcttacgcaccgctccttacgtaagcgcttacgcaccgctccttacgtaagcgcttacgcccatcccttctgccagctgctatttatacgcctcgccccgctccagtctcagcagaaccaccagtcctccactccatcttctactccgaccacaaccacagcgaccacgaccgtgcacgtacgtacatcagcacaccaggcaacggc

SEQ ID NO: 34 - DNA, 1398 bp, synthetic construct
  source 1..1398: mol_type = other DNA
acacaggaccaagaacttgaagatgcatttgaaggcctttatcttgttgactcccaagggccctagactttgtaatcttgcatttgtgctctgctgatctggtctgatactgatgtaactgatcaatgaactaattgtattagaactggattgtactctttttttcctttatatggttttctcataaggcgagtttttacctagaaaggtttttaataagacagccattgcacaaacagctataatattttatttaaagtctatgagactgactccgtgtgtgctactgcctactggctactactatctgtgaaattgtgacctgtgaactttgaaatgtgaaatttgtgacttgagaactatgattttatgacatatgaagttgtgaactgtgtatttgatacctgtgtgaatttatgacctatttaggccttgttcgtttacaccaatccagctctggattgacatggattggaattaaatacatgtcacaatctatgtcccaaaataatccaagcctactcatttttttatttggttaaacccatcatagattataacccaaggatttaggaaatttttaaactatggaagacatgaattctattcatagcttattaggtatggaataaatccatgaatatattgcacaagtttatattagaattcatgaatcaaaagaataactagttttgagagatacatggattaaatggtagatttaatctcactatgggattgagtgtgatatatggatttattcaatccaaatccggattaaatccttggtggatctatatatattggtgtgctcttagctcggttgtgtaggtgggccatgtttgacgtgccgagctggcacgatcggaccttttacccgtgccgtgctcgtgcaaggggtgttgcccgtcaggaggcaccgtgagttaatcggactcaattggaccggtctcctcggatcgcgccgtgccgccgtttggatttctatacctgcacctgtggcctgtggggagtggggactgcgaatgacattcttgcatccctcctcaccaatcaaggcggcaacataccggccctttggccttccatgaacatgaacgcggcggaacgccacgccggcgtgcactactcacctgcatgaattcgccgcccactcacagcgccaacccaacttgattgcacgcactaccatcaattcgccgccgcggacgtaagcgcttacgcaccgctccttacgtaagcgcttacgcccatcccttctgccagctgctatatataacgcctcgccccgctccagtctcagcagaaccaccagtcctccactccatcttctactccgaccacaaccacagcgaccacgaccgtgcacgtacgtacatcagcacaccaggcaacggc

SEQ ID NO: 35 - DNA, 1371 bp, synthetic construct
  source 1..1371: mol_type = other DNA
acacaggaccaagaacttgaagatgcatttgaaggcctttatcttgttgactcccaagggccctagactttgtaatcttgcatttgtgctctgctgatctggtctgatactgatgtaactgatcaatgaactaattgtattagaactggattgtactctttttttcctttatatggttttctcataaggcgagtttttacctagaaaggtttttaataagacagccattgcacaaacagctataatattttatttaaagtctatgagactgactccgtgtgtgctactgcctactggctactactatctgtgaaattgtgacctgtgaactttgaaatgtgaaatttgtgacttgagaactatgattttatgacatatgaagttgtgaactgtgtatttgatacctgtgtgaatttatgacctatttaggccttgttcgtttacaccaatccagctctggattgacatggattggaattaaatacatgtcacaatctatgtcccaaaataatccaagcctactcatttttttatttggttaaacccatcatagattataacccaaggatttaggaaatttttaaactatggaagacatgaattctattcatagcttattaggtatggaataaatccatgaatatattgcacaagtttatattagaattcatgaatcaaaagaataactagttttgagagatacatggattaaatggtagatttaatctcactatgggattgagtgtgatatatggatttattcaatccaaatccggattaaatccttggtggatctatatatattggtgtgctcttagctcggttgtgtaggtgggccatgtttgacgtgccgagctggcacgatcggaccttttacccgtgccgtgctcgtgcaaggggtgttgcccgtcaggaggcaccgtgagttaatcggactcaattggaccggtctcctcggatcgcgccgtgccgccgtttggatttctatacctgcacctgtggcctgtggggagtggggactgcgaatgacattcttgcatccctcctcaccaatcaaggcggcaacataccggccctttggccttccatgaacatgaacgcggcggaacgccacgccggcgtgcactactcacctgcatgaattcgccgcccactcacagcgccaacccaacttgattgcacgcactaccatcaattcgccgccgcggacgtaagcgcttacgcccatcccttctgccagctgctatagcgccgcctcgccccgctccagtctcagcagaaccaccagtcctccactccatcttctactccgaccacaaccacagcgaccacgaccgtgcacgtacgtacatcagcacaccaggcaacggc

SEQ ID NO: 36 - DNA, 1397 bp, synthetic construct
  source 1..1397: mol_type = other DNA
acacaggaccaagaacttgaagatgcatttgaaggcctttatcttgttgactcccaagggccctagactttgtaatcttgcatttgtgctctgctgatctggtctgatactgatgtaactgatcaatgaactaattgtattagaactggattgtactctttttttcctttatatggttttctcataaggcgagtttttacctagaaaggtttttaataagacagccattgcacaaacagctataatattttatttaaagtctatgagactgactccgtgtgtgctactgcctactggctactactatctgtgaaattgtgacctgtgaactttgaaatgtgaaatttgtgacttgagaactatgattttatgacatatgaagttgtgaactgtgtatttgatacctgtgtgaatttatgacctatttaggccttgttcgtttacaccaatccagctctggattgacatggattggaattaaatacatgtcacaatctatgtcccaaaataatccaagcctactcatttttttatttggttaaacccatcatagattataacccaaggatttaggaaatttttaaactatggaagacatgaattctattcatagcttattaggtatggaataaatccatgaatatattgcacaagtttatattagaattcatgaatcaaaagaataactagttttgagagatacatggattaaatggtagatttaatctcactatgggattgagtgtgatatatggatttattcaatccaaatccggattaaatccttggtggatctatatatattggtgtgctcttagctcggttgtgtaggtgggccatgtttgacgtgccgagctggcacgatcggaccttttacccgtgccgtgctcgtgcaaggggtgttgcccgtcaggaggcaccgtgagttaatcggactcaattggaccggtctcctcggatcgcgccgtgccgccgtttggatttctatacctgcacctgtggcctgtggggagtggggactgcgaatgacattcttgcatccctcctcaccaatcaaggcggcaacataccggccctttggccttccatgaacatgaacgcggcggaacgccacgccggcgtgcactaacgtaagcgcttacgcaccgctccttacgtaagcgcttacgcctcacctgcatgaattcgccgcccactcacagcgccaacccaacttgattgcacgcactaccatcaattcgccgccgcggccatcccttctgccagctgctatttatacgcctcgccccgctccagtctcagcagaaccaccagtcctccactccatcttctactccgaccacaaccacagcgaccacgaccgtgcacgtacgtacatcagcacaccaggcaacggc

SEQ ID NO: 37 - DNA, 2000 bp, Triticum aestivum
  source 1..2000: mol_type = genomic DNA
ttagaagctttggcgtgtcgagaagctcaagcgcttgcacttgatctcatggaaaatagaatcgttgttgcgagtgattcgaagacggtggtttcagatattgaaaaaggtatagaagggtcgcattcttccattgtaaaggagattgcaatgcgggcgatggagtttggtacatgtgactttattttcgagggtcgagcactcaattgggaagctcacagtttagctaaatttgctagttctttagatgttggacgccatgtttggctaggggctcctcatgacccctaattatccctgtaaaccttgttactattcaataaagcttggtttactgctaaaaagactattcttttcttttcttctcagggaaattttttccatcgtggcttcagccgacaaagaccaaatgtcgagactgaatgcattgtgtgccactcgaggaggagatgtagacgacggcctcccattctgcaccgcgtttgaagcgttctagtctgatgagtatgagttgagatatgacggtgacgacacccaatgcctatttcctcaccaccgtaagtttgccgataccatctttcgaatgtcggtggctagactgcttgagtccaatggcccatttgacctgtagcaaccacgagtcggtgggcacatagcatactcagcatataacccactaggggtacatcagaggtgaaaaatatactttcttcctacactaaataggttggtccttaatagaaaaaaacgataccagaggggcgagcacccctcagcatatttttatacaaggagcaagaaacgtggttgaattattcgctccccatggggatcaagttggtcttgaaaaaaattgtatgggctacttaacaatgcactccgtggagtagtgttttgtacctctcgagtctctaccgatcttcgtgcacggttttgtcagagctttgatcatgcgaactttgaacatccagactccggttttcaagtcctagacctgacattggtggtcgcattttcctggttttattttaggccattcgacgatgtgcgttcagtgggaagaagacgttctcgccgactacgaggacgtttatggcggcttcgtcaatctcaaaatattgtgtcgtgtgcatgtccttcgcatggttgagtgtatgtgcgtgtagataagcatccgcatttgtattgtgtccaaaaaattcaggttcttttaatcaccgcactagctgttcaattgatcaatgctaaaggacaggatcgttcccaatttgcaatggtgctgatggagattaaaaggctttttttttgcgcgcggtgagattaaaaggcttttggttaacggaagtgtagaactactcatatttcgagagcaggataatgttagccatgttttagaacttcaggagaacgaaggaccatattgtggtctggatccattctggccttgttaacattcctagtttgtatagggatgcattcctttgctcttgagtaatacatttcccttacccgccagaaaaaaaaacttttacaacttactcgcaaaataaaatgtggcacggttaacagtgagtttatggcgcccttttacattcgttccgatcacgatgttcatatgtacgtacctgtgtacgtgcacgtggatcgtgatccatgcaacatgatcaaacgctgcaggcgcaaatcatcactgagccgatcggttaccgccgttcccgcgatcaattatcaagttagctagcggggcatcaatacgcataatcaagctaaggcacctgcatgcatgcatgcatgcatggcgcagcaacgcccgcgcgcccgtgcgtctcccccgcatccctaacagtgaaacgtacgcgcgccttctccacttcgccttccaggcacgtcgatccactgtataaacacgccgctggtgcgtacgcagagcacgcacacggccttgcattgctagctaccttgcgaccacaaatcgccgggcttctctcgccggtcgatcc

SEQ ID NO: 38 - DNA, 1783 bp, Triticum aestivum
  source 1..1783: mol_type = genomic DNA
acactttacaaagtttatcaccctagctaacttagcaaacttatgggcaacaacgttggccgaacgactcatccaagaaactttaaaatcatacctagaatgcaaaagctgctttatctccttgatccataggccgacaacagagtagtccacccctttgtgattgatcttgttgacaaccaccctgctatcgagctttacatgaagcttctcaacgttcatctgaatagccagctacaccgcgcgtcggcaggccagaacctccgacgactccggatccgtgatgctcaggaagaaatggcaggccctggcaaggagcgctccattgtgatctcttgcaacgacgcctccacctcctttatctctagctctactagtagcaccattggcgttgaacttgacccagccctcgactggggccccccatctcggtacttgttttgccatcaccgtctgctccttcgtttgatgcacattcttccattcctgcataaaactgcacatagtttttgtaatttcatgcggcgaggcaatcctctttccatccctcgcatcatttctagtcatccataaggtatatacaacatggatcatagcttccttctcttcctcactcgcgacagctaaccaggtaagcatccatcgagcctgtgctccatgatcatccatcggcgtgggaggagcagtaacatcaatgtccttctccgttcgaagaaatttccaaaaaagctgagcatgtggacaagaccaaaacttgtgcataatagtctcctctctcccacacgccacatagaaaaccccaggtttgataccgcgccgatggacttcagacccaatagcaagaccatctaactaacatccacatataaatttttcgctttacctggagcattaggccttgtacaatgggaggtgcttagggaagtgcttagaaaaataaaccagacttttcttaagcaccggtgcttatttgtacaggatagacgcttaactaagcgtttctcctgtagaaataggcaccggtgctttagaaaaatccggtttatttttctaagcatctctctaagcacctttcattgtacaaggccttagtgtcccaaagagccggtcaatagaacttgagggctcgggccggctcaacgcacatttcttcatcgacatcctgagatgatatgcggattgaacattgaaggttccattctggccttgttaacattcccagtcgtgcagcgatgcattcctttgctcttgagtaatacatttcctttaccctgtaaaaaaaactttagtatttgtaaaaggcaacgtggcacggttaacatactcacaaaataaaacgtggcacggttaactgtcagtttatggcgcccttttacattcgttccgcacgcaatctcgatgttcatatatgtagtactacgtgtgtacgtgcacgtggatcgtgatccatgcaacatgatcagacgctgcaggcgctaatcatcagtgagccgatcggctaccgccgttcccgcgatcaatcgagttcgctagcggggcatctatacgcacaatcaagctaggcaccatgcatgcatgcatgcatggcgcagcaacgcccgcccgtgcgtctcccccgcggcattcctgaaacgtacgcgcgcctcctccacttcgccttccaggcacgtcgatccactatataaacacgccgctagtgcgtctgcagagcacgcacacgaccttgcattactagagctaccttccagccacaaatcgccgggcatctctcgccggtcgatcg

SEQ ID NO: 39 - DNA, 1986 bp, Triticum aestivum
  source 1..1986: mol_type = genomic DNA
gtgtcgagaagctcaagcgcttgcacttgatctcatggaaaatagaatcgttgttgcgagtgattcgaagacggtggtttcagatattgaaaaaggtatagaagggtcgcattcttccattgtaaaggagattgcaatgcgggcgatggagtttggtacatgtgactttattttcgagggtcgagcactcaattgggaagctcacagtttagctaaatttgctagttctttagatgttggacgccatgtttggctaggggctcctcatgacccctaattatccctgtaaaccttgttactattcaataaagcttggtttactgctaaaaagactattcttttcttttcttctcagggaaattttttccatcgtggcttcagccgacaaagaccaaatgtcgagactgaatgcattgtgtgccactcgaggaggagatgtagacgacggcctcccattctgcaccgcgtttgaagcgttctagtctgatgagtatgagttgagatatgacggtgacgacacccaatgcctatttcctcaccaccgtaagtttgccgataccatctttcgaatgtcggtggctagactgcttgagtccaatggcccatttgacctgtagcaaccacgagtcggtgggcacatagcatactcagcatataacccactaggggtacatcagaggtgaaaaatatactttcttcctacactaaataggttggtccttaatagaaaaaaacgataccagaggggcgagcacccctcagcatatttttatacaaggagcaagaaacgtggttgaattattcgctccccatggggatcaagttggtcttgaaaaaaattgtatgggctacttaacaatgcactccgtggagtagtgttttgtacctctcgagtctctaccgatcttcgtgcacggttttgtcagagctttgatcatgcgaactttgaacatccagactccggttttcaagtcctagacctgacattggtggtcgcattttcctggttttattttaggccattcgacgatgtgcgttcagtgggaagaagacgttctcgccgactacgaggacgtttatggcggcttcgtcaatctcaaaatattgtgtcgtgtgcatgtccttcgcatggttgagtgtatgtgcgtgtagataagcatccgcatttgtattgtgtccaaaaaattcaggttcttttaatcaccgcactagctgttcaattgatcaatgctaaaggacaggatcgttcccaatttgcaatggtgctgatggagattaaaaggctttttttttgcgcgcggtgagattaaaaggcttttggttaacggaagtgtagaactactcatatttcgagagcaggataatgttagccatgttttagaacttcaggagaacgaaggaccatattgtggtctggatccattctggccttgttaacattcctagtttgtatagggatgcattcctttgctcttgagtaatacatttcccttacccgccagaaaaaaaaacttttacaacttactcgcaaaataaaatgtggcacggttaacagtgagtttatggcgcccttttacattcgttccgatcacgatgttcatatgtacgtacctgtgtacgtgcacgtggatcgtgatccatgcaacatgatcaaacgctgcaggcgcaaatcatcactgagccgatcggttaccgccgttcccgcgatcaattatcaagttagctagcggggcatcaatacgcataatcaagctaaggcacctgcatgcatgcatgcatgcatggcgcagcaacgcccgcgcgcccgtgcgtctcccccgcatccctaacagtgaaacgtacgcgcgccttctccacttcgccttccaggcacgtcgatccactgtataaacacgccgctggtgcgtacgcagagcacgcacacggccttgcattgctagctaccttgcgaccacaaatcgccgggcttctctcgccggtcgatcc

SEQ ID NO: 40 - DNA, 1132 bp, Triticum aestivum
  source 1..1132: mol_type = genomic DNA
tgtagctgcccaatgacttggaggctcgatttgagccttcaagatttgtccgtgtgtctcatgtagcatgtgttaactcaaaaatcttgcgcactgtcaaaactagtactagtatctctgaagttgtgcaaattcgtactacgttgtagaggactccatgcatactccacttggctcgggggggatatagctcagttggtagagctccgctcttgcaattgggtcgttgcgattacgggttggctgtctaattgtccaggcggggttaggatctgaggaagcttagtttggatgatggaacattcgctgttctgattgttgagataccgcaaatccacttatccaacaatttcagtccattttcctactttttctgcagcccgttgcaaggaaggaaagagcaagaactagggctaagtgctgagttacggttgttgactcggaattgtaagccgaagaagcattcctgtgacctggccgtatgaatggtttctttctcaataccagagctaaataattaggcatcaaaattcaaagaaggtgatacttttcacgtactcctttggtttggaggaatttcataggaattctagatgataggattcttataggatttttttcctatagagccatttggttcataggaatggattcctatttctacataggattggttcctatccttcatatttaataggaaaataaaaataaacctagactcaatggaaaaattcctttggtgtcaaccaaatgacatctcgtttcctattcctactcataggatttgagatacatatcatctcatttcctataaaattcatattcctacgataattttatcctatgaaccaaaagaggcctcaactactcgtaccatgaaagaaaggggattttggggggagatgagaaacgaggggatcacgccccgattaattttctgtggatagagtaacgtaggaaggagaggaaggggcaacgcattcgcgtttaattaagataattttgattcgtttggacttccacgttctcgtaaagccttttcgcttacctgacgtttcggttacttgctatgataagtagtagtacgtcagcctcctcactcggtagcaccagctagagtcgacctcagatc

SEQ ID NO: 41 - DNA, 1863 bp, Triticum aestivum
  source 1..1863: mol_type = genomic DNA
atggcggagcctaagctggaggacttcctcggcggcatatcctactccgaccaccaccaccaccacaagcaggccggcggcaacaacatggtcgtccctgcaggtagcggcagcggcggcgccgcatgctacacgacctcgggcggcagtagcgtgggctacctgtaccaccccagctcggcctccctccagtttgccgactccgtcatggtggcttcctccggcggtggcgtccaccacgacggcgctggcatcatggcgaacaccacagccaacggtgaccttaataatggcagcggcgggggtggcatcgggctgtccatgatcaagagctggctgcggagccagccatccccggcacaaccaccggagcagcagagggcggatgcggcggcacagggtctctcgctgtccatgaacatggcggcgtgcatgccgccgctcgttggcggggagcgtggtgtgccggagttggcgattgtccggaaggatgacacggccggtggcagcagcgctggttctggcgccgtggtgtcggccggtggtgcggatagcactggcggaagcagtggcgtggtggtggagacgccggcgtccaggaagacggctgacacgtttgggcagcggacctcgatttaccgtggtgtgaccaggcatagatggactgggagatatgaggctcacttatgggacaacagttgcagaagagaagggcaaacacgcaagggtcgtcaagtctatctaggtggctatgacaaagaggagaaagctgcaagagcttatgatttggctgctctcaaatattggggtgcgacgacgacgacaaatttcccagtaaacaactacgaaaaagagctagaggagatgaagcacatgacaaggcaggagtttgtggcgtctttgcgaaggaagagcagtggtttctccagaggcgcatccatttatcgtggagtaactaggcaccaccaacatgggagatggcaagcgaggatagggagagttgcagggaacaaggatctctacttaggcaccttcagcacgcaggaggaggctgcggaggcgtacgacatcgcggcgatcaagttccgggggctcaatgctgtcacaaacttcgacatgacgcgctacgatgtcaagagcatcctcgacagcaccgcgctccccatcggtagcgctgccaagcgcctcaaggacgccgaggcggctacggcgtcgtccctcgcgctgcagcagcagcagcacgcaggcgtggtgagcggctatgacatgggcgccctggccgcctacggtgccgcataccaccaccatcacccctctgtcgccggcgctgttgcgtggccgaccatcgcgttccaggcgccgcctcatcaggtttccagcggcggtggtggacacatgtaccacccctacgcccatgcgcagccgctgcgcgggtggtgtaagcaggagccggaccacgcggtgatcgcggctgcgcacagcctgcaggagctccaccacctcaacctcggcgccggtggcggcgcacatgacttcttctcgcagcatgcccaggcgatgcagcagcagcacggtggcctcggcagcatccacaacgcggccggcgcctcgctcgagcacagcaccggctccaactccgtcgtgtacaacaacggcggcgtcgagagctacatgcttcccatgagcaccaccaccaccgcgacggcgagccacgcccgcgagcaggtggcagcgcatgcccacgcacgcgcggtggcggcacaggcatacagcggcggcaacatgagcagggatcacgacgacgggaagatcgcctacgagaactacctcgtgagcaccgcagaagcctacggcggggggtag

SEQ ID NO: 42 - protein, 620 aa, Triticum aestivum
MAEPKLEDFLGGISYSDHHHHHKQAGGNNMVVPAGSGSGGAACYTTSGGSSVGYLYHPSSASLQFADSVMVASSGGGVHHDGAGIMANTTANGDLNNGSGGGGIGLSMIKSWLRSQPSPAQPPEQQRADAAAQGLSLSMNMAACMPPLVGGERGVPELAIVRKDDTAGGSSAGSGAVVSAGGADSTGGSSGVVVETPASRKTADTFGQRTSIYRGVTRHRWTGRYEAHLWDNSCRREGQTRKGRQVYLGGYDKEEKAARAYDLAALKYWGATTTTNFPVNNYEKELEEMKHMTRQEFVASLRRKSSGFSRGASIYRGVTRHHQHGRWQARIGRVAGNKDLYLGTFSTQEEAAEAYDIAAIKFRGLNAVTNFDMTRYDVKSILDSTALPIGSAAKRLKDAEAATASSLALQQQQHAGVVSGYDMGALAAYGAAYHHHHPSVAGAVAWPTIAFQAPPHQVSSGGGGHMYHPYAHAQPLRGWCKQEPDHAVIAAAHSLQELHHLNLGAGGGAHDFFSQHAQAMQQQHGGLGSIHNAAGASLEHSTGSNSVVYNNGGVESYMLPMSTTTTATASHAREQVAAHAHARAVAAQAYSGGNMSRDHDDGKIAYENYLVSTAEAYGGG

SEQ ID NO: 43 - DNA, 1473 bp, Triticum aestivum
  source 1..1473: mol_type = genomic DNA
atggcggagcctaagctggaggacttcctcggcggcatatcctactccgaccaccaccaccaccacaagcaggccggcggcggcagcggcgggggtggcatcgggctgtccatgatcaagagctggctgcggagccagccatcctcggccggtggtgcggatagcactggcggaagcagtggcgtggtggtggagacgccggcgtccaggaagacggctgacacgtttgggcagcggacctcgatttaccgtggtgtgaccaggcatagatggactgggagatatgaggctcacttatgggacaacagttgcagaagagaagggcaaacacgcaagggtcgtcaagtctatctaggtggctatgacaaagaggagaaagctgcaagagcttatgatttggctgctctcaaatattggggtgcgacgacgacgacaaatttcccagtaaacaactacgaaaaagagctagaggagatgaagcacatgacaaggcaggagtttgtggcgtctttgcgaaggaagagcagtggtttctccagaggcgcatccatttatcgtggagtaactaggcaccaccaacatgggagatggcaagcgaggatagggagagttgcagggaacaaggatctctacttaggcaccttcagcacgcaggaggaggctgcggaggcgtacgacatcgcggcgatcaagttccgggggctcaatgctgtcacaaacttcgacatgacgcgctacgatgtcaagagcatcctcgacagcaccgcgctccccatcggtagcgctgccaagcgcctcaaggacgccgaggcggctacggcgtcgtccctcgcgctgcagcagcagcagcacgcaggcgtggtgagcggctatgacatgggcgccctggccgcctacggtgccgcataccaccaccatcacccctctgtcgccggcgctgttgcgtggccgaccatcgcgttccaggcgccgcctcatcaggtttccagcggcggtggtggacacatgtaccacccctacgcccatgcgcagccgctgcgcgggtggtgtaagcaggagccggaccacgcggtgatcgcggctgcgcacagcctgcaggagctccaccacctcaacctcggcgccggtggcggcgcacatgacttcttctcgcagcatgcccaggcgatgcagcagcagcacggtggcctcggcagcatccacaacgcggccggcgcctcgctcgagcacagcaccggctccaactccgtcgtgtacaacaacggcggcgtcgagagctacatgcttcccatgagcaccaccaccaccgcgacggcgagccacgcccgcgagcaggtggcagcgcatgcccacgcacgcgcggtggcggcacaggcatacagcggcggcaacatgagcagggatcacgacgacgggaagatcgcctacgagaactacctcgtgagcaccgcagaagcctacggcggggggtag

SEQ ID NO: 44 - protein, 490 aa, Triticum aestivum
MAEPKLEDFLGGISYSDHHHHHKQAGGGSGGGGIGLSMIKSWLRSQPSSAGGADSTGGSSGVVVETPASRKTADTFGQRTSIYRGVTRHRWTGRYEAHLWDNSCRREGQTRKGRQVYLGGYDKEEKAARAYDLAALKYWGATTTTNFPVNNYEKELEEMKHMTRQEFVASLRRKSSGFSRGASIYRGVTRHHQHGRWQARIGRVAGNKDLYLGTFSTQEEAAEAYDIAAIKFRGLNAVTNFDMTRYDVKSILDSTALPIGSAAKRLKDAEAATASSLALQQQQHAGVVSGYDMGALAAYGAAYHHHHPSVAGAVAWPTIAFQAPPHQVSSGGGGHMYHPYAHAQPLRGWCKQEPDHAVIAAAHSLQELHHLNLGAGGGAHDFFSQHAQAMQQQHGGLGSIHNAAGASLEHSTGSNSVVYNNGGVESYMLPMSTTTTATASHAREQVAAHAHARAVAAQAYSGGNMSRDHDDGKIAYENYLVSTAEAYGGG

SEQ ID NO: 45 - DNA, 1656 bp, Triticum aestivum
  source 1..1656: mol_type = genomic DNA
atggccgagaccgagcccaagcttgaggacttcctcgttggcaactcgttcgtctccgagcaagatcaggccggtggcttcttgttctccggtgtcccgatggcgaccagcaccaacagcaacagcaggagcaacaccatggagctctctatgatcaagagctggctccgtgacaaccaggtaccccaagcgccgcacccgcagtcgcagaccggggcgcccgcgcagcagccgcaaccccacgaggagatgggcaccgacgcgagcagcttcgatccgctggggagaaacggcgcgttggtggtggcggggagctcgcagagcctggcgctctcgatgagcacgggctcaggatcgcacttgccgatggctgtgctcggcggcagtcccagtgggggagtctcagagagcacctcctcggagaacaagcgggccagcggcgccatggattcgccgggcggtgcggtagaagctgtcgcgaggaagtccatcgacacattcgggcaaaggacgtcgatatatcgaggtgtaacaagacatagatggacagggcgttatgaggctcatctctgggacaatagctgcagaagagaagggcagagtcgcaagggtaggcaagtttatctgggtggctatgacaaggaggacaaggcagcaagggcttatgatttggcagcactcaagtattggggtacaacaacaacaacaaatttcccaattaatacctacgaaaaagaggtggatgaaatgaaacatatgactaggcaggaatacatcgcatacctaagaaggaatagcagcggattttcccgtggtgcgtcaaaatatcgtggtgtaactaggcaccatcagcatgggagatggcaagcaagaattgggagggttgcaggaaacaaggatctctacttaggcaccttcagcaccgaggaggaggcggcggaggcctacgacatcgcggcgatcaagttccgtgggctgaacgccgtcaccaacttcgacatgagtcgctacgacgtcaagaccatcctcgagagcagcaccctgccggtgggtggcgcggcgaggcgccccaaggaggtcgctgatcatccagaggctggcgccacgatctggcgggccggcatggacggcggcgtcatctcccagctgaccgacgtcgggatgggcacgtacgcgtcgtaccaccacggctggccaaccatcgcattccagcagccctcgccgctgtcggtgcactacccatacgtcgcccagccgtctcgcgggtggtgcaagcccgagcaggacgccagcgtcgccacgcaaagcctgcaggacctccagcagctgcacctcgggaccgccgctcacaactatttccaggcatcgtcgagctccacggtctacaacggcggcgggtaccagcaaggcaccagtggcaacgccttcttgatgccggctagcaccgtggtggatgaacaggggcacagcagcactgccaccaaccagggaagcacatgcagctacggggacgaagaggggaagctcaatctcgggtacgatgccatggcgatggcgagcaccggcgccgacccatacgcggcggcgccaactgtgagcattgcgagggcgaacgggtactccaacaactggagctcgccgttcaatggcatgtga

SEQ ID NO: 46 - protein, 551 aa, Triticum aestivum
MAETEPKLEDFLVGNSFVSEQDQAGGFLFSGVPMATSTNSNSRSNTMELSMIKSWLRDNQVPQAPHPQSQTGAPAQQPQPHEEMGTDASSFDPLGRNGALVVAGSSQSLALSMSTGSGSHLPMAVLGGSPSGGVSESTSSENKRASGAMDSPGGAVEAVARKSIDTFGQRTSIYRGVTRHRWTGRYEAHLWDNSCRREGQSRKGRQVYLGGYDKEDKAARAYDLAALKYWGTTTTTNFPINTYEKEVDEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKTILESSTLPVGGAARRPKEVADHPEAGATIWRAGMDGGVISQLTDVGMGTYASYHHGWPTIAFQQPSPLSVHYPYVAQPSRGWCKPEQDASVATQSLQDLQQLHLGTAAHNYFQASSSSTVYNGGGYQQGTSGNAFLMPASTVVDEQGHSSTATNQGSTCSYGDEEGKLNLGYDAMAMASTGADPYAAAPTVSIARANGYSNNWSSPFNGM

SEQ ID NO: 47 - DNA, 1587 bp, Triticum aestivum
  source 1..1587: mol_type = genomic DNA
atggccgagaccgagcccaagcttgaggacttcctcgttggcaactcgttcgtctccgagcaagatcaggccggtggcttcttgttctccggtgtcccgatggcgaccagcaccaacagcaacagcaggagcaacaccatggagctctctatgatcaagagctggctccgtgacaaccaggtaccccaagcgccgcacccgcagtcgcagaccggggcgcccgcgcagcagccgcaaccccacgaggagatgggcaccgacgcgagcagcttcgatccgctggggagaaacggcgcgttggtggtggcggggagctcgcagagcctggcgctctcgatgagcacgggctcaggatcgcacttgccgatggctgtgctcggcggcagtcccagtgggggagtctcagagagcacctcctcggagaacaagcgggccagcggcgccatggattcgccgggcggtgcggtagaagctgtcgcgaggaagtccatcgacacattcgggcaaaggacgtcgatatatcgaggtgtaacaagacatagatggacagggcgttatgaggctcatctctgggacaatagctgcagaagagaagggcagagtcgcaagggtaggcaagtttatctgggtggctatgacaaggaggacaaggcagcaagggcttatgatttggcagcactcaagtattggggtacaacaacaacaacaaatttcccaattaatacctacgaaaaagaggtggatgaaatgaaacatatgactaggcaggaatacatcgcatacctaagaaggaatagcagcggattttcccgtggtgcgtcaaaatatcgtggtgtaactaggcaccatcagcatgggagatggcaagcaagaattgggagggttgcaggaaacaaggatctctacttaggcaccttcagcaccgaggaggaggcggcggaggcctacgacatcgcggcgatcaagttccgtgggctgaacgccgtcaccaacttcgacatgagtcgctacgacgtcaagaccatcctcgagagcagcaccctgccggtgggtggcgcggcgaggcgccccaaggaggtcgctgatcatccagaggctggcgccacgatctggcgggccggcatggacggcggcgtcatctcccagctgaccgacgtcgggatgggcacgtacgcgtcgtaccaccacggctggccaaccatcgcattccagcagccctcgccgctgtcggtgcactacccatacgtcgcccagccgtctcgcgggtggtgcaagcccgagcaggacgccagcgtcgccacgcaaagcctgcaggacctccagcagctgcacctcgggaccgccgctcacaactatttccaggcatcgtcgagctccacggtctacaacggcggcgggtaccagcaaggcaccagtggcaacgccttcttgatgccggctagcaccgtggtggatgaacaggggcacagcagcactgccaccaaccagggaagcacatgcagctacggggacgaagaggggaagctcaatctcgggtacgatgccatggcgatggcgagcaccggcgccgacccatacgcggcgtga

SEQ ID NO: 48 - protein, 528 aa, Triticum aestivum
MAETEPKLEDFLVGNSFVSEQDQAGGFLFSGVPMATSTNSNSRSNTMELSMIKSWLRDNQVPQAPHPQSQTGAPAQQPQPHEEMGTDASSFDPLGRNGALVVAGSSQSLALSMSTGSGSHLPMAVLGGSPSGGVSESTSSENKRASGAMDSPGGAVEAVARKSIDTFGQRTSIYRGVTRHRWTGRYEAHLWDNSCRREGQSRKGRQVYLGGYDKEDKAARAYDLAALKYWGTTTTTNFPINTYEKEVDEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKTILESSTLPVGGAARRPKEVADHPEAGATIWRAGMDGGVISQLTDVGMGTYASYHHGWPTIAFQQPSPLSVHYPYVAQPSRGWCKPEQDASVATQSLQDLQQLHLGTAAHNYFQASSSSTVYNGGGYQQGTSGNAFLMPASTVVDEQGHSSTATNQGSTCSYGDEEGKLNLGYDAMAMASTGADPYAA

SEQ ID NO: 49 - DNA, 20 bp, synthetic construct
  source 1..20: mol_type = other DNA
gtaatcctaaaataatccgt

SEQ ID NO: 50 - DNA, 20 bp, synthetic construct
  source 1..20: mol_type = other DNA
gagtaagtggactctctgct

SEQ ID NO: 51 - DNA, 20 bp, synthetic construct
  source 1..20: mol_type = other DNA
gcacgacatgtccaaagcgt

SEQ ID NO: 52 - DNA, 20 bp, synthetic construct
  source 1..20: mol_type = other DNA
gtagtgtcgacaccagtgta

SEQ ID NO: 53 - DNA, 20 bp, synthetic construct
  source 1..20: mol_type = other DNA
gggctagtacatccaaatgt

SEQ ID NO: 54 - DNA, 20 bp, synthetic construct
  source 1..20: mol_type = other DNA
ggctagtacatccaaatgta

SEQ ID NO: 55 - DNA, 20 bp, synthetic construct
  source 1..20: mol_type = other DNA
gttaaaccttgtcagggatt

SEQ ID NO: 56 - DNA, 20 bp, synthetic construct
  source 1..20: mol_type = other DNA
gtccgacataggagtgttaa

SEQ ID NO: 57 - DNA, 3937 bp, Triticum aestivum
  source 1..3937: mol_type = genomic DNA
atggcggggagtgggaagaggaggcgcgcgacggtgctcgacgacgacgagcggcgggggcggcggaggctggaggaggcggccctgctcctccacaagatcaaaggtgcgaatccccttccctctcctcgatcgactgtaaaccctcgtaatccccttcgaattggggttgcaatcggagttaacgcgggtttgcgccgccgcgcgcgtgggctgtcggcgcagggctggtgggctgggtggtcgcggaggtcagcgccggccgatccccttccctggctctccaccgctaccagaactactgcgcctgcgcctccgccgccgccgccgccgcgtccctgtccacatggtttggctccccctccctctctcgccttcaaatctctgcacaccaaacctgtcagatccttaccggtgtgtgcgcgtccgcggggtgcgggcgcgcgtgcagcgcctgcagctacgacgcccccgtcggcacggacgtcctctccctcctccgcaaggaattccacgcctcccgactcagtacggactcgcgtagaccaaactggctcgattctcatcaatctctcgttgttactggtcatgctaatcagtttgggatgcgtcgttgatgtgcagatgtgcttctcagggtcctgctcgtggtgcagcaactcctgcaggagaataagcactgctccaagagggacatctactacatgtacccctccatgttcgtaggtctgtgctggtctgcggttgccatgtctgatccatctatgcgtagtgtgagtttcaggtgttctctttctttgctgtgtgagtgacaattacaagtctcactgcagaacaagcgattgttgatcgtgccatcaatgatatctgcatactcttcaagtgcagccggcataatctcaacgtggtatgtgctaccgcttgtctgccatctttgcagaagcttcagttatgtgtttagaagtcatttgtgcattggagaataagcctctggctgttattagattatatatggagtacaatgtgccgcactgcgacttgttactccctggttgttcctccattatggtgtaccactgtcctatctgatgtaggattattttcttaatctgtcttttgtgatgcattatttcttgaggtcttcatttctccatgaatcgctaggagctgttttcattagttctctcagtgatttctgctgtatttatttcaccgccttttcacttctcttttaataaccacctgcaatttctgctcacttggctgcttgcaggttcctgtggcaaaagggtatggattcttattgagttgtgagtgtttgattttgataacatgtttaatccttctatcttgtcccatgccgttacttaactaatcagcatttttgttccttcatcctccctaccacttgtcttgctttacttaaaacatgatctggctaagcagtgtgatgctgcgcttttagcagacatttagagcatcataatctcctgtgtccatacgatggtcttagttatttctgaacattttcccgctatgtactcgtcaatatctttattgttccagtttggtgatgggctggataagatttgtggagggtgaaaagaaagtgtattgtataacaaacgtcaatgctgtaagcttctatgattctatcgaaatctatctaattactcaatatgatctgtttgtactatgcattaatttggctagcctttttccttcttcaaatgcaggctttccccattccagttagcattgaggcaatcaaaggttgtttaacaatttgaaaactcataataatctttgccattcatagacttttattatttcatatctgtttccttaggaaaatcagttttaacatctcttagcaagtcaatgttcagtttaaatttgtaatagatagtgattgatgtatcagaaagtgggatttcgacaaagcaccaaattttggacatcatgtgaaagagtaatgagaattcatctttattttttgtctcaaacatatatggagaggagtatccttgttattccacaaactatatggcataggtttgctgtctttttatacatacactatatttctttcatattctcaattactatgcagtaaacttaagctgtctaagttttttcttgcagatgttgttagtgttgctcactacatccttgttgttgagaaggaggcaggtgtgaggattaaagaccccctatctgcatatgatttttctattccttgcagtcttcctgcagtttgattctgtatgccatttgcagtgttccagcgtttggccaatgacaagttctgcgaaaagaatcgctgcattgttattacagtaactgctaacgctgcatctagtgttatactgccattatttttcacatagcatgtttgatggttttcttctgtttactcagggaagaggctacccagatattccaacaagaaggcatgtgctctttcgtatatttgtttctcctgtatattaaaactgaattgctatttgaaacgtacattccttgtaatgttgcagattcttgcgctaccttgtcgaacagctgcacttgcctgcttattgcttagtggattcagatccgtatggttttgatattctggctacctataaatttggttccatggtagggccaaaatatctatctaatatctagttatttattgaagtcctaatagcatcaaaggcagtgtaaatttatcagtgcaatttcaatttttttgttaagccaagtgaaatgtgattttgtacatttattcaaaatttagtcccattttgttgatatgtttcgagtgttaactgacaaacacttcctgctcccttgtccaattctcattctatttaagcaattggcatatgatgcaaatgttctgcgtgtgcctgagatacgatggcttggggtcttcacatctgactttgaggagtattgtcttccagactgctgtcagcttcaattgtcatctgaaggtcactaaattggagggatataaatttcatttctacttatcttggaacttgatttgttgtttaatatcaaatgacatgcatcctgcttcagataggaggaagcttgaaggaattctaaccagatgttacttgcacaaggaagccccagaatggaggtaaaggaacaggaagttgtatttcctattacacgtattccaataactttatggctatgatgtgggttgcaagaatagttagtgcatctagaattaaatagaagttctcacaatatctttcttacaaaatggcaaatgcaacagttaaactcagtgatgtaagtgaaatcttcttccaagcacaatgatcaatcctattatttagcccctgtattccaagcacttcagggatatgatgtgggttgcaaataataatagttcaagtccaactacaagaatttgttatttgatttgtttaatcaactagtagttaaatagagcttttcatattttcttcttacaaaatgccaaatgcaacaacaaaattcactgatacgagtgaaaccttcttccagtacacagtcttatgattttatcccgtgtccgtaatatgtaagtgctttcagcatcagttccttagtttcaaaaactccatatctgcgccaccttgtcgactctaacagcagtaggtgctccattgttccaaaaggtctatgactgctttcacctggtcagttctaatgtcaacgggtttgtgtttaaaattagaatcatttatgtagttaatagccggttctttttcctgaagatttttcctcctgaaatattcacgactatataatccttggcaggttgaagttggaagccatgctggaaatgggtgtcaagtttgagattgaagcattgtcagcaagttccatttcgtttctgtcgcaagagtacatcccccaacagatcaggctagggaggtatatatag

SEQ ID NO: 58 - DNA, 3928 bp, Triticum aestivum
  source 1..3928: mol_type = genomic DNA
atggcggggagtgggaagaggaggcgcgcgacggtgctcgacgacgacgagcggcggggccggcggaggctggaggaggcggccctgctcctccacaagatcaaaggtgcagatcccccttccctctcctcgatcgactgtaaaccctcgtactccccttcgaattggggttgcaatcggagttaacgcgggttcgcgccgccgcgcgcgtgggctgtcggagcagggctggtgggctgggtggtcgcggagatcagcgccggccgatccccttccctggcgctccaccgctaccagaactactgcgcctgcgcctccgccgccgccgcggcgtccccgtccacatggtttggctccctcttccccactctctcgccttcaaaatctctgccgaccaagcctgtcagatcctcaccggcgtgtgcgcgttcgcggggtgcgggcgcgcgtgcagcgcctgcagctacgacgcccccgtcggcacggacgtcctctccctcctccgcaaggaattccacgcctcccgcctcagtacggattcgcgtaccccaaactggctcggttctcatcgatctctcgttgtttctgatcatgctaatcagtttgggatgcgtcgttgatgtgcagatgtgcttctcagggtcctgctcgtggtgcagcagctcctgcaggagaataagcactgctccaagagggacatctactacatgtacccctccatgttcgtaggtctgtgctgctctgcggttgcgatgcctgatccatctatgcgtagtgtgagtttcaggtgttctgtttctttgctgtgtgagtgacaattacaagtctcactgcagaacaagcgattgttgatcgtgcgatcaacgatatctgcatactcttcaagtgcagccggcataatctcaacgtggtatgtgctaccgcttgtctgccgtctttgcagaagcttcagttatgtgtttagaagtcatttgtgcattggagaataagcctctggctgttatttcattatggtgtagtggtgtacacaatgtgccacactgccacttgttactccctggttgttctctgtcctatctgatgtaggagcattttcttaatctgtcttttgtgatgcattattcattgaggtcctcatttcttcatgaatcgtcaggagctgttttcattagttctctcagcagttctgctgcatttatttcatcgccttttcacttcgcttttagtaaccatctacaatttctgctcacttggctgcttgcaggttcctgtggcaaaagggtatggattcctattgcgttgtgagtgtttaattttgataacatgtttaatccttcgatgctgttacttaactaatcagcatttttgttccttcatcctccctaccgcttgtcctgctttacttaaaaaatgatctgggtgtaaacagtgggatgctgctcttttagcagagatttagagcatcacaatctcctatgtccatacaatggtcttagttatttccgaacattttcccgctgtgtactcgtcatgtctttattgttccagtttggtgatgggctggataagatttgtggagggtgaaaagaaagtgtactgcataacaaacgtcaatactgtaagcttctatgattccatctaatcacccagtatgatatgtttctagtatgcattaattttgctagcctttttccttcttcaaatgcaggctttccccattccagttagcattgaagcaatcaaaggttgtttaacaatttgaaaactcataacaatctttgtcttcatagttttttattatttcatatctgtttccttaggaaaaacagttttataaatcgtgatgtacacaattagcttatatctcttagcaagtcaatgttcagtttaaatttgtaatagatagtgattgatgtattagaaagtgggatttcgacaaacaaccaaattttggacagcatgtgaaagagtaatgaggattcatctttattttttgtctcaaacaaatatggagaagagtatccttgttattccacaaactgtatggcataggtttgctgtcttgttatatgtacactatatttctttcatattctcaattactatgcagtaaacttaagctgtctaagttttttcttgcagatgttgttagtgttgctcactacatccttgttgttgagaaggaggcaggtgtgaggatgaaaggccccctgtctgcatatgatttttctattctttgcagtcttcctacagttttcttctgtatgccatttgcagtattccagcgtttggccaatgacaagttctgcgaaaagaatcgctgcattgttattacagtaactgctaacgctgcatctagtgttatactgccattatttttcacatagcatgtttgatggttttcttctgtttactcagggaagaggctacccagatattccaacaagaaggcatgtgctctttcatatatttgtttctcttgtatattagaactgaattgctatttgaaatgtacattccttgtaatgttgcagattcttgcgctaccttgtcgaacagctgcacttgcctgcttattgcttagtggattcagatccgtatggttttgatattctggccacctataaatttggttccatggtagggccaaaaatcttatttattgaagtcctaatagcatcaaaggcagtgtaaaattatcagtgcaatttcaaattttttgttaagccaagtgaagtgtgattttgtacatctattcaaaatttagtcccattttgttgatatgtttctagtgttaactgaccaactcttcctgcttccttgtccaattctcattctatttaagcaattggcatatgatgcaaatattctgcgtgtgcctgagatacgatggcttggggtcttcacatctgactttgaggagtattgtcttccagactgctgtcaacttcaattgtcatctgaaggtcactaaattggagggatataaatttcatttctacttcttatctcagaacttgatttgttgttaaatatcaaatgtgatgtatcctgcttcagataggaggaagcttgaaggaattctcaccagatgttacttgcacaaggaagccccagaatggaggtaaaggaacaggaagttgtatttcctattacgcgtattccaataactttgtagctatgatgtgggttgcaagaatagttcaagtgcatctagaattaaatagaagttttcacaatatctttcttacaaaatggcaaatgcaacagttaaactcagtgatgtaagtgaaatcttcttccaagcacaaggatcaatcctattatttagcccctgtattccaagcacttcagggatatgatgtgggttgcaaataataatagttcaagtccaactctagaatttgtaatttgatttgtttaatcaactagtagttaaatagagcttttcatgttttattcttacaaaatgccaaatgcaacaacaaaactcactgatatgagtgaaatcgtcttccagtacaattatcagtcttatgagtttatcccgtgtacgtaatctgtaagtgctttcagcatcagttccttagtttcaaaaactccatatctgcgtcaccttgccgactctaatggcagtaggtgctccactgttccaaaaggtttatgactgctttcacctggtcagttctaaagtcaacgggtttgtgtttaaaattagaatcatttgtgtagttaatagccggttctttttcctgaagatttttcttcctgaaatattcatgactatataatccttggcaggttgaagttggaagccatgctggaaatgggtgtcaagtttgagattgaagcattgtcagcaagttccatttcgtttctgtcgcaggagtacatcccccaacagatcagactagggaggtacatatag

SEQ ID NO: 59 - DNA, 3970 bp, Triticum aestivum
  source 1..3970: mol_type = genomic DNA
atggcggggagtgggaagcggaggcgcgcgacggtgctcgacgacgacgagcggcgggggcggcggaggctggaggaggcggccctgctcctccacaagatcaaaggtgcgaatccccttccctctcctcgatcgactgtaaaccctcgtaatccccttcgaattggggttgcaatcggagttaacgcggtttgcgccgccgcgcgcgtgggctgtcggagcagggctggtgggctgggtggtcgcggagatcagcgccggccgatccccttccctggcgctccaccgctaccagaactactgcgcctccgccgacgccgccgccgccgccgcgtccccgtccacatggtttggctccctccctccctctctcgccttcaaatctctgccgaccaagcctgtcagatcctcaccggtgtgtgcgcgttcgcggggtgcgggcgcgcgtgcagcgcctgcagctacgacgcccccgtcggcacggacgtcctctccctcctccgcaaggaattccacgcttcccgcctcagtacggattcgcgtaccccaaactggctcgattctcatcaatctctcgttgtttctgatcatgctaatcagttcgagatgcgtcgttgatgtgcagatgtgcttctcagggtcctgctcgtggtgcagcaactcctgcaggagaacaagcactgctccaagagggacatctattacatgtacccctccatgttcgtaggtcggtgctggtctgcggttgccatgtctgatccatctatgcgtagtgtgagtttcaggtgttctgtttctttgctctgtgtgtgacaattacaagtctcactgcagaacaagcgattgttgatcgtgccatcaacgatatctgcatactcttcaagtgcagccggcataatctcaacgtggtatgtgctaccgcttgtctgccatctttgcagaagctttagttatgtgtttagaagtcatttgtgcattggagaataagcctctggctgttatttgattatatggtgtacaatgtgccacactgcgacttgctactccctggttgttcgtccgttatggtgtaccactgtcctatctgatgtaggattattttcttaatctgtcttttgtgatgattccttgaggtcttcattttttcatgaatcgctgggagctgtttctgttagttctctataagtgatttctactgtatttatttcaccacctcttcacttctcttttaataaccacctacaatttctgctcatttggctgtttgcaggttcctgtggcaaaagggtatggattcttattgagttgtgagtgtttgattttgataacatgtttaatccttccatcttgtcccatgccgttacttaactaatcacatttttgttccttcatcctccctaccacttgtcctgctttacttaaaacatgagctggctaaacagtgggatgctgcacttttagcagacatttagagcatcataatctcctgtgtccatacaatggtcttagttatttctgaacattttcccgctatgtactcttcaatgtctttattgttccagtttggtgatgggctggataagatttgtggagggtgaaaagaaagtgtattgtataacaaacgtcaatgctgtaagcttctatgattctatctatctaattactcaatatgatctgtttgtactatgcattaatttggctagcttttttccttcttcaaatgcaggctttccccattccagttagcattgaggcaatcaaaggttgtttaacaatttgaaaacttataataatctttgccatttatagatttttattatttcaaatctgtttctttaggaaaatcagttttaaaaatcgtgatgtgcacaattagcttatatctcttagcaagtcaatgttcattttaaattttaatagatagtgattgatgtattaggaagtgggatttcgacagaccactaaattttggacagcatgtgaaagcgtaatgataatttatctttattttttgtctcaaacaaatatggagaggagtatccttgttattccacaaactatatggcataggtttgctatcttgttatatgtacactataattctttcatataatattctcaattgctatgcagtaaacttaagctgtctaagttttttcttgcagatgttgttagtgttgctcactacatccttgttgttgagaaggaggcaggtgtgaggattaaaggccccctatctgcatatgatttttctattctttgcagtcttcctacagtttgcttctgtatgccatttgcagtgttccagcgtttggccaatgacaagttctgcgaaaagaatcgctgcattgttattacagtaactgctaacgctgcatctagtgttatactgccattatttttcacatagcatgtttgatggttttcttctgtttactcagggaagaggctacccagatattccaacaagaaggcatgtgctctttcgtatatttgtttctcttgtatattagaactgaattgccatgtgaaacgtacatgccttgtaatgttgcagattcttgcgctaccttgtcgaacagctgcacttgcctgcttattgcttagtggattcagatccatatggttttgatattctggccacctataaatttggttccatggtagggccaaaaatctatctaatatctaattatttatttgaagtcctaatagcatcaaaggcagtgtaaatttatcagtgcaatttcaatttttttttgttaagccaagtgaaatgtgattttgtacattttattcgaaatttagtcccattttgttggtatttttctagtgttaactgaccaactcttcctgctcccttgtccaattctcattctattttagcaattggcatatgatgcaaatgttctgcgtgtgcctgagatacgatggcttggggtcttcacatctgactttgaggagtattgtcttccggactgctgtcaattacaattgtcatctgaaggtcactaaattggagggatataaatttcatttctacttcttatctcagaaattgatttgttgttaaatgtcaaatgccatgtatcctgcttcagataggaggaagcttgaaggaattctcaccagatgttacttgcacaaggaagccccagaatggaggtaaaggaacaggaagttgtatttcctattacgcgcatcccaataactttatggctatgatgtgggttgcaagaatacttcaagtgcatctagaattaaatagaagttttcacaatatctttcttacaaaatggcaaatgcaacagttaaactcagtgatgtaagtgaaatcttcttccaagcacaatgatcaatcctatatttaggccctgtattccaagcacttcagggatatgatgtgggttgcaaataataatagttcgagtccaactgctagaatttgttatttgatttgtttgttctactagtagttaaatagagcttttcatattttgttcttacaaaatgtcaaatgcaacaacaaaactcactgatatgagtgaaatcttcttcctgtacaattatcagtcttatgattttatcccgtgtaagtaatctgtaagtgctttcagcatcaattccttagtttcgaaaactccatatctgcgtcacctagtcgactcttaacggcagtaggtactccattgttccaaaaggtttatggctgctttcacctggtcagttctaatgtcaacgggtttgtgtttaaaattagaatcatttgtgtagttaatagccggttctttttcctgaagatttttcttcctgaaatattcatgactatataatccttggcaggttgaagttggaagccatgctggaaatgggtgtcaaatttgagattgaagcattgtcagcaagttccatttcgtttctgtcgcaagagtacatcccccaacagatcagactagggaggtacatatag

SEQ ID NO: 60 - DNA, 6271 bp, Triticum aestivum
  source 1..6271: mol_type = genomic DNA
atgttctactcgcaccagctcctcgcgcggaaggcgccgctcggccagatatggtgcgtcccctcccctccctcctcgccggccgccgccgctcggtcccctcgtcgcggccgccggccaccgatttcgcctgtcccgcgtttttctgctcggttccgggcgtttcccgacgcgtccgtccgatccgctcccaatgcgggctcctcgtcgcctcgcctccatctcctctgaccctcctatgttctgcaggatggccgccacgctccacgccaagatcaaccgcaagcgcctcgacaagctcgacatcatcaaaatctggtgagccaccgacccgatcccgccggtttccctctgctacgacctcgcggttcgcccgattttaattcgcgtgtggtgcgatgtgtctgatgctgctgcagcgaggagatcctcaacccgtcggtgcccatggcgctcaggctctccgggatcctcatgggtgagccgtccgggttatttcgctccttttctcgacggatttgttcttgttctccctcggggatgtgacgcgtgtttgttgcctgtgtgtttgggtggcgtgatgagcaggtggcgtggtgatcgtgtacgagaggaaggtgaagcttctctacggtcagtccatctgttgccttccctcgctgcttgccgcacttttttcttgtcgaaatcttcagattctcaccgtttccgactgttcctcgcagatgacgtgtcccgtctcctggtaaacttcgacttccagtttcctctgcatcctgcttggtgatttggtgcttgggttcatgtttgtgctgattccttacgcagattgagatcaacgaggcgtggaagatcaggccggccgtggaccacaccgtcctccccaagggcaaggctcaagccaagtgagtgctctccgtctccatgccctgccacaagtttctgaatgtgcttgcgttgtgataatgtagagccggcgtccatttctctctggagatggggttttccttgtgtgtttagaccgtgtggtgttagcctgatggtttacattgggggcttttgcaaggtaccaggtggctctatgtatggacagcttaactttgtctgtttatgaaatttcttagaggctcgatagcaatgcagtctctgtcagcttgcttgccaccaattggtgtgttctgcattgtacagtattgagtttacgatgatatgtgatttgtgatgttttcagatgtttctaatagaagattgtgaatgtgtttgcgtggtgatgatggagagttggtgtccatttctctgtggaaatggaattttctttgtgtctttagaccgtgtggtgttagtctgatggtttagattggaggctgttgtaaggttccaaagtagccgaatgaacaactgaactttctttgtttatgaaatttcttaagatgcctaataacactgtcagtctgtcagcttgcttggtaactattggtgtgtgctgcattccgcactactgattttacaatgaccaacggcactactttgtggtgttttcagatgtttctagaaaatgccatatcactgattgcgtgaaatgtaactgggatgcacagccctagccgtgggtcctttctgtgtgatcctgctgtaacttgactctgttcttgtgtttctggggcagcaggtattcatatagaccttcatatgattttgtggctcctgtcttgatattctgattgtttgattaaattcatgggttgcaactttggctaagtggggtcgtttgggtgttatagctgttggtgcaagggttcatgcgggtattcatgtctctgtatgttgctgattgttatgtgtgggttagccaattttaatcagctccaggttgcaaggtggtttttccggccaatatgtatgtgtattctatgcaagactttatgaattttatagtttctccagcttttttaggacatagtttgtccatttgttatcttaacaattttaataatggggtggctggctgggacagcgagatattggttggttttgggttgttcagtgctgacttattattttggtattagtttcttattcagtatcagtcaatctcttgagaatgtatgatctaattatagcagatctgggggttgtatggtacttgagtaataatgctatgttcagaggggatctttatcaccttggaaaactgataacatatcttagcattctgtcatgatcagagagcggactcttgttgctcgcctttgaaagtttgttagcactccaaatgagatatttaacgtcttgctactgtgccattgccattgagaaaaggggtttatgtagaatgatttgtcttaagattaagaaagcagtgtgtcattttatctgaaagttctgcttcttgcaggtatgaagcagtaacactgccagagaacgcgatggatatggaggtggagcagcccgtgcttttcacagatactgatactgccaggttccggggaatggtaatcattgtcatctgtgtattctggaagttgaaactctgattaaatgcatggatggatttttctgtctcctagtttctgtctgaattgcctgcactcatcttgactgaagtctatgcttgtgcagcgcctggaggatttggatgaacagtatgtcaatgtcaacctggatgatgatgacatctctcgcgccgaccgtcatcatcaaggtatttctttctgctctttggtattgctctttgagggttcttaagggctaatgattattaactcttggttctttatgatcgtatctcagctgaggcagtcaacattaccctggtcgataattttgagtctggtcttgctgaaactgacatcttcaatcgttttgagaggtaaaggagacttaaatttccctaattaaaagaaaagaaaagttattcttattttagtgtgccattctcgtatcatcaatgttgcgttttcttttaatttggtggtgagtaccaatattcattgtgcaatactaccttgtcaccagatttgacatagcagatgatgacaccacagtccatattactctcgatgaacacccagaggctccaagtacactagttccctctccaccaaggccagaagaccctcctcaacaacaggaacagtgtgctgccccatcctccatccgcgaagaacctcaacaaggtgaaattaagcacatgactagtatatttgatgatgctacagtgggaaatggcttatcacatagtaaaattggaaatgatttcggtttaacgctaatagacaccaattttcatctcatttcgagtctgcgacacatttaccatgttatttcaattcagggaaacttccaccaagacttgtttgaacacattgactttttctttgagtttcttctggaatagtacaactttaaggttatttgtgagagcttaagatttgcactggtgcttagcatattgtgcgctatggctattcggtcaagtagcgctggttctaagcatatcatcttcgctcattatccccagtcggttttgtaaatggccaatttgaatgtggcaggactatatattatagattaagtggataagtagtgtagtattgattgcagctgaatttactacagatttttggtaattttctatgttatgactgatcatgagcagcacttataatacgtactggatttgcaagtaaactgatgtggaacctttctatttgaaacgaatcttgcatgtagcttactgtcctttggctagttcactggttgcatgttgtattagaagttgtaccactggacccagttcagctactcatatactatagggcaagtatcttctgcaggggattcattgaaggagcaagaggagcagaagacgacggtctgtaccatttacctgatgaatagttggctttagttgaaactcttatatggaaaaggaatttgatgttgtttacatgaggttgcttttcgcaatataggagcaacaaccaactaaacgagcaaagaggaaagcacgcggcaagggcccccaagtgatcatggacaaccagataatgatcccaggaaatgtatatcagtcatggttgaaggatccatcaagcctcatctctaaaaggcgtcaagtcaggagtgtatgtcaacgtgttattgattggaaccacctattttccaatagttctgaactcatgcattgtacttaccgaattgttgttttgctgtttactatttgcagaaaatcaatcctattaaggcaattaagataggcgagctcatggacttgccaccgtctgccctaatgtcttgctccgatgactcacaagagatatattaccctcagcagcttatgcagctctggaaggaatgcaccaaagtcaagcccccaaagccctcatcttcttcaggtaactataacactgaactttgtgtcacccaccttttaacttgcaaaagtgctaattccaagctgcacactatctcatataggagataaatcatcatcatcatcacaagaaaagcagccgagaaactctccgcctcaggttgtttcagaacacatttctctattgatttattttcatcaacatttatctcataattggttaaactggcagcctcaaggagatcagaatgaaatgggagctcaaccaatgggcttcacaccaatggacttcacagatggcattgaaaagatgagagcaaacaagagtggagaatttgaaggtgtttttgatggtccgcatggtgaccctagtgttacacctggaagtcctggtaagttgaaaacctttcttttttaagttccttcttctttagccgcttcattacatcaactatgctgtagggctaagtcgcaggtcagcttcaagctctggtggctctggaagggggggatttctgccattggatccagagatactgttacaatctggaagcggaaggtgactgttgatactgatagtaaccttgttctttattagttgcacgaataattgactatgtgatcttgtgatgaaaaaaaaaactcggtgattaagaaagttttctctttgtactgggttatccttttatttctatgctctgtctagaaactgttagagctgtctccctgacgttgcgttgtacataaaggaaaacgactgatcattgatgttaaccatcataagtggcagcttcctgtggtttttcattgtcttgcttagtttcttagtgtaactgtctgaggactattgtgcagggccaagaggaggcagctttcatctggacggagcttggggaaccttgatccagttgaagaggaattcccaatggagcaggaagggagggagtgcaagctgagaaggctttcagatattgaaccaactcctggttggttgcatttctgcctttctgtccttaccttaaactgttgtttatggatatgagtatttcaatcctgtttcctccacgaaaacagcagttaaaaacatttcttgtttcagatcttatggtagaaacagaacccactcaaaccccgttcacaaagcaatccagtcctcccgatcacatcactgaatcaattcactcgtaagtatcctgagaacattgctgataattcctgggtggcttatgtttgactcgcggagtacaatgtgtacatcatgtgatgctggagttaaattcatgcaggtacctaaagctccacttcgagagcccggatgccccgccgtctgaatcgctaagccagctaacttatgggatgaatacagcacaggctgcccgcctattctatcaaacatgtggtatggaatagttctacattgtgaataaatcgtgcaatcactagagtaaataagaaatcaaaggtcttactgcatgattttatatagttgctttgctgtttattgagtattcataatttccatggacagtaactataattacacacattacatgccatgcttgcttgttctttttcattatactgggtcttggctgggttaaaaaattctgacaggttaacttagctgcggtgttaaaagcagatcatatatgtgtcatagcatgagaaccggggaagaaaccaccaagtttccttgatagcatcgactagacagataaccccctaggacattgagtgcaactcttttaaccgttctgtttggtcatgtcactgaatgtaaactattgttcacagcgggttttatcttgtgcagtcttagcaacgctcgacagcatcaaggtcacgcaggtggaaccatacgggcccatcctgatctcgaggggcacaaacatgtga

SEQ ID NO: 61 - DNA, 6427 bp, Triticum aestivum
  source 1..6427: mol_type = genomic DNA
atgttctactcgcaccagctcctcgcgcggaaggcgccgctcggccagatatggtgcgtcccctccccctccccctcccctccctccccgccggccgccgccgctcccgtcccctcggtcgcggccgccggccaccgattccgcctgtcccgcgtttctccgctcggtccggggcgtttccgggcgcccccgttcggtccgctcccaatgcggcgggctccccgtcgcctcgcctcgatctgctctgacgctcctctgctcctctgcccccgccgcaggatggccgccacgctccacgccaagatcaaccgcaagcgcctcgtcaagctcgacatcatcaaaatctggtgagcccccgacccgaccccgccaatttctctctgctttgagggcccgcggtctcatggcgctgtgtctgacgccgctgcagcgaggagatcctgaacccgtcggtgcccatggcgctcaggctctccgggatcctcatgggtgagccgtccctccgggccatttcgctcctttttttcccctcgccggattcgttcctgttctccccggggatgtgacgcgtgtttgttgctgttgttgcctcctgtgtgcgcgtgatgagcaggcggcgtggtgatcgtgtacgagaggaaggtgaagcttctctacggtgagtccacctgccgccttccctcgctgctcgccgcacttattttcttgtcgaaatcttcagattctcaccgtttccaactgtgtggttcctcgcagatgatgtgtcccgtctcctggtaaacttggacttcgagttgcgcccgaccggtttctctgcatcctgctgcttggtgatttggtggctgggtttacgcttgtgctgatttcttctgcagattgagatcaacgaggcatggaagatcaggccggccgtcgaccacaccgtcctccccaagggcaaggctcaagccaagtgagtgctctctccctctccctctccatgccctgccacaagtttctggatgtgcttgcgtggtgataatgtggagccggcgtccatttctctctggagatggggttttccttatgtgtttagactttccttgtgtgtttagaccgtgtggtgttagcctgatggtttacattgggggcttctgcaaggtaccaggtggctgtatgtatggacagcttaacttcgtctgtttatgaaatttcttagaggctagatagcaatgtcagtctccgtcagcttgcttgctaccaattggtgtgttctgcattgtacggtattgagtttacgatgatatgtggtttgtgatgtttttcagatgtttctagtagaagtttctgaatgtgtttgcgtggtgatgatggagagttggtgtccatttctctgtggaaatggaatttcctttgtgtgtttagaccgtgtggtattagtctgatggtttagattggagggtgttgcaaggtgccaaagtggccgtatgagcaactgaactttctgtgtttatgaaatttcttaagatgcctaataacaacactgtcagtctgtcagcttgcttggtaactattgctgtgtcctccattgtgcactactgattttacaatgaccaacggcactactttgtggtgttttcagatgtttctataagtttattgatgatgccgtatcactgactgcgtgaaatgtaactgggatgcacagtcctagccatgggtccatgctgtgaaatgtaactgggacgcgcagccctagccgtgggcccttgctgtgtgatcctacgtaacttgactctgttcttgtgtttctggggcagcagctattcatatagaccatcatatgattttgtgtctcctgtcttgatattctcattgcttgattaaattcatgggttgcaactttgcctaaggggggtcgtttgggtgttatagctgttgatgcaagggttcatgcaggtatttatgtctctgtatgttgctgattgttatgtctgggttagtcaattttaatctgctccaggttgcaaggtggtttttctggccaatatgtatgtgtactctatgcaggactttatgcattttatagtttctccagcctttttaggacatagtttgtccatttgttatcttaacaattttaataatggggtggctgactgggacagtgagatatcggttggttttggactgttcagtgctgacttattattttggtattggtttcttattcaatatcagtcaatctcttgagactggatgatctaattatagcagatctgggggttgtaatggtacttgagaaataatgctatgttcagaggggatctttatcaccttggaaaactgttaacatatcttaacatgctgtcatgttcagagcgcggactcttgtcgcttgcctttgaaagtttgttagcactccaaatgagatatttaacctcttgctactgtgtcattgccattgagaaaagggttttatggagaatgatatgtcttaagattaagaaaacagtgtgtcatttttatctgaaagtgctgcttcttgcaggtatgaagcagtaacactgccagagaacgcggtggatatggaggtggagcagcccgtgtttttcacagatacttatactactaggttccggggaatggtaatcgttgtcatctgtgtattctggaagttgaaactctgattaaatgaatggatggattttcctgtctcctagcttctgtctgaattgcctgcactcatcttgactgacgtctatgcttgtgcagcgcctggaggatttggatgaacaatatgtcaatgtcaacctggatgatgatgacatctctcgcgccgaccgtcatcatcaaggtatttctttctgctctttggtattgctctgtgagggttcttaagggctactgattattaactcttggttctttatgattatatctcagctgaggcagtcaacattaccctggtcgataattttgagtctggtcttgctgaaactgacatcttcaatcgttttgagaggtaaaggagacttaagtttccctaattaaaagaaaagaaaagttattcttattttagtgtgccattctcgtatcatcaatgttgcgtcttcttttaatttgacggtgagtaccaatattcattgtgcaatactaccttgtcaccagatttgacatagcagatgatgacaccacagtccatattactctcgatggacacccagaggctccaagtacactagttccctctccaccaaggccagaagaccctcctcaacaacaggaacagtgtgctgccccatcccccatccgcgaagaacctcaacaaggtgaaattaagcacatgactagtatatttgacgatgctacagtgggaaatggcttatcacattgtaaaattggaaatggttccggtttaacgctaatagacaccaattttcatctcattttgagtctgcgacacatttaccatgttttttcaattcagggaaacttccaccaatgcattgactttttctttgagtttcttctggaatagtataactttaaggttatttgtgagagcttaagatttgcactggtgcttagcatattgtgcgctatggctattcggtcaagtagcgctggttctaagcatattgttttcgctcattatccccagtcggttttgtaaatggccaatttgaatgcggcaggactatatgttatagattaagtggataagtagtgtagtattgattgcagctgaatttactacagaattttggtgatttgataataccattcactctattttatgactgatcatgggcagcacttataatactggatttgcaagtaaactgatgtggaacctttctatttgaaacgaatcttgcatgtagcttattgtcctttggctagttcactggttgcatgttatattagaagttgtaccactggacccagttcagctactcatatactatagggcaagtatcttctgcaggggattcattaaaggagcaagaggagcagaagacgacggtctgtaccatttacctgctgaatagttggctttagttgaaactcttatatggaaaaggaatttgatgttgtttacatgagattgcttttcgcaatacaggagcaacaaccaactaaacgagcaaagaggaaagcacgcggcaagggcccccaagtgatcatagataaccagataatgatcccaggaaatgtatatcagtcatggttgaaggacccatcaagcctcacctctaaaaggcgtcaagtcaggagtgtatgtcaacgtgttattgattggaaccacctattttccgatagttctgaactcatgcattgtacttaccgaattgctgttttgctgtttactatttgcagaaaatcaatcctattaaggcaattaagataggcgagctcatggacttgccaccgtctgccctaatgtctttctccaatgactcacaagagatatattaccctcagcagcttatgcagctctggaaggaatgcaccaaagtcaagaccccaaagccctcatcttcttcaggtaactataacactgaactttgtgtcacccaccttttaacttgcaaaagtgctaattccaagctgcacactatctcatacaggagataaatcatcatcatcatcacaagaaaagcagccgagaaacccttcgcctcaggttgtttcagaacacatttctctattgatttattttcatcaacatttatctcataattggttttcactggcagcctcaaggagatcagaatgaaatgggagctcagccaatggacttcacaccaatggacttcacagatggcattgaaaagatgagagcaaacaagagtggagaatttgaaggtgtttttgatggtccgcatggtgaccctagtgttacacctggaagtcctggtaagttggagatctttcttttttaagttccttcttctttagccgcttcatgacgtcaactctgctgtagggctaagtcgcaggtcagcttcaagctctggtggctctggaaggggggcatttctgccattggatccagaaatacagttacaatctggaagtggaaggtgactgttgatactgatagtaaccttgttctttattagttgcacgaataattgactatgtgatcttgttatgaaaaaaaaaactcggtgattaagaaagttttctctttgtactgggttttccttccatttctatgctctgtctagaaactgttagagctgtctccctgatgttgcgttggatataaaggaaaacacctgatcattgatgttaaccatcataagtggctcccgcaagaaaaaaagaaaccatcataagtggcagcttcctgtggttttttcattgtcttgcttagtttcttagtgtaactgtctgaggactattgtatagggccaagaggaggcagctttcatctggacgaagcttggggaaccttgatccagttgaagaggaattcccaatggagcaagaagagagggaattcaagctgagaaggctttcagatatggaaccaactcctggtgggttgcatttctgcctttctgtccttaccttaaactgttgtccctgaaaatagcagttaaaaacatttcttgtttcagatcttatggtagaaacagaacccactcaaaccccgttcatgaagcaatccagtcctcccgatcacatcactgaatcaattcactcgtaagtatcctgagcacattggtgataattccggggtggcttatgtttgactcgcggagtacaagtgtgttcgtcgtgtgacgctggagttaaattcatgcaggtacctaaagctccacttcgagagcccggatgccccgccatctgaatcgctaagccagctaacttatgggatgaacacagcacaggctgcccgcctattctatcaaacatgtggtatggaacagttctacattgtgaataaatcgtgcagtcactagagtaaataagaaatcaaaggtcttaccgcatgattttatatagttgctttgctgttgattgattattcataatttccatggacaatgactataattacacactttgcatgacatgcttgctttctctcatattgctgggttatatactagtagtatgttaattacacactttgcatgacatgctggccttcctgataagttaactttgctgcggtgctaaaagcagaacatatatagcttgaaactggggaagaaacaccaagtttccttgatagcatcgactagatggataacccctaggacgttgagtgcaattcttttaaccgttctgtttggtcatgtcactgaatgtaaactattgttcactgagggttttatcttgtgcagtcttggcaacgctcgatcgcatcaaggtcacacaggtggaaccatacggacccatcctgatctcgaggggggccaacatgtga

SEQ ID NO: 62 - DNA, 6264 bp, Triticum aestivum
  source 1..6264: mol_type = genomic DNA
atgttctactcgcaccagctcctcgcgcggaaggcgccgctcggtcagatatggtgcgtcccctcccctccctcaccgccggccgccgccgctcggtcccctcggtcgcggccgccggcgccgatttcgcctgtctcgcgtttttccgctcggtcccgggcgtttccggacgcgtccgttcgacccgctcccaatgcgggctcctcgccgcctcgatctcctctgacgctcctctgttctgcaggatggccgccacgctccacgccaagatcaaccgcaagcgcctcgacaagctcgacatcatcaaaatctggtgagccgcccgcttcccccgatcccaacgatttccctctgctgcgagctcgcggtccaccccgtcctcgtgcgattctaatccgcgtgtgctgccatgtgtctgatgctgcagcgaggagatcctcaacccgtcggtgcccatggcgctcaggctctccgggatcctcatgggtgagccgtccggccgggttatttagctccttcttttctcgccggatttcttcttgttctccctcggggatgtgacgcgtgtttgttgttgcctcctgtgtgcgcgtgcgtttgggtggcgtggtgagcaggtggcgtggtgatcgtgtacgagaggaaggtgaagcttctctacggtcagtccatctcttgcctgcttgctgcacttttttgttgtcgaaatgttcagatgctcatcgtttccaactgttccttgcagatgatgtgtcccgtctcctggtaaacttcgacttccagtttcgcctgatttctctgcatcctgcttggtaattgggtggttcggtttacgctcgtgctgattccttccgcagattgagatcaacgaggcatggaagatcaggccggccgtggaccacaccgtcctccccaagggcaaggctcaagccaagtgagtgctctctccgtctccatgccctgccacaagtttctgaatgtgcttgcgtggtgatactgtagagccggcgtccatttctctctggaatgggttttctttgtgtgtttagattagaccgtgtggtgttagcctgatggtttgcattggaggcttttgcagggtaccaaggtggctctatctatggacagcttaactttgtctgtttatgaaatttctgagaggcttgatagcaatgcagtctctgtcagcttgcttgctaccaattggtgtgttctgcattgtacagtattgagtttacgatgatatgtgatttgtgatgttttcagatgtttctagtacaagtttctgaatgtgtttgcgtggtgatgatggagagttggtgtccatttctctgtggaaatggaattttctttgaccgtgtggtgttagtctgatggtttagattggaggctgttgcaaggtgccaaagtagccgtatgaacaactgaactttctttgtttatgaaatttcttaagatgcctaataacaccgtcagtctgtcagcttgcttggtaactattggtgcgtgctgcattccgactactgattttacaatgaccaacggcactactttgtggtgttttcagatgtttctagaattttattgatgatgccatatcactgattgcgtgaaatgtaactgggtccttgctgtgtgatcctgctgtaacttgactctgttcttgtgtttctggggcagcagctattcatatagaccttcatatgattttgtggctcctgtcttgatattctgattgtttgattaaattcatgggttgcaactttggctaagtggggtcgtttgggtgttatagctgttgatgcaagggttcatgcgggtattcatgtctctgtatgttgctgattgttatgtgtgggttagccaattttaatcagctccaggttgcaaggtggtttttccggccaatatgtatgtgtattctatgcaagactttatgcattttatagtttctccagcttttttaggacatagtttgtccatttgttatcttagcaattttaataatggggtggctggctgggacagtgagatattggttggttttgggttgttcagtgctgacttattattttggtattggtttcttattcaatatcagtcaatctgttgagaatgtatgatctaattatcagatctgggggttgtatggtacttgagtaataatgctatgttcagaggggatcttgatcaccttggaaaactgatgacatatcttagcattctgtcatgatcagagagcggactcttgtcgctcgcctttgaaagtttgtcactccaaatgagatatttaacctcttgctactgtgccattgccattgagaaaaggggtttatggagaatgatttgtcttaagattaagaaagaagtgtgtcacttttatctgaaagttctgctttttgcaggtatgaagcagtaacactgccagagaacgcgatggatatggaggtggagcagcccgtgcttttcacagatactgatactgccaggttccggggaatggtaatcattgtcacctgtgtattctggaagttgaaactctgattaaatgcatggatggatttttctgtctcctagtttctgtctgaattgcctgcactcatcttgactgaagtctatgcttgtgcagcgcctggaggatttggatgaacagtatgtcaatgtcaacctggatgatgatgacatctctcgcgccgaccgtcatcatcaaggtgtttctttctgctctttggtattgctctttgagggttcttaagggctactgattattaactcatggttctttatgatcgtatctcagctgaggcagtcaacattaccctggtcgataattttgagtctggtcttgctgaaactgacatcttcaatcgttttgagaggtaaaggagacttaagtttccctaattaaaagaaaagaaaagttattcttattttagtgtgccattcttgtatcatcaatgttgcgtcttcttttaatttggcgtgagtaccaatattcattgtgcaatactaccttgtcaccagatttgacatagcagatgatgacaccacagtccatattactctcgatggacacccagaggctccaagtacactagttccctctccaccaaggccagaagaccctcctcaacaacaggaacagtgtgctgccccatcccccatccgcgaagaacctcaacaaggtgaaattaagcacatgactagtatatttgatgatgctacagtgggaaatggcaaatggcttatcacattgtaaaattcgaaatgatttcggtttaacgctaatagacaccaattttcatctcatttcgagtctgcgacacatttaccatgttatttcaattcagtaaacttccaccaagacttgtttgaacgcattgactttttcttagagtttcttctggaatagtacaactttaaggttatttgtgagagcttaagatttgcactggtgcttagcatattgtgcgctatggctattcggtcaagtagcgctggttctaagcatatcgtcttcgctcattatccccagtcggttttgtaaatggccaatttgaatgcggcaggactatatattatagattaagtgggtaagtagtgtagtattgattgcagctgaatttactacagatttttggtaatttgataataccattcactctattttatgactgatcatgagcagcacttataatactggatttgcaagtaaactgatgtggaacctttctatttgaaacgaatcttgcatgtagcttattgtcctttggctagttcactggttgcatgttatattagaagttgtaccactggacccagttcagctactcatatactatagggcaagtatcttctgcaggggattcattgaaggagcaagaggagcagaagacgacggtctgtaccatttacctgatgaatagttggctttagttgaaactcttatatggaaaaggaatttgatgttgtttacatgagattgcttttcgcaatacaggagcaacaaccaactaaacgagcaaagaggaaagcacgcggcaagggcccccaagtgatcatggataaccagataatgatcccaggaaatgtatatcagtcatggttgaaggacccatcaagcctcatctctaaaaggcgtcaagtcaggagtgtatgtcaactgttattgattggaaccacctattttccgatagttctgaactcatgcattgtacttaccgaattgctgttttgctgtttactatttgcagaaaatcaatcctattaaggcaattaagataggcgagctcatggacttgccaccgtctgccctaatgtcttgctccgatgactcacaagagatatattaccctcagcagcttatgcagctctggaaggaatgcaccaaagtcaagcccccaaagccctcatcttcttcaggtaactataacactgaactttgtgtcacccaccttttaacttgcaaaagtgctaattccaagctgcacactatctcatacaggagataaatcatcatcatcatcacaagaaaagcagccgagaaactctccgcctcaggttgtttcagaacacatttctctattgatttattttcatcaacatttatctcattattggttcaactggcagcctcaaggagatcagaatgaaatgggagctcaaccaatggacttcacaccaatggacttcacagatggcattgaaaagatgagagcaaacaagagtggagaatttgaaggtgtttttgatggtccgcatggtgaccctagtgttacacctggaagtcctggtaagttggaaacctttcttttttaagttccttcttctttagccgcttcatgacatcaactatgctgtagggctaagtcgcaggtcagcttcaagctctggtggctctggaagggggggatttctgccattggatccagaaatactgttacaatctggaagtggaaggtgactgttgatactgatagtatccatgttctttattggttgcacgaataattgactgtgtgatcttgttatgaaaaaaaaaactcggtgattaagaaaattttctctttgtactgggttatccttttatttctatgctctgtctagaaactgttagagctgtctccctgacgttgcgttgtacataaaggaaaacgactgatcattgatgttaaccatcacaagtggcagcttcctgtgattttcattgtcttgcttagtttcttagtgtaactgtctgaggactattgtgcagggccaagaggaggcagctttcatctggacgaagcttggggaaccttgatccagttgaagaggaattcccaatggagcaagaagggagggagttcaagctgagaaggctttcagatattgaaccaactcctggttggttgcatttctgcctttctgtccttaccttaaactgttgtccctgaaaatagcagttaaaaacatttcttgtttcagatcttatggtagaaacagaacccactcaaaccccgttcacaaagcaatccagtcctcccgatcacatcactgaatcaattcactcgtaagtatcctgagcacattgctgataattcctgggtggcttatgtttgactcgcggagtacaagtgtgtacatcgtatgacgctggagttaaatccatgcaggtacctaaagctccacttcgagagcccggatgccccgccgtctgaatcgctaagccagctaacttatgggatgaatacagcacaggctgcccgcctattctatcaaacatgtggtatggaatagttctacattgtgaatagatcgtgcaatcactagagtaaataagaaatcaaaggtcttactgcatgattttatatagttgctttgctgtttattgatcatttgtaatttccatggacagataactataattaaagcacattacatgccatgcttgcttgttctctttcattatgctgggtcttggatgggttataagattctgacaagttaacttgactgcggtgttaaaagcagatcatatattttgtcatagcatgagaactggggaagaaaccaccaagtttccttgatagcattggctagaaggataacccctaggacgctgagtgcaactcttaactgctttgtttggtcatgtcactgaatgcaaactgttgcttactgagggttttatgccgtgcagtcttagcaacgctcgatcgcatcaaggtcacacaggtggaaccatacggacccatcctgatctcaaggggggccaacatgtga

SEQ ID NO: 63 - DNA, 2842 bp, Triticum aestivum
  source 1..2842: mol_type = genomic DNA
atggctgaagtgaggattgccagcaggacggctgtagccgatcgctctggcggtggattcttcgtcaggagggtggcgtctcccggagccgtggtggacaagggcgccgtcaagccacttactcgtcgagtcccgtcaccgtccagcaacaaggagaacgtgccaccagcgtgctctgcgtggactgcacccaagaggaggagctccctgcctgagtggtgcccaaggaccccactccgtgatatcacatcagtcatcaaggtgataaacatttcctcttgtgggtctgcttcatggatcaatggatcatgacagttttacattgtatgatcatgtgcattttagctgtgcctagtctatggcgtccaaagttactattcacaacagtatgatctagcttatgctagttttgtaccttgctgagattcatgcaacatctgacctggtgatgtagaccgaacctcgatcccgagatccgagctgttgctgcggcccgacctttcacgacacttcaccttcagctgtagtagcttctcgcccgcgcacgtgatgtacgcaaagtagagggattgaaccttacggtccagcacgagaaaaccaatctcgacgacggtactcgcgcgcaaaacaatttcctcaaagagaaatcgcaccacagatattttctaaagcttccttcaaaacttaggattttttagatagcttccccaaaactcgatacgggtatttaccctaaaaacacatcgtgtctattcataaaaatatatcctccctttacctgaacatgatccggctatttatactagtatagccgacccacctaacaatgattaaacacacgcaagcttttgtatctattcctgacttccaaacggaaacaactaaagataggaactaactcaatatttttcctaaacaaaactctaaacttacacaatatcttgccgatctctatggggtgggccccaggcaacagaacacaatgaaagatattcacgcacgtcaggactcctcctggaagcttcacgtgtcttctctttccatggtagaaaaataaatagctctctatttttaatcctcttgaccgaaccagaccaatctgatctcgtcttagtctttgtatcatagtaggactccttttctttccatgatgtaatgcacgtcagtattgccttgcaaaactcaacacatccatcggagtaattcggccaacatgcagacttgcatgcatgcacatctacaagcagtaatattccaccaagctcttcctcacgtgacatcatatttagttgttggtttctctctttaaactgaacaaagcacatctcttttagtgtctcatcacctgtacaagttacattatctagctcctgctcatcttcacagatctgtaatattctgggcacatatatatctgcgatcttaagcggtacaacagtgtgtgtggccatatcacctggtgtatcgaatttgtacatcaatagtgtagtttgtgggccgcaatctcagtttacttttcatgtgttctctttcagcaagatatttgtgtatcaagttttactccacgttcagtcatatatccattataaactctcatattttggaacgacactccagttgctctgtaatgtggtgctcacacagtggatataatgttatttaggtgatgcaatattagataactgaactttgatgttttcctgtgtcaattttttgtccatgttcagttatctacccatcttttgttttaaaacctttagaattatactccctccggccctttttactctgcacattggatttgccgaaagtcaaactttactaagtttaaccaaatttatattaaaaattattagcatctataatatctaataaatataatatgaaaatatattccaagatgaatctaatgatattggtgttgttatgtgagtgtctataattttttatataaacttggtcaaagttggatgagattgacttcgaataaacctaatatgcaaagtaaaaaggaccggagggagtactccagtttgttatgtaatgtgttgtttacactggattttcttttcttcccgattatgctatgttataacctggatgctctctttcagcaatctgtttgtgtgtcaaattttaatcccatgttcagtcatttacccatcttttttctaatattttggaattataaatttgcttcactgcttcccttgagtatgcatcgaaaaagcatgcaacatttctctgaaatagatagatgttcatgccttttattcctgctcatatttctgtttgcttgagggttgctattcttcttgtagactgtcaactttagcatggtgaaaatggaaatgcagtttcacccattaagatgatatgtttccatggcagtctggtgctatactaactaaaatctctgcaggctgttgagaggaaaagtcggctgcgagatgctgcggctcggcagcagctccagtgggatgaagaagactcctcggagcctgaggatccagcacaaacagatgagggtattcatggaagcacagcaccaacaaatgaaaccctgggtgttggctcggctaaagttgttgaaaccaccgcagcatcagcaacctgcttgggcgagggcgagagcaccacgatggtgaaggcatctgatgactgctccttgcagtctccatccagaccaagtggagaaaacgacgtggagaagcaactagccaactccatacaagagattgagaagatggtgagccggaacctgaagcgagctgatcccaaggctgctcggccttccaagaccccggctgtccagaggcgcaccctgatgtctatgcgctga

SEQ ID NO: 64 - DNA, 4203 bp, Triticum aestivum
  source 1..4203: mol_type = genomic DNA
atggctgaagtgaggattgccagcaggacggctgtagccgatcgctctggcggtggattcttcgtgaggagggtggcgtctcccggagccgtggtggacaagggcgccgtcaagccgcttgctcgtcgggtcccgtcaccgtcgagcaacaaggagaacgtgccaccagcgtgggctgcgtggactgcacccaagaggaggagctccctgcccgagtggtacccaaggaccccactccgcgatatcacatcagtcatcaaggtgataaacatttcctcttgtggtgcctagtccagggttatacaggaactattcacgacagtatcatctagcttatgctagttttgtaccttgctgagatttatgcaacatctgacctggtgtatcgaatttgtacatcaacagtgtagtttgtgggccgcaatctcagtttacttttcaggtgttctctttcagcaagatatttgtgtatcaagttttactccacgttcagtcatatatccattataaattatcatattttggaacgatactccagttgctctgtaatgtagtgcttacacaatggatatatactgttatttaggtgatgcaatattagataactgaactttgatgttttcctgtgtcaagttttagtccatgttcagttatctacccatcttttgttttaagtttttaagaattatactccagtttgttctgtaatgtgttgtttacagtggattttcttttcttcccgattatgctacgttataacctggatgctctctttcagcaatgtgtttgtgtgtcaagttttaatctcatgttcagtcatttacccgagttggtcgcgagatcttatgggtttcacctgtagcctaccccaacttgtttgggactaaaggctttgttgttgttgttgttgagtcatttaccattgtttattctaatattttggaattataaatttgctgcttacaatggatataatcatataaacttgctagaacaatcatcagttaatgtagacttgacaacaaattttgcttcactgcttccgttgagtaaaacacatgcaacatttctctgaaatagttagcttttcatgacttttattcgttgttggagttgtgtcgaatatagggtacaaggtaggttacagttggacttgtagttgtattgtatttagataggatatggagtcgtgtcctagtaggacacttgtatcctaggcctctcacatatagcggggtagacacacgatgtaacctatgccaacataatagcaccggaacgcaggggaagccggcggcatgtgccggtgtccagggcgaccgggtgcggtattgtggcggtgtcatggggaggagcgcccatagtcaggcctcggggatgtagccatatcggtgaacctcgttaacgaatctcggtgtcgtgcttgtgtgattgcttgatcctcagatgatcgacggagtgcctcggatttattctaacaagtggtatcatgagctaggttgttcggaggctgtgaattgttgatctagaggagaacacgtggaagttgtcgatgggatctagcgagatgcaacgagtatgattggagacatggcagcagatgttgtcgcgtcggtggatcaaagttgctcttgaagatttggaaacgacggcgtcgtgtttgaaagaagcacaaggcgatgcgcgcgtacgtatggcagtctcggcgatctgtttagggcgacggcggcgtagcagcaggctcggacggcgcgttgcagcacgtggcgtggcagccggcaggaaacccaagcaccgttgtggtgggccatcatggtcttgcgcggagcggcccttgacgaacagtggcctggcgtgcgcatactcgcgtacatgggcgtggtgcagtacggtcaaagcaatttagtaacggacagtgatttgtttggaaacaaaaggagaccgagtccgagcggtagatgtgcaacataaggcagcctaatcagatccaaaggaaatccatcaggaggacaaagcaagctagcattgggattgagtcaggagctacaccacggttagtcgtacaaagaatctggcgccagcgaataccaggtttgaggtgcagaagtttgacagaactgaaaaccttgggttatgacagacagcggtgaaaatttgctgacacatcaaggatgcttgaaggcgttgcgggatgtcaaccagttaagatggatgatgaggctgcgtggagttgtagatggttgcagtcagaaaagtcggctcgggtcagacacatagggcagttggcctggacggtctgacggatcgatgcaagtcggttggtacagaagacggtgcgggatcagtgacggcgacataggagcgtgatgctgatggtgaccgacttctgggcgtggaaacacgtggcgcaggcccgaggcttgtgtggcttcgacaagactatgacgcggggttgattcaaggtgatgtacacacggagcttgaagtcgacggggcgtgagggtggactgatcatctaccatggagtcatgttgaaggtggagctggattgaggggctacggtgtaaggatccagagaatcgaagcctattcagcgggaaaaagcgagtgacacgtagttcggactgaagcccaatggtctgatggaagcgcgaaactcgtcatcggtcggtgttgatcggtggtactctgcaatggggtttgagtggcgtgggttcgcggcccttgagactcgaccgggatagcagaggctcgacgcggtaatagcggcgaggcgtgcggtacgcacgggcatggagacgggccagggctctggtggtcatatgtggtgagacaactgcgaatttgactcggggtgactacaagcaatggtgaaatttcttcaagtttcagacaggcagtcaagaaaggagtggtgatgttgagttcaggtaactcttatgtgtgacatccaatatgtgagttgttcactttcacgcaggtcagtgatcggtgtgtgatggtattgacggatactctggaagttgggagcacaaactagagtaacaaggaacttaattttgctcgagtgttgaccgtggtcaagaaaagaagggactacaagttgcaggtggagtcatacggagtctttggagtagcagcggtactcatggggtaagctcaagtccaatgtacatggaagtttgacgcatagacaaatccaggatggtggagtatattcgccaaggtggagtttgttggagttgtgtcgaatatagtgtacaaggtaggttacagttggacttgtagttgtattgtatttagataggatatggagtcatgtcctagtaggacacttgtatcctaggcctctcatatatagcggggtagacacacgatgtaacctatgccaatataatagtaccggaacgcaggggaagccggcggcatgtgccggtgtccagggcgaccgggtgcagtattgtggcggtgtcatggggaggagcgcccatagtcaggccccggggatgtagccatatcggtgaacctcgttaacaaatctcggtgtcgtgcttgtgtgattgcttgattctcagatgatcgacggagtgcctcggatttattccaacattcgtgctcatagttccgtttgctttaagggttaaaattctgcttgtagactgtctgtcaactttagcatgatgaaaatggaaatgcagtttcacaacctttaagatgatatctttccatggcaattctactcggcctttaagatgatatctatactaactaaaatctgtgcaggctgttgagaggaaaagtcggctgcgagatgctgtggctcggcagcagctccagtgggatgaagaagactcctcggagcctgaggatccagcacaaacagatgagggtattcatggaagcacagcaccaacaaacgaaaccctgggtgttggctcggctcaagttgttgaaaccaccgcagcatcagcaacctgcttgggcgagggcgagagcaccacggtggtgaaggcatctgatgactgctccttgcagtctccatccagaccaagtggagaagacgacgtggagaagcaactggccaactccatacaagagattgagaatatggtgagccggaacctgaagcgaaccgatcccaaggctactcggccttccaagaccccggctgtccagaggcgcaccctgatgtctatgcgctga

SEQ ID NO: 65 - DNA, 1559 bp, Triticum aestivum
  source 1..1559: mol_type = genomic DNA
atggctgaagtgaggattgccagcaggacggctgtagccgatcgctctggcggtggattcttcgtcaggagggtggcgtctcccggagccgtggtggacaagggcgccgtcaagccgcttgctcgtcgggtcccgtcaccgtccagcaacaaggagaacgtgccaccagcgtgggctgcgtgggctgcacccaagaggaggagctccctgcccgagtggtacccaaggaccccactccgtgatatcacatcagtcatcaaggtgataaacatttcctcttgtgggtctgcttcatggatcatgacagttttacattgtatgatcatgtgcattttagctgtgcctagtctatggcgtccaaagttagtattcacaacagtatgatctagcttatgctagttttgtaccttgctgagattcatgcaacatctgacctggtgtatgaatttgtacatcaatagtgtagtttgtgggccgcaatctcagtttacttttcatgtgttctctttcagcaagatatttgtgtatcaagttttactccacgttcagtcatatatccattaataaactctcatattttggaacgacactccagttgctctgtaatgtagtgcttacacaatggatataatgttatttaggtgatgcaattttagataactgaactttgatgttttcctgtgtcaagtttttgtccatgttcagttatctacccatctttttttttaaaccttcagaattatactccagtttgttctgtaatgtgttgtttacactggattttcttttcttcccgattatgctatgttattataacctggatgctctctttcagcaatgtgtttgtgtgtcaagttttaatcccatgttcagtcatttacccatcttttttctaatattttggaattataaatttgcctcactgcttcccttgagtatgcatcgaaaaaaatgcaacatctctgaaatagttagctgttcatgccttttattcctgctcatagttctgtttgcttcagggttgatatctgcttgtagactgtcaactttagcatggtgaaaatggaaatgcagtttcaccctttaagatgatatgtttccatggcagtctggtgctatactaactaagttctctgcaggctgttgagaggaaaagccggctgcgagatgctgcggctcggcagcagctccagtgggatgaagaagactcctcggagcctgaggatccagcacaaacagacgagggtattcatggaagcacagcaccaacaaatgaaaccctgggtgttggctgggctaaagttgttgaaaccaccgcagcatcagcaacctgcttgggcgagggcgagagcaccacggtggtgaaggcatctgatgactgctccttgcagtctacatccagaccaagtggagaagtaggcgtggagaagcaactggccagctccatagatgcgattgagaagatggtgagccggaacctcaagcgaactgatcccgaggctactcggccttccaagaccccggctgtccagaggcgcaccctgatgtctatgcgctga

SEQ ID NO: 66 - DNA, 20 bp, synthetic construct
  source 1..20: mol_type = other DNA
gccccaagccatcgtatctc

SEQ ID NO: 67 - DNA, 20 bp, synthetic construct
  source 1..20: mol_type = other DNA
gtgttcctttacctccattc

SEQ ID NO: 68 - DNA, 20 bp, synthetic construct
  source 1..20: mol_type = other DNA
gagactcaaaattatcgacc

SEQ ID NO: 69 - DNA, 20 bp, synthetic construct
  source 1..20: mol_type = other DNA
gtcgagagtaatatggactg

SEQ ID NO: 70 - DNA, 20 bp, synthetic construct
  source 1..20: mol_type = other DNA
gatgactgatatacatttcc

SEQ ID NO: 71 - DNA, 20 bp, synthetic construct
  source 1..20: mol_type = other DNA
gctctaaaaggcgtcaagtc

SEQ ID NO: 72 - DNA, 20 bp, synthetic construct
  source 1..20: mol_type = other DNA
tgctggatcctcaggctccg

SEQ ID NO: 73 - DNA, 20 bp, synthetic construct
  source 1..20: mol_type = other DNA
gaatccaccgccagagcgat

SEQ ID NO: 74 - DNA, 7658 bp, Triticum aestivum
  source 1..7658: mol_type = genomic DNA
tgcataatttcacaaggcaaagttgtttctcctagatatagcttgcacataagaccaaataacatatagtgtggcaacttgtcacgacatgtccaaagcgtaggcacgtagagttagtcgttaaggctttcatcgctccatgccatacactggtgtcgacactaaacacatagcaatgggttgtctcttaagctgtttttgcatgaggagtgagacactagcacacaaagacattgatgttgatcattctagggttttggaattttggctttgatgcgtcagatttgttccttctcctccacatcaggtatgatcatatgtgacagagtgaagaaccaatttatgccctgtctcacgttcatatacataccctatatattgtcatggtgactggcactaagagaaaaaagatcatgtacaataagaagattgtcgccagatcatactatatgtgtatgttaggcgcacaagagatggatcgatcaaccgatgaagcaagcaattagcatagtggcgatcatgtgggggtggtatccctctggtaccgtgggagactaacccgatcatctaagaggcacacgtgtgcgtgtccagacttgagtagaagaagggcatgcatgcacatgccaatggcctcgcacatgcactttatcgttccaagagaagattgggcggtcgaggtcgacgacggtggactgtgatcagtgtgtgcacgtgagaagcccaacttgatcgctcacacttaaacaacaccctccattttcttcctacatatcatatcatatcatatcattaggcgtacgtacgtgtctaattagtccacaatgcacttagtagatatatacgtacgcttatgaatatacatgtgtagcgtacgtatgcttcatttctgagagaatctcgaggagtactgctaatcgagtagtactgtaccagtaattaactaaccacgcggttaatcagtaaaagatgatgaggctccagtctgaacgctgaacagaataggcaattaaagagacacgcgcgtacacacacctctcgcacgcttgcttgcggtttgaatacgatccagctttgacggtcaaagttttaggcgggggcaagcggtgcaattaccaaggtgaaaacacacacacccggcctatcttaacctccgcttctccttctccccataaagagagagaagaagaagaaagcaacacaggcattgttaatcccaccctctctctctatctctggtactggcgcttgcttctctctctagtcttgttagggagagagagaggttagctgggtgggtcttgttagtactggtaacaagttgtcatgggggatcctatgcgaggtttagggttccgctcgcgcgtttctctatgcgtttctggagaggatcgatgccaaggtttttctatggccggaagacgagggtccgtgtcctcttctagggtgcaggtagcacattatctcctctcctcttcctccccactccccgctcacccatcactctaccggtatgagacaagtgatgattttctttcctttccccatttccagcctctctttgttctgctatactagctatatgtagctagcttatgctttccattgaggcgaaatatgttttgaaattttatatccctactatcaaataataagtatttagtaaactatataagttgttatgtgcgaattacagagcttaatctcatcatgtgtattgtcacttgcgaatcagtattattttcatgagatttttatcggcgatttgtagggggggggggctagtacatccaaatgtagggcgcctgccatgcactgtgcatgccacaccctttgtgtcattgtgtgtgttggtgtgtgtcaggaggagggaggcagagcctagggctttgaaagaaattaaaccttgtcagggattcggcttctgcagcggcgagtggttttagggtttgagagacgaagccggccaagacttaattgttcatacgtggattcatcactgttgaacagtgttttggcgcatagttagcaattcattaacaatgttaccagtactagttatatacacacgcacatatgcatatgtcaactgcccaactgcgtgcatatatgtccagggcacccaaccgtgcgcctgcatgtgcacctccgcaattgaaaaagagaggagatactatgtacgcaaagaacatctgctcgcgatcgaattgagatcctagacaaatagaatgaagctagatcttgcgacatttcttaattttttcaagggggtgatcagttttctaatcacctgtgaataccagctttccagtcaaaaaagggcatgacaactcatactgttatcgagtaaagggcgccgctttagtagtacatgtacatgcaggcaacagggtgcactcatgtaggcatatatcttgacatgtttttgggggaaatgagacaaaatatggtggactcgatctcccaccagaaagcacaagcaaaaagatgtccctgctcagttaaaccatgccactgttttttgtattgcacaaagatactaaaacatagtacccttttttatgtgtggaaaaagcttcctactacgcagagtacatttcactaattaatcaaggagtagcctctctgtagaattaatatcatacatgcattccccgcaaaaaaaaaatcatacgtgcataaccatccacagtagtctcacactccgacataggagtgttaatggcgttaaggattagtactataatcatttgtataaatatctgccccagtactagtacttactacttgtactactagtagcacagttgctcaagtctccaaactccaaagctctcttgggtcttggcctctccagcttctcctaaccttgggctcccagcaacaatggcttcttgatctgctgctctccctttcccggcctatccaacagcaagcttaagtagagaccagttaatcacttccacgtactgtgttgcttttaggtagcttcgaccatggccaccgtgaacaactggttgggcttctccctctccccgcaggagctccctccctccgccgccgcctcaggtgacgtctccggcgccgacgtctgctacaacatcccacaaggtagatacatacacatcgtagtattcttctgattggtttaagtgcatcaagcagttgtatgacaatcttgtgattggtttgtttcgcagattggaacatgaggggatcagagctatctgccctggtggcggagcctaagctggaggacttcctcggcggcatatcctactccgaccaccaccaccaccacaagcaggccggcggcaacaacatggtcgtccctgcaggtagcggcagcggcggcgccgcatgctacacgacctcgggcggcagtagcgtgggctacctgtaccaccccagctcggcctccctccagtttgccgactccgtcatggtggcttcctccggcggtggcgtccaccacgacggcgctggcatcatggcgaacaccacagccaacggtgaccttaataatggcagcggcgggggtggcatcgggctgtccatgatcaagagctggctgcggagccagccatccccggcacaaccaccggagcagcagagggcggatgcggcggcacagggtctctcgctgtccatgaacatggcggcgtgcatgccgccgctcgttggcggggagcgtggtgtgccggagttggcgattgtccggaaggatgacacggccggtggcagcagcgctggttctggcgccgtggtgtcggccggtggtgcggatagcactggcggaagcagtggcgtggtggtggagacgccggcgtccaggaagacggctgacacgtttgggcagcggacctcgatttaccgtggtgtgaccaggtatgtagtatacgtactatagtgcagtgcaaccattcatgaatgtatttgaacataatatatatgaaaagtggaagatttgattgttttttgtgtcattatactgagcatccaggtaatgtatacagtttcggccatttttgttttctagctaataatcaactcaaacactgatattgctaagcatggatctagctaatgtggtctctttcctttttttgtttttacgatatatgatgctagtatcgttttcatatttttctcttcttgacttctttgcatcaaatgagtgccatttgcttttgaggataaattaataatgtagatgcatgggaaatgtggaatgccgttatgtgggcatgattatgataggcttacaaagtgtgattttttgtttttattattggctttaataggcatagatggactgggagatatgaggctcacttatgggacaacagttgcagaagagaagggcaaacacgcaagggtcgtcaaggtactgatgtgccatctttttacatatatgcttacatatatatgatgaaccccctccaatgttgctaattaaaattactctttgattgtttatccaactttgtttgaactcggcctttcgactccatacagtctatctaggtaagcaagatccatattgaagtaattacatttgttttaatcatattttgtagatgcattcattgctatcagttctttaagctactttttttctatgccttctaatctttaaatgcatacacaggtggctatgacaaagaggagaaagctgcaagagcttatgatttggctgctctcaaatattggggtgcgacgacgacgacaaatttcccagtaagcatcacactcatctttttgtcattgttttggaagaagccatattgtcatttatcaccatttgatcatttaaccgtcaaaaaaaaccatttgatcattcgttgatcaatgtaatggtgaaccacatagctgcatatatatctctaattattctattttgggaagcctagcttactgaagttaaagacatgcatctgtttctctttctttttctaggtaaacaactacgaaaaagagctagaggagatgaagcacatgacaaggcaggagtttgtggcgtctttgcgaaggttggtctcttatcttttatatatggtgaatgtaggtacatcgctagcaaatcaatcttcaaatcaactaatgctaatgttgtttaatgtgtatatgtgcaggaagagcagtggtttctccagaggcgcatccatttatcgtggagtaactaggtatgtgtttccttttcttggttgtttgcatcgtatcgtgtagaggttgagaggacaatacttacattagctacataaaaatttgattattgctgctgatccatttgaattttgtataataaaggcaccaccaacatgggagatggcaagcgaggatagggagagttgcagggaacaaggatctctacttaggcaccttcagtaagtatttatactcaacatctgtccacgtccagtacaaaatatatacatttcgttaaactgaaattaccacctcaacatcagagacatgagagcatatgcctttgcattgtgtttgtcaaaaaaaaacagctagatacattttgtgctgacatttgttataaacctctagtgcaaactagttatgatctttatacagatggtactcgttgattttccgaattctgattccaaaatatatatactttttttgaggaaaaaaagatacatttggttacactcaaattaccaccttgactatcagagccattagagtgtatgcgtttgtactgtgtttcttaaaaaaaacagctagattcattttctgctcgcatttattgataatcctcctccagtgcagactagttatgatctctatacatatggtactcgttgattttccaaaatttaaaagataaacatttggttgccatgaaattacaacctcggctttcagagacattagagcgtatgcgtttgtactgtgtgtttctcaaaaaaacaaatacagttagatgcattttatgctgacatttatatcctccagtgcaaactagttatgagctctatacggacggtactcgttcattttccaaattccaactgatgtgtccataaaatgccaatgaatggatcgacaaaatggtagatttcccgttgaaaatggacttgcaagtcatgtcaagttgcatcgagcatattactattatactttgtgcatgagtgcatgctttattaagcgtagcttccatgtttagtgctgccgctgtctccccctgccggtgcatctagtcaaaatattcgagctgcgtctgcatgcatgcatgcatttgcagtaaactaatgatcaactaaaataattaagctagcctgtcacaccttgcatgcatgcatggcgtcgagtggtagattgcgttggcacctaactatggtcgtccatgtgtaatgtaggcacgcaggaggaggctgcggaggcgtacgacatcgcggcgatcaagttccgggggctcaatgctgtcacaaacttcgacatgacgcgctacgatgtcaagagcatcctcgacagcaccgcgctccccatcggtagcgctgccaagcgcctcaaggacgccgaggcggctacggcgtcgtccctcgcgctgcagcagcagcagcacgcaggcgtggtgagcggctatgacatgggcgccctggccgcctacggtgccgcataccaccaccatcacccctctgtcgccggcgctgttgcgtggccgaccatcgcgttccaggcgccgcctcatcaggtttccagcggcggtggtggacacatgtaccacccctacgcccatgcgcagccgctgcgcgggtggtgtaagcaggagccggaccacgcggtgatcgcggctgcgcacagcctgcaggagctccaccacctcaacctcggcgccggtggcggcgcacatgacttcttctcgcagcatgcccaggcgatgcagcagcagcacggtggcctcggcagcatccacaacgcggccggcgcctcgctcgagcacagcaccggctccaactccgtcgtgtacaacaacggcggcgtcgagagctacatgcttcccatgagcaccaccaccaccgcgacggcgagccacgcccgcgagcaggtggcagcgcatgcccacgcacgcgcggtggcggcacaggcatacagcggcggcaacatgagcagggatcacgacgacgggaagatcgcctacgagaactacctcgtgagcaccgcagaagcctacggcggggggaggatggctgcctgggcacaggcctcagctccaccggcgacaagcagcagcgacatgaccggcgcgcagctcttcagcgtctggaacgactctaattaggtcagctagagagagaaacactaacatcacatagcacttagggttcatgcatgatatggccattaaggtgttactgtcgtgttcaattattgttactcggtagctagttgggacggtgttcttctggaggaaggtaatggcctgtgagggggctatgcattgcagtgcattgttcttggatctgctgagctgttatcattttggggttaagttcagaagaacatatatgggggcatattgactagggaattagatggtttagaaatcaatggaggtaaaactagcctgcttgtaaattttgaattgctagggtggatggcactgcatgatggcgatcaatgagcaaacgatttttttt

SEQ ID NO: 75 - DNA, 4304 bp, Triticum aestivum
  source 1..4304: mol_type = genomic DNA
atggccaccgtgaacaactggttgggcttctccctctccccgcaggagctccctccctccgccgccgcctcaggtgacgtctccggcgccgacgtctgctacaacatcccacaaggtagatacatacacatcgtagtattcttctgattggtttaagtgcatcaagcagttgtatgacaatcttgtgattggtttgtttcgcagattggaacatgaggggatcagagctatctgccctggtggcggagcctaagctggaggacttcctcggcggcatatcctactccgaccaccaccaccaccacaagcaggccggcggcaacaacatggtcgtccctgcaggtagcggcagcggcggcgccgcatgctacacgacctcgggcggcagtagcgtgggctacctgtaccaccccagctcggcctccctccagtttgccgactccgtcatggtggcttcctccggcggtggcgtccaccacgacggcgctggcatcatggcgaacaccacagccaacggtgaccttaataatggcagcggcgggggtggcatcgggctgtccatgatcaagagctggctgcggagccagccatccccggcacaaccaccggagcagcagagggcggatgcggcggcacagggtctctcgctgtccatgaacatggcggcgtgcatgccgccgctcgttggcggggagcgtggtgtgccggagttggcgattgtccggaaggatgacacggccggtggcagcagcgctggttctggcgccgtggtgtcggccggtggtgcggatagcactggcggaagcagtggcgtggtggtggagacgccggcgtccaggaagacggctgacacgtttgggcagcggacctcgatttaccgtggtgtgaccaggtatgtagtatacgtactatagtgcagtgcaaccattcatgaatgtatttgaacataatatatatgaaaagtggaagatttgattgttttttgtgtcattatactgagcatccaggtaatgtatacagtttcggccatttttgttttctagctaataatcaactcaaacactgatattgctaagcatggatctagctaatgtggtctctttcctttttttgtttttacgatatatgatgctagtatcgttttcatatttttctcttcttgacttctttgcatcaaatgagtgccatttgcttttgaggataaattaataatgtagatgcatgggaaatgtggaatgccgttatgtgggcatgattatgataggcttacaaagtgtgattttttgtttttattattggctttaataggcatagatggactgggagatatgaggctcacttatgggacaacagttgcagaagagaagggcaaacacgcaagggtcgtcaaggtactgatgtgccatctttttacatatatgcttacatatatatgatgaaccccctccaatgttgctaattaaaattactctttgattgtttatccaactttgtttgaactcggcctttcgactccatacagtctatctaggtaagcaagatccatattgaagtaattacatttgttttaatcatattttgtagatgcattcattgctatcagttctttaagctactttttttctatgccttctaatctttaaatgcatacacaggtggctatgacaaagaggagaaagctgcaagagcttatgatttggctgctctcaaatattggggtgcgacgacgacgacaaatttcccagtaagcatcacactcatctttttgtcattgttttggaagaagccatattgtcatttatcaccatttgatcatttaaccgtcaaaaaaaaccatttgatcattcgttgatcaatgtaatggtgaaccacatagctgcatatatatctctaattattctattttgggaagcctagcttactgaagttaaagacatgcatctgtttctctttctttttctaggtaaacaactacgaaaaagagctagaggagatgaagcacatgacaaggcaggagtttgtggcgtctttgcgaaggttggtctcttatcttttatatatggtgaatgtaggtacatcgctagcaaatcaatcttcaaatcaactaatgctaatgttgtttaatgtgtatatgtgcaggaagagcagtggtttctccagaggcgcatccatttatcgtggagtaactaggtatgtgtttccttttcttggttgtttgcatcgtatcgtgtagaggttgagaggacaatacttacattagctacataaaaatttgattattgctgctgatccatttgaattttgtataataaaggcaccaccaacatgggagatggcaagcgaggatagggagagttgcagggaacaaggatctctacttaggcaccttcagtaagtatttatactcaacatctgtccacgtccagtacaaaatatatacatttcgttaaactgaaattaccacctcaacatcagagacatgagagcatatgcctttgcattgtgtttgtcaaaaaaaaacagctagatacattttgtgctgacatttgttataaacctctagtgcaaactagttatgatctttatacagatggtactcgttgattttccgaattctgattccaaaatatatatactttttttgaggaaaaaaagatacatttggttacactcaaattaccaccttgactatcagagccattagagtgtatgcgtttgtactgtgtttcttaaaaaaaacagctagattcattttctgctcgcatttattgataatcctcctccagtgcagactagttatgatctctatacatatggtactcgttgattttccaaaatttaaaagataaacatttggttgccatgaaattacaacctcggctttcagagacattagagcgtatgcgtttgtactgtgtgtttctcaaaaaaacaaatacagttagatgcattttatgctgacatttatatcctccagtgcaaactagttatgagctctatacggacggtactcgttcattttccaaattccaactgatgtgtccataaaatgccaatgaatggatcgacaaaatggtagatttcccgttgaaaatggacttgcaagtcatgtcaagttgcatcgagcatattactattatactttgtgcatgagtgcatgctttattaagcgtagcttccatgtttagtgctgccgctgtctccccctgccggtgcatctagtcaaaatattcgagctgcgtctgcatgcatgcatgcatttgcagtaaactaatgatcaactaaaataattaagctagcctgtcacaccttgcatgcatgcatggcgtcgagtggtagattgcgttggcacctaactatggtcgtccatgtgtaatgtaggcacgcaggaggaggctgcggaggcgtacgacatcgcggcgatcaagttccgggggctcaatgctgtcacaaacttcgacatgacgcgctacgatgtcaagagcatcctcgacagcaccgcgctccccatcggtagcgctgccaagcgcctcaaggacgccgaggcggctacggcgtcgtccctcgcgctgcagcagcagcagcacgcaggcgtggtgagcggctatgacatgggcgccctggccgcctacggtgccgcataccaccaccatcacccctctgtcgccggcgctgttgcgtggccgaccatcgcgttccaggcgccgcctcatcaggtttccagcggcggtggtggacacatgtaccacccctacgcccatgcgcagccgctgcgcgggtggtgtaagcaggagccggaccacgcggtgatcgcggctgcgcacagcctgcaggagctccaccacctcaacctcggcgccggtggcggcgcacatgacttcttctcgcagcatgcccaggcgatgcagcagcagcacggtggcctcggcagcatccacaacgcggccggcgcctcgctcgagcacagcaccggctccaactccgtcgtgtacaacaacggcggcgtcgagagctacatgcttcccatgagcaccaccaccaccgcgacggcgagccacgcccgcgagcaggtggcagcgcatgcccacgcacgcgcggtggcggcacaggcatacagcggcggcaacatgagcagggatcacgacgacgggaagatcgcctacgagaactacctcgtgagcaccgcagaagcctacggcggggggaggatggctgcctgggcacaggcctcagctccaccggcgacaagcagcagcgacatgaccggcgcgcagctcttcagcgtctggaacgactctaattag

SEQ ID NO: 76 - DNA, 8398 bp, Triticum aestivum
  source 1..8398: mol_type = genomic DNA
gtcacacaattctaagagcttaagctccgtctcattgctttcgtatgtgtgatatcgcaagtgcaccaacaaaccactacaatgacatgttgagaacacccccaccttctgacaatatatatgcttaaggaccacatgtatggagaagggagaagtccctaaatgaagaagctacatgttttgacaaatgattgatgtacgtagccactacaatgtaaaaaccgaatgacatgggttggaaaacgggagcaaattttgtgtaagttgtctgaaataaccagatgaacatgtgtaagttcttaatcattcccaaaaaaaagacgggatacatattggtggatatagtatgaacataaaaatttgatcaaacaatatatgattgtgttatgtaagaaaaaaaagggaaggaagagtgccataagcactgaatttgttctacgaacacattattggacgaaactttgcaggttcaaaattcatagatgcacccatggattattttagaatttgaattttttgaaatgaggggactgaaggaggagcatacacacgtatgcaatactctactgcttaagcaccttcacaaccaacattggaatgtgatgtgacctggtattgcaatttaagaggattagacatgcaaatttatattcttctgcacatgacctgacacctagctgacatatatggttcatcatactgaagtggactctctgcttggcccctatatggttatgcacatcaatataatgagtgaaaacaacaaaaatctcaaggaattcatgcataattccataaggcaaagttgtttctcctagatatagcttgcacataagatcaaataacatatagtctggcaacttgtcacggcgtgaccaaagggtaggcacgtagagttagtcgttgacgctttcattgctccatatgccatctactagcgttgacactaaacacatagcaatgggttgtctcttaagctgttttcccatgaggtgtgagacactagcacacaagagatattgatgttgatcactctagggttttcgggattttggctttgatgcgtcatatttgttccttctcctcatccacatcaagtatgatcatatgtgacagagtgaagaacaaatttatgtcctgtcccatgttcatatacataccctatatatagtcagggtgactggctctaagagaaaaaagatcatgtacaataagaagattgtcactagatcatactatatgtgtatgttgggcacaagagatggatcgatcaaccgatgaagcaagcaattagcataatggcgatcacgtgggggtggtatccctctggtaccgtgggagactaacccgatcatccaagaggcacacgtgtgcgtgtccagacttgagtagaagaagagcatgcatgcacatgccaatggcctcgcacatgcactttatcgttccaagagaagattgggcggtcgaggtcgacgacggtggactgtgatcagtgtgtgcacgtgagaagcccaacttgatcgtcgctcacagttaacaagaccctccattttcttcctaaatcatatcatatcattatcatcaggcgtacctccatgtctaattagtctgcaatgaatgtcctcgctagctagctacgctgattgtatatacatctgtatttttcacagaaagaagattatacatgtgtacgagtacgtatatatactgcatttctgagagaatctcgaggagtacgtctccaccttatgatagcatattcacactactgatcgagtagtactgtaccagtaattaactaaccacgcggttaatcagtaaaagatgatgaggttccagtctgaacgctgagcagaataggcaattaaagagacacgcgcgtacatacctctcgcacgcttgcttgcttccagtttgaatacgatccagctttgacggtcaaagttttaggcgggggcaaggggtgcaattaccaaggtgaaaacacacacacccggcctatcttaacctccgcttctccttctctccataaagagagagaagaagaagaaagcaacacaggcattgttaatccctccctctctctctctctagtactggcgcttgcttctctctctagtcttgttagcgggagagagggagagagattagctgggtaggtcttgttagtagtactggtaacaagttgtcatgggggatcctatgcgaggtttagggttccgctcgcgcgtttctctatgcgtttctggagaggatcgacgccaaggtttttctatggccggaagacgggggtccgtgtcctcttctagggtgcaggtagcacattatctcctctcctcttcctccccactccccgctcacccatcactctaccggtatgagacaagtgatgattttctttcccccttttccagcctctctttgttctgctactagctatatatatgtagctagcttatgctttccactgaggcgaaatgaatccatggctagcaccgcaagcaaaattatattttgcaatgctatattcctactatcaaataataacaacagtaatttgtttactatttaagttgttatgtgcgaatcacagagtttaatctcatcatgagtattgtcacttgcaaatttgcagtgccattagtattatttttcatgagagtttatcggcaattttcgtcttaagttgttgaaatattgtatagtagctgttgtaggggagagtactagtacatccaaatgtggggcgcctgtcactgtgcgtgcatgccaccctttgtgccattgtgtgtgtgggtgtgtgtcaggaggagggaggcagagcctagggctttgaaagaaataaaccttgtcagggattcggcttctgcagcggcgagtggttttagggtttgagagacgaagccggccaagacttaatcgtccatagttagcaattaattaacaatgttaccggtgctagttatatatacacgcacatatgcatatgtcaactgccgaactgcgtgcatatatatccagggcacccaaccgtgcgcctgcatgtgcacctccgcaattgaaaaagagaggagatactatgtacgcaaagaacatctgctcgcgatcgaattgagatcctagacaaatagaatgaagctagatcttgcaacatttcttaatttttccaagggagtgttagttttctaatcacctgtgaataccagctttccagtcaaaaaagggcatgacaactcatactgttatcgagtaaagggcgccgctttagtactacatgtacatgcaggcaacagggtgcactcatgtaagcatatcttgacatggtttttggggaaaatgagacaaaatatggtggactcgatctccccccagaaagcacaagcaaaaagatgtccctgctcagttaaaccatgccactgttttttgtattgcacaaagatacaaaaatatagtacccttttttatgtgtggaaaaagcttcctactactactactagcacagttcctgtagaattaatatcatacatgcataaccatccacagtagtctcacactctgacatagtaggagtgttaatggcgttaaggattagtgtactataataatttgtataaatatctgccccagtagtacttgtctactactaccagtagcacagttcctcaagtctccaaactccaaatctctcttgggtcttggcctctccagcttctcctaaccttgggctcccagcaacaatggcttcttgatctgctgccctccctttcccggcctatccagcagcaagcttaagtagagaccagttaatcacttccacgtactgtgttgcttttaggtagcttcgagcatggccaccgtgaacaactggttgggcttctccctctccccgcaggagctccctccctccgccgccgcctcaggtgacgtctccggcgccgacgtctgctacaacatcccgcaaggtagatacatacacatcgtagtattcttctgattggtttaagtgcatcaagcagttgtatgaaaatcttgtgattggtttgtttcgcagattggaacatgaggggatcggagctatcagcgctggtggcggagccgaagctggaggacttccttggcggcatatcttattccgaccatcaccaccaccagcacaagcagcctggcggcaacaacatggtcgtccctgcaggcagcgccagtggcggtgccgcttgctacgcgaactctggcggcagtagcgtgggctacctgtaccaccccagctcggcctccctccagtttgccgactccgtcatggtggcttcctccggcggtggcgtccaccacgacggcgctggcatcatggcgaacaccacagccaacggtgaccttaatggcggcggcgggggtggcatcgggctgtccatgatcaagagctggctgcggagccagccatccccggcacaagcacaaccaccggagcaacagagggcagaggcggcggcgcaggggctctcgctgtccatgaacatggcggcgtgcataccgccgcttgttggcggggagcgtggcgtgccggagttggcgattgtacggaaggatgacacggctggtggcagcagtgctggttctggcgccgtagtgtcggccggtgcggacagcactggcggaagcagtggcgcggtggtcgagactccagcgtcgaggaagacagccgacacgtttgggcagcggacttcgatttatcgtggcgtgaccaggtatatagtagtagtgtagtactagtgcattgcaaatttgcaataaacatgaaagtatctgaacatactctctatttttcaaaatggaatatttgattgcgttttgtgtcattatactgagcatccagctaatgcatagtttcaggcatttttgttttctcgctaatcatcaacttggacactgatattgctaattaagcatggatcaatctagtttctttcctttttttctgtttttccgatgacgctagtatcactttttgtatttttctcttattttcttccttgaatcaagtgagtgttatttgcttttgaggctcttcttgaataatgtagatgcataggaaatgtggaatgccattatgtgggcatgattgtggtaggcttacaacgtgtgattcttttttgcgattggctttaataggcatagatggaccgggagatatgaggctcacttgtgggacaacagttgcagaagagaagggcaaactcgcaagggtcgtcaaggtactgatgtgttatctttaaatctgcttacatacatatatatgatcaattccctccaatgttgctaactaaagttactctttgattgattattcaactttgtttgaacttggccttttcgactccacacagtctatctaggtaagcaagatccagattaaagtaattacatttgtttttgtcacattttgttgatgcattcattgctatcaattctttaaactacattttttgtacatgttctaatattgaaatacatatataggtggctatgacaaagaggagaaagctgcaagagcttatgatttggctgctctcaagtattggggtgccacgacgacgacaaatttcccagtaagcattacgctagctgctattgttgtcatgcttgtggaagaagtcatttttctttattgctgccaatgtaaactattataagatttttagatctttgaaatcactaaagtagtgatttaaaagatcttatattagttcacagagggagtaccattttattattcgttgatcaatgtaatggtgaaccacatagccacatctctctaattattatcttttgggaagcctagcttactgaagttgtacttaaagtcatgcatattttctccattttttctaggtaaacaactatgaaaaagagctagacgagatgaagcacatgacaaggcaggagtttgtggcatctctgcgaaggtcggtgtcttatcttttatatataaacgtaagtacgtggttagcaaatcaactaatgctaattttatttgatgtgtgtatgtgcagaaagagcagtggtttctccagaggtgcatccatttatcgtggagtaactaggtatgcacgcgtttccttttcttggttgcgtgcatcatatggtgcagagatcggcaagatagacctaacattagctacataaaatctgattattgatgctgatccatttgaattttgtataataaaggcaccaccaacatgggagatggcaagcgaggatagggagagttgcagggaacaaggacctctacttgggcaccttcagtaagtatagatactcactatcaattacactgaatacgtttggttacactgaaattaccacctcaactatcagagacattagagcatatatgtttgtactatgcttcttaaaaaagacagccgggtacatttattaataatcctccattgcgaactactccctccgttcctaaatatttgtctttctagagatttcaacaagtgactacatacaaagcaaaatgagttaatctacattctaaaatatgtctacatacattcatatgttgtagtccatttgaaatgtccagaaagacaaatatttaggaacggagggagtagttataatctcgttgattttccatattcaaatctatgttcagctgacgtgtgggtccaaggccaacccatacatgccatgcgagaaatggagtgacaaaccataaatgcctatgaatggcgcgacaaaatcgtagatttcatgttgaaaatggacatgcaagtcatgccaagttgcatcgagcatattactattgtactttgtgcatgagtgcacatgctttattaaacgtagcttccatgtttagtgctgccgctgtctccaatcaccggcgcatctagtcaaaatattcgagctgcgtctgcatgcatgcatgcatttgcagtaaactaatgatcaattaaaataattaagcttaccctaaaaaaattaaaataattaagctagcttgtcacaccttgcatgcatgcatggcgtctactggtagattgcgttgttggtccctaaccatgggcatcgcctgtgtaatgtaggcacgcaggaggaggctgccgaggcgtacgacatcgcggcgatcaagttcaggggccttaacgctgtcaccaacttcgatatgacgcgctacgacgtcaagagcatcctcgacagcaccgcgctccccatcggcagcgccgccaagcgcctcaaggacaaagaggcagccacggcgtcgtccctcgcgctgcagcagcagcagcacgcaggcgtggtgagcggctacgacatgggcgccctggccgcctacggtgcggcgtaccaccaccatcacccctccgtcgccggcgctgttgcgtggccgaccatcgcgttccagacgccgcctcagcaggtttccggcggcggtggtggacacatgtaccacccctacgcccatgcgcagccgctgcgcgggtggtgcaagcaggagccggaccacgcggtgatcgcggccgcgcacagcctgcaggagctccaccacctcaacctcggcgccggtggcggcgcacacgacttcttctcgcagcatgcccaggcgatgcagcagcagcatggcggccttggcagcatcgacaacggggccggcgcctcgctggagcacagcactggctccaactccgtagtgtacaacaacggcgcggtcgagagctatatgctgccaatgagcaccaccaccactgcggcggtgagccacgcccacgagcaggcggcagcgcatgcccacgcacgggcggtggcggcacaggcgtatagcggcggcaacatgagcagggatcacgacgacgggaagatcgcctacgagaactacctcgtcagcaccgcagaagcctacggtggggggaggatggctgcgtgggcgccggcctcagctccgccggcgacaagcagcagcgacatgaccggcacgcagctcttcagcgtctggaacgacactaattaggtcagctagagagaaacactaacatcacaaagcacttagggtgttcatgcatgagatggccattaaggtgttactgtcgtgttcaattattgttactcggtagctagctagttgggatggtgttcttctggaggaaggtaatggcctgtgagagggctatgcattgcactgcattgttcttggatcagttgagctgttatcattttggggttaagttcagaagaacatgtatgggggcattgactagggaattagatggtttagaaatcaatggagctaaaactagcctgcttgtaaattttgaattgctagggtagatggcactgcattgagggcgatcaatgcgcaaatgagttctttttttaacaaaattcttaactt

SEQ ID NO: 77 - DNA, 5827 bp, Triticum aestivum
  source 1..5827: mol_type = genomic DNA
atgggggatcctatgcgaggtttagggttccgctcgcgcgtttctctatgcgtttctggagaggatcgacgccaaggtttttctatggccggaagacgggggtccgtgtcctcttctagggtgcaggtagcacattatctcctctcctcttcctccccactccccgctcacccatcactctaccggtatgagacaagtgatgattttctttcccccttttccagcctctctttgttctgctactagctatatatatgtagctagcttatgctttccactgaggcgaaatgaatccatggctagcaccgcaagcaaaattatattttgcaatgctatattcctactatcaaataataacaacagtaatttgtttactatttaagttgttatgtgcgaatcacagagtttaatctcatcatgagtattgtcacttgcaaatttgcagtgccattagtattatttttcatgagagtttatcggcaattttcgtcttaagttgttgaaatattgtatagtagctgttgtaggggagagtactagtacatccaaatgtggggcgcctgtcactgtgcgtgcatgccaccctttgtgccattgtgtgtgtgggtgtgtgtcaggaggagggaggcagagcctagggctttgaaagaaataaaccttgtcagggattcggcttctgcagcggcgagtggttttagggtttgagagacgaagccggccaagacttaatcgtccatagttagcaattaattaacaatgttaccggtgctagttatatatacacgcacatatgcatatgtcaactgccgaactgcgtgcatatatatccagggcacccaaccgtgcgcctgcatgtgcacctccgcaattgaaaaagagaggagatactatgtacgcaaagaacatctgctcgcgatcgaattgagatcctagacaaatagaatgaagctagatcttgcaacatttcttaatttttccaagggagtgttagttttctaatcacctgtgaataccagctttccagtcaaaaaagggcatgacaactcatactgttatcgagtaaagggcgccgctttagtactacatgtacatgcaggcaacagggtgcactcatgtaagcatatcttgacatggtttttggggaaaatgagacaaaatatggtggactcgatctccccccagaaagcacaagcaaaaagatgtccctgctcagttaaaccatgccactgttttttgtattgcacaaagatacaaaaatatagtacccttttttatgtgtggaaaaagcttcctactactactactagcacagttcctgtagaattaatatcatacatgcataaccatccacagtagtctcacactctgacatagtaggagtgttaatggcgttaaggattagtgtactataataatttgtataaatatctgccccagtagtacttgtctactactaccagtagcacagttcctcaagtctccaaactccaaatctctcttgggtcttggcctctccagcttctcctaaccttgggctcccagcaacaatggcttcttgatctgctgccctccctttcccggcctatccagcagcaagcttaagtagagaccagttaatcacttccacgtactgtgttgcttttaggtagcttcgagcatggccaccgtgaacaactggttgggcttctccctctccccgcaggagctccctccctccgccgccgcctcaggtgacgtctccggcgccgacgtctgctacaacatcccgcaaggtagatacatacacatcgtagtattcttctgattggtttaagtgcatcaagcagttgtatgaaaatcttgtgattggtttgtttcgcagattggaacatgaggggatcggagctatcagcgctggtggcggagccgaagctggaggacttccttggcggcatatcttattccgaccatcaccaccaccagcacaagcagcctggcggcaacaacatggtcgtccctgcaggcagcgccagtggcggtgccgcttgctacgcgaactctggcggcagtagcgtgggctacctgtaccaccccagctcggcctccctccagtttgccgactccgtcatggtggcttcctccggcggtggcgtccaccacgacggcgctggcatcatggcgaacaccacagccaacggtgaccttaatggcggcggcgggggtggcatcgggctgtccatgatcaagagctggctgcggagccagccatccccggcacaagcacaaccaccggagcaacagagggcagaggcggcggcgcaggggctctcgctgtccatgaacatggcggcgtgcataccgccgcttgttggcggggagcgtggcgtgccggagttggcgattgtacggaaggatgacacggctggtggcagcagtgctggttctggcgccgtagtgtcggccggtgcggacagcactggcggaagcagtggcgcggtggtcgagactccagcgtcgaggaagacagccgacacgtttgggcagcggacttcgatttatcgtggcgtgaccaggtatatagtagtagtgtagtactagtgcattgcaaatttgcaataaacatgaaagtatctgaacatactctctatttttcaaaatggaatatttgattgcgttttgtgtcattatactgagcatccagctaatgcatagtttcaggcatttttgttttctcgctaatcatcaacttggacactgatattgctaattaagcatggatcaatctagtttctttcctttttttctgtttttccgatgacgctagtatcactttttgtatttttctcttattttcttccttgaatcaagtgagtgttatttgcttttgaggctcttcttgaataatgtagatgcataggaaatgtggaatgccattatgtgggcatgattgtggtaggcttacaacgtgtgattcttttttgcgattggctttaataggcatagatggaccgggagatatgaggctcacttgtgggacaacagttgcagaagagaagggcaaactcgcaagggtcgtcaaggtactgatgtgttatctttaaatctgcttacatacatatatatgatcaattccctccaatgttgctaactaaagttactctttgattgattattcaactttgtttgaacttggccttttcgactccacacagtctatctaggtaagcaagatccagattaaagtaattacatttgtttttgtcacattttgttgatgcattcattgctatcaattctttaaactacattttttgtacatgttctaatattgaaatacatatataggtggctatgacaaagaggagaaagctgcaagagcttatgatttggctgctctcaagtattggggtgccacgacgacgacaaatttcccagtaagcattacgctagctgctattgttgtcatgcttgtggaagaagtcatttttctttattgctgccaatgtaaactattataagatttttagatctttgaaatcactaaagtagtgatttaaaagatcttatattagttcacagagggagtaccattttattattcgttgatcaatgtaatggtgaaccacatagccacatctctctaattattatcttttgggaagcctagcttactgaagttgtacttaaagtcatgcatattttctccattttttctaggtaaacaactatgaaaaagagctagacgagatgaagcacatgacaaggcaggagtttgtggcatctctgcgaaggtcggtgtcttatcttttatatataaacgtaagtacgtggttagcaaatcaactaatgctaattttatttgatgtgtgtatgtgcagaaagagcagtggtttctccagaggtgcatccatttatcgtggagtaactaggtatgcacgcgtttccttttcttggttgcgtgcatcatatggtgcagagatcggcaagatagacctaacattagctacataaaatctgattattgatgctgatccatttgaattttgtataataaaggcaccaccaacatgggagatggcaagcgaggatagggagagttgcagggaacaaggacctctacttgggcaccttcagtaagtatagatactcactatcaattacactgaatacgtttggttacactgaaattaccacctcaactatcagagacattagagcatatatgtttgtactatgcttcttaaaaaagacagccgggtacatttattaataatcctccattgcgaactactccctccgttcctaaatatttgtctttctagagatttcaacaagtgactacatacaaagcaaaatgagttaatctacattctaaaatatgtctacatacattcatatgttgtagtccatttgaaatgtccagaaagacaaatatttaggaacggagggagtagttataatctcgttgattttccatattcaaatctatgttcagctgacgtgtgggtccaaggccaacccatacatgccatgcgagaaatggagtgacaaaccataaatgcctatgaatggcgcgacaaaatcgtagatttcatgttgaaaatggacatgcaagtcatgccaagttgcatcgagcatattactattgtactttgtgcatgagtgcacatgctttattaaacgtagcttccatgtttagtgctgccgctgtctccaatcaccggcgcatctagtcaaaatattcgagctgcgtctgcatgcatgcatgcatttgcagtaaactaatgatcaattaaaataattaagcttaccctaaaaaaattaaaataattaagctagcttgtcacaccttgcatgcatgcatggcgtctactggtagattgcgttgttggtccctaaccatgggcatcgcctgtgtaatgtaggcacgcaggaggaggctgccgaggcgtacgacatcgcggcgatcaagttcaggggccttaacgctgtcaccaacttcgatatgacgcgctacgacgtcaagagcatcctcgacagcaccgcgctccccatcggcagcgccgccaagcgcctcaaggacaaagaggcagccacggcgtcgtccctcgcgctgcagcagcagcagcacgcaggcgtggtgagcggctacgacatgggcgccctggccgcctacggtgcggcgtaccaccaccatcacccctccgtcgccggcgctgttgcgtggccgaccatcgcgttccagacgccgcctcagcaggtttccggcggcggtggtggacacatgtaccacccctacgcccatgcgcagccgctgcgcgggtggtgcaagcaggagccggaccacgcggtgatcgcggccgcgcacagcctgcaggagctccaccacctcaacctcggcgccggtggcggcgcacacgacttcttctcgcagcatgcccaggcgatgcagcagcagcatggcggccttggcagcatcgacaacggggccggcgcctcgctggagcacagcactggctccaactccgtagtgtacaacaacggcgcggtcgagagctatatgctgccaatgagcaccaccaccactgcggcggtgagccacgcccacgagcaggcggcagcgcatgcccacgcacgggcggtggcggcacaggcgtatagcggcggcaacatgagcagggatcacgacgacgggaagatcgcctacgagaactacctcgtcagcaccgcagaagcctacggtggggggaggatggctgcgtgggcgccggcctcagctccgccggcgacaagcagcagcgacatgaccggcacgcagctcttcagcgtctggaacgacactaattag

SEQ ID NO: 78 - DNA, 8220 bp, Triticum aestivum
  source 1..8220: mol_type = genomic DNA
aaattttgaaatgagagaacccaaggaggagcctattcacccgtatgcaatactctactgcttaagcacattcgaaaccagccttggaatgtgatgtgacctggtattacaattttagaggattagacaggcaaaattatattcttttgcacatgatgtgagacctggctgccatatatggttcatcatagtgaagtggactctctgcttggcccatatatggttatgtacatcaatataatgagaaagaacaacaaaaaatctcaaggaattcatgcataatttcataaggcgaagttgtttctccctagatatagcttgcacataagaccaaataacatatagtgtggcaacttgtcacgacatgaccaaagggtaggcacgtagagttagtcgttaacgctttcatcgctccatgccatccactagtgttgacactaaacacatagcaatgggttgtctcttaagctgtttttgcatgaggagtgagacattagcacacaaagatattgatgttgatcactctagggttttgggattttggctttgatgcatcagatttgtccttctccacctccacaggcatgatcatatgtgacagagtgaagaaccaatttatgtcctatctcacattgatatacataccctatatattgtcatggtgactggctctaagagaaaaaagatcatgtacaataagaagattgtcactagatcatactatatgtgtatgttaggcacaagagatggatcgatcaaccgatgaagcaagcaattagcatagtggcgatcacgtgggggtggtatccctctggtaccgtgggagactaacccgatcatccaagaggcacacgtgtgcgtgtccagacttgagtagaagaagagcatgcatgcacatgccaatggcctcgcacatgcactttatcgttccaagagaagattgggcggtcgaggtcgacgacggtggactgtgatcagtgtgtgcacgtgagaagcccaacttgatcgtcgctcacagttaacaacaccctccattttcttcctaaatcatatcatatcataccatcaagcgcacgtacgtgtctagctaattagtcaacaatacacttagtagatatatacgctcatgaatatacatgtgtacgcgtacgtatacttcatttctgagggaatctcgaggagtacgtctccaccttatgatagcatattcacactgccgagtagtactagtagtactggtaattaattaacagcgcggttaatcagtaaaagatgatgaggttccagtctgaacgctgagcagaataggcaattaaagagacacgcgcgtacatacctctcgcacgcttgcttgcttccggtttgaatacgatccagctttgacggtcaaagttttaggcgggggcaagcggtgcaattaccaaggtgaaaacacacacacccggcctatcttaacctccgcttctccttctccccataaagagagagaagaagaagaaagcaacacaggcattgttaatccctcccctctctctctagtactggcgcttgcttctcactctagtcttgttagggagagagagagagattagctgggtgggtcttgttagtactggtaacaagttgtcatgggggatcctatgcgaggtttagggttccgctcgcgcgtttctctatgcgtttctggagaggatcgacgccaaggtttttctatggccggaagacgagggtccgtgtcctcttctagggtgcaggtagcacattatctcctcccctcttcctccccagcgccctctcatccatcaatcactctactactagtagtatgattttctttcccccttttccagcctctcgttgttctgctactagctatatgtagctagcttatgctttccattgaagccaaatgaatccttggctagcttcatttccatcatgttttcatcttcaccacaagcaaaagtatattttgcaatatgttatattactactatctatcaaatgataagtaattagtaaactatataagtggttatgtgcgaattacagagtttaatctcatcatgtgtattgtcacttgcggtgctattagcatttttcatgagattattatcagcgatttcgtctaagttgttgaaatattgtagtacatccaaatgtagggcgcctgtcactgtgcgtgcatgccaccctttgtgccattgtgtgtgtgttgctgtgtcaggaggaggggggcagggcctagggctttgaaagaaataacccttgtcagggattcagcttctgcagcggcgagtggttttagggtttgagagaggccggccaagacttaattgttcatacgtgggttcaccactgttgaacagtgttttggcgcatagttagcaattgattaactatgttaccagtactagttatacacacacatatgcatatgtcaactgccgaactgcgtgcatatatgtccagggcacccaaccgtgcgcccgcatgtgcacccccgcaattgaaaaagagaggagatactatgtacgcaaagaacatctgctcgcgatcgaattgagatcctagacaaatagaatgaagctagatcttgcgacatttcttaaatttttcaagggggtgatcagttttctaatcacctgtgaataccagctttccagtcaaaaaagggcatgacaactcatactgttatcgagtaaagggcgccgctttagtagtacatgtacatgcaggcaacagggtgtactcatgttggcatatcttacatgtttttgggggaaatgagacaaaatatggtggactcgatctccccccagaaagcacaagcaaaaagatgtccctgctcagttaaaccatgccactgttttttgtattgcacaaagatacaaaaatatagtacccttttttatgtgtggaaaaagcttcctactacgtagagtacatttcactaattaatcaaggagtacatgcataatcatccacagtagtctcacactctgacataggagtgttaatggcgttaaggattagtgtactataataatttgtataaatatctgccccagtagtacttgtctactactactactagtagcacacagttcctcaagtctccaaactccaaagctctcttgggtcttggcctctccagcttctcctaaccttgggctcccggcaacaatggcttcttgatctgctgctctccctttcccggcctatccaacagcaagcttaagtagagacaagttaatcacttccacgtactgtgttgcttttaggtagcttcgaccatggccaccgtgaacaactggttgggcttctccctctccccgcaggagctccctccctccgccgccgcctcaggtgacgtctccggtgccgacgtctgctacaacatcccgcaaggtagatacatacacatcgtagtattcttgtgattggtttaagtgcatcaagcagttgtatgaaaatcctgtgattggtttgtttcgcagattggaacatgaggggatcggagctatcagctctggtggcggagccgaagctggaggacttcctcggcggcatatcctattcggaccatcaccaccaccagcacaagcagcccggcggcaacaatatggtcgtccctgcaggcagcgccagcggcggtgccgcttgctacgcgagctctggcggcagtagcgtgggctacctgtaccaccccagctcggcctccctccagtttgccgactccgtcatggtggcttcctccggcggtggcgtccaccacgacggcgctggcatcatggcaaacaccacagccaacggtgaccttaataatggcggcggcgggggtggcatcgggctgtccatgatcaagagctggctgcggagccagccatccccggcacaaccaccggagcagcagagggccgacgcggcggcgcagggtctctcgctgtccatgaacatggcggcgtgcatgccgccgctcgcttgcggggagcgtggcgtgccggagttagcgattgtcaggaaggatgacacggccggtggcagcagcgctggttctggggctgtggtgtcggccggtgcggatagcaccggcggaagcagtggcgcggtggtggagaccccagcatcgaggaagacggccgatacgtttgggcagcgaacttcgatttaccgtggcgtgaccaggtatagtactagtgcattgcaataaacatgaaagtatctgaacatactctctatttttcaaaatgggatatttgattgctttttgtgtcattagactgagcatccagctaatgcatagtttcaggcatttttgttttctagctaatcatcaacttagacactgatattgctaagcatggatcaatgtagtttctttcctttttttctgttttttcgatgacactagtatcactttttatatttttctcttatttatttctttgaatcaagtgagtatgtcatttgcttttgagtctctttttgaataatgtaggtgcataggaaatgtggaatgccattatgtgggcatgattgtggcaggcttacaacgtgtgattcttttttgcaattggctttaataggcatagatggactgggagatatgaggctcacttatgggacaacagttgcagaagagaagggcaaactcgcaagggtcgtcaaggtactgatgtgttatcttttaatctgtttacagatatacctatatatgatcaatccccttcaatgttgctaattaaatttactctttgattgattatccaactttgtttgaactcggccttctcaactccacacagtctatctaggtaagcaagatccagatcggagtaattacatttgtttttgacacattttgtcgatgcattcattgctatcaattctttaaactacatgttttgtgcatgttctaatcttgaaatacatatataggtgggtatgacaaagaggagaaagctgcaagagcttatgatttggctgctctcaagtattggggtgccacgacgacgacaaatttcccagtaagcattacgctagctgctcttgttgtcattgttgttgtcattgtttttggaagaagtcattgttatttattactacctctgtaaacaaatacaagatttttagatcttttagatcactaaagtagtgatctaaaagatcttatattagtttacagagggggtaccattttattattcgttgatcaatgtaatggtgaactacatagccacatctctctaattattatcttttgggaagcctagcttactgaagtcgtacttaaagtcatgcatattttctccattttttctaggtaaacaactatgaaaaagagctagaggagatgaaacacatgacaaggcaggagtttgtggcgtctctgcgaaggtcggtctcttatcttttatatatgaatccaggtacatggttagcaaatcaatcttcaaatcaactaatgttaatgttatttgatgtgtgtatgtgcaggaagagcagtggtttctccagaggtgcatccatttatcgtggagtaactaggtatgcacgtgcttccttttcttggttgcgtgcatcgtatggtgcagaggtcgtcaggataatcctaacattagctacataaaaatctgattattgctgctgatccatttgaattttgtataataaaggcaccaccaacatgggaggtggcaagcgaggatagggagagttgcagggaacaaggatctctacttgggcaccttcagtaagtatagatacgcacaatcaattgcactgaatatgttaatctggttacactgaaattgccaccttaactatcagaggcattagagcgtatgcgtttgtactgtgtttcttaaaaaaaacaaccggatacattttatgctgacatttattaatagtcattcattgcgaactaatctctttacggatggtactcgttgattatccaaattcaaatccatgtccagctgacgtgtgagcccaaaggccaacccatacgtaggcatgtgagaaatggagtgacaaaccgtaaaatgccaatgaatggcgcgacaaaatcgtagatttcatgttgaaaatggacctgcaagtcatgccaagttgcatcgagcatattactattgtactttgtgcatgagtgcacatgctttattaaacgtagcttccatgtttagtgctgccgctgtctccaatcaccggcgcatctagtcaaaatattcgagctgcgtctgcatgcatgcatgcatttgcagtaaactaatgatcaattaaaataactaagctagcttgtcacaccttgcttgcatgcatgcatgcatggcgtcgactggtagattgcgttgttggtacctaaccatgggcatcgcctgtgtaatgtaggcacgcaggaggaggctgccgaggcgtacgacatcgcggcgatcaagttccgggggctcaacgctgtcaccaacttcgatatgacgcgctacgacgtcaagagcatcctcgacagcaccgcgctccccataggcagcgccgccaagcgcctcaaggacgcagaggcggccacggcgtcgtccctcgcgctgcagcagcagcagcacgcaggcgttgtgagcggctacgacatgggcgccctggccgcctacggtgcggcgtaccaccaccatcacccctccgtcgccggcgctgttgcgtggccgaccatcgcgttccaggcgccgcctcagcaggtttctggcggcggtggtggacacatgtaccacccctacgcccatgcgcagccgctgcgcgggtggtgcaagcaggagccggaccacgcggtgatcgcggccgcgcacagcctgcaggagctccaccacctcaacctcggtgctggtggcggcgcgcacgacttcttctcgcagcatgcccaggcgatgcagcagcagcacggcggccttggcagcatcgacaacggggccggcgcctcgctggagcacagcactggctccaactccgtagtgtacaacaacggcgccgtcgagagctatatgctgccaatgagcaccaccagcaccgcggcggtgagccacgcccacgagcaggcggcagcgcatgcccacgcacgcgcggtggctgcacaggcgtacagcggcggcaacatgagcagggatcacgacgacgggaagatcgcctacgagaactacctcgtcagcaccgcagaagcctacggtgggggaaggatggctgcgtgggcgccggcctcagctccgcccgcgacaagcagcagcgacatgaccggcacgcagctcttcagcgtctggaacgacactaattaggtcagctagagagagaaacactaacatcacaaagcacttagggtgtttatgcatgagatggccattaaggtgttactgtcgtgttcaattattgttactcggtagctagttgggatggtgttcttctgcaggaaggtaatggcctgtgagagggctatgcattgcactgcattgttcttggatcagttttgctattatcattttggggttaagttcagaagaacatatattggggcattgactagggaattagatggtttagaaatcaatggagctaaaactagcctgcttgtaaattttgaattgctagggtggatggcactgcatgatggcggtcaatgcgcaaatgagttatttttttaacaaaattctcaactttatttctgtttcctttagtgttttactagcaaaaatgcccgtgtgttacaacggggtaataataataatatgaaaaccccctcctctgtcacactgttgacgccatttattccccctgacacccccatcatagtggttgttatcctgcttcccccatcaatgacgttggtctcccatctcatcaacaagtctaaatgtggtcaatctcaacacacaaatgcccgcgcgttgcaacggaaaaaactttgacgtggattgaaatataataagaattgtgaaatgttgctcattttacaaatcgacttggtgcgccacttcttctatccaactctaacaaattttctgtgaatatagatagaagaagggaacgtaatcctaaaaacatatagctcacttcatatttctcctattcctccttccttgagtagcgggtattccttacatttgtagtgatatagtataaaaataatgttattgctgtcatgtccaccagagtttcagtaaaagaacaaaaaagaaaattagc

SEQ ID NO: 79 - DNA, 5688 bp, Triticum aestivum
  source 1..5688: mol_type = genomic DNA
atgggggatcctatgcgaggtttagggttccgctcgcgcgtttctctatgcgtttctggagaggatcgacgccaaggtttttctatggccggaagacgagggtccgtgtcctcttctagggtgcaggtagcacattatctcctcccctcttcctccccagcgccctctcatccatcaatcactctactactagtagtatgattttctttcccccttttccagcctctcgttgttctgctactagctatatgtagctagcttatgctttccattgaagccaaatgaatccttggctagcttcatttccatcatgttttcatcttcaccacaagcaaaagtatattttgcaatatgttatattactactatctatcaaatgataagtaattagtaaactatataagtggttatgtgcgaattacagagtttaatctcatcatgtgtattgtcacttgcggtgctattagcatttttcatgagattattatcagcgatttcgtctaagttgttgaaatattgtagtacatccaaatgtagggcgcctgtcactgtgcgtgcatgccaccctttgtgccattgtgtgtgtgttgctgtgtcaggaggaggggggcagggcctagggctttgaaagaaataacccttgtcagggattcagcttctgcagcggcgagtggttttagggtttgagagaggccggccaagacttaattgttcatacgtgggttcaccactgttgaacagtgttttggcgcatagttagcaattgattaactatgttaccagtactagttatacacacacatatgcatatgtcaactgccgaactgcgtgcatatatgtccagggcacccaaccgtgcgcccgcatgtgcacccccgcaattgaaaaagagaggagatactatgtacgcaaagaacatctgctcgcgatcgaattgagatcctagacaaatagaatgaagctagatcttgcgacatttcttaaatttttcaagggggtgatcagttttctaatcacctgtgaataccagctttccagtcaaaaaagggcatgacaactcatactgttatcgagtaaagggcgccgctttagtagtacatgtacatgcaggcaacagggtgtactcatgttggcatatcttacatgtttttgggggaaatgagacaaaatatggtggactcgatctccccccagaaagcacaagcaaaaagatgtccctgctcagttaaaccatgccactgttttttgtattgcacaaagatacaaaaatatagtacccttttttatgtgtggaaaaagcttcctactacgtagagtacatttcactaattaatcaaggagtacatgcataatcatccacagtagtctcacactctgacataggagtgttaatggcgttaaggattagtgtactataataatttgtataaatatctgccccagtagtacttgtctactactactactagtagcacacagttcctcaagtctccaaactccaaagctctcttgggtcttggcctctccagcttctcctaaccttgggctcccggcaacaatggcttcttgatctgctgctctccctttcccggcctatccaacagcaagcttaagtagagacaagttaatcacttccacgtactgtgttgcttttaggtagcttcgaccatggccaccgtgaacaactggttgggcttctccctctccccgcaggagctccctccctccgccgccgcctcaggtgacgtctccggtgccgacgtctgctacaacatcccgcaaggtagatacatacacatcgtagtattcttgtgattggtttaagtgcatcaagcagttgtatgaaaatcctgtgattggtttgtttcgcagattggaacatgaggggatcggagctatcagctctggtggcggagccgaagctggaggacttcctcggcggcatatcctattcggaccatcaccaccaccagcacaagcagcccggcggcaacaatatggtcgtccctgcaggcagcgccagcggcggtgccgcttgctacgcgagctctggcggcagtagcgtgggctacctgtaccaccccagctcggcctccctccagtttgccgactccgtcatggtggcttcctccggcggtggcgtccaccacgacggcgctggcatcatggcaaacaccacagccaacggtgaccttaataatggcggcggcgggggtggcatcgggctgtccatgatcaagagctggctgcggagccagccatccccggcacaaccaccggagcagcagagggccgacgcggcggcgcagggtctctcgctgtccatgaacatggcggcgtgcatgccgccgctcgcttgcggggagcgtggcgtgccggagttagcgattgtcaggaaggatgacacggccggtggcagcagcgctggttctggggctgtggtgtcggccggtgcggatagcaccggcggaagcagtggcgcggtggtggagaccccagcatcgaggaagacggccgatacgtttgggcagcgaacttcgatttaccgtggcgtgaccaggtatagtactagtgcattgcaataaacatgaaagtatctgaacatactctctatttttcaaaatgggatatttgattgctttttgtgtcattagactgagcatccagctaatgcatagtttcaggcatttttgttttctagctaatcatcaacttagacactgatattgctaagcatggatcaatgtagtttctttcctttttttctgttttttcgatgacactagtatcactttttatatttttctcttatttatttctttgaatcaagtgagtatgtcatttgcttttgagtctctttttgaataatgtaggtgcataggaaatgtggaatgccattatgtgggcatgattgtggcaggcttacaacgtgtgattcttttttgcaattggctttaataggcatagatggactgggagatatgaggctcacttatgggacaacagttgcagaagagaagggcaaactcgcaagggtcgtcaaggtactgatgtgttatcttttaatctgtttacagatatacctatatatgatcaatccccttcaatgttgctaattaaatttactctttgattgattatccaactttgtttgaactcggccttctcaactccacacagtctatctaggtaagcaagatccagatcggagtaattacatttgtttttgacacattttgtcgatgcattcattgctatcaattctttaaactacatgttttgtgcatgttctaatcttgaaatacatatataggtgggtatgacaaagaggagaaagctgcaagagcttatgatttggctgctctcaagtattggggtgccacgacgacgacaaatttcccagtaagcattacgctagctgctcttgttgtcattgttgttgtcattgtttttggaagaagtcattgttatttattactacctctgtaaacaaatacaagatttttagatcttttagatcactaaagtagtgatctaaaagatcttatattagtttacagagggggtaccattttattattcgttgatcaatgtaatggtgaactacatagccacatctctctaattattatcttttgggaagcctagcttactgaagtcgtacttaaagtcatgcatattttctccattttttctaggtaaacaactatgaaaaagagctagaggagatgaaacacatgacaaggcaggagtttgtggcgtctctgcgaaggtcggtctcttatcttttatatatgaatccaggtacatggttagcaaatcaatcttcaaatcaactaatgttaatgttatttgatgtgtgtatgtgcaggaagagcagtggtttctccagaggtgcatccatttatcgtggagtaactaggtatgcacgtgcttccttttcttggttgcgtgcatcgtatggtgcagaggtcgtcaggataatcctaacattagctacataaaaatctgattattgctgctgatccatttgaattttgtataataaaggcaccaccaacatgggaggtggcaagcgaggatagggagagttgcagggaacaaggatctctacttgggcaccttcagtaagtatagatacgcacaatcaattgcactgaatatgttaatctggttacactgaaattgccaccttaactatcagaggcattagagcgtatgcgtttgtactgtgtttcttaaaaaaaacaaccggatacattttatgctgacatttattaatagtcattcattgcgaactaatctctttacggatggtactcgttgattatccaaattcaaatccatgtccagctgacgtgtgagcccaaaggccaacccatacgtaggcatgtgagaaatggagtgacaaaccgtaaaatgccaatgaatggcgcgacaaaatcgtagatttcatgttgaaaatggacctgcaagtcatgccaagttgcatcgagcatattactattgtactttgtgcatgagtgcacatgctttattaaacgtagcttccatgtttagtgctgccgctgtctccaatcaccggcgcatctagtcaaaatattcgagctgcgtctgcatgcatgcatgcatttgcagtaaactaatgatcaattaaaataactaagctagcttgtcacaccttgcttgcatgcatgcatgcatggcgtcgactggtagattgcgttgttggtacctaaccatgggcatcgcctgtgtaatgtaggcacgcaggaggaggctgccgaggcgtacgacatcgcggcgatcaagttccgggggctcaacgctgtcaccaacttcgatatgacgcgctacgacgtcaagagcatcctcgacagcaccgcgctccccataggcagcgccgccaagcgcctcaaggacgcagaggcggccacggcgtcgtccctcgcgctgcagcagcagcagcacgcaggcgttgtgagcggctacgacatgggcgccctggccgcctacggtgcggcgtaccaccaccatcacccctccgtcgccggcgctgttgcgtggccgaccatcgcgttccaggcgccgcctcagcaggtttctggcggcggtggtggacacatgtaccacccctacgcccatgcgcagccgctgcgcgggtggtgcaagcaggagccggaccacgcggtgatcgcggccgcgcacagcctgcaggagctccaccacctcaacctcggtgctggtggcggcgcgcacgacttcttctcgcagcatgcccaggcgatgcagcagcagcacggcggccttggcagcatcgacaacggggccggcgcctcgctggagcacagcactggctccaactccgtagtgtacaacaacggcgccgtcgagagctatatgctgccaatgagcaccaccagcaccgcggcggtgagccacgcccacgagcaggcggcagcgcatgcccacgcacgcgcggtggctgcacaggcgtacagcggcggcaacatgagcagggatcacgacgacgggaagatcgcctacgagaactacctcgtcagcaccgcagaagcctacggtgggggaaggatggctgcgtgggcgccggcctcagctccgcccgcgacaagcagcagcgacatgaccggcacgcagctcttcagcgtctggaacgacactaattag

SEQ ID NO: 80 - DNA, 6140 bp, Triticum aestivum
  source 1..6140: mol_type = genomic DNA
gaagagggcaaactcaggagcaagatgcattcgtcagcttcagcatggggacctcatcgttgcaccatcaacctgttgccaaagaggacctcctaccccctcgccctggcacgaagggcttgatccgtcagacgatgaagagatcttaaagcacggaagttagctaaacaccagacaatcgaggaagaagggactgtgagcacgaccaccatcgccagccatgccatgccaccgtgaacaacccccacgatgacacaatgaagagacctagcaggctcctcttaatattgtgatttcacaatcatttaagctttgtatgagatatttaggatcaaaactcaattttgatcaatagatgtacgcgttgtattctgaaacaaatgaatatatgtttatgcgacttaagaaatcctagctgaccttacgttttgattccttttccacttcttttctattccttctcctactgcattgtagtagtaacatttttcttattgttatttgtcatttctttaatacagtacagatgcagacactcatacacatgcacatgcactcatccctatgaatccacgcacgtacaacctacccctatgagcatattcgagagattgagccaacaaatcatcttgagaatgacaaagtcgccataaacgcctttgtagtcgacgggaatatttttcccctaaacgcgctggaaggtctagaataaatccagaaaaatgtgagtcatgacccgtagatctacacacaatttattttcatggaaaattgtttatatgtcctatagagaaccaaaaacacaactaccaccactgttacgttctcatataaatacttacctctcctagcacattccccccttctctcatgtggccaggatatttttctggccactgctgcactcttctcacattgacccaaatgtagctagtgttctaggtgcttctcaaaaaggggtataccctcctcccctatcagcttccaccttacctcgccaatgcttccagtgctctgatcaggaagaaaccaactattgatctatcgcagctagccccgcgggctgttctatcaatcgcccgtcccataatggcttctgggaacgactggttggggttctcgctctccggccaagagaacccacagccgcatcaggatagctcgccgcccgccgacatcgacatcgctggcgccagtggcttctatggcctacccacgcagccagctcccgacgctcaactcggtgtgccaggtcatcatcagccttcatacgggatcacggaggccttcaataggggagctcatgaaatccacggtgagaggaagaagctagccagtttgccgcgtgcactccgatccaatgttatctagctaggttgtaggtatgtatcacttctcccgttcaaggtttcttgttatttgatgtggctgtagattggaacatgaggggtgatctggactacaacggcggtgcgtccgagctctcgatgctcgtcgggtcgagcgcagttggcggcaagagggcggccatggaggccgagaccgagcccaagcttgaggacttcctcgttggcaactcgttcgtctccgagcaagatcaggccggtggcttcttgttctccggtgtcccgatggcgaccagcaccaacagcaacagcaggagcaacaccatggagctctctatgatcaagagctggctccgtgacaaccaggtaccccaagcgccgcacccgcagtcgcagaccggggcgcccgcgcagcagccgcaaccccacgaggagatgggcaccgacgcgagcagcttcgatccgctggggagaaacggcgcgttggtggtggcggggagctcgcagagcctggcgctctcgatgagcacgggctcaggatcgcacttgccgatggctgtgctcggcggcagtcccagtgggggagtctcagagagcacctcctcggagaacaagcgggccagcggcgccatggattcgccgggcggtgcggtagaagctgtcgcgaggaagtccatcgacacattcgggcaaaggacgtcgatatatcgaggtgtaacaaggttagaacattatattattaattactggttgatttcttatattgttactagtcgatttcttcacattaattgttgatccatgacctacactaattagtccatttccaattgggctagtacactggcatttcaaattgggcgatcaatctagcgaggaactctttaaacagacataagggatctatgctagtcaggataatacaagctagagtactttgatttcgtgcctgtaatttggtgattatttgagcacatatatataattcgttgaccgagaaaaggtgacaaatcgacttagttattatatccaatgattaatatatagatgctcccatttttcatccacgagttaggtaaaagactttcaccctgcttaaaagatataaaagaaccaccacatacatacaacaaagttcaagcacaggaaaaaataaacataagccccgctggggcaacaacacaaacgtgccctaataaataaataaaatgaaagggccaccaagtggctggcagatcaatggctagggggtgaagcaatacgtgtctcgccgtaatcagcgcccatcatgccgcctaaccgatcaatggtcaggtatatataactaagaagtaatatgtagctcactggctaattaagaaacgacttatttataattgaattgaattacttgttcatacgagtacacgacatgattcatactatagctaccacttagtacttaccacacaatttgaactagtattaagtgttgttctttttctccggaactcttatgatatcttctacactaatctacaccactataacccgagtgcaccaaaataagaggtataactaaattaactgttctagctgacccaagtacccaacacaaatagatagtgtggctctttgctttttctctggataaacacagaagtttttatatagagctcttttatagacctctcaaaacgaatgtgcttcaatggatgatgaatacaaagtcatataaaatgagaaaatgatgctctttttctctgatcaacatactcttgcacatacatctacattatattagaaatggatgtattttttttaacaacaacatgtacattttgaatgtgctaactatatatattcttatgttcttcaatattccttttcagacatagatggacagggcgttatgaggctcatctctgggacaatagctgcagaagagaagggcagagtcgcaagggtaggcaaggtaataaaaatggctatatgcttcctctttctctctccattgcacaacaatatatacacccgtgatgtgctatttacgcattttatcttgtgtttccctcaacactctgcatgactgcatcaccatcgcctccgggaatgatactgtattgtcctccacaatcagtttatctgggtatgcttgccttactctctggccatttttatttattggagtttaccaatttagttattccaccttatgattgtgatcatgaaaccttaggtggctatgacaaggaggacaaggcagcaagggcttatgatttggcagcactcaagtattggggtacaacaacaacaacaaatttcccagtaagcaagttctgatactatttttatggcgctttttccttgcattgctattttcatttctgcaaaacatctcaacctatttttattggaacataatagttttttttacaatggtagattaatacctacgaaaaagaggtggatgaaatgaaacatatgactaggcaggaatacatcgcatacctaagaaggtactatcgcgagccgacgttcatctcatattgaactactatcacacaagtaaatggaatattgcactatcaataagttgatccgacaaatgcaggaatagcagcggattttcccgtggtgcgtcaaaatatcgtggtgtaactaggtatgttactaggtaattcggtctgttatgtttaccataataatttatgtcatgtggaaatagctaatgtccgatatgtattcttagaattagtaggcatgcacgtaagcaaacatgtatgttagatacgttatccaaatggatgttatatcttttttcgattttctaagttctaactatataaggctgagatctttaccaggcaccatcagcatgggagatggcaagcaagaattgggagggttgcaggaaacaaggatctctacttaggcaccttcagtaagttattgatcagatataattaaccctctcttcggatcctatctaattgctggactaatgaagatccttaagactaaattatgactagtcatagtgttatagctccaagatctaagtcatcgaccagtactacttatttagttacttttttcttgaactcttcgtctcgtaaagctgaaggttagatcttctatatatatgttgccctatggggcaataaaatagtggtcactgcactaactatgtcccaaatgtaccttttcaagtttttgtctcaaagttgtctaagaaaagagaaaaccttggcatggagtcaagggcaaagttgaaaaccctagtgatttgaatcacacacttgcatcctagagccgcctatgtatagctgcatgcatgcaatatgaaacatatatatacatcgtcacatatgcatgcaatgtggggggaccagccctagctagctaaccatgcatcctaatctcgtacatccgtaggcaccgaggaggaggcggcggaggcctacgacatcgcggcgatcaagttccgtgggctgaacgccgtcaccaacttcgacatgagtcgctacgacgtcaagaccatcctcgagagcagcaccctgccggtgggtggcgcggcgaggcgccccaaggaggtcgctgatcatccagaggctggcgccacgatctggcgggccggcatggacggcggcgtcatctcccagctgaccgacgtcgggatgggcacgtacgcgtcgtaccaccacggctggccaaccatcgcattccagcagccctcgccgctgtcggtgcactacccatacgtcgcccagccgtctcgcgggtggtgcaagcccgagcaggacgccagcgtcgccacgcaaagcctgcaggacctccagcagctgcacctcgggaccgccgctcacaactatttccaggcatcgtcgagctccacggtctacaacggcggcgggtaccagcaaggcaccagtggcaacgccttcttgatgccggctagcaccgtggtggatgaacaggggcacagcagcactgccaccaaccagggaagcacatgcagctacggggacgaagaggggaagctcaatctcgggtacgatgccatggcgatggcgagcaccggcgccgacccatacgcggcggcgccaactgtgagcattgcgagggcgaacgggtactccaacaactggagctcgccgttcaatggcatgtgatgatggtgaagagggcagacgctagtaatagcgggattcattagctagcaagacagtcgaaaagttaggtcagggtattgctcgttgttgcgtttcagctcagggttccggctagctcttcttggccgggtggatgacacacgtaaaggtgctgtgcatgcatgccttctggaagctggtaatggcaagtcaggataactaaagctaacttgtcctgacttgctgttagttttgggtagtttcagaaggatcaagttaatctctaggacaaaagctgattgtgcgatggcaataactttataattccagtgattcgacttacattggctttccatttagttagcgcatctacagccgcatacgataaatatgacccctcaaagaaaagggcctggtcatcctttattttgcatttgttgcaatcgtataccagaatttgatacctcaaatccatactacgccatgtaattacgtactatcaaaacctgcttaatgtttttatttaataatatggttgcatgcatcgattgatgcagaggccgaggtaatcctcatttttccaaaaaaaacttgaaacatgcttaaactactcctcgtcggagatgtatatgatgtactaatgtctacaaaaaatgttaaa

SEQ ID NO: 81 - DNA, 4411 bp, Triticum aestivum
  source 1..4411: mol_type = genomic DNA
atggcttctgggaacgactggttggggttctcgctctccggccaagagaacccacagccgcatcaggatagctcgccgcccgccgacatcgacatcgctggcgccagtggcttctatggcctacccacgcagccagctcccgacgctcaactcggtgtgccaggtcatcatcagccttcatacgggatcacggaggccttcaataggggagctcatgaaatccacggtgagaggaagaagctagccagtttgccgcgtgcactccgatccaatgttatctagctaggttgtaggtatgtatcacttctcccgttcaaggtttcttgttatttgatgtggctgtagattggaacatgaggggtgatctggactacaacggcggtgcgtccgagctctcgatgctcgtcgggtcgagcgcagttggcggcaagagggcggccatggaggccgagaccgagcccaagcttgaggacttcctcgttggcaactcgttcgtctccgagcaagatcaggccggtggcttcttgttctccggtgtcccgatggcgaccagcaccaacagcaacagcaggagcaacaccatggagctctctatgatcaagagctggctccgtgacaaccaggtaccccaagcgccgcacccgcagtcgcagaccggggcgcccgcgcagcagccgcaaccccacgaggagatgggcaccgacgcgagcagcttcgatccgctggggagaaacggcgcgttggtggtggcggggagctcgcagagcctggcgctctcgatgagcacgggctcaggatcgcacttgccgatggctgtgctcggcggcagtcccagtgggggagtctcagagagcacctcctcggagaacaagcgggccagcggcgccatggattcgccgggcggtgcggtagaagctgtcgcgaggaagtccatcgacacattcgggcaaaggacgtcgatatatcgaggtgtaacaaggttagaacattatattattaattactggttgatttcttatattgttactagtcgatttcttcacattaattgttgatccatgacctacactaattagtccatttccaattgggctagtacactggcatttcaaattgggcgatcaatctagcgaggaactctttaaacagacataagggatctatgctagtcaggataatacaagctagagtactttgatttcgtgcctgtaatttggtgattatttgagcacatatatataattcgttgaccgagaaaaggtgacaaatcgacttagttattatatccaatgattaatatatagatgctcccatttttcatccacgagttaggtaaaagactttcaccctgcttaaaagatataaaagaaccaccacatacatacaacaaagttcaagcacaggaaaaaataaacataagccccgctggggcaacaacacaaacgtgccctaataaataaataaaatgaaagggccaccaagtggctggcagatcaatggctagggggtgaagcaatacgtgtctcgccgtaatcagcgcccatcatgccgcctaaccgatcaatggtcaggtatatataactaagaagtaatatgtagctcactggctaattaagaaacgacttatttataattgaattgaattacttgttcatacgagtacacgacatgattcatactatagctaccacttagtacttaccacacaatttgaactagtattaagtgttgttctttttctccggaactcttatgatatcttctacactaatctacaccactataacccgagtgcaccaaaataagaggtataactaaattaactgttctagctgacccaagtacccaacacaaatagatagtgtggctctttgctttttctctggataaacacagaagtttttatatagagctcttttatagacctctcaaaacgaatgtgcttcaatggatgatgaatacaaagtcatataaaatgagaaaatgatgctctttttctctgatcaacatactcttgcacatacatctacattatattagaaatggatgtattttttttaacaacaacatgtacattttgaatgtgctaactatatatattcttatgttcttcaatattccttttcagacatagatggacagggcgttatgaggctcatctctgggacaatagctgcagaagagaagggcagagtcgcaagggtaggcaaggtaataaaaatggctatatgcttcctctttctctctccattgcacaacaatatatacacccgtgatgtgctatttacgcattttatcttgtgtttccctcaacactctgcatgactgcatcaccatcgcctccgggaatgatactgtattgtcctccacaatcagtttatctgggtatgcttgccttactctctggccatttttatttattggagtttaccaatttagttattccaccttatgattgtgatcatgaaaccttaggtggctatgacaaggaggacaaggcagcaagggcttatgatttggcagcactcaagtattggggtacaacaacaacaacaaatttcccagtaagcaagttctgatactatttttatggcgctttttccttgcattgctattttcatttctgcaaaacatctcaacctatttttattggaacataatagttttttttacaatggtagattaatacctacgaaaaagaggtggatgaaatgaaacatatgactaggcaggaatacatcgcatacctaagaaggtactatcgcgagccgacgttcatctcatattgaactactatcacacaagtaaatggaatattgcactatcaataagttgatccgacaaatgcaggaatagcagcggattttcccgtggtgcgtcaaaatatcgtggtgtaactaggtatgttactaggtaattcggtctgttatgtttaccataataatttatgtcatgtggaaatagctaatgtccgatatgtattcttagaattagtaggcatgcacgtaagcaaacatgtatgttagatacgttatccaaatggatgttatatcttttttcgattttctaagttctaactatataaggctgagatctttaccaggcaccatcagcatgggagatggcaagcaagaattgggagggttgcaggaaacaaggatctctacttaggcaccttcagtaagttattgatcagatataattaaccctctcttcggatcctatctaattgctggactaatgaagatccttaagactaaattatgactagtcatagtgttatagctccaagatctaagtcatcgaccagtactacttatttagttacttttttcttgaactcttcgtctcgtaaagctgaaggttagatcttctatatatatgttgccctatggggcaataaaatagtggtcactgcactaactatgtcccaaatgtaccttttcaagtttttgtctcaaagttgtctaagaaaagagaaaaccttggcatggagtcaagggcaaagttgaaaaccctagtgatttgaatcacacacttgcatcctagagccgcctatgtatagctgcatgcatgcaatatgaaacatatatatacatcgtcacatatgcatgcaatgtggggggaccagccctagctagctaaccatgcatcctaatctcgtacatccgtaggcaccgaggaggaggcggcggaggcctacgacatcgcggcgatcaagttccgtgggctgaacgccgtcaccaacttcgacatgagtcgctacgacgtcaagaccatcctcgagagcagcaccctgccggtgggtggcgcggcgaggcgccccaaggaggtcgctgatcatccagaggctggcgccacgatctggcgggccggcatggacggcggcgtcatctcccagctgaccgacgtcgggatgggcacgtacgcgtcgtaccaccacggctggccaaccatcgcattccagcagccctcgccgctgtcggtgcactacccatacgtcgcccagccgtctcgcgggtggtgcaagcccgagcaggacgccagcgtcgccacgcaaagcctgcaggacctccagcagctgcacctcgggaccgccgctcacaactatttccaggcatcgtcgagctccacggtctacaacggcggcgggtaccagcaaggcaccagtggcaacgccttcttgatgccggctagcaccgtggtggatgaacaggggcacagcagcactgccaccaaccagggaagcacatgcagctacggggacgaagaggggaagctcaatctcgggtacgatgccatggcgatggcgagcaccggcgccgacccatacgcggcggcgccaactgtgagcattgcgagggcgaacgggtactccaacaactggagctcgccgttcaatggcatgtga

SEQ ID NO: 82 - DNA, 7647 bp, Triticum aestivum
  source 1..7647: mol_type = genomic DNA
cgtccgcctctgggagctcttgtcggatatccagcttcatcctgacactgaggactccattacctggtccttgacccctaatgggtgctactcggccacctccgcctacaaggtctagttccttgcctctttgccctgccagttcgggaacatcgtgtggaagaactgggcccctccaaagtgtcgtttcttcacctggctcgctatccagaaccgtttatggaccgccgatcgcttggcgaaacgcgaatggccgcaccaacctacttgtcagctctgccgtacctcccctgagacggcacggcacattctttttgagtgccgcttctccagacgtatctggacggccgcggcctcttggctgtcttgtccagacctcatcagcagcatcggggagggtaggcctaaagtcgtggactattggttggccgtttcgaagtccaccacctcctccccaaatggcttgcgctcagctatcacgctcatcacttgggagatctggaaagagaggaacgaacgagttttcaacaacaagtcctccctgccatcggttgtcatgcataagattagggaagaggggaaggactggatcctcgctggtgctaaaaatctggcggaacttgtaggctgatcctcctccccctgtgtcgggggtggtggtcgcccccttcctcgttttttttgttgtttttggctcttgccatacccccgtttgcttctcctatatcaatatgaggcaaaagcttttttgcccgtttcaaaaaaaaaatacttgcctctcctagcacattccccccttctctcatgtggccaggataattttctggccactcctacactcttcccacattgacccaaatgtagctagtgttctaggtgcctctgaaaaaggtataccctcctcccctatcagcttccaccttacctcaccaatgcttccattgctctcatcaggaagaaaccaactattaatctatcgtagctagccccgcgggctgttctatcgatcggcggccttcccataatggcttctggaaacgactggttggggttctcgctctccggccaagaaaacccacagccgcatcaggatagctcgccgcccgccggcatcgacatctccggcgccagtgacttctatggcctacccacgcagccagcccccgacgctcaacttggcgtgccaggccatcagccttcgtacgggatcacggaggccttcactcatgaaacccacggtgagtggaagaagctagccagtttgccgcatgcgccccgatccattgttatctaagtatgtatctatcacttctcccattcaaggtttcttgttatttgatgtggctgtagattggaacatgaggggtgatctggactataacggcggtgcgtccgagctctcgatgctcgtcgggtcgagcgcagtcggcggcaagagggcggccatggaggccgagaccgagcccaagcttgaggacttcctcggtggcaactcgttcgtctccgagcaagatcaggccggcggcttcttgttctccggagtcccgatggcgaccagcgccaacagcaacagcgggagcaacaccatggagctctctatgatcaagagctggctccgtaacaaccaggtaccccaagcgccgcacccgcagtcgcataccggggcgcccgtgcagcagccgcagccccacgaggagatgagcaccgacgcaagcgcgagcagcttcgatgcgctggggagaaacggcgcgttggtggtggcggggagctcgcagagcctggcgctctcgatgagcacgggctcaggatcgcacttgccgatggctgcggtcggcggcagtgccactgggggagtctcagagagcacctcctcggagaacaagcgggccagcggcaccatggattcgccgggcggtgccgtagaagctgtcgcgaggaagtccatcgacacattcgggcaaaggacgtcgatatatcgaggtgtaacaaggttagaacattatattattgataattactggttgatttcttcacattaatttgttgatccatgacttacactaattagtccatttcaaattgggctagtatactagcatttcaaattgggcaatcaatctagcgcgagaaactctttaaacagacataaaggatctatgctagtcgggatactgcaagctactttgattttgtgcctgtaatttggtgattatttgagcacatatataattcgttgacagagaaaaggtgacaaatcgacttattatccaatgattagtatatagatgctaccattttccatccaccagttaggtaaaaggctttcaccctgctttatagataaaagaaccaccacatccatacaacaaagctcaagcacatgaaaaaataaacataaaccccgctggggcaacaacacaaatgtgccctaataaaaagaaaaagaaaaaggaagggccaccaagtggctggcagatcaatggctagggggcgaagcaatacgtgtcttgccgccatcagcacatccatcatgccacctaaccgatcaatggtcgggtatgtataactaacaagtaatatgtagctcactgggtaattaagaaacgttgattcataattggattgaattacttgttcatacaagtttttttttcgataaagggcgattttattaattcaaaatgtagcatcaagtggatacaaagtattatgagtaacacccggcctctgcataactaggatgcatacaaccaaatccgaaagtccgaccaaaaaaatcattaaataaaaaccgacaaaacggcaacagtagagtcctgtagaccgatactatgcctatgtcgaaggaggtggtgggtcgatccgaaggttatgctgccacccatgtcgggaaaaaacctccatagccacctgctccaatctcgtacacaccgtcttgaacagcgattggtactccggtcgttgtagaatagaccacgtatgtagcgagtgcgtacaccggaaaataacctgcaaagaagaagcattttttccattgaaaatcaaatcatttctacatagccaaagcgaccaaattaaggcatacgctcccacccttattagcgttttgaaaactatttggaataccgtccaaccaatgaccaaaaatattggcaacacttgtgggtggatgcaaatttgatgctatttggatgactgaccacgtagaacgtgcaaacttgcattgaaaaaagaggtgtttgattgtctcgtcatgagtacaaaaacaacactttttactcccttgccaattgcgtcatgcgaggttgtctttagttaacacaactcccttacgaagataccacatgaaaattttcacttttaatggaatcttggacttccaaattttcttgttattactaacaggtaccttagaatgtgtgagcgcacggtacatatagtgcagtctacagtgaaagacccggatgtagtaaggttccagcgaaacacatcacggccttctgccaggttaatcgaatccaacctggataaaagattatgccatgacacaagtcgggggccaatcaaatcccgtctgaacgaaatattcggtggggatgaattgagcacctgcgcaatagtattattcttatcgcgagcaatgttgtataaggttggatattgttctcggagactggcattgtctagccatatgtcttcccaaaaacgaatctccgacccgccctttatcgcgaaagacccaaagcgaaagagatgtttctttgtcgccattaggccagcccaaaagtgtgagtcgccgggtttccaaaatgcacaagtatacaacatgattcgtactatagctaatttgaactgcttcttctccggaactcttatgatatcttctacactaatctacaccactataacccgaacgcgccaaaataagaggtataactaatttaactgttctagctaacccaacacaaatagatagtgcggatctttgctttttctctggataaacacagaagtttttatatagagatctcttttatagacctctcaaaacgaatgtgtttcaatgaatgatgaatacaaattcatataaaatgaaattgatgcttttattttctctgatcaacatactcttgcacatacatgtacattagaaatggatgtatttttaacgacaacatgtacattttgaatgtgctaactatatatattcttatgttcttcaatattccttctcagacatagatggacagggcgttatgaggctcatctctgggacaatagctgcagaagagaagggcagagtcgcaagggtaggcaaggtaataaaaatggctatatacttcctatttctctctccattgcacaacgatatatacacccgtgatgtgctatttaagcattttatcttgtgttgcctcaacactctgcatgactgcatcaccatcacctctgggaatgataatatattgtcccccacaatcagtttatctaggtatgcttgccttactctctggccatttttattcattggagtttaccaattcagttattccaccttatgattgtgatcatgaaaccttaggtggctatgacaaggaggacaaggcagcaagggcttatgatttggcagcactcaagtattggggtacaacaacaacaacaaatttcccagtaagcaagttctgaaactatttttatggcgctttttccttgcattgctatttttgtttctgcaaaacatctcaacctatttttattggaacataatattttttttacaatggtagattaatacctacgaaaaagaggtggatgaaatgaaacatatgactaggcaggaatacatcgcatacctaagaaggtactattgctagccgacgttcatctcatactgaactatcacacacaagtaaatggaatattgcactatcaataacttgatccgacaaatgcaggaatagcagcggattttctcgtggtgcgtcaaaatatcgtggtgtgactaggtatgttactaggtaattcggtctgttatgttttacaaaccataataatttatgtcatttggaaatagctaatgtccgatatgtattcttagaattagtaggcatgcatgtaagcaaacatgtatgttagatagttatccaaatggttgttatatctttttttttctaagttctaactatataaggtgagatcgttaccaggcaccatcagcatgggagatggcaagcaagaattgggcgggttgcaggaaacaaggatctctacttaggcaccttcagtaagttattgatcagatataattaaccctctcttcggagcctatctaattgctggactaatgaagatccttaagactaaattatgactagtcatagtggtatagctccaagatctaagtcatcgaccagtactacttatttagttacttttttcttgaactcttcgtctcgtagagctgaaggttagatattctatatgttgccctatggggcaataaaatagtggtaactgcattaactctgtcccaaatgtactttttcaagtttttgtctcaaagttgtctaagaaaaccttggcatggagtcaagggcaaagttgaaaacactagtgatttgaatcacacacttgcatcctagagccacctatgtatacatgcatgcatgcaatatgaaacgtacatcgtcacaacacatgcatgcaattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaacatgcatcctaatatcgtgcacatcctctcgtacgtccgtaggcaccgaggaggaggcggcggaggcctacgacatcgcggcgatcaagttccgtgggctcaatgccgtcaccaacttcgacatgagtcgctacgacgtcaagaccatcctccagagcagcaccctaccggtggcggtgagtggcgcggcgaggcgcctcaaggaggccgctgatcatccagaggctggcgccacgatctggcgggccggcatggatggcggcgtcatctcccagctgaccgacgtcgggatgggcgcttacgcgtcgtaccaccacggctggccgacaatcgcattccagcagccgtcgccgctctcggtgcactacccatacgtcgcccagccgtctcgcgggtggtgcaagcccgagcaggacgccaccgtcgccacgcaaaacctgcaggacctccaggagctgcacctcgggatcgccgctcacaactatttccaggcatcgtcgagctccacggtctacaacggcggcggcggcggcgggtaccagcagggcaccggtggcaacgccttcctgatgccggctagcgccgtcgtggacgaacaggggcacagcagcactgccaccaaccagggaagcacctgcagctacggggacgaagaagggaagctcaatattgggtacgacgccatggcgatggcgagcaccggcgccgacccatacgcggcggcgtcaacggtgagcatcgcgagggcgaacgggtactccaacaactggagctcgccgttcaatggcatgtgatgatggtgaagagggcagaagctagtaatagcgggatcaattagctagctagagaatcgaagttaggtcaggggtcttgctcgctcgttgttgcgtttcggctcagggttccggctagctctccttggccgggtggatgacactcgtaaaggtgctgtgcatgcatgccttctggaagctggtaatggcaagtcaggataactaaaggaggcttgtgtcgttgtaagttatcctgacttgctgttagttttgggtagtttcagaaggatcaagattcgacttattttggctttccatttagttagagcatctacatacgcatatgacaaatataactcctcatacatccgtagacgcaccattttgtgtctgttgcactgcatacctcaaatttgataccttaaattcatattacactatgcaactgaaaagtacatactaaataacttccgactagtcaaaatttaaacaacaaatccgaacgaacgctaaaaataacgcatgcaatcccgccttttatccttagcgcacccgtgcatgcagcatggttggcacatgcttgaaactaataaaaatcaggatgatgtcagcaaagattctctttattttaaaaatttaaaatactttgtagctcaaattgtcgctccggttaaaaaaagttttcacataaaagatttctcacgacgaaaccttcgaaactagatccattgttggtatgttccgacgacttttttttggtaaaatgttaccacatgtgtgctacgcaagttatcatgcgagttgagacaag

SEQ ID NO: 83 - DNA, 5833 bp, Triticum aestivum
  source 1..5833: mol_type = genomic DNA
atggcttctggaaacgactggttggggttctcgctctccggccaagaaaacccacagccgcatcaggatagctcgccgcccgccggcatcgacatctccggcgccagtgacttctatggcctacccacgcagccagcccccgacgctcaacttggcgtgccaggccatcagccttcgtacgggatcacggaggccttcactcatgaaacccacggtgagtggaagaagctagccagtttgccgcatgcgccccgatccattgttatctaagtatgtatctatcacttctcccattcaaggtttcttgttatttgatgtggctgtagattggaacatgaggggtgatctggactataacggcggtgcgtccgagctctcgatgctcgtcgggtcgagcgcagtcggcggcaagagggcggccatggaggccgagaccgagcccaagcttgaggacttcctcggtggcaactcgttcgtctccgagcaagatcaggccggcggcttcttgttctccggagtcccgatggcgaccagcgccaacagcaacagcgggagcaacaccatggagctctctatgatcaagagctggctccgtaacaaccaggtaccccaagcgccgcacccgcagtcgcataccggggcgcccgtgcagcagccgcagccccacgaggagatgagcaccgacgcaagcgcgagcagcttcgatgcgctggggagaaacggcgcgttggtggtggcggggagctcgcagagcctggcgctctcgatgagcacgggctcaggatcgcacttgccgatggctgcggtcggcggcagtgccactgggggagtctcagagagcacctcctcggagaacaagcgggccagcggcaccatggattcgccgggcggtgccgtagaagctgtcgcgaggaagtccatcgacacattcgggcaaaggacgtcgatatatcgaggtgtaacaaggttagaacattatattattgataattactggttgatttcttcacattaatttgttgatccatgacttacactaattagtccatttcaaattgggctagtatactagcatttcaaattgggcaatcaatctagcgcgagaaactctttaaacagacataaaggatctatgctagtcgggatactgcaagctactttgattttgtgcctgtaatttggtgattatttgagcacatatataattcgttgacagagaaaaggtgacaaatcgacttattatccaatgattagtatatagatgctaccattttccatccaccagttaggtaaaaggctttcaccctgctttatagataaaagaaccaccacatccatacaacaaagctcaagcacatgaaaaaataaacataaaccccgctggggcaacaacacaaatgtgccctaataaaaagaaaaagaaaaaggaagggccaccaagtggctggcagatcaatggctagggggcgaagcaatacgtgtcttgccgccatcagcacatccatcatgccacctaaccgatcaatggtcgggtatgtataactaacaagtaatatgtagctcactgggtaattaagaaacgttgattcataattggattgaattacttgttcatacaagtttttttttcgataaagggcgattttattaattcaaaatgtagcatcaagtggatacaaagtattatgagtaacacccggcctctgcataactaggatgcatacaaccaaatccgaaagtccgaccaaaaaaatcattaaataaaaaccgacaaaacggcaacagtagagtcctgtagaccgatactatgcctatgtcgaaggaggtggtgggtcgatccgaaggttatgctgccacccatgtcgggaaaaaacctccatagccacctgctccaatctcgtacacaccgtcttgaacagcgattggtactccggtcgttgtagaatagaccacgtatgtagcgagtgcgtacaccggaaaataacctgcaaagaagaagcattttttccattgaaaatcaaatcatttctacatagccaaagcgaccaaattaaggcatacgctcccacccttattagcgttttgaaaactatttggaataccgtccaaccaatgaccaaaaatattggcaacacttgtgggtggatgcaaatttgatgctatttggatgactgaccacgtagaacgtgcaaacttgcattgaaaaaagaggtgtttgattgtctcgtcatgagtacaaaaacaacactttttactcccttgccaattgcgtcatgcgaggttgtctttagttaacacaactcccttacgaagataccacatgaaaattttcacttttaatggaatcttggacttccaaattttcttgttattactaacaggtaccttagaatgtgtgagcgcacggtacatatagtgcagtctacagtgaaagacccggatgtagtaaggttccagcgaaacacatcacggccttctgccaggttaatcgaatccaacctggataaaagattatgccatgacacaagtcgggggccaatcaaatcccgtctgaacgaaatattcggtggggatgaattgagcacctgcgcaatagtattattcttatcgcgagcaatgttgtataaggttggatattgttctcggagactggcattgtctagccatatgtcttcccaaaaacgaatctccgacccgccctttatcgcgaaagacccaaagcgaaagagatgtttctttgtcgccattaggccagcccaaaagtgtgagtcgccgggtttccaaaatgcacaagtatacaacatgattcgtactatagctaatttgaactgcttcttctccggaactcttatgatatcttctacactaatctacaccactataacccgaacgcgccaaaataagaggtataactaatttaactgttctagctaacccaacacaaatagatagtgcggatctttgctttttctctggataaacacagaagtttttatatagagatctcttttatagacctctcaaaacgaatgtgtttcaatgaatgatgaatacaaattcatataaaatgaaattgatgcttttattttctctgatcaacatactcttgcacatacatgtacattagaaatggatgtatttttaacgacaacatgtacattttgaatgtgctaactatatatattcttatgttcttcaatattccttctcagacatagatggacagggcgttatgaggctcatctctgggacaatagctgcagaagagaagggcagagtcgcaagggtaggcaaggtaataaaaatggctatatacttcctatttctctctccattgcacaacgatatatacacccgtgatgtgctatttaagcattttatcttgtgttgcctcaacactctgcatgactgcatcaccatcacctctgggaatgataatatattgtcccccacaatcagtttatctaggtatgcttgccttactctctggccatttttattcattggagtttaccaattcagttattccaccttatgattgtgatcatgaaaccttaggtggctatgacaaggaggacaaggcagcaagggcttatgatttggcagcactcaagtattggggtacaacaacaacaacaaatttcccagtaagcaagttctgaaactatttttatggcgctttttccttgcattgctatttttgtttctgcaaaacatctcaacctatttttattggaacataatattttttttacaatggtagattaatacctacgaaaaagaggtggatgaaatgaaacatatgactaggcaggaatacatcgcatacctaagaaggtactattgctagccgacgttcatctcatactgaactatcacacacaagtaaatggaatattgcactatcaataacttgatccgacaaatgcaggaatagcagcggattttctcgtggtgcgtcaaaatatcgtggtgtgactaggtatgttactaggtaattcggtctgttatgttttacaaaccataataatttatgtcatttggaaatagctaatgtccgatatgtattcttagaattagtaggcatgcatgtaagcaaacatgtatgttagatagttatccaaatggttgttatatctttttttttctaagttctaactatataaggtgagatcgttaccaggcaccatcagcatgggagatggcaagcaagaattgggcgggttgcaggaaacaaggatctctacttaggcaccttcagtaagttattgatcagatataattaaccctctcttcggagcctatctaattgctggactaatgaagatccttaagactaaattatgactagtcatagtggtatagctccaagatctaagtcatcgaccagtactacttatttagttacttttttcttgaactcttcgtctcgtagagctgaaggttagatattctatatgttgccctatggggcaataaaatagtggtaactgcattaactctgtcccaaatgtactttttcaagtttttgtctcaaagttgtctaagaaaaccttggcatggagtcaagggcaaagttgaaaacactagtgatttgaatcacacacttgcatcctagagccacctatgtatacatgcatgcatgcaatatgaaacgtacatcgtcacaacacatgcatgcaattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaacatgcatcctaatatcgtgcacatcctctcgtacgtccgtaggcaccgaggaggaggcggcggaggcctacgacatcgcggcgatcaagttccgtgggctcaatgccgtcaccaacttcgacatgagtcgctacgacgtcaagaccatcctccagagcagcaccctaccggtggcggtgagtggcgcggcgaggcgcctcaaggaggccgctgatcatccagaggctggcgccacgatctggcgggccggcatggatggcggcgtcatctcccagctgaccgacgtcgggatgggcgcttacgcgtcgtaccaccacggctggccgacaatcgcattccagcagccgtcgccgctctcggtgcactacccatacgtcgcccagccgtctcgcgggtggtgcaagcccgagcaggacgccaccgtcgccacgcaaaacctgcaggacctccaggagctgcacctcgggatcgccgctcacaactatttccaggcatcgtcgagctccacggtctacaacggcggcggcggcggcgggtaccagcagggcaccggtggcaacgccttcctgatgccggctagcgccgtcgtggacgaacaggggcacagcagcactgccaccaaccagggaagcacctgcagctacggggacgaagaagggaagctcaatattgggtacgacgccatggcgatggcgagcaccggcgccgacccatacgcggcggcgtcaacggtgagcatcgcgagggcgaacgggtactccaacaactggagctcgccgttcaatggcatgtga

SEQ ID NO: 84 - DNA, 6962 bp, Triticum aestivum
  source 1..6962: mol_type = genomic DNA
cttcttctcctcggcggcacctcccgactttgcctctacccgctggccgggtagccggtggtgaagaggggcgcagaggtggctctcaggtagatggcgtcacttcatcttcaagctggtcctccgggcttcgatccttctcgagtttgctcgtcgggatggagacgatggagctccgacgtagattcctgttgtctccttgggacggcgaggattaggtttctcatcatgcgtacgcaacaacaagatttggtgtcagacactttagatcgattcaagggttcaacgtctaacaaaggcgctacaaacaaacatgcaaaaatataaatgatgtacagtgaagtgtcacttcataccgtcgaagccctggtccactgaaatgctcgctgatctagccagaggcctcatcaaagccaatgaatggaagagggcaaactcaggagcaagacaccttcgtcagctagcttcaacatggggacctcatcgttgcaccatcaacctgttgcccaaagagggcctcctatcccctcgccctggcacgaagggcttgatccgtcggacgacggagagctcttaaagcatggaagttagctgaacaccagaccaccgaggaagaagtgactctgagcacaacaaccatcgccagccatgccacgccaccgtaaacaacccccatgacgacgcaatgaagagatctaggctcgtcttaatattgtgatttcacaatcatttaagctttgtatgagatatttacgatcaaagcccaattttgatcaacagatgtatgtgttgtattctgaaacaggtgaatatatgtttatgtgacttaacaaatcgtagctgatcttactttttgattccttttcctacttcttttgtacttgtaacaactttcttattgttatttgtcttttttttaaacactatggacgcgaatactcatacatacgtacgtacactcatccctacgaatggacacacgcacaccctattcctataagcacctccgagagactgagccggcacatcatctttgagattgacgaagtcgccatagacgccttcgtagtcgatgggaacgtctctttccactgaacgcacatcaacggaagggctgaaataaatccagaaacatgcgagtcatgactagtagatctacacatgatttattttccatggaaaactgtttatatatcctatagagaaccaaaaacacaactaccaccacggttacgttctcatataaatacttacctctcctagcacatttcccccttctctcatgtggccaggatatttttctggccactgctgcactcttctcacattgacccaaatgtagctagtgttctaggtgcttctcaaaaaggggtataccctcctcccctatcagcttccaccttacctcgccaatgcttccagtgctctgatcaggaagaaaccaactattaatctatcgcagctagccccgcgggctgttctatcaatcggccatcccagaatggcttctggaaacgactggttggggttctcgctctccggccaagaaaacccacagccgcatcaggatagcccgccgcccgccggcatcgacatctccggcgccagtgacttctatggcgtacccacgcagccagctcccgacgctcaactcggcgtgccaggtcatcagccttcatacgggatcacggaggacttcaataggggagctcatgaaacccacggtgagaggaagaagctagccagtttgccgcatgcactccgatccaatgttatctaagctagcttgtatgtagacttctcccgttcaaggtttcttgttatttgatgtggctgtagattggaacatgaggggtgatctggactataacggcggtgcgtccgagctctcgatgctcgtcgggtcgagcgcagtcggcggcaagagggcggccatggaggccgagaccgagcccaagcttgaggacttcctcggttgcaactcgttcgtctccgagcaagatcaggccggcggcttcttgttctccggagtcccgatgaccaccagcagcaacagcaacaccgggagcaacaccatggagctctctatgatcaagagctggctccgtgacaaccagctaccccaagcgccgcacccgcagtcgcagaccggggcgcccgcgcagcagccgcaaccccacgaggagatgggcaccgacgcgagcagcttcgatccgcgggggagaaacggcgcgttggtggtggcggggagctcgcagagcctggcgctctcgatgagcacgggttcaggatcgcacttgccaatggctgtggtcagcggcagtgccagtgggggagtctcagagagcacctcctcggagaacaagcgagccagcggcgccatggattcgccgggcggtgcggtagaagctgtcgcgaggaagtccatcgacacattcgggcaaaggacgtcgatatatcgaggtgtaacaaggtcagaacattatattattactggttgatttcttcacattaatttgttgatccatgacgtagttactagttagtccattccaaattgggctagtacactagcattttaaattgggcgatcaatctagcgaggaactctttaaacagacatacgaggtctatatgctagtcatgataatgcaagctactttgattttgcgcttctaatttggtgattatttgaggacatatatataatttgttgaccgagaaaagttgacaaatcgacttggttgttcatatccaatgattaatatatgaacatcttatctggcgactgttcgctgggagaggggtggcaaacgaaccacttttgatggcagttttagttttgaaacgagatcaaatggtgacagttttaaatgaaaaattgtttcctctaacaaaaactgctatgttgctcagaagaaactgccatccctcacaactaaaattgccagtgaaattgttcgcaaatgccatcccctcctggggaacattcgccggctaaaggggtccttaaaatacactaccattttccgttcaccagttaggtaaaaggcgtttgcctcgctttatagataaatcaaccaccacatccgtacaataaagctcaagtatagaaaaaataaacataagcctgttgtgccacaagcacaacacaaacatgccctaataaaaagaaaaagataaagtcaccaagtggctggtagatcaatggctagggggtggagcaagacgcgtcctgccgcgatcagcacgtccatcatgccgcctaaccgatcaatgttcaggtatatattactaagaagtaatatgtaactcattggctaattcagaaacaaggattcacagtggaattgaatttcttgttcatacgagtatacgacatgattcgtactatagctacgacttagtacttaccacacaatttgcactgcaaagtgttgtttttcttctccagaactcttatgatatcgtgtacactaatctacaccactataacccgggcgcatcaaaataagaggtgtaactaatttaacttttctagctgacccaacacaaataaatagtgtggatctttgcttttctctggatatacgcataattttttatatagagatctcttttatagacctcttaaaacgaatgtgtttaaatgaataatgaatacaaaagtcatataaaatgggaaaatgatgttttttcctctgatcaacatatatactctggcacatacatgtgcattagaaattgattatttttttagcgccaacatgcacattttgaaagtgctaactatatatattcttatgttcttcaatattcattttcagacatagatggacaggccgttatgaggctcatctctgggacaatagctgcagaagagaagggcagagtcgcaagggccgacaaggtaataaaaatggctatatacctatatacttcctctttctctccattggacaacaatatatatacgcttgatgtgctatttaagcattatatctcgtgttgcatcaacactctacatgactgcatcaccatcgcctctgggaatgatactgtatcgtcctcgacaatcagtttatcttggtatgctcaccttactctgtggcctttttacgttgcagtttaccattttaattattccaccttatgattgtgatcatgaaaccttaggtggctatgacaaggaggacaaggcagcaagggcttatgatttggcagcactcaagtattggggtacaacaacaactacaaatttcccagtaagcaagttctgatactttttttacggatgatttttccttgcattgctatttttgtatctgcaaaacatctcaagctatttttattggaatataatagttttttacaatggcagattaatacctacgaaaaagaggtggatgaaatgaaacatatgactaggcaggaatatatcgcatacttaagaaggtactattgctagccgatgttcatctcatactgaactatcacaggagtaaatggaatattgcactatcaataacttgatccaacaaatgcaggaatagcagcggattttctcgtggtgcgtcaaaatatcgtggtgtaactaggtatgttactaggtaattcagtctgttatgttttacagctaataatttatgtcatgtggaaatagctaatgtccgatatgtactcttatagaattagtaggcatgcatgtaagtaaacatgtatgttacatagttatccaaatggttgttatatctttattcaatgttctaagttctaactatataaggctgagatctttaccaggcaccatcagcatgggagatggcaagcaagaattgggcgggttgcaggaaacaaagatctctacttaggcaccttcagtaagttatgatctgatataatcaaccctctcttccgagcctatctaattgctagactaatgaaggtccttaagactaaattatgactagtcatagtgttatagctccaagatctaagtcatcaaccagtacttatttagttattttttcttgaactcttcgtctcgtagagctgaaggttagatattctatatgttgccctatggggcaataaaatagtggtcactgcattaactatgtcccaaatgtaccttttcaagtttttgtctcaaagttgtctaagaaaaccttggcatggagtcaagggcaaagttgaaaaccctagtgatttgaatcacacacttgcatcctagagccgcctatgtatagctgcatgcatgcaatatgaaacgtacatcgtcacaatacatgcatcctaatcgtgcacatcctctcgtacgtccgtaggcaccgaggaggaggcggcggaggcctacgacatcgcggcgatcaagttccgcgggctcaacgccgtcaccaacttcgacatgagtcgctacgacgtcaagaccatcctcgagagcagcaccctgccggtgggtggcgcggctaggcgcctcaaggaggccgctgatcatccggaggccggcgccacgatctggcgagccggcatggacggcgccatcatctcccagctgaccgacgtcgggatgggcgcgtacgcgtcgtaccaccacggctggccgaccatcgcattccagcagccgtcgccgctctcggtgcactacccatacgtcgcccagccgccccgcgggtggtgcaagcccgagcaggacgccaccgtcgccacgcaaagcctgcaggacctccagcagctgcacctcgggaccgccgctcacaactatttccaggcatcgtcgagctcaacggtctacaacggcggcggcgtcggcgggtaccagcagggcaccggtggcaacgccttcttgatgccggctagcaccgttgtggatgaacaggggcacagcagcactgccgccaaccagggaagcacctgcagctacggggacgaagaggggaagctcaatcttgggtacgatgccatggcgatggcgagcaccggcgccgacccatacgcggcggcgtcaactgtgagcatcgcgagggcgaacgggtactccaacaactggagctcgccgttcagtggcatgtgataatggtgaagagggcagaagctagtaatagcgggattaattagctagctagagaatcgaagttaggtcaggggtcttgctcgttgttgcgtttcggctcagggttccggcttagctcttcttggccgggtggatgacactcgtaaaggtgctacgcatgcatgccttctggaagctggtaatggcaagtcaggataggtaaagcttgtgtcgttgtaagttaattatcctgacttgctgttagttttgggtagtttcagaaggatcaagttaatccctagaggacaaaagctgatcgtgcgatggcaataacttcgtaattccaatgattcgacttactttggcttttcatttagttagagcatctacatccgacatatgagaaacgcgtccgccgaaagggcctatcacccttcaaccccatacctcacttaatccatatactatgtcatgcaactcaaaagttacatactattccctcccttcctttatataaggtgtatttgttttttaaaaagtcaaacgtgtgcatgtttgatcaagtttttagaaaaatactccctctgtaaataaatataagagcgtttagatcactactttagtgatctaaacgctcttatattcttttcggagggagtatgtcactatccacaaccaaatttatatagtttaatccatcttgaaaagtattttcatattttattgattatatattgtggatattgatatttttattctataatcttggtcaaacttttaaatggttgacttgcacgaaaatcaatacaccttatattctggaatggagggagtatgcatagataaacctagcctaaactactcattgtcagatatgtacacgacgtcctgttgtatataaaaaatattg

SEQ ID NO: 85 - DNA, 4572 bp, Triticum aestivum
  source 1..4572: mol_type = genomic DNA
atggcttctggaaacgactggttggggttctcgctctccggccaagaaaacccacagccgcatcaggatagcccgccgcccgccggcatcgacatctccggcgccagtgacttctatggcgtacccacgcagccagctcccgacgctcaactcggcgtgccaggtcatcagccttcatacgggatcacggaggacttcaataggggagctcatgaaacccacggtgagaggaagaagctagccagtttgccgcatgcactccgatccaatgttatctaagctagcttgtatgtagacttctcccgttcaaggtttcttgttatttgatgtggctgtagattggaacatgaggggtgatctggactataacggcggtgcgtccgagctctcgatgctcgtcgggtcgagcgcagtcggcggcaagagggcggccatggaggccgagaccgagcccaagcttgaggacttcctcggttgcaactcgttcgtctccgagcaagatcaggccggcggcttcttgttctccggagtcccgatgaccaccagcagcaacagcaacaccgggagcaacaccatggagctctctatgatcaagagctggctccgtgacaaccagctaccccaagcgccgcacccgcagtcgcagaccggggcgcccgcgcagcagccgcaaccccacgaggagatgggcaccgacgcgagcagcttcgatccgcgggggagaaacggcgcgttggtggtggcggggagctcgcagagcctggcgctctcgatgagcacgggttcaggatcgcacttgccaatggctgtggtcagcggcagtgccagtgggggagtctcagagagcacctcctcggagaacaagcgagccagcggcgccatggattcgccgggcggtgcggtagaagctgtcgcgaggaagtccatcgacacattcgggcaaaggacgtcgatatatcgaggtgtaacaaggtcagaacattatattattactggttgatttcttcacattaatttgttgatccatgacgtagttactagttagtccattccaaattgggctagtacactagcattttaaattgggcgatcaatctagcgaggaactctttaaacagacatacgaggtctatatgctagtcatgataatgcaagctactttgattttgcgcttctaatttggtgattatttgaggacatatatataatttgttgaccgagaaaagttgacaaatcgacttggttgttcatatccaatgattaatatatgaacatcttatctggcgactgttcgctgggagaggggtggcaaacgaaccacttttgatggcagttttagttttgaaacgagatcaaatggtgacagttttaaatgaaaaattgtttcctctaacaaaaactgctatgttgctcagaagaaactgccatccctcacaactaaaattgccagtgaaattgttcgcaaatgccatcccctcctggggaacattcgccggctaaaggggtccttaaaatacactaccattttccgttcaccagttaggtaaaaggcgtttgcctcgctttatagataaatcaaccaccacatccgtacaataaagctcaagtatagaaaaaataaacataagcctgttgtgccacaagcacaacacaaacatgccctaataaaaagaaaaagataaagtcaccaagtggctggtagatcaatggctagggggtggagcaagacgcgtcctgccgcgatcagcacgtccatcatgccgcctaaccgatcaatgttcaggtatatattactaagaagtaatatgtaactcattggctaattcagaaacaaggattcacagtggaattgaatttcttgttcatacgagtatacgacatgattcgtactatagctacgacttagtacttaccacacaatttgcactgcaaagtgttgtttttcttctccagaactcttatgatatcgtgtacactaatctacaccactataacccgggcgcatcaaaataagaggtgtaactaatttaacttttctagctgacccaacacaaataaatagtgtggatctttgcttttctctggatatacgcataattttttatatagagatctcttttatagacctcttaaaacgaatgtgtttaaatgaataatgaatacaaaagtcatataaaatgggaaaatgatgttttttcctctgatcaacatatatactctggcacatacatgtgcattagaaattgattatttttttagcgccaacatgcacattttgaaagtgctaactatatatattcttatgttcttcaatattcattttcagacatagatggacaggccgttatgaggctcatctctgggacaatagctgcagaagagaagggcagagtcgcaagggccgacaaggtaataaaaatggctatatacctatatacttcctctttctctccattggacaacaatatatatacgcttgatgtgctatttaagcattatatctcgtgttgcatcaacactctacatgactgcatcaccatcgcctctgggaatgatactgtatcgtcctcgacaatcagtttatcttggtatgctcaccttactctgtggcctttttacgttgcagtttaccattttaattattccaccttatgattgtgatcatgaaaccttaggtggctatgacaaggaggacaaggcagcaagggcttatgatttggcagcactcaagtattggggtacaacaacaactacaaatttcccagtaagcaagttctgatactttttttacggatgatttttccttgcattgctatttttgtatctgcaaaacatctcaagctatttttattggaatataatagttttttacaatggcagattaatacctacgaaaaagaggtggatgaaatgaaacatatgactaggcaggaatatatcgcatacttaagaaggtactattgctagccgatgttcatctcatactgaactatcacaggagtaaatggaatattgcactatcaataacttgatccaacaaatgcaggaatagcagcggattttctcgtggtgcgtcaaaatatcgtggtgtaactaggtatgttactaggtaattcagtctgttatgttttacagctaataatttatgtcatgtggaaatagctaatgtccgatatgtactcttatagaattagtaggcatgcatgtaagtaaacatgtatgttacatagttatccaaatggttgttatatctttattcaatgttctaagttctaactatataaggctgagatctttaccaggcaccatcagcatgggagatggcaagcaagaattgggcgggttgcaggaaacaaagatctctacttaggcaccttcagtaagttatgatctgatataatcaaccctctcttccgagcctatctaattgctagactaatgaaggtccttaagactaaattatgactagtcatagtgttatagctccaagatctaagtcatcaaccagtacttatttagttattttttcttgaactcttcgtctcgtagagctgaaggttagatattctatatgttgccctatggggcaataaaatagtggtcactgcattaactatgtcccaaatgtaccttttcaagtttttgtctcaaagttgtctaagaaaaccttggcatggagtcaagggcaaagttgaaaaccctagtgatttgaatcacacacttgcatcctagagccgcctatgtatagctgcatgcatgcaatatgaaacgtacatcgtcacaatacatgcatcctaatcgtgcacatcctctcgtacgtccgtaggcaccgaggaggaggcggcggaggcctacgacatcgcggcgatcaagttccgcgggctcaacgccgtcaccaacttcgacatgagtcgctacgacgtcaagaccatcctcgagagcagcaccctgccggtgggtggcgcggctaggcgcctcaaggaggccgctgatcatccggaggccggcgccacgatctggcgagccggcatggacggcgccatcatctcccagctgaccgacgtcgggatgggcgcgtacgcgtcgtaccaccacggctggccgaccatcgcattccagcagccgtcgccgctctcggtgcactacccatacgtcgcccagccgccccgcgggtggtgcaagcccgagcaggacgccaccgtcgccacgcaaagcctgcaggacctccagcagctgcacctcgggaccgccgctcacaactatttccaggcatcgtcgagctcaacggtctacaacggcggcggcgtcggcgggtaccagcagggcaccggtggcaacgccttcttgatgccggctagcaccgttgtggatgaacaggggcacagcagcactgccgccaaccagggaagcacctgcagctacggggacgaagaggggaagctcaatcttgggtacgatgccatggcgatggcgagcaccggcgccgacccatacgcggcggcgtcaactgtgagcatcgcgagggcgaacgggtactccaacaactggagctcgccgttcagtggcatgtga

SEQ ID NO: 86 - DNA, 1499 bp, Zea mays
  source 1..1499: mol_type = genomic DNA
actaccagcagatgatcagagtgaggatagttgctacttcaatggcctaggtttgataactactgtcgatccatctgtcgacaattgtttgtttcctctcacaattcacgtttctgccgccacttctttgcacctaacattgcccgtgtcgctgttattagttttgtggttttgcattgtctgtatatagtctgatgaggctatagattgagccttcaaaagtgtttcgttgggcctcttgttgaatgtactccaaagtctcggtgcaccatcagacgtattcctagaatggcacaatctggctatcagaactggtaggtttagtccaaatgtaatgatgtgttctaaagtagtcgggctgagtcaaacagcagcttcttctcttgttcaaaattttgaacctggtaatgttgtcatttcatgatagatcatagatacattcttggtttcactatatcttagaaagtccagccaacagaagtactcaagtacaggtgaacctttcagtttagctggagacatgggcagcagtgtacaaagtctaatccacagctgtgaccctgtgagctgggggttgggcctgcctcggaggggatcgtgtcttttctgtttgtgcaattatcagtgcctaaagaacatgtgttataacagttatacacggtcaattattcaatccatcttttaacgtgtgtttggttgtacgtataagaagggatcaaagagggcggccacattttctataacatttggatgagagtcacacgagaacgagatgaacaccgaagtaattttttatgacagcatgatccaaaaaaatcgagagaacggtgttatccccgtctcatcctattttgatctcaaaccaaacacataattagtcaatgagcactgacttgatgtagaagaacttggtacatataccaggaaatgactctgacttgctgagatgccttgtagtccctcggtcccgtaaattatacagtagtatttaaacttcctcgcacaaaggaaaactatttgtttttgcatgccttgtaaattagctatcaagtttttttttaaaaaaaacgacctttgaaaaccagtaaggaggactcgttcggtattcagttttaaagttcaaggttaaaaatcgaactgacgtgcaaaacaccctgggggttaaaatcaaaacacacaaatttggaaaatgagcaggcatgacttatgttgtgtccgttgaagtgccgtggctccatggcacggtggaactttacattagtgcggtagtgtcactgtggcagtaaagcccacttattttccattagagagagggagattaaaaacgtggctttacacattctgaaactaattgtgagtttgtgactcttggcttgcctgcagtgtccgcctgcaccttctataagtacagtcgcgagcctcagctatacaaaccaactagcgcatagtatctcctgagctccattcatcatcccaagtcttcactggtcttagatcac

SEQ ID NO: 87 - DNA, 3845 bp, Zea mays
  source 1..3845: mol_type = genomic DNA
atggccactgtgaacaactggctcgctttctccctctccccgcaggagctgccgccctcccagacgacggactccacactcatctcggccgccaccgccgaccatgtctccggcgatgtctgcttcaacatcccccaaggtagcatctatctatctgtcgacttacgtgcataaataagtgctcatgtatatcttctgcagtttgtggtacgaatacttattgaggctagcatgaaatgtcgtttgttctttcagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggtaagggggtgaatgaatcaagtaatcatgaaattttgaaaagccattggtaatccaaggaactgtcatgatagatttgattgcatctagacacagttccgatcgaatcaaatgagtaggccaatgtttagcctttggggatctcgctgattattaggatttaggagtaccattgtattgggcatggttgtggtatagtagtagacaattaacaaaaaagctaccacttttgaattattttaggcatagatggactgggagatatgaggcacatctttgggataacagttgcagaagggaagggcaaactcgtaagggtcgtcaaggtatacaaatataatgcaacatactgtgtcattaaatacgctttttctgtaagcttaatatttcaccaatgatgttgttattgttaactgacattgcttcacactatcaattttggattcggcgccatgatttgtgggattgaaatcaaatcttaaatcaacagtctatttaggtacgcgatttctcttcaactacttaatgcagtttgtttctccctataaccatattctttttcatctcaaatctcactcgactctttttttttaatcttgtaccattgataggtggctatgataaagaggagaaagctgctagggcttatgatcttgctgctctgaagtactggggtgccacaacaacaacaaattttccagtatgtatatgtaggatgcggttttactttactgaagttcatatctcgttatggcctacaaatatgtatcaaatgctgtacattagctagtgatctggagtgaaggttctatagtaaagtaaacgctgtgtgcggagtgcattagcgggaggtctctcttttattttctaaaaaaatggacattactgaaattgtacttaaagtcgtttttttgtatttccaggtgagtaactacgaaaaggagctcgaggacatgaagcacatgacaaggcaggagtttgtagcgtctctgagaaggtcggtctaacagcattgattaatcagtaccaactctactgaataaaatctgctgctctttgttaaattttgagcgaggtcaactgcatatttgatcttattagaccactgtatatgaatgcaggaagagcagtggtttctccagaggtgcatccatttacaggggagtgactaggtatgaatccatatataatagctaggaacatcaacaaacacacatacacttgggttgatgtggcagatgcatgcatggattgaaaatgcgtgcatggtgttttacttgaactctctgtaattataggcatcaccaacatggaagatggcaagcacggattggacgagttgcagggaacaaggatctttacttgggcaccttcagtaagtagcaaacaaatatgtttttgcattgtatatttaaaattcaccacatatacaagcaagttacagtcaactaacacaatctcaacgcaatgagaaagcaagtgttccagctgatagtacacatttgtagaccagctgcatatggttgttttgtatgcatgatgactattaaaaatgtgaccatcgcattaagtcatgcaaagttgcattgcagtagtacattgcttagtgcatgctcctcaagtggcttttttcaaacctgatcccatgtctggtgctatggttgtctcccattcacccgtgcatcaggtcaaaatagtactatgcctgaataagaaaaacaaaacgagtatgcactggcagcagcagactaataaacaaagtttcagcatttactaataaactaattaggctacagcatccaaaagattcttccaattaagccacaactgttctctgttcatgcatacatgggtatgccacccagcataccatgcatgcatgcatgcaccgtgcacgacgaaagcgaaacgctcgttctcggaatatttgaactgacgaagccgagcgcaaccttccatcgtggatgcaggcacccaggaggaggcagcggaggcgtacgacatcgcggcgatcaagttccgcggcctcaacgccgtcaccaacttcgacatgagccggtacgacgtgaagagcatcctggacagcagcgccctccccatcggcagcgccgccaagcgcctcaaggaggccgaggccgcagcgtccgcgcagcaccaccacgccagcgtggtgagctacgacgtcggccgcatcgcctcgcagctcggcgacggcggagccctggcggcggcgtacggcgcgcactaccacggcgccgcctggccgaccatcgcgttccagccgggcgccgccaccaccacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggtgcgcacgactttttctcggcagggcagcaggccgccgccgccgcgatgcacggcctgggtagcatcgacagtgcgtcgctcgagcacagcaccggctccaactccgtcgtctacaacggcggggtcggcgatagcaacggcgccagcgccgtcggcggcggcggcggcggtggctacatgatgccgatgagcgctgccggagcgaccaccgcatcggcaatggtggtgagccacgagcaggtgcacgcacgggcctacgacgaagccaagcaggctgctcagatggggtacgagagctacctggtgaacgcggagaacaatggtggcggaaggatgtctgcatgggggactgtcgtctctgcagccgcggcggcggcagcaagcagcaacgacaacatggccgccgacgtcggccatggcggcgcgcagctcttcagtgtctggaacgacacttaa

SEQ ID NO: 88 - DNA, 4099 bp, Zea mays
  source 1..4099: mol_type = genomic DNA
atggcttcagcgaacaactggctgggcttctcgctctcgggccaggataacccgcagcctaaccaggacagctcgcctgccgccggtatcgacatctccggcgccagcgacttctatggcctgcccacgcagcagggctccgacgggcatctcggcgtgccgggcctgcgggacgatcacgcttcttatggtatcatggaggcctacaacagggttcctcaagaaacccaaggtgaggagcagaggctgcaactagccaactaacaatctctttggtctgattgatcgatgacctctcttgctagtttttctatcgtatggtgcactgcacgcgatattctatggggtttttttacggtttgtttcttgatatttgatgtgggtgctctcagattggaacatgaggggcttggactacaacggcggtggctcggagctctcgatgcttgtggggtccagcggcggcggcgggggcaacggcaagagggccgtggaagacagcgagcccaagctcgaagatttcctcggcggcaactcgttcgtctccgatcaagatcagtccggcggttacctgttctctggagtcccgatagccagcagcgccaatagcaacagcgggagcaacaccatggagctctccatgatcaagacctggctacggaacaaccaggtggcccagccccagccgccagctccacatcagccgcagcctgaggaaatgagcaccgacgccagcggcagcagctttggatgctcggattcgatgggaaggaacagcatggtggcggctggtgggagctcgcagagcctggcgctctcgatgagcacgggctcgcacctgcccatggttgtgcccagcggcgccgccagcggagcggcctcggagagcacatcgtcggagaacaagcgagcgagcggtgccatggattcgcccggcagcgcggtagaagccgtaccgaggaagtccatcgacacgttcgggcaaaggacctctatatatcgaggtgtaacaaggttagaactttgattagtttgtgtcttcgataccttttgttattgttcttcgtttcgtttcaaattggtccagtgaggtataactatattcagagaacataactttagcatgaccttgttctagtactactcgagcaaaataccatggccttgttgagcgatcaatccatggagaacataaaccatcagatctatgctagctaatgcaactttggagacagtagccatgctatttttcttcaatttttctctagtagttcatttatatatgatacttagaagataaggtgactagttctgaagtataccatttgtgtactaacaagtggatgcaatatactgatctatttttcaatatgtgctgcgatgacggcaacgtatgccactgatcacttaggtggttgagaacggagtatatatatatgcatgtacaatttgaaatggtactccttgtgtggactgttatagttgcttttattctatttgcaagggaacattggaagctatatacacatatgcaagcgtatttttgcttagtttttggataattaccatatatatatatatatatatatatatatatatatatatatatatatatatatatatatatatatatatatatatatatatatatatatacgtgttgaggttggtaataacaaatgtttttgaaaacgcactatatatatgcataattttgagtattcttgtttacaggcatagatggacagggcggtatgaggctcatctatgggataatagttgtagaagggaagggcagagtcgcaagggtaggcaaggtaatgaaacatatatccataacaaccactgcttatatcgtcatagaaaaaacatatatggtttgtacaacttgagctactgataaagtattgtgtcttttggttccattgcacacaccctggcttcaaacattgcctaccggaatgacaccaacaatatgcagtttaccttggtatgcattcaccttagctcctgcccttacatttttgttgttgtatagatcttcctattttaacaatcattttgctatgccattgtatatccactgtatacacttaggtggctatgacaaggaggacaaggcagcaagggcttacgatttggcagctctcaagtattggggcactacgacaacaacaaatttccctgtacgcaaagttctatatggtgtttctttgggtgcttataacagaagcattttgacttgttttttaaacactatatatattaatctttgttcttgaatttatttttttgccaaaatttcattgttgcagataagcaactacgaaaaagagctagaagaaatgaaacatatgactagacaggagtacattgcatacctaagaaggtattattttgatgcttgtgtccttgtcattggttcacttccatgaatgatttgactaacagttaatatgatcttatacattcagaaatagcagtggattttctcgtggggcatcaaagtatcgtggagtaactaggtaattaaacttcactagtaggcaatgtatgatttgtgtgcatacaaaaaaattgtccttttatttcacattatcattagatgcctgttgtgggaaaaacataactttacaagagatttatggaacttgtttttgtttcctttttttatattgacatcaatgttgttaatcgatcagacatcatcagcatgggagatggcaagcaaggatagggagagttgcaggaaacaaggatctttacttgggcacattcagtaagctagctagctgccttcttttcttgtttgattgttttgagaactcagtgaagctagcacccattgattagtgattaatgcaactcataaaaactaattgctcggttaatggagatccttaggttagttaatgactactcatagtgttatatatgtccccaagatctaagtcatctcgccccgccgtacttgtttagtcaaatttcttgaaatcttcgtctcgtagaactgaagattagatattctatatgttgccctatgggagcataaaatagtgactacaccattaactatgtcccaaatgtacctttccaagtttttgtctcaaagttgtctgagaaaatcggggcatgcggtcaaaggggtcgagcagaaaaccctagtgctttgaatggcacagtgtgcacccggcctgctgcatgcatgcaatatcgtcgtgcattggggaacgacaaccctaatcatgcaccctgtgctgtgaagctaatcgtcgtgcacattccctcgtacgtaggcaccgaggaggaggcggcggaggcctacgacatcgccgcgatcaagttccgcggtctcaacgccgtcaccaacttcgacatgagccgctacgacgtgaagagcatcctcgagagcagcacactgcctgtcggcggtgcggccaggcgcctcaaggacgccgtggaccacgtggaggccggcgccaccatctggcgcgccgacatggacggcgccgtgatctcccagctggccgaagccgggatgggcggctacgcctcgtacggccaccacggctggccgaccatcgcgttccagcagccgtcgccgctctccgtccactacccgtacggccagccgtcccgcgggtggtgcaaacccgagcaggacgcggccgccgccgcggcgcacagcctgcaggacctccagcagctgcacctcggcagcgcggcccacaacttcttccaggcgtcgtcgagctccacagtctacaacggcggcgccggcgccagtggtgggtaccagggcctcggtggtggcagctctttcctcatgccgtcgagcactgtcgtggcggcggccgaccaggggcacagcagcacggccaaccaggggagcacgtgcagctacggggacgaccaccaggaggggaagctcatcggttacgacgccgccatggtggcgaccgcagctggtggagacccgtacgctgcggcgaggaacgggtaccagttctcgcagggctcgggatccacggtgagcatcgcgagggcgaacgggtacgctaacaactggagctctcctttcaacaacggcatggggtga

SEQ ID NO: 89 - DNA, 2002 bp, Zea mays
  source 1..2002: mol_type = genomic DNA
aaccagccttcaccgcgtaagtatgaacaggtaactgcatttaattactactgccctctattagtttgaaaccctctccagtagacctctctaggtttgacggtcaaagttttaggcgaggggagggggaggcccagctagcggtgcagttaccgagataaaaacacacaaggccggaggcccctatcttaacctccacttctccttcccccgtaaagagagattaaaaaaggattagttcttctgtactagcctggcgtccgagagagagagagagagagagcacccggccttgttaacaattcgtcctggaggatcctatgcgaggtttttagggtgttttttgctcacttctccatgcgcggcgttgaagctagacgatggacttgcttgggaacgccaagcttttctatggccggaagacaagggttcgtgtcctcttctaggatgcaggtggcacgtactctccgtccagcatgcatgaacttggtcctcgtcttttcatctttttctgcctgtcgaccttcaaatcatcttttgaagcaaattatagtgtagtatttctttcccttctttttgaagcatgcattgcaagtagggattgtcgtgcaccatctacaatactccattgagagtattgacgatcgactgactatcgattgcattgaaagaatttttcctatctcacacagtataatatgcatttggatatatacagaccatttcattttactatagctagggtatgaattctctagttctagctaaatcttgatcatgactggagttttggaatggttgtgtgaaatattacacggtataatgattgtagtacttgtgttagatggtgagataatggagctaggaagggcgagcctagggttttctgaaaaaaaacccatatcagatatatattcagcttctgcatgtgactgtgaatggattagggttaatataaagagaggcctagaccatactaggagtaattgctcatatgcttatttcagcaggcttgaacagtgttttggctcgtagctggtgcagctagcttgatgctgtaatatacagttggagaagcgtgtatacatgcatgcatatgtcaactggatgtgcataaatgtccaggcactggtgtactgtacccgtgtgcgtgcatgtgcatctatcatacaattgaaaaaaatcctaccagaacatctgcttactaattgagattctagacaaatagaacaaaacttagatctcgtgtcatttcttttttttccttcttccaagaagggttcaatttcttaatcgttcttgacagcaacctgccagtcaaatggccgtgacaacgtatactattatcgagtaaaaggtcgccactttagtagtacatgtacatgcatgcgcagatacatcatcaggtactcatatatgggcacacatatagacatgttttgaggaaaatgagacaaagtatagtggagacttccctagaaagcagaagaaaaataagtggtttatgttccgttaaatcatactacaactttttttattatactctccattttgtcatcattaggtactcatatatgggcacacatatagtactgccaatttttcttgctaaaaaaagttccactatatatatatatatccacaaataaactaattttcttgaaaaaatgctgaaattctttagaaaaccggtacgttttcaaactagccctttagtactaaagggctagtttggaaacaccggttttctaaaggatttcattttttccaaaaaaatagtgccactaatcttctggagtgtaccagttgtataaatatctaccagtatcagcactgtttgctgaatgccccaaaactctctgcttgacttcttctcttccctaacctttgcactgtccaaaatggcttcctgatcccctcacttcctcgaatcaatctaagaagaaactcaagccgcaaccattaggggcagattaattgctgcactttcagataatcaacc

SEQ ID NO: 90 - DNA, 2001 bp, Zea mays
  source 1..2001: mol_type = genomic DNA
tttggtggtttttccatccgaggtagtttgaaaaatttaaaattcaaaattcaaacatagttttgcatggcaagatgatttcaaaccaaaacattgtcaactacaaagtttcataactcttcaatacctacaactttcatgttggtggttttttctttcgaggttgttttcaaaattcaaattttaaattttttaatttcaggcatagttttcgtttacaatatgacttcaaatgaaaaagttgtcaactacaaacttctataacttttcaagatctacaaagtttattttggttgtttgatcatttgttcatctcacatgatagttctaacaatatgcacaaattctatacatatctctcgtagttccataaactacgagagagatataggttttatgaacaaatttatttttagtttatcatatgaagaaatgttcaaaatataaaatgtacatcatgatgagttatacaaatttgtagttgaaaactttttcatttgaattaatttactgctttaaaatgtgtatgcagttggcttgatgtgagacaataagctaaaccacgggttttccatgggtttccacccaattacgggttggtttgggatcaataatgttattggatgggttgggtttaactttcctctagtatttttggttgggtgtgggatggatatcaaagtaattagatttgggtatgggtgggagcggattggattttttgccattagcaggcctatgcacttgtcggcgcacacggctggcgcgtgcacacaccttatcggcagacgccgccgctgctgctgctgatgcttcttcttcttccaccgattgattgagatccaaggaaatgctgggcgcgtttttaaactggctgatcggcttcgtcccatgatctgagatttccttttcagactgttgtatatgtagcggcgacatggataggatatgtatggaccgttttaattaatactaaatatcaatggtttaactcttcactattactcctagctagatgtgaaagctcgccctggaatgcacatgaacaactacaccgagttgggcagtttggcaatgcatgcagttcaagagccacgccggttaacccttcactttgtagttattgcctgcattttgtaaatttggtgtgaacttaattctaatctgtaattctgtatagagaatggtgcatgtactgaactcctgactgatcaaaacgtggggtccactttcctcgccgaaaaagaaaagagaaaaaaaggtttattgtccactttattaaaaactggacttatatatatgcttggaacaaatacttagggggtgtttggttttaggaatctctcaatctaaaataaggtggtgtatcataggtttattactcaaatttggtgggatgatctcattgctcatattagtactaactaactaactatgaggaatgagatggtgatggatcaacccattacattctacaaaccaaacaaaaaagtgaggagtgagaagatggtgaactagctcatttctcaaaccaaacactctattaataaaccaatcgcgtagctaataaatatatgtaacctcgacattctagctacagcttgtggttggtagaaaagcaaatcatgctcaaaataatgtgctccgaaacttcattacatttttcttcacttgagtgtacatacattttttttcccttggccctcttatccttaactaagaagcataaaacgggatttctcagccagttcttagcttctcttataaatacagaccttgtacaacactttcacctcctctcaggtggccaggatattttttctggccccttcctgccctcttcacactgacccatgtgttgttctatccctggaaagctatacctccttacccctatcagcttctcctcacatctcctctcgtcgccacccatgctatcaccgctctgatcacaagcaaggcaaaccctcactgttctatcaacgcccctcccttagctag

SEQ ID NO: 91 - DNA, 20 bp, synthetic construct
  source 1..20: mol_type = other DNA
tgttcatacttacgcggtga

SEQ ID NO: 92 - DNA, 20 bp, synthetic construct
  source 1..20: mol_type = other DNA
gttacctgttcatacttacg

SEQ ID NO: 93 - DNA, 20 bp, synthetic construct
  source 1..20: mol_type = other DNA
ccagttgttcacagtggcca

SEQ ID NO: 94 - DNA, 20 bp, synthetic construct
  source 1..20: mol_type = other DNA
agcgagccagttgttcacag

SEQ ID NO: 95 - DNA, 20 bp, synthetic construct
  source 1..20: mol_type = other DNA
tatgcacttgtcggcgcaca

SEQ ID NO: 96 - DNA, 20 bp, synthetic construct
  source 1..20: mol_type = other DNA
cacttgtcggcgcacacggc

SEQ ID NO: 97 - DNA, 20 bp, synthetic construct
  source 1..20: mol_type = other DNA
tgccttgcttgtgatcagag

SEQ ID NO: 98 - DNA, 20 bp, synthetic construct
  source 1..20: mol_type = other DNA
gggcgttgatagaacagtga

SEQ ID NO: 99 - DNA, 20 bp, synthetic construct
  source 1..20: mol_type = other DNA
ggagcccttccttcccgcca

SEQ ID NO: 100 - DNA, 20 bp, synthetic construct
  source 1..20: mol_type = other DNA
gcgtcatcagcaccgcgagg

SEQ ID NO: 101 - DNA, 20 bp, synthetic construct
  source 1..20: mol_type = other DNA
atctaagaccagtgaagact

SEQ ID NO: 102 - DNA, 20 bp, synthetic construct
  source 1..20: mol_type = other DNA
tgggatgatgaatggagctc

SEQ ID NO: 103 - DNA, 20 bp, synthetic construct
  source 1..20: mol_type = other DNA
cgtacgtacaggagcacacc

SEQ ID NO: 104 - DNA, 20 bp, synthetic construct
  source 1..20: mol_type = other DNA
agcacaccaggcaacggcaa

SEQ ID NO: 105 - DNA, 20 bp, synthetic construct
  source 1..20: mol_type = other DNA
ggtaccagctcaccccctca

SEQ ID NO: 106 - DNA, 20 bp, synthetic construct
  source 1..20: mol_type = other DNA
agctggctgccgtagccgtg

SEQ ID NO: 107 - protein, 10 aa, Triticum aestivum
LSMIKNWLRS

SEQ ID NO: 108 - protein, 13 aa, Triticum aestivum
KLEDFLGGISFSE

SEQ ID NO: 109 - protein, 10 aa, Triticum aestivum
LSMIKTWLRN

SEQ ID NO: 110 - protein, 13 aa, Triticum aestivum
KLEDFLVGNSFVS

SEQ ID NO: 111 - protein, 202 aa, Triticum aestivum
RKSIDTFGQRTSIYRGVTRHRWTGRYEAHLWDNSCRREGQXRKGRQVYLGGYDKEXKAARAYDLAALKYWGTTTTTNFPXSNYEKELEEMKHMTRQEXXAXLRRXSSGFSRGASXYRGVTRHHQHGRWQARIGRVAGNKDLYLGTFSTZEEAAEAYDIAAIKFRGLNAVTNFDMSRYDVKSILXSSXLPVGGAAXRLKXAEX

SEQ ID NO: 112 - protein, 690 aa, synthetic construct
MAXXNNWLGFSLSXQXNPXPXQDSSPXAAIDXXXXXXXXXXXXDXXXXXXXXXEAXBXCXNXPQDWNMRGXXXXXXSELSXLXXXXVAEPKLEDFLGGXSFXSZQDXXXGGXLFSGVPXAXXXXXXXXXXXXXXXXXXXSXNSNSGSXTXXLSMIKXWLRSXPXPQXPXXXXAXAQXLXXSXBXXMXGCXXXXXXXXLXAGXSSQSLALSMSTGXXXHLPXVVXGGXXXXXSGGXSESXSXXNXRASGAMDSXXXPXXAXEAVARKSIDTFGQRTSIYRGVTRHRWTGRYEAHLWDNSCRREGQXRKGRQVYLGGYDKEXKAARAYDLAALKYWGTTTTTNFPXSNYEKELEEMKHMTRQEXXAXLRRXSSGFSRGASXYRGVTRHHQHGRWQARIGRVAGNKDLYLGTFSTZEEAAEAYDIAAIKFRGLNAVTNFDMSRYDVKSILXSSXLPVGGAAXRLKXAEXXXEAGATIWRAXXYDXGXVXSQLXGMGAYAAXYXXHXHHXWPTIAFQQPXPLXVXXPYAQXXPSRGWCKXEQDXAVIAAAHSLQDLQXLXLGAAXAHBFFXASXXXXXXXXXXXXXSXSXVYNGGGGXXXGXXGGGYFLMPMSTVVAXXXQGHSSXXNQGXTCSYGDXXXXXXXIGYDAYLVXAXAXXGGGRMXSWGXASTVSIARAXXXXFSXWXXPFNGXMG

SEQ ID NO: 113 - protein, 782 aa, synthetic construct
MAXXNBWLGFSLSXQEXPXXXXXSXXXXXXDXXGAXXXYXJPXXXXXXXXXXXXXXXXXXXXXXXXXXXXXXXXXDWNMRGXXXXXXXXXXXSXLXXXXXXXXXXXXXMAEXEPKLEDFLXGXSXXXXXXXXXXXXXXXXXXXXXXXXXXXXXXXXXXXXXXXXXXXXXXXXXXXXXXXXXXXXXGGXXXXGXXXXXXXXXXXXXNXXSXXXXXXLSMIKSWLRXXXXPXXPXXQXXXXAXAQXXXXXXXMXXXXXXXXPLXXXXXXXVXGXXXXXXXXXXXXXXXXXXXAXXXXXXXXGGXSXXXXSXXXXXXGXXDSXGGXXXXXXXXXXXRKXXDTFGQRTSIYRGVTRHRWTGRYEAHLWDNSCRREGQXRKGRQVYLGGYDKEXKAARAYDLAALKYWGXTTTTNFPXNXYEKEXXEMKHMTRQEXXAXLRRXSSGFSRGASXYRGVTRHHQHGRWQARIGRVAGNKDLYLGTFSTZEEAAEAYDIAAIKFRGLNAVTNFDMXRYDVKXILXSXXLPXGXAAXRXKXXXXXXXXXXXJXXXXXXXGVXSXXXXXXXXXYXAXYXXXXXXXXXXXXWPTIAFQXPXXXXXXXXXXXXXHXXXXAQPXRGWCKXEXDXXVXXXXXSLQXLXXLXLGXXXXAHBXFXXXXXXXXXXXGGXXXXXXXXXXXXXXXXXXXXXXXXXXXXXXXXXPXSTXXXXXXXXXXXXXXXXXXXXXXXXXXXXGXXXSXXXXXGKJXXXYXXXXXXXXXAXXYXXGGYSNNWSSPFNGMQLFSVWNDSN

SEQ ID NO: 114 - DNA, 495 bp, Zea mays
  source 1..495: mol_type = genomic DNA
atggaagcaccacgcccgcgtctccccatcgtcgccgcgctgtcggcggcgctgctcctcgtcctcgcccacgcccacggcgctgcctcctcccccgctggcggcccgacgcccgtcgccgtgcggccctgggtggcgcggctgcaggcactccacgacctgtccgtgtccggcgagcagcagcagggcggcgggttcggggagtgctggggcgccgtgatggggctgagctcgtgctacggcgagatcctgctcttcttcgtcaacggcgagtcgtacatcgggcccgactgctgcgtcgccatccgcggcgccacgcgctactgctggcccgccatgctcgcctccgtcggcttcaccgccgaggaggccgacgtgctgcgcggcttctgcgacggcgaggaggcggaggccacccgcgcgccgccgcctgctgtgcccgctcctcccgcccggggactgggccagctggctgccgtagccgtgagggggtga

SEQ ID NO: 115 - DNA, 483 bp, Zea mays
  source 1..483: mol_type = genomic DNA
atggcattccctcggctgtccctcgccgtgctcagctgcctggcgctgctcgccgcgacggccgcgcaccgggcgcccatcacggggccgtcgccctcggctcctcgcggcctgacgacactggcggagcggctggagggcgcggagacgcagcagtgctgggaggcgctggttgagatcaagtcgtgcacgggcgagatcatcatcctcttcatcaggggcgaggcgttcctggggcccggctgctgccgcgccatccgcgtcatcgagcaaagctgctgggccgccgactcgatgatgtctatcatcggtttcaccccgcaggaaggggacatgctcaaggggtactgcgacgcgggcgacgacaacgccaccggcgggcagagcggttcgccgccgcctcgcggtgctgatgacgcggtcggtgctgtccgcggaagcttcgccgccgtggcgggaaggaagggctccatgcaccgttag

SEQ ID NO: 116 - protein, 10 aa, synthetic construct
LSMIKXWLRS

SEQ ID NO: 117 - protein, 10 aa, synthetic construct
LSMIKTWLRN

SEQ ID NO: 118 - protein, 10 aa, synthetic construct
LSMIKNWLRS

SEQ ID NO: 119 - protein, 13 aa, synthetic construct
KLEDFLGGXSFXS

SEQ ID NO: 120 - protein, 13 aa, synthetic construct
KLEDFLVGNSFVS

SEQ ID NO: 121 - protein, 13 aa, synthetic construct
KLEDFLGGISFSE

SEQ ID NO: 122 - protein, 202 aa, synthetic construct; identical to SEQ ID NO: 111